(19) United States
(12) Patent Application Publication
(10) Pub. No.: US 2021/0189439 A1
(43) Pub. Date: Jun. 24, 2021

(54) Multifunctional Fatty Acid Derivatives And Biosynthesis Thereof

(71) Applicant: Genomatica, Inc., San Diego, CA (US)

(72) Inventors: Andreas W. SCHIRMER, San Diego, CA (US); Risha Lindig BOND, South San Francisco, CA (US); Erin Frances PERRY, San Marcos, CA (US)

(73) Assignee: Genomatica, Inc., San Diego, CA (US)

(21) Appl. No.: 17/053,190

(22) PCT Filed: May 3, 2019
(86) PCT No.: PCT/US2019/030530

§ 371 Date: Nov. 5, 2020

Related U.S. Application Data
(60) Provisional application No. 62/669,912, filed on May 10, 2018.

(51) Int. Cl. C12P 7/18; C12N 15/52

(52) U.S. Cl. CPC C12P 7/18; C12N 15/52

(57) ABSTRACT

The disclosure relates to the field of specialty chemicals and methods for their synthesis. In embodiments, the disclosure provides novel multifunctional fatty acid derivative molecules such as e.g., fatty triols, fatty tetrols, dihydroxy fatty acids, etc. The disclosure further provides derivatives of the disclosed multifunctional molecules which are useful e.g., in the production of personal care products, surfactants, detergents, polymers, paints, coatings, and as emulsifiers, emollients, and thickeners in cosmetics and foods, as industrial solvents and plasticizers, etc. The disclosure further provides biochemical pathways, recombinant microorganisms and methods for the biological production of various multifunctional fatty acid derivatives.

### FIELD

The disclosure relates to the field of specialty chemicals and methods for their synthesis. The disclosure provides novel multifunctional fatty acid derivative compounds such as e.g., fatty triols, dihydroxy fatty acids, etc. derivatives thereof. The disclosure further provides biochemical pathways, recombinant microorganisms and methods for the biological production of various multifunctional fatty acid derivatives.

### BACKGROUND

Hydrocarbon based organic chemicals are employed by almost every industry. The many commercial and industrial uses of hydrocarbon based organic chemicals include e.g., emollients and thickeners in cosmetics and foods, pharmaceuticals, industrial solvents, surfactants, plasticizers, lubricants, emulsifiers, building blocks of polymers, etc., (see e.g., H. Maag (1984) Journal of the American Oil Chemists' Society 61(2): 259-267). Thus, hydrocarbon based organic chemicals play an indispensable role in modern society.

Historically, most organic chemicals have been obtained as by-products from the petrochemical industry. Although more than 90 percent of the organic chemical industry is based on petroleum, the production of organic chemicals from petroleum has many disadvantages and limitations. In particular, the synthesis of medium- to long-chain hydrocarbons (C6 to C18) with multiple hydroxy(—OH), oxo (═O), amino- (—NH₂) or carboxyl (CO₂H) groups in selective positions is extremely difficult and in many cases impractical or impossible starting from petrochemical feedstocks.

Clearly then, what is needed in the art are novel functionalized organic chemical compounds and methods for making them. In particular, a need exists for the production of medium- to long-chain hydrocarbons (C6 to C18) that have pre-designed, or “tailored,” specifications and properties useful for the preparation detergents, lubricants, pharmaceuticals, polymers and other valuable applications.

Fortunately, as will be clear from the detailed description that follows, the present disclosure provides for this and other needs.

### SUMMARY

One aspect of the disclosure provides a multifunctional molecule having a chemical formula according to

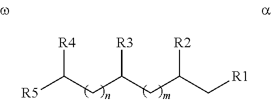

wherein
R1=-OH; —O₂H; —CH₂; —CH₂OH; —CHO; —CH₂NH₂; —CO₂H; —CO₂CH₃; —CO₂C₂H₅; —CO₂C₃H₇; —CO₂C₂H₃
R2=-H; —OH;
R3=-H; —OH;
R4=-H; —OH;
R5=-CH₃; —CH₂; —CH₂OH; —CHO; —CH₂NH₂; —CO₂H; —CO₂CH₃; —CO₂C₂H₅; —CO₂C₃H₇; —CO₂C₂H₃;

wherein
m=1-10 and n=0-9 and

wherein
if R1 is OH or —O₂H then 3<m+n≤10; and if R1 is other than OH or —O₂H, then 2<m+n≤10, and

wherein
the multifunctional molecule has at least three functional groups comprising a heteroatom, and

wherein
at least two of R2, R3 and R4 are OH; or when R5 is other than CH₃, CH₂ or C₃H₇ CH₂H₅ then at least one of R2, R3 and R4 are OH; and

wherein
when R5=CH₃ and R4=H then n=0 or 1; and

wherein
when R2=H, then R4 does not=H; and

wherein
when R3=H, then n+m=p and p=3-10; unless R1=-OH or —O₂H in which case when R3-H then n+m=p and p=4-10; and

wherein
when the multifunctional molecule comprises a double bond that is not terminal, the double bond is in a position corresponding to an omega7 (ω-7) position; and wherein
the multifunctional molecule is not 1,3,12-dodecane triol; is not 3,11-dihydroxy-tetradecanoic acid; is not 3,11-dihydroxy-tetradecanoic acid methyl ester and is not naturally occurring.

In one embodiment, the multifunctional fatty acid derivative molecule is a multifunctional alcohol. In one embodiment, the multifunctional fatty acid derivative molecule has R1=CH₂OH and R2=OH. In one embodiment, the multifunctional fatty acid derivative molecule is a member selected from the group consisting of 1,3,11-dodecane triol, 1,3, 10-dodecane triol, 1,3,9-dodecane triol, 1,3,12-dodecene triol, 1,3,11-dodecene triol, 1,3,10-dodecene triol, 1,3,9-dodecene triol, 1,3,11,12-dodecane tetrol, 1,3,10,12-dodecane tetrol, 1,3,9,12 dodecane tetrol, 1,3,7-decane triol, 1,3,8-decane triol, 1,3,9-decane triol.

In one embodiment, the multifunctional fatty acid derivative molecule is a multifunctional fatty acid ester. In one embodiment, the multifunctional fatty acid derivative molecule has R1=CO₂CH₃ and R2=OH. In one embodiment, the multifunctional fatty acid derivative molecule is a member selected from the group consisting of 3,12-dihydroxy dodecanoic acid methyl ester, 3,14-dihydroxy tetradecanoic acid methyl ester, 3,16-dihydroxy hexadecanoic acid methyl ester, 3,12-dihydroxy dodecenoic acid methyl ester, 3,14-dihydroxy tetradecenoic acid methyl ester, 3,16-dihydroxy hexadecenoic acid methyl ester, 3,11-dihydroxy dodecanoic acid methyl ester, 3,10-dihydroxy dodecanoic acid methyl ester, 3,9-dihydroxy dodecanoic acid methyl ester, 3,11-dihydroxy dodecenoic acid methyl ester, 3,10-dihydroxy dodecenoic acid methyl ester, 3,9-dihydroxy dodecenoic acid methyl ester, 3,13-dihydroxy tetradecanoic acid methyl ester, 3,12-dihydroxy tetradecanoic acid methyl ester, 3,13-dihydroxy tetradecenoic acid methyl ester, 3,12-dihydroxy tetradecenoic acid methyl ester, 3,11-dihydroxy tetradecenoic acid methyl ester, 3,15-dihydroxy hexadecanoic acid methyl ester, 3,14-dihydroxy hexadecanoic acid methyl ester, 3,13-dihydroxy hexadecanoic acid methyl ester, 3,15-dihydroxy hexadecenoic acid methyl ester, 3,14-dihydroxy hexadecenoic acid methyl ester and 3,13-dihydroxy hexadecenoic acid methyl ester.

In one embodiment, the multifunctional fatty acid derivative molecule has R1=CO₂CH₂CH₃ and R2=OH. In one embodiment, the multifunctional fatty acid derivative molecule is a member selected from the group consisting of 3,12-dihydroxy dodecanoic acid ethyl ester, 3,14-dihydroxy tetradecanoic acid ethyl ester, 3,16-dihydroxy hexadecanoic acid ethyl ester, 3,12-dihydroxy dodecenoic acid ethyl ester, 3,14-dihydroxy tetradecenoic acid ethyl ester, 3,16-dihydroxy hexadecenoic acid ethyl ester, 3,11-dihydroxy dodecanoic acid ethyl ester, 3,10-dihydroxy dodecanoic acid ethyl ester, 3,9-dihydroxy dodecanoic acid ethyl ester, 3,11-dihydroxy dodecenoic acid ethyl ester, 3,10-dihydroxy dodecenoic acid ethyl ester, 3,9-dihydroxy dodecenoic acid ethyl ester, 3,13-dihydroxy tetradecanoic acid ethyl ester, 3,12-dihydroxy tetradecanoic acid ethyl ester, 3,11-dihydroxy tetradecanoic acid ethyl ester, 3,13-dihydroxy tetradecenoic acid ethyl ester, 3,12-dihydroxy tetradecenoic acid ethyl ester, 3,11-dihydroxy tetradecenoic acid ethyl ester, 3,15-dihydroxy hexadecanoic acid ethyl ester, 3,14-dihydroxy hexadecanoic acid ethyl ester, 3,13-dihydroxy hexadecanoic acid ethyl ester, 3,15-dihydroxy hexadecenoic acid ethyl ester, 3,14-dihydroxy hexadecenoic acid ethyl ester and 3,13-dihydroxy hexadecenoic acid ethyl ester.

In one embodiment, the multifunctional fatty acid derivative molecule is a multifunctional fatty acid. In one embodiment, n≠4. In one embodiment, the multifunctional fatty acid derivative molecule has R2=H.

In one aspect, the disclosure provides a multifunctional fatty acid derivative molecule wherein the multifunctional fatty acid derivative molecule is a multifunctional fatty acid selected from the group consisting of: 10,14-dihydroxyhexadecanoic acid, 10,13-dihydroxyhexadecanoic acid, 9,10,15-trihydroxy hexadecanoic acid; 9,10,14-trihydroxy hexadecanoic acid; and 9,10,13-trihydroxy hexadecanoic acid.

In one aspect the disclosure provides a multifunctional fatty acid derivative molecule wherein the multifunctional fatty acid derivative molecule is an unsaturated multifunctional fatty acid selected from the group consisting of: 9,10,15 trihydroxy hexadecenoic acid; 9,10,14 trihydroxy hexadecenoic acid; 9,10,13 trihydroxy hexadecenoic acid; 9,10,15 trihydroxy octadecenoic acid; 7,10,16-trihydroxy-(8e)-hexadecenoic acid; and 7,10,14-trihydroxy-(8e)-hexadecenoic acid.

In one aspect the disclosure provides multifunctional fatty acid derivative molecule wherein the multifunctional fatty acid derivative molecule is a multifunctional polyol selected from the group consisting of 1,12,16-hexadecene triol, 1,9,10 hexadecane triol; 1,7,10 hexadecene triol and 1,7,10-(8e)-octadecene triol.

In one aspect the disclosure provides a carbonate derivative of a multifunctional fatty acid derivative molecule. In one embodiment, the carbonate derivative has a chemical structural formula according to:

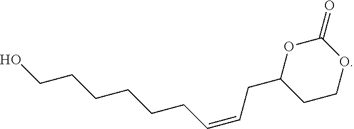 [SMILES: O=C1OCCC(C/C=C\CCCCCCO)O1]

In one embodiment, the carbonate derivative has a chemical structural formula according to:

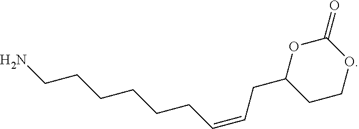 [SMILES: NCCCCCC/C=C\CC1CCOC(=O)O1]

In one aspect the disclosure provides a method for preparing a multifunctional fatty acid derivative molecule having an acyl chain length of 8-16 carbons the method comprising: culturing a recombinant microbe that comprises a heterologous enzyme pathway capable of producing a bifunctional fatty acid derivative molecule, and at least one heterologous hydroxylating enzyme, in a culture medium comprising a simple carbon source. In one embodiment, the at least one heterologous hydroxylating enzyme is selected from a heterologous hydroxylase enzyme and a heterologous hydratase enzyme or a combination thereof. In one embodiment, the heterologous hydroxylase enzyme is a member selected from the group consisting of omega-hydroxylases (ω-hydroxylases), mid-chain hydroxylases, and subterminal hydroxylases. In one embodiment, the recombinant microbe is selected from recombinant microbes that comprise: a heterologous enzyme pathway capable of producing a 3-hydroxy fatty acid; a heterologous enzyme pathway capable of producing a 3-hydroxy fatty ester; a heterologous enzyme pathway capable of producing a 1,3-fatty diol; a heterologous enzyme pathway capable of producing a hydroxy fatty acid; a heterologous enzyme pathway capable of producing a hydroxy fatty ester; and a heterologous enzyme pathway capable of producing a fatty diol. In one embodiment, the recombinant microbe is a recombinant bacterial cell.

In one aspect, the disclosure provides method for preparing 1,3,12 dodecanetriol, (z5)1,3,12 dodecenetriol or a combination thereof, the method comprising: culturing in a culture medium comprising a simple carbon source, a recombinant microbe that comprises: a heterologous enzyme pathway capable of producing a 1,3-fatty diol, and at least one heterologous hydroxylating enzyme, wherein the heterologous enzyme pathway capable of producing a 1,3-fatty diol comprises: (i) a heterologous plant FatB1 thioesterase and (ii) a heterologous CarB carboxylic acid reductase; and wherein the at least one heterologous hydroxylating enzyme is a heterologous co-hydroxylase selected from a cyp153A family co-hydroxylase and an alkB ω-hydroxylase or a combination thereof. In one embodiment, the cyp153A family comprises a cyp153A ω-hydroxylase protein selected from the group consisting of: SEQ ID NO:2, SEQ ID NO:4, SEQ ID NO:6, SEQ ID NO:8, SEQ ID NO: 10, SEQ ID NO: 12, SEQ ID NO: 14, SEQ ID NO: 16, SEQ ID NO: 18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46 and SEQ ID NO:48. In one embodiment, the cyp153A protein is a chimeric hybrid-fusion protein selected from the group consisting of: a SEQ ID NO:4, SEQ ID NO:6 and SEQ ID NO:8. In one embodiment,

the heterologous enzyme pathway capable of producing a 1,3-fatty diol further comprises a heterologous alcohol dehydrogenase. In one embodiment, the heterologous alcohol dehydrogenase is a heterologous AlrA dehydrogenase from Acinetobacter baylyi. In one embodiment, the at least one heterologous hydroxylating enzyme is a heterologous alkB ω-hydroxylase. In one embodiment, the at least one heterologous hydroxylating enzyme is a cyp153A family ω-hydroxylase. In one embodiment, the at least one heterologous hydroxylating enzyme is the heterologous ω-hydroxylase cyp153A from Marinobacter aquaeolei. In one embodiment, the at least one heterologous hydroxylating enzyme is a heterologous ω-hydroxylase cyp153A chimeric hybrid-fusion protein selected from the group consisting of: a SEQ ID NO:4, SEQ ID NO:6 and SEQ ID NO:8 In one embodiment, the at least one heterologous hydroxylating enzyme is the combination of a heterologous cyp153A family ω-hydroxylase and an alkB ω-hydroxylase. In one embodiment, the cyp153A family ω-hydroxylase is a cyp153A ω-hydroxylase from Marinobacter aquaeolei, and the heterologous alkB ω-hydroxylase is an alkB ω-hydroxylase from Pseudomonas putida. In one embodiment, the heterologous enzyme pathway capable of producing a 1,3-fatty diol comprises a heterologous FatB1 thioesterase from Umbellaria californica, and a heterologous CarB carboxylic acid reductase from Mycobacterium smegmatis; and the at least one heterologous hydroxylating enzyme is a heterologous cyp153A family ω-hydroxylase from Mannobacier aquaeolei, an alkB from Pseudomonas putida or a combination thereof. In one embodiment, the wherein the heterologous enzyme pathway capable of producing a 1,3-fatty diol further comprises a heterologous AlrA dehydrogenase from Acinetobacter baylyi.

In one aspect the disclosure provides a method for making a multifunctional fatty acid derivative molecule having a chemical formula according to:

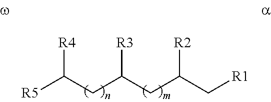

wherein
R1=-OH; —O₂H; —CH₂; —CH₂OH; —CHO; —CH₂NH₂; —CO₂H; —CO₂CH₃; —CO₂C₂H₅; —CO₂C₃H₇; —CO₂C₂H₃
R2=-H; —OH;
R3=-H; —OH;
R4=-H; —OH;
R5=-CH₃; —CH₂; —CH₂OH; —CHO; —CH₂NH₂; —CO₂H; —CO₂CH₃; —CO₂C₂H₅; —CO₂C₃H₇; —CO₂C₂H₃;

wherein
m=1-10 and n=0-9 and

wherein
if R1 is OH or —O₂H then 3<m+n≤10; and if R1 is other than OH or —O₂H, then 2<m+n≤10, and

wherein
the multifunctional molecule has at least three functional groups comprising a heteroatom, and

wherein
at least two of R2, R3 and R4 are OH; or when R5 is other than CH₃, CH₂ or C₃H₇ CH₂H₅ then at least one of R2, R3 and R4 are OH; and

wherein
when R5=CH₃ and R4=H then n=0 or 1; and

wherein
when R2=H, then R4 does not=H; and

wherein
when R3=H, then n+m=p and p=3-10; unless R1=-OH or —O₂H in which case when R3-H then n+m=p and p=4-10; and

wherein
when the multifunctional molecule comprises a double bond that is not terminal, the double bond is in a position corresponding to an omega7 (co-7) position; and

wherein
the multifunctional molecule is not 1,3,12-dodecane triol; is not 3,11-dihydroxy-tetradecanoic acid; is not 3,11-dihydroxy-tetradecanoic acid methyl ester and is not naturally occurring,
   the method comprising:
      culturing
         a recombinant microbe that comprises a heterologous enzyme pathway capable of producing a bifunctional fatty acid derivative molecule, and at least one heterologous hydroxylating enzyme,
      in a culture medium comprising a simple carbon source,
wherein
   the heterologous enzyme pathway capable of producing bifunctional fatty acid derivative molecule is selected from:
   a heterologous enzyme pathway capable of producing a 3-hydroxy fatty acid;
   a heterologous enzyme pathway capable of producing a 3-hydroxy fatty ester;
   a heterologous enzyme pathway capable of producing a 1,3-fatty diol
   a heterologous enzyme pathway capable of producing a hydroxy fatty acid;
   a heterologous enzyme pathway capable of producing a hydroxy fatty ester; and
   a heterologous enzyme pathway capable of producing a fatty diol; and
wherein
   the at least one heterologous hydroxylating enzyme is selected from a heterologous hydroxylase enzyme and a heterologous hydratase enzyme or a combination thereof.

In one embodiment, the heterologous hydroxylase enzyme is a member selected from the group consisting of omega-hydroxylases (co-hydroxylases), mid-chain hydroxylases, and subterminal hydroxylases. In one embodiment, the heterologous enzyme pathway capable of producing bifunctional fatty acid derivative molecule is the heterologous enzyme pathway capable of producing a 1,3-fatty diol. In one embodiment, the heterologous enzyme pathway capable of producing a 1,3-fatty diol comprises; a heterologous thioesterase and a heterologous carboxylic acid reductase. In one embodiment, the heterologous the heterologous enzyme pathway capable of producing a 1,3-fatty diol further comprises; a heterologous alcohol dehydrogenase. In one embodiment, the heterologous the heterologous enzyme pathway capable of producing a 1,3-fatty diol further comprises: a heterologous PhaG thioesterase from Pseudomonas putida, a heterologous CarB carboxylic acid reductase from Mycobacterium smegmatis, and a heterologous AlrA alcohol dehydrogenase from Acinetobacter baylyi. In one embodiment, the heterologous hydroxylase enzyme is a cyp102A subterminal-hydroxylase from Bacillus licheniformis, and the method produces multifunctional molecules selected from the group consisting of 1,3,9 decanetriol, 1,3,8 decanetriol, 1,3,7 decanetriol, 1,3,11 dodecanetriol, 1,3,10 dodecanetriol, 1,3,9 dodecanetriol, (z5)1,3,11 dodecenetriol, (z5)1,3,10 dodecenetriol and (z5)1,3,9 dodecenetriol.

In one aspect the disclosure provides method for preparing a multifunctional fatty acid derivative molecule selected from the group consisting of 9,10,16-trihydroxyhexadecanoic acid and 9,10,18-trihydroxyoctadecanoic acid, the method comprising: culturing a recombinant microbe that expresses a heterologous biochemical pathway comprising: (i) a delta 12 fatty acid epoxygenase and an epoxide hydrolase, (ii) a heterologous FatA thioesterase and (iii) a cyp153A ω-hydroxylase from Marinobacter aquaeolei in a culture medium comprising a simple carbon source.

In one aspect the disclosure provides a method for preparing a multifunctional fatty acid derivative molecule selected from the group consisting of 9,10,16-trihydroxyhexadecanoic acid methyl ester and 9,10,18-trihydroxyoctadecanoic acid methyl ester, the method comprising: culturing a recombinant microbe that expresses a heterologous biochemical pathway comprising: (i) a delta 12 fatty acid epoxygenase and an epoxide hydrolase, (ii) a heterologous FatA thioesterase, (iii) an acyl-CoA synthetase, (iv) an ester synthase and (v) a cyp153A ω-hydroxylase from Marinobacter aquaeolei in a culture medium comprising a simple carbon source and methanol.

In one aspect the disclosure provides method for preparing a multifunctional fatty acid derivative molecule selected from the group consisting of 9,10,16-trihydroxyhexadecanoic acid ethyl ester and 9,10,18-trihydroxyoctadecanoic acid ethyl ester, the method comprising: culturing a recombinant microbe that expresses a heterologous biochemical pathway comprising: (i) a delta 12 fatty acid epoxygenase and an epoxide hydrolase, (ii) a heterologous FatA thioesterase, (iii) an acyl-CoA synthetase, (iv) a ester synthase and (v) a cyp153A ω-hydroxylase from Marinobacter aquaeolei in a culture medium comprising a simple carbon source and ethanol.

In one aspect the disclosure provides method for preparing a multifunctional fatty acid derivative molecule selected from the group consisting of 1, 9,10,16-hexadecanetetrol and 1, 9,10,18-octadecanetetrol, the method comprising: culturing a recombinant microbe that expresses a heterologous biochemical pathway comprising of (i) a delta 12 fatty acid epoxygenase and an epoxide hydrolase, (ii) a heterologous acyl-ACP reductase (AAR) and (iii) a cyp153A ω-hydroxylase from Marinobacter aquaeolei in a culture medium comprising a simple carbon source.

In one aspect the disclosure provides multifunctional fatty acid derivative molecule having a general formula according to:

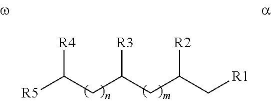

wherein
ω indicates the reduced end of the fatty acid derivative molecule and a indicates the carboxyl end of the fatty acid derivative molecule, and
   wherein
R1=-OH; —O₂H; —CH₂; —CH₂OH; —CHO; —CH₂NH₂; —CO₂H; —CO₂CH₃; —CO₂C₂H₅; —CO₂C₃H₇; —CO₂C₂H₃
R2=-H; —OH; —NH₂
R3=-H; —OH; —NH₂
R4=-H; —OH; —NH₂
R5=-CH₃; —CH₂; —CH₂OH; —CHO; —CH₂NH₂; —CO₂H; —CO₂CH₃; —CO₂C₂H₅; —CO₂C₃H₇; —CO₂C₂H₃;

wherein
m=1-10 and n=0-9 and wherein
if R1 is OH or —O₂H then . . . 3<m+n≤10; and if R1 is other than OH or —O₂H, then 2<m+n≤10, and

wherein
the multifunctional molecule has at least three functional groups comprising a heteroatom, and

wherein
when R5=CH₃ and R4=H then n=0 or 1; and

wherein
when R2=CH₃, then R4 does not=H; and

wherein
when R3=H, then n+m=p and p=2-10; and

wherein
when the multifunctional molecule comprises a double bond that is not terminal, the double bond is in a position corresponding to an omega7 (co-7) position; and

wherein
the multifunctional molecule is not 1,3,12-dodecane triol; is not 3,11-dihydroxy-tetradecanoic acid; is not 3,11-dihydroxy-tetradecanoic acid methyl ester and is not naturally occurring.

In one embodiment, R2=NH₂. In one embodiment, R1=CO₂H. In one embodiment, the multifunctional molecule is selected from the group consisting of: 3-amino, 12-hydroxy-dodecanoic acid and 3-amino, 12-hydroxy-dodecenoic acid. In one embodiment, R1=CH₂OH. In one embodiment, the multifunctional molecule is selected from the group consisting of: 3 amino dodecene 1,12 diol and 3-amino-dodecane 1,12-diol. In one embodiment, R5=CH₂NH₂. In one embodiment, the multifunctional molecule is selected from the group consisting of: 12-amino dodecane-1,3-diol, 12-amino dodecane-1,9-diol, (z5)12-amino dodecene-1,3-diol, (z5)12-amino dodecene-1,9-diol, 3-hydroxy, 12-amino dodecanoic acid and (z5)3-hydroxy, 12-amino dodecenoic acid.

In one aspect the disclosure provides a method for preparing a multifunctional molecule comprising an amino group, the method comprising: culturing a recombinant microbe that expresses a heterologous biochemical pathway comprising a heterologous thioesterase, and at least one heterologous hydroxylating enzyme, a heterologous alcohol dehydrogenase or oxidase and a heterologous transaminase, in a culture medium comprising a simple carbon source. In one embodiment, the thioeserase has enzyme activity according to EC3.1.2. In one embodiment, the thioeserase is selected from FatB1 from Umbellularia californica (Q41635) or PhaG from Pseudomonas putida (AAN67031).

In one aspect the disclosure provides a method for preparing 3-hydroxy, 12-amino dodecanoic acid, 3-amino, 12-hydroxy dodecanoic acid, (z5)3-hydroxy, 12-amino dodecenoic acid and (z5) 3-amino, 12-hydroxy dodecenoic acid, the method comprising: culturing, a recombinant microbe comprising a heterologous FatB1 thioesterase from Umbellularia californica, an AlkJ alcohol oxidase from Pseudomonas putida, a CV_2025 transaminase from Chromobacterium violaceum and a cyp153A ω-hydroxylase from Marinobacter aquaeolei on a simple carbon source.

In one aspect the disclosure provides a method for preparing 12-amino dodecane-1,3-diol, 3-amino dodecane-1,12-diol, 12-amino dodecane-1,9-diol, (z5) 12-amino dodecene-1,3-diol, (z5)3-amino dodecene-1,12-diol and (z5) 12-amino dodecene-1,9-diol, the method comprising: culturing, a recombinant microbe comprising a heterologous FatB1 thioesterase from Umbellularia californica, an heterologous AlkJ alcohol oxidase from Pseudomonas putida, a heterologous CV 2025 transaminase such as from Chromobacterium violaceum, a heterologous CarB carboxylic acid reductase from Mycobacterium smegmatis and a heterologous cyp153A ω-hydroxylase from Mannobacier aquaeolei on a simple carbon source.

### BRIEF DESCRIPTION OF THE FIGURES

FIG. 1 Illustrates some biochemical pathways that convert 3-hydroxy acyl-ACPs into trifunctional fatty acid derivatives. In particular, FIG. 1A Illustrates schematically production of a trifunctional fatty acid derivative from a 3-hydroxy fatty acid. FIG. 1B Illustrates schematically production of a trifunctional fatty acid derivative from a 3-hydroxy fatty ester.

FIG. 1C Illustrates schematically production of a trifunctional fatty acid derivative from a 1,3-diol. Although FIG. 1 depicts the enzymatic hydroxylations as the last steps of the biochemical pathway, they can occur at earlier steps of the pathways, e.g. in FIG. 1C the fatty acid intermediate may be hydroxylated before it is converted to a fatty alcohol by carboxylic acid reductase (CAR).

FIG. 2 Illustrates biochemical pathways that convert acyl-ACPs into trifunctional fatty acid derivatives. In particular, FIG. 2A Illustrates schematically production of a trifunctional fatty acid derivative from a fatty acid. FIG. 2B Illustrates schematically production of a trifunctional fatty acid derivative from a fatty ester. FIG. 2C Illustrates schematically production of a trifunctional fatty acid derivative from a fatty alcohol. Although FIG. 2 depicts the enzymatic hydroxylations as the last steps of the biochemical pathway, they can occur at earlier steps of the pathways, e.g. in FIG. 2C the fatty acid intermediate may be hydroxylated before it is converted to a fatty alcohol by carboxylic acid reductase (CAR).

FIG. 3 Illustrates an exemplary biochemical pathway to produce fatty triols employing a ω-hydroxylase. Fatty acid derivatives with 12 carbon atoms are depicted.

FIG. 4 Illustrates an exemplary biochemical pathway to produce fatty triols employing a “subterminal”-hydroxylase. Fatty acid derivatives with 12 carbon atoms are depicted.

FIG. 5 Illustrates GC/MS chromatographs of extracts from recombinant E. coli strains. FIG. 5A Illustrates GC/MS chromatographs of extracts from recombinant E. coli without expression of a cyp153A ω-hydroxylase when fed with C12 diols. FIG. 5B Illustrates GC/MS chromatographs of extracts from recombinant E. coli with expression of a cyp153 A ω-hydroxylase when fed with C12 diols, which were efficiently converted to C12 triols.

FIG. 6A and FIG. 6B Illustrates the mass spectrum and ion fragmentation pattern of (z5) 1,3,12-trimethylsilyloxy dodecene (peak at 12.33 minutes), which is derivatized (z5)1,3,12 dodecane triol. The mass spectrum is from an extract of a recombinant E. coli strain expressing a cyp153 A ω-hydroxylase.

FIG. 7A and FIG. 7B Illustrates the mass spectrum and ion fragmentation pattern of 1,3,12-trimethylsilyloxy dodecane (peak at 12.48 minutes), which is derivatized (z5)1,3,12 dodecane triol. The mass spectrum is from an extract of a recombinant E. coli strain expressing a cyp153 A ω-hydroxylase

FIG. 8 Illustrates a GC/MS chromatograph of an extract from recombinant E. coli strains with expression of a cyp153A ω-hydroxylase when fed with 3-hydroxy dodecanoic acid, which was efficiently converted to 3,12-dihydroxy dodecanoic acid.

FIG. 9 Illustrates the mass spectrum and ion fragmentation pattern of 3,12-trimethylsilyloxy dodecanoic acid (peak at 13.25 minutes), which is derivatized 3,12-dihydroxy dodecanoic acid. The mass spectrum is from an extract of a recombinant E. coli strain expressing a cyp153 A ω-hydroxylase.

FIG. 10A and FIG. 10B Illustrates GC/MS chromatographs of extracts from recombinant E. coli strains without (A) and with (B) expression of a cyp102A “subterminal”-hydroxylase when fed with C12 diols, which were efficiently converted to various C12 triols

FIG. 11A and FIG. 11B Illustrates the mass spectrum and ion fragmentation pattern of (z5) 1,3,9-trimethylsilyloxy dodecene (peak A in FIG. 10), which is derivatized (z5)[3]9-dodecenetriol. The mass spectrum is from an extract of a recombinant E. coli strain expressing a cyp102A “subterminal” hydroxylase

FIG. 12A and FIG. 12B Illustrates the mass spectrum and ion fragmentation pattern of 1,3,9-trimethylsilyloxy dodecane (peakB in FIG. 10), which is derivatized 1,3,9-dodecanetriol. The mass spectrum is from an extract of a recombinant E. coli strain expressing a cyp102A “subterminal” hydroxylase

FIG. 13A and FIG. 13B Illustrates the mass spectrum and ion fragmentation pattern of (z5) 1,3,10-trimethylsilyloxy dodecene (peak C in FIG. 10), which is derivatized (z5) 1,3,10-dodecenetriol. The mass spectrum is from an extract of a recombinant E. coli strain expressing a cyp102A “subterminal” hydroxylase

FIG. 14A and FIG. 14B Illustrates the mass spectrum and ion fragmentation pattern of (Z5)1,3,11-trimethylsilyloxy dodecene (peak D in FIG. 10), which is derivatized (z5) 1,3,11-dodecenetriol. The mass spectrum is from an extract of a recombinant E. coli strain expressing a cyp102A “subterminal” hydroxylase

FIG. 15A and FIG. 15B Illustrates the mass spectrum and ion fragmentation pattern of 1,3,10-trimethylsilyloxy dodecane (peak E in FIG. 10), which is derivatized 1,3,10-dodecanetriol. The mass spectrum is from an extract of a recombinant E. coli strain expressing a cyp102A “subterminal” hydroxylase

FIG. 16A and FIG. 16B Illustrates the mass spectrum and ion fragmentation pattern of 1,3,11-trimethylsilyloxy dodecane (peak F in FIG. 10), which is derivatized 1,3,11-dodecanetriol. The mass spectrum is from an extract of a recombinant E. coli strain expressing a cyp102A “subterminal” hydroxylase.

FIG. 17A and FIG. 17B Illustrates peaks indicative of 10,16-dihydroxy hexadecanoic acid production in a recombinant E. coli strain grown on a simple carbon source.

FIG. 18A and FIG. 18B Illustrates mass spectrum and ion fragmentation pattern of trimethylsilyl-10,16-dihydroxy hexadecanoic acid.

FIG. 19A and FIG. 19B Illustrates mass spectrum and ion fragmentation pattern from trimethylsilyl derivatized 10,13 dihydroxy hexadecanoic acid.

FIG. 20A and FIG. 20B Illustrates mass spectrum and ion fragmentation pattern of trimethyl silyl derivatized 10,14-dihydroxy hexadecanoic acid.

FIG. 21A and FIG. 21B Illustrates mass spectrum and ion fragmentation pattern of trimethyl silyl derivatized 10,15-dihydroxy hexadecanoic acid.

### DETAILED DESCRIPTION

### Definitions

As used herein and in the appended claims, singular articles such as “a” and “an” and “the” and similar referents in the context of describing the elements are to be construed to cover both the singular and the plural, unless otherwise indicated herein or clearly contradicted by context.

As used herein, “about” is understood by persons of ordinary skill in the art and may vary to some extent depending upon the context in which it is used. If there are uses of the term which are not clear to persons of ordinary skill in the art given the context in which the term “about” is used, “about” will mean up to plus or minus 10% of the particular term.

As will be understood by one skilled in the art, for any and all purposes, all ranges disclosed herein also encompass any and all possible subranges and combinations of subranges thereof. Furthermore, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Unless defined otherwise, technical and scientific terms used herein have the same meaning as commonly understood by a person of ordinary skill in the art. In particular, this disclosure utilizes routine techniques in the field of recombinant genetics, organic chemistry, fermentation and biochemistry. Basic texts disclosing the general terms in molecular biology and genetics include e.g., Lackie, Dictionary of Cell and Molecular Biology, Elsevier (5^{th} ed. 2013). Basic texts disclosing methods in recombinant genetics and molecular biology include e.g., Sambrook et al., Molecular Cloning-A Laboratory Manual, Cold Spring Harbor Press 4^{th} Edition (Cold Spring Harbor, N.Y. 2012) and Current Protocols in Molecular Biology Volumes 1-3, John Wiley & Sons, Inc. (1994-1998) and Supplements 1-115 (1987-2016). Basic texts disclosing the general methods and terms in biochemistry include e.g., Lehninger Principles of Biochemistry sixth edition, David L. Nelson and Michael M. Cox eds. W.H. Freeman (2012). Basic texts disclosing the general methods and terminology of fermentation include e.g., Principles of Fermentation Technology, 3rd Edition by Peter F Stanbury, Allan Whitaker and Stephen J Hall. Butterworth-Heinemann (2016). Basic texts disclosing the general methods and terms organic chemistry include e.g., Favre, Henri A. and Powell, Warren H. Nomenclature of Organic Chemistry. IUPAC Recommendations and Preferred Name 2013. Cambridge, UK: The Royal Society of Chemistry, 2013; Practical Synthetic Organic Chemistry; Reactions, Principles, and Techniques, Stephane Caron ed., John Wiley and Sons Inc. (2011); Organic Chemistry, 9th Edition—Francis Carey and Robert Giuliano, McGraw Hill (2013).

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as a mixture of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

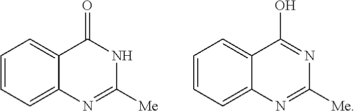 [SMILES: Cc1nc2ccccc2c(=O)[nH]1 ; Cc1nc(O)c2ccccc2n1]

As another example, guanidines may exhibit the following isomeric forms in protic organic solution, also referred to as tautomers of each other:

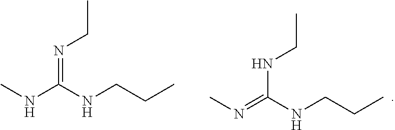

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

Geometric isomers can be represented by the symbol which denotes a bond that can be a single, double or triple bond as described herein. Provided herein are various geometric isomers and mixtures thereof resulting from the arrangement of substituents around a carbon-carbon double bond. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The term “cis” represents substituents on the same side of the plane of the ring, and the term “trans” represents substituents on opposite sides of the plane of the ring. Mixtures of compounds wherein the substituents are disposed on both the same and opposite sides of plane of the ring are designated “cis/trans.”

In certain embodiments, the pharmaceutically acceptable form thereof is an isomer. “Isomers” are different compounds that have the same molecular formula. “Stereoisomers” are isomers that differ only in the way the atoms are arranged in space. As used herein, the term “isomer” includes any and all geometric isomers and stereoisomers. For example, “isomers” include cis- and trans-isomers, E- and Z-isomers, R- and S-enantiomers, diastereomers, (d)-isomers, (l)-isomers, racemic mixtures thereof, and other mixtures thereof, as falling within the scope of this disclosure.

“Enantiomers” are a pair of stereoisomers that are non-superimposable mirror images of each other. A mixture of a pair of enantiomers in any proportion can be known as a “racemic” mixture. The term “(+−)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R)- or (S)-. The present chemical entities, pharmaceutical compositions and methods are meant to include all such possible isomers, including racemic mixtures, optically pure forms and intermediate mixtures. Optically active (R)- and (S)-isomers can be prepared, for example, using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

The term “fatty acid” as used herein, refers to an aliphatic carboxylic acid having the formula RCOOH wherein R is an aliphatic group having at least 4 carbons, typically between about 4 and about 28 carbon atoms. The aliphatic R group can be saturated or unsaturated, branched or unbranched. Unsaturated “fatty acids” may be monounsaturated or polyunsaturated.

A “fatty acid” or “fatty acids”, as used herein, can be produced within a cell through the process of fatty acid biosynthesis, through the reverse of fatty acid degradation or beta-oxidation, or they can be fed to a cell. As is well known in the art, fatty acid biosynthesis is generally a malonyl-CoA dependent synthesis of acyl-ACPs or acyl CoAs, while the reverse of beta-oxidation results is acetyl-CoA dependent and results in the synthesis of acyl-CoAs. Fatty acids fed to cell are converted to acyl-CoAs and can be converted to acyl-ACPs. Fatty acids can be synthesized in a cell by natural fatty acid biosynthetic pathways or can be synthesized from heterologous fatty acid biosynthetic pathways that comprise a combination of fatty acid biosynthetic and/or degradation enzymes that result in the synthesis of acyl-CoAs and/or Acyl-ACPs.

Fatty acid biosynthesis and degradation occur in all life forms, including prokaryotes, single cell eukaryotes, higher eukaryotes, and Archaea. The tools and methods disclosed herein are useful in the production of fatty acid derivatives that are derived through any one or more of fatty acid synthesis, degradation, or feeding in any organism that naturally produces alkyl thioesters.

The term “fatty acid derivative” as used herein, refers to a product derived from a fatty acid. Thus, a “fatty acid derivative” includes “fatty acids” as defined above. In general, “fatty acid derivatives” include malonyl-CoA derived compounds including acyl-ACP or acyl-ACP derivatives. “Fatty acid derivatives” also include malonyl-CoA derived compounds such as acyl-CoA or acyl-CoA derivatives. Thus, a “fatty acid derivatives” include alky-thioesters and acyl-thioesters. Further, a “fatty acid derivative” includes a molecule/compound that is derived from a metabolic pathway that includes a fatty acid derivative enzyme. Exemplary fatty acid derivatives include fatty acids, fatty acid esters (e.g., waxes, fatty acid esters, fatty acid methyl esters (FAME), fatty acid ethyl esters (FAEE)), fatty alcohol acetate esters (FACE), fatty amines, fatty aldehydes, fatty alcohols, hydrocarbons e.g., alkanes, alkenes, etc, ketones, terminal olefins, internal olefins, 3-hydroxy fatty acid derivatives, bifunctional fatty acid derivatives (e.g., ?-hydroxy fatty acids, 1,3 fatty-diols, ?-diols, -3-hydroxy triols, ?-hydroxy FAME, ?-OH FAEE, etc.), and unsaturated fatty acid derivatives, including unsaturated compounds of each of the above mentioned fatty acid derivatives. Fatty acid derivatives also include multifunctional fatty acid derivatives, as defined below.

The term “multifunctional fatty acid derivatives” or equivalently “multifunctional molecules” as used herein, refers to fatty acid derivative molecules having a carbon chain length of between 8 and 16 carbons that have at least three functional groups which comprise a heteroatom. Exemplary functional groups which comprise a heteroatom include e.g. a hydroxy or equivalently, hydroxyl (—OH), oxo ( ), carboxyl (CO2H), amino (NH2), O-acetyl (CO2C2H₃), methoxy (OCH3) or ester (CO2CH3, CO2C2H5, CO2C3H7, CO2C2H3) group.

“Multifunctional fatty acid derivatives” disclosed herein may be saturated or unsaturated multifunctional fatty acid derivatives. Typically, unsaturated “multifunctional fatty acid derivatives” or “multifunctional molecules” that are not exclusively terminal olefins have a double bond located at the omega-7 (-7) position on the hydrocarbon chain. That is to say, the double bond is located between the seventh and eighth carbons from the reduced end of the fatty acid from which the multifunctional fatty acid derivative is derived. For example, (9E)-1,3,16-trihydroxy-hexadecene has a 16-hydroxyl group that is added by a hydroxylase to the reducing end of (9E)-1,3 dihydroxy hexadecane, a fatty diol unsaturated at the omega-7 position.

The expression “fatty acid derivative composition” as used herein, refers to a composition of fatty acid derivatives, for example a fatty acid composition produced by an organism. A “fatty acid derivative composition” may comprise a single fatty acid derivative species or may comprise a mixture of fatty acid derivative species. In some exemplary embodiments, the mixture of fatty acid derivatives includes more than one type of fatty acid derivative product (e.g., fatty acids, fatty acid esters, fatty alcohols, fatty alcohol acetates, fatty aldehydes, fatty amine, bifunctional fatty acid derivatives, and multifunctional fatty acid derivatives, etc.). In other exemplary embodiments, the mixture of fatty acid derivatives includes a mixture of fatty acid esters (or another fatty acid derivative) with different chain lengths, saturation and/or branching characteristics. In other exemplary embodiments, the mixture of fatty acid derivatives comprises predominantly one type of fatty acid derivative e.g., a multifunctional fatty acid derivative composition. In still other exemplary embodiments, the mixture of fatty acid derivatives comprises a mixture of more than one type of fatty acid derivative product e.g., fatty acid derivatives with different chain lengths, saturation and/or branching characteristics. In still other exemplary embodiments, the mixture of fatty acid derivatives comprises a mixture of fatty esters and 3-hydroxy esters. In still other exemplary embodiments, a fatty acid derivative composition comprises a mixture of fatty alcohols and fatty aldehydes, in particular a mixture of multifunctional fatty alcohols or fatty aldehydes. In still other exemplary embodiments, a fatty acid derivative composition comprises a mixture of FAME and/or FAEE, in particular a mixture of multifunctional FAME and/or FAEE. In still other exemplary embodiments, a fatty acid derivative composition comprises a mixture of fatty alcohol acetate esters (FACE), in particular a mixture of multifunctional fatty alcohol acetate esters (FACE). In other exemplary embodiments, the mixture of fatty acid derivatives includes a mixture of multifunctional fatty acid derivatives with different chain lengths, saturation and/or functional group characteristics. In other exemplary embodiments, the mixture of fatty acid derivatives comprises predominantly one type of fatty acid derivative e.g., a multifunctional fatty acid derivative composition comprising predominantly 1,3,12-dodecane triol.

The term “malonyl-CoA derived compound” as used herein refers to any compound or chemical entity (i.e., intermediate or end product) that is made via a biochemical pathway wherein malonyl-CoA functions as intermediate and/or is made upstream of the compound or chemical entity. For example, a malonyl-CoA derived compound may include, but is not limited to, a fatty acid derivative such as, for example, a fatty acid; a fatty ester including, but not limited to a fatty acid methyl ester (FAME) and/or a fatty acid ethyl ester (FAEE); a fatty alcohol; a fatty aldehyde; a fatty amine; an alkane; an olefin or alkene; a hydrocarbon; a beta hydroxy fatty acid derivative, a bifunctional fatty acid derivative, a multifunctional fatty acid derivative and/or an unsaturated fatty acid derivative.

As used herein “alkyl-thioester” or equivalently an “acyl thioester” is a compound in which the carbonyl carbon of an acyl chain and the sulfydryl group of an organic thiol forms a thioester bond. Representative organic thiols include Cystein, beta-cysteine, glutathione, mycothiol, pantetheine, Coenzyme A (CoA) and the acyl carrier protein (ACP). Thus “acyl-ACP” refers to an alkyl thioester formed between the carbonyl carbon of an acyl chain and the sulfhydryl group of the phosphopantetheinyl moiety of an ACP. An “Acyl-CoA” refers to an alkyl thioester formed between the carbonyl carbon of an acyl chain and the sulfhydryl group of the phosphopantetheinyl moiety of CoA. In some exemplary embodiments an alkyl thioester, such as acyl-ACP or acyl CoA, is an intermediate in the synthesis of fully saturated acyl-thioesters. In other exemplary embodiments an alkyl thioester, such as acyl-ACP or acyl CoA, is an intermediate in the synthesis of unsaturated acyl thioesters. In some exemplary embodiments, the carbon chain of the acyl group of an acyl thiester has 5, 6, 7, 8, 9, 10, 11, 12, 13, 14, 15, 16, 17, 18, 19, 20, 21, 22, 23, 24, 25, 26, 27 or 28 carbons. In other exemplary embodiments, the carbon chain of the acyl group of acyl-thioester is a medium-chain and has 6, 7, 8, 9, 10, 11, 12, 13, 14, 15, or 16 carbons. In other exemplary embodiments the carbon chain of the acyl group of acyl-thioester is 10 carbons in length. In still other exemplary embodiments, the carbon chain of the acyl group of acyl-thioester is 12 carbons in length. In still other exemplary embodiments, the carbon chain of the acyl group of acyl-thioester is 14 carbons in length. In still other exemplary embodiments, the carbon chain of the acyl group of acyl-thioester is 16 carbons in length. Each of these acyl-thioesters are substrates for fatty acid derivative enzymes such as e.g., thioesterases, acyl ACP reductases, ester synthases and their engineered variants that convert the acyl-thioester to fatty acid derivatives.

As used herein, the expression “fatty acid derivative biosynthetic pathway” refers to a biochemical pathway that produces fatty acid derivatives. The enzymes that comprise a “fatty acid derivative biosynthetic pathway” are thus referred to herein as “fatty acid derivative biosynthetic polypeptides” or equivalently “fatty acid derivative enzymes”. As discussed supra, the term “fatty acid derivative,” includes a molecule/compound derived from a biochemical pathway that includes a fatty acid derivative enzyme. Thus, a thioesterase enzyme (e.g., an enzyme having thioesterase activity EC 3.1.1.14) is a “fatty acid derivative biosynthetic peptide” or equivalently a “fatty acid derivative enzyme.” In addition to a thioesterase, a fatty acid derivative biosynthetic pathway may include additional fatty acid derivative enzymes to produce fatty acid derivatives having desired characteristics. Thus the term “fatty acid derivative enzymes” or equivalently “fatty acid derivative biosynthetic polypeptides” refers to, collectively and individually, enzymes that may be expressed or overexpressed to produce fatty acid derivatives. Non-limiting examples of “fatty acid derivative enzymes” or equivalently “fatty acid derivative biosynthetic polypeptides” include e.g., fatty acid synthetases, thioesterases, acyl-CoA synthetases, acyl-CoA reductases, acyl ACP reductases, alcohol dehydrogenases, alcohol O-acyltransferases, fatty alcohol-forming acyl-CoA reductases, fatty acid decarboxylases, fatty aldehyde decarbonylases and/or oxidative deformylases, carboxylic acid reductases, fatty alcohol O-acetyl transferases, ester synthases, etc. “Fatty acid derivative enzymes” or equivalently “fatty acid derivative biosynthetic polypeptides” convert substrates into fatty acid derivatives. In exemplary embodiments, a suitable substrate for a fatty acid derivative enzyme may be a first fatty acid derivative, which is converted by the fatty acid derivative enzyme into a different, second fatty acid derivative.

The term “polyol” as used herein, refers to compounds, typically fatty alcohols, which have more than one hydroxy group. Thus, as referred to herein, a polyol may have two hydroxy groups, three hydroxy groups, four hydroxy groups, etc. In general, a “polyol” that has two hydroxy groups is referred to herein as a “diol”, a “polyol” that has three hydroxy groups is referred to herein as a “triol”, a “polyol” that has four hydroxy groups is referred to herein as a “tetrol” and so on.

The expression “hydroxy group”, “hydroxyl group”, “alcohol group” are used interchangeably herein and refer to a chemical functional group containing one oxygen atom covalently bonded to one hydrogen atom (—OH).

Sequence Accession numbers throughout this description were obtained from databases provided by the NCBI (National Center for Biotechnology Information) maintained by the National Institutes of Health, U.S.A. (which are identified herein as “NCBI Accession Numbers” or alternatively as “GenBank Accession Numbers” or alternatively a simply “Accession Numbers”), and from the UniProt Knowledgebase (UniProtKB) and Swiss-Prot databases provided by the Swiss Institute of Bioinformatics (which are identified herein as “UniProtKB Accession Numbers”).

The term “enzyme classification (EC) number” refers to a number that denotes a specific polypeptide sequence or enzyme. EC numbers classify enzymes according to the reaction they catalyze. EC numbers are established by the nomenclature committee of the international union of biochemistry and molecular biology (IUBMB), a description of which is available on the IUBMB enzyme nomenclature website on the world wide web.

As used herein, the term “isolated,” with respect to products (such as multifunctional fatty acid derivatives disclosed herein) refers to products that are separated from cellular components, cell culture media, or chemical or synthetic precursors. The multifunctional fatty acid derivatives disclosed herein produced by the methods disclosed herein can be relatively immiscible in the fermentation broth, as well as in the cytoplasm. Therefore, in exemplary embodiments, the multifunctional fatty acid derivatives disclosed herein collect in an organic phase extracellularly and are thereby “isolated”.

As used herein, the terms “polypeptide” and “protein” are used interchangeably to refer to a polymer of amino acid residues that is typically 12 or more amino acids in length. Polypeptides less than 12 amino acids in length are referred to herein as “peptides”. The terms apply to amino acid polymers in which one or more amino acid residue is an artificial chemical mimetic of a corresponding naturally occurring amino acid, as well as to naturally occurring amino acid polymers and non-naturally occurring amino acid polymers. The term “recombinant polypeptide” refers to a polypeptide that is produced by recombinant techniques, wherein generally DNA or RNA encoding the expressed protein is inserted into a suitable expression vector that is in turn used to transform a host cell to produce the polypeptide. In some exemplary embodiments, DNA or RNA encoding an expressed peptide, polypeptide or protein is inserted into the host chromosome via homologous recombination or other means well known in the art, and is so used to transform a host cell to produce the peptide or polypeptide. Similarly, the terms “recombinant polynucleotide” or “recombinant nucleic acid” or “recombinant DNA” are produced by recombinant techniques that are known to those of skill in the art (see e.g., methods described in Sambrook et al. (supra) and/or Current Protocols in Molecular Biology (supra).

When referring to two nucleotide or polypeptide sequences, the “percentage of sequence identity” between the two sequences is determined by comparing the two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The “percentage of sequence identity” is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

Thus, the expression “percent identity,” or equivalently “percent sequence identity” in the context of two or more nucleic acid sequences or peptides or polypeptides, refers to two or more sequences or subsequences that are the same or have a specified percentage of nucleotides or amino acids that are the same (e.g., about 50% identity, preferably 55%, 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured e.g., using a BLAST or BLAST 2.0 sequence comparison algorithm with default parameters (see e.g., Altschul et al. (1990) J. Mol. Biol. 215(3):403-410) and/or the NCBI web site at ncbi.nlm.nih.gov/BLAST/) or by manual alignment and visual inspection. Percent sequence identity between two nucleic acid or amino acid sequences also can be determined using e.g., the Needleman and Wunsch algorithm that has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6 (Needleman and Wunsch (1970) J. Mol. Biol. 48:444-453). The percent sequence identity between two nucleotide sequences also can be determined using the GAP program in the GCG software package, using a NWSgapdna.CMP matrix and a gap weight of 40, 50, 60, 70, or 80 and a length weight of 1, 2, 3, 4, 5, or 6. One of ordinary skill in the art can perform initial sequence identity calculations and adjust the algorithm parameters accordingly. A set of parameters that may be used if a practitioner is uncertain about which parameters should be applied to determine if a molecule is within a homology limitation of the claims, are a Blossum 62 scoring matrix with a gap penalty of 12, a gap extend penalty of 4, and a frameshift gap penalty of 5. Additional methods of sequence alignment are known in the biotechnology arts (see, e.g., Rosenberg (2005) BMC Bioinformatics 6:278; Altschul et al. (2005) FEBS J. 272(20):5101-5109).

Two or more nucleic acid or amino acid sequences are said to be “substantially identical,” when they are aligned and analyzed as discussed above and are found to share about 50% identity, preferably 55%, 60%, 65%, 70%, 75%, 80%, 85%, 90%, 91%, 92%, 93%, 94%, 95%, 96%, 97%, 98%, 99%, or higher identity over a specified region. Two nucleic acid sequences or polypeptide sequences are said to be “identical” if the sequence of nucleotides or amino acid residues, respectively, in the two sequences are the same when aligned for maximum correspondence as described above. This definition also refers to, or may be applied to, the compliment of a test sequence. Identity is typically calculated over a region that is at least about 25 amino acids or nucleotides in length, or more preferably over a region that is 50-100 amino acids or nucleotides in length, or over the entire length of a given sequence.

The expressions “hybridizes under low stringency, medium stringency, high stringency, or very high stringency conditions” describes conditions for hybridization and washing. Guidance for performing hybridization reactions can be found e.g., in Current Protocols in Molecular Biology, John Wiley & Sons, N.Y. (1989), 6.3.1-6.3.6. Aqueous and non-aqueous methods are described in the cited reference and either method can be used. Specific hybridization conditions referred to herein are as follows: (1) low stringency hybridization conditions—6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions—6×SSC at about 45° C., followed by one or more washes in 0.2×SSC, 0.1% SDS at 60° C.; (3) high stringency hybridization conditions—6×SSC at about 45° C., followed by one or more washes in 0.2.×SSC, 0.1% SDS at 65° C.; and (4) very high stringency hybridization conditions—0.5M sodium phosphate, 7% SDS at 65° C., followed by one or more washes at 0.2×SSC, 1% SDS at 65° C. Very high stringency conditions (4) are the preferred conditions unless otherwise specified.

The term “endogenous” as used herein refers to a substance e.g., a nucleic acid, protein, etc. that is produced from within a cell. Thus, an “endogenous” polynucleotide or polypeptide refers to a polynucleotide or polypeptide produced by the cell. In some exemplary embodiments an “endogenous” polypeptide or polynucleotide is encoded by the genome of the parental cell (or host cell). In other exemplary embodiments, an “endogenous” polypeptide or polynucleotide is encoded by an autonomously replicating plasmid carried by the parental cell (or host cell). In some exemplary embodiments, an “endogenous” gene is a gene that was present in the cell when the cell was originally isolated from nature i.e., the gene is “native to the cell”. In other exemplary embodiments, an “endogenous” gene has been altered through recombinant techniques e.g., by altering the relationship of control and coding sequences. Thus, a “heterologous” gene may, in some exemplary embodiments, be “endogenous” to a host cell.

The term “endogenous” as used herein refers to a substance e.g., a nucleic acid, protein, etc. that is produced from within a cell. Thus, an “endogenous” polynucleotide or polypeptide refers to a polynucleotide or polypeptide produced by the cell. In some exemplary embodiments an “endogenous” polypeptide or polynucleotide is encoded by the genome of the parental cell (or host cell). In other exemplary embodiments, an “endogenous” polypeptide or polynucleotide is encoded by an autonomously replicating plasmid carried by the parental cell (or host cell). In some exemplary embodiments, an “endogenous” gene is a gene that was present in the cell when the cell was originally isolated from nature i.e., the gene is “native to the cell”. In other exemplary embodiments, an “endogenous” gene has been altered through recombinant techniques e.g., by altering the relationship of control and coding sequences. Thus, a “heterologous” gene may, in some exemplary embodiments, be “endogenous” to a host cell.

In contrast, an “exogenous” polynucleotide or polypeptide, or other substance (e.g., fatty acid derivative, small molecule compound, etc.) refers to a polynucleotide or polypeptide or other substance that is not produced by the parental cell and which is therefore added to a cell, a cell culture or assay from outside of the cell.

As used herein the term “native” refers to the form of a nucleic acid, protein, polypeptide or a fragment thereof that is isolated from nature or a nucleic acid, protein, polypeptide or a fragment thereof that is without intentionally introduced mutations.

As used herein, the term “fragment” of a polypeptide refers to a shorter portion of a full-length polypeptide or protein ranging in size from two amino acid residues to the entire amino acid sequence minus one amino acid residue. In certain embodiments of the disclosure, a fragment refers to the entire amino acid sequence of a domain of a polypeptide or protein (e.g., a substrate binding domain or a catalytic domain).

The term “gene” as used herein, refers to nucleic acid sequences e.g., DNA sequences, which encode either an RNA product or a protein product, as well as operably-linked nucleic acid sequences that affect expression of the RNA or protein product (e.g., expression control sequences such as e.g., promoters, enhancers, ribosome binding sites, translational control sequences, etc). The term “gene product” refers to either the RNA e.g., tRNA, mRNA and/or protein expressed from a particular gene.

The term “expression” or “expressed” as used herein in reference to a gene, refers to the production of one or more transcriptional and/or translational product(s) of a gene. In exemplary embodiments, the level of expression of a DNA molecule in a cell is determined on the basis of either the amount of corresponding mRNA that is present within the cell or the amount of protein encoded by that DNA produced by the cell. The term “expressed genes” refers to genes that are transcribed into messenger RNA (mRNA) and then translated into protein, as well as genes that are transcribed into other types of RNA, such as e.g., transfer RNA (tRNA), ribosomal RNA (rRNA), and regulatory RNA, which are not translated into protein.

The level of expression of a nucleic acid molecule in a cell or cell free system is influenced by “expression control sequences” or equivalently “regulatory sequences”. “Expression control sequences” or “regulatory sequences” are known in the art and include, for example, promoters, enhancers, polyadenylation signals, transcription terminators, nucleotide sequences that affect RNA stability, internal ribosome entry sites (IRES), and the like, that provide for the expression of the polynucleotide sequence in a host cell. In exemplary embodiments, “expression control sequences” interact specifically with cellular proteins involved in transcription (see e.g., Maniatis et al., Science, 236: 1237-1245 (1987); Goeddel, Gene Expression Technology: Methods in Enzymology, Vol. 185, Academic Press, San Diego, Calif. (1990)). In exemplary methods, an expression control sequence is operably linked to a polynucleotide sequence. By “operably linked” is meant that a polynucleotide sequence and an expression control sequence(s) are functionally connected so as to permit expression of the polynucleotide sequence when the appropriate molecules (e.g., transcriptional activator proteins) contact the expression control sequence(s). In exemplary embodiments, operably linked promoters are located upstream of the selected polynucleotide sequence in terms of the direction of transcription and translation. In some exemplary embodiments, operably linked enhancers can be located upstream, within, or downstream of the selected polynucleotide.

As used herein, the phrase “the expression of said nucleotide sequence is modified relative to the wild type nucleotide sequence”, refers to a change e.g., an increase or decrease in the level of expression of an native nucleotide sequence or a change e.g., an increase or decrease in the level of the expression of a heterologous or non-native polypeptide-encoding nucleotide sequence as compared to a control nucleotide sequence e.g., wild-type control. In some exemplary embodiments, the phrase “the expression of said nucleotide sequence is modified relative to the wild type nucleotide sequence,” refers to a change in the pattern of expression of a nucleotide sequence as compared to a control pattern of expression e.g., constitutive expression as compared to developmentally timed expression.

The term “overexpressed” as used herein, refers to a gene whose expression is elevated in comparison to a “control” level of expression. In exemplary embodiments, “overexpression” of a gene is caused by an elevated rate of transcription as compared to the native transcription rate for that gene. In other exemplary embodiments, overexpression is caused by an elevated rate of translation of the gene compared to the native translation rate for that gene. Methods of testing for overexpression are well known in the art, for example transcribed RNA levels can be assessed using rtPCR and protein levels can be assessed using SDS page gel analysis.

In other embodiments, the polypeptide, polynucleotide, or hydrocarbon having an altered level of expression is “attenuated” or has a “decreased level of expression.” As used herein, “attenuate” and “decreasing the level of expression” mean to express or cause to be expressed a polynucleotide, polypeptide, or hydrocarbon in a cell at a lesser concentration than is normally expressed in a corresponding control cell (e.g., wild type cell) under the same conditions.

A polynucleotide or polypeptide can be attenuated using any method known in the art. For example, in some exemplary embodiments, the expression of a gene or polypeptide encoded by the gene is attenuated by mutating the regulatory polynucleotide sequences which control expression of the gene. In other exemplary embodiments, the expression of a gene or polypeptide encoded by the gene is attenuated by overexpressing a repressor protein, or by providing an exogenous regulatory element that activates a repressor protein. In still other exemplary embodiments, DNA- or RNA-based gene silencing methods are used to attenuate the expression of a gene or polynucleotide. In some embodiments, the expression of a gene or polypeptide is completely attenuated, e.g., by deleting all or a portion of the polynucleotide sequence of a gene.

The degree of overexpression or attenuation can be 1.5-fold or more, e.g., 2-fold or more, 3-fold or more, 5-fold or more, 10-fold or more, or 15-fold or more. Alternatively, or in addition, the degree of overexpression or attenuation can be 500-fold or less, e.g., 100-fold or less, 50-fold or less, 25-fold or less, or 20-fold or less. Thus, the degree of overexpression or attenuation can be bounded by any two of the above endpoints. For example, the degree of overexpression or attenuation can be 1.5-500-fold, 2-50-fold, 10-25-fold, or 15-20-fold.

As used herein, “modified activity” or an “altered level of activity” of a protein/polypeptide e.g., of a variant ChFatB2 enzyme, in a recombinant host cell refers to a difference in one or more characteristics in the activity the protein/polypeptide as compared to the characteristics of an appropriate control protein e.g., the corresponding parent protein or corresponding wild type protein. Thus, in exemplary embodiments, a difference in activity of a protein having “modified activity” as compared to a corresponding control protein is determined by measuring the activity of the modified protein in a recombinant host cell and comparing that to a measure of the same activity of a corresponding control protein in an otherwise isogenic host cell. Modified activities can be the result of, for example, changes in the structure of the protein (e.g., changes to the primary structure, such as e.g., changes to the protein's nucleotide coding sequence that result in changes in substrate specificity, changes in observed kinetic parameters, changes in solubility, etc.); changes in protein stability (e.g., increased or decreased degradation of the protein) etc. In some exemplary embodiments, a polypeptide having “modified activity” is a mutant or a variant ChFatB2 thioesterase disclosed herein.

The term “recombinant” as used herein, refers to a genetically modified polynucleotide, polypeptide, cell, tissue, or organism. When used with reference to a cell, the term “recombinant” indicates that the cell has been modified by the introduction of a heterologous nucleic acid or protein or has been modified by alteration of a native nucleic acid or protein, or that the cell is derived from a cell so modified and that the derived cell comprises the modification. Thus, for example, “recombinant cells” or equivalently “recombinant host cells” may be modified to express genes that are not found within the native (non-recombinant) form of the cell or may be modified to abnormally express native genes e.g., native genes may be overexpressed, underexpressed or not expressed at all. In exemplary embodiments, a “recombinant cell” or “recombinant host cell” is engineered to express a heterologous enzyme pathway capable of producing a bifunctional fatty acid derivative molecule. A recombinant cell can be derived from a microorganism such as a bacterium, a virus or a fungus. In addition, a recombinant cell can be derived from a plant or an animal cell. In exemplary embodiments, a “recombinant host cell” or “recombinant cell” is used to produce one or more multifunctional fatty acid derivatives including, but not limited to, multifunctional fatty acids, multifunctional fatty esters (e.g., waxes, fatty acid esters, fatty esters, fatty acid methyl esters (FAME), fatty acid ethyl esters (FAEE)), multifunctional fatty acyl acetate esters (FAce), multifunctional fatty alcohols (e.g., polyols), multifunctional fatty aldehydes, multifunctional fatty amines, multifunctional terminal olefins, multifunctional ketones, etc. Therefore, in some exemplary embodiments a “recombinant host cell” is a “production host” or equivalently, a “production host cell”. In some exemplary embodiments, the recombinant cell includes one or more polynucleotides, each polynucleotide encoding a polypeptide having fatty acid biosynthetic enzyme activity, wherein the recombinant cell produces a multifunctional fatty acid derivative composition when cultured in the presence of a carbon source under conditions effective to express the polynucleotides.

When used with reference to a polynucleotide, the term “recombinant” indicates that the polynucleotide has been modified by comparison to the native or naturally occurring form of the polynucleotide or has been modified by comparison to a naturally occurring variant of the polynucleotide. In an exemplary embodiment, a recombinant polynucleotide (or a copy or complement of a recombinant polynucleotide) is one that has been manipulated by the hand of man to be different from its naturally occurring form. Thus, in an exemplary embodiment, a recombinant polynucleotide is a mutant form of a native gene or a mutant form of a naturally occurring variant of a native gene wherein the mutation is made by intentional human manipulation e.g., made by saturation mutagenesis using mutagenic oligonucleotides, through the use of UV radiation, mutagenic chemicals, chemical synthesis etc. Such a recombinant polynucleotide might comprise one or more point mutations, deletions and/or insertions relative to the native or naturally occurring variant form of the gene. Similarly, a polynucleotide comprising a promoter operably linked to a second polynucleotide (e.g., a coding sequence) is a “recombinant” polynucleotide. Thus, a recombinant polynucleotide comprises polynucleotide combinations that are not found in nature. A recombinant protein (discussed supra) is typically one that is expressed from a recombinant polynucleotide, and recombinant cells, tissues, and organisms are those that comprise recombinant sequences (polynucleotide and/or polypeptide).

As used herein, the term “microorganism” refers generally to a microscopic organism. Microorganisms can be prokaryotic or eukaryotic. Exemplary prokaryotic microorganisms include e.g., bacteria, archaea, cyanobacteria, etc. An exemplary bacterium is Escherichia coli. Exemplary eukaryotic microorganisms include e.g., yeast, protozoa, algae, etc. In exemplary embodiments, a “recombinant microorganism” is a microorganism that has been genetically altered and thereby expresses or encompasses a heterologous nucleic acid sequence and/or a heterologous protein.

A “production host” or equivalently a “production host cell” is a cell used to produce products. As disclosed herein, a “production host” is typically modified to express or overexpress selected genes, or to have attenuated expression of selected genes. Thus, a “production host” or a “production host cell” is a “recombinant host” or equivalently a “recombinant host cell”. Non-limiting examples of production hosts include plant, animal, human, bacteria, yeast, cyanobacteria, algae, and/or filamentous fungi cells. An exemplary “production host” is a recombinant Escherichia coli cell.

The term “acetyl-CoA derived compound” refers to any compound or chemical entity (i.e., intermediate or end product) that is made via a biochemical pathway wherein acetyl-CoA functions as intermediate and/or is made upstream of the compound or chemical entity. For example, a acetyl-CoA derived compound may include, but is not limited to, a fatty acid derivative such as, for example, a fatty acid; a fatty ester including, but not limited to a fatty acid methyl ester (FAME) and/or a fatty acid ethyl ester (FAEE); a fatty alcohol; a fatty aldehyde; a fatty amine; an alkane; an olefin or alkene; a hydrocarbon; a 3-hydroxy fatty acid derivative, a bifunctional fatty acid derivative, a multifunctional fatty acid derivative, an unsaturated fatty acid derivative, etc.

As used herein, the terms “purify,” “purified,” or “purification” mean the removal or isolation of a molecule from its environment by, for example, isolation or separation. “Substantially purified” molecules are at least about 60% free (e.g., at least about 65% free, at least about 70% free, at least about 75% free, at least about 80% free, at least about 85% free, at least about 90% free, at least about 95% free, at least about 96% free, at least about 97% free, at least about 98% free, at least about 99% free) from other components with which they are associated. As used herein, these terms also refer to the removal of contaminants from a sample. For example, the removal of contaminants can result in an increase in the percentage of malonyl-CoA derived compounds including multifunctional fatty acid derivatives or other compounds in a sample. For example, when a malonyl-CoA derived compound including a multifunctional fatty acid derivative or other compound is produced in a recombinant host cell, the malonyl-CoA derived compound including the multifunctional fatty acid derivative or other compound can be purified by the removal of host cell proteins. After purification, the percentage of malonyl-CoA derived compounds including multifunctional fatty acid derivatives or other compounds in the sample is increased. The terms “purify,” “purified,” and “purification” are relative terms which do not require absolute purity. Thus, for example, when a malonyl-CoA derived compound (including a multifunctional fatty acid derivative disclosed herein or other compound) is produced in recombinant host cells, a malonyl-CoA derived compound (including a purified multifunctional fatty acid derivative or other compound) is a malonyl-CoA derived compound (including a multifunctional fatty acid derivative or other compound) that is substantially separated from other cellular components (e.g., nucleic acids, polypeptides, lipids, carbohydrates, or other hydrocarbons).

As used herein, the term “attenuate” means to weaken, reduce, or diminish. For example, a polypeptide can be attenuated by modifying the polypeptide to reduce its activity (e.g., by modifying a nucleotide sequence that encodes the polypeptide).

As used herein, the term “carbon source” refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, and gases (e.g., CO and C02). Exemplary carbon sources include, but are not limited to, monosaccharides, such as glucose, fructose, mannose, galactose, xylose, and arabinose; oligosaccharides, such as fructo-oligosaccharide and galacto-oligosaccharide; polysaccharides such as starch, cellulose, pectin, and xylan; disaccharides, such as sucrose, maltose, cellobiose, and turanose; cellulosic material and variants such as hemicelluloses, methyl cellulose and sodium carboxymethyl cellulose; succinate, lactate, and acetate; alcohols, such as ethanol, methanol, and glycerol, or mixtures thereof. The carbon source can also be a product of photosynthesis, such as glucose. In certain embodiments, the carbon source is biomass. In other embodiments, the carbon source is glucose. In other embodiments the carbon source is sucrose. In other embodiments the carbon source is glycerol. In other embodiments, the carbon source is a simple carbon source. In other embodiments, the carbon source is a renewable carbon source. In other embodiment, the carbon source is natural gas. In other embodiments the carbon source comprises one or more components of natural gas, such as methane, ethane, or propane. In other embodiments, the carbon source is flu gas or synthesis gas. In still other embodiments, the carbon source comprises one or more components of flu or synthesis gas such as carbon monoxide, carbon dioxide, hydrogen, etc. As used herein, the term “carbon source” or “simple carbon source” specifically excludes oleochemicals such as e.g., saturated or unsaturated fatty acids.

As used herein, the term “biomass” refers to any biological material from which a carbon source is derived. In some embodiments, a biomass is processed into a carbon source, which is suitable for bioconversion. In other embodiments, the biomass does not require further processing into a carbon source. The carbon source can be converted into a composition comprising multifunctional fatty acid derivatives.

An exemplary source of biomass is plant matter or vegetation, such as corn, sugar cane, or switchgrass. Another exemplary source of biomass is metabolic waste products, such as animal matter (e.g., cow manure). Further exemplary sources of biomass include algae and other marine plants. Biomass also includes waste products from industry, agriculture, forestry, and households, including, but not limited to, glycerol, fermentation waste, ensilage, straw, lumber, sewage, garbage, cellulosic urban waste, and food leftovers (e.g., soaps, oils and fatty acids). The term “biomass” also can refer to sources of carbon, such as carbohydrates (e.g., monosaccharides, disaccharides, or polysaccharides).

I. Introduction

Hydrocarbon molecules with multiple functional groups have many industrial applications, e.g. as high performance chemicals, lubricants, personal care products, fragrances, adjuvants, polymers, etc. These functional groups provide useful properties themselves, for instance adding hydrophilicity for use in formulations, or as handles for a next step in chemistry, for instance polymerization. Thus, such molecules are useful for the preparation detergents, lubricants, pharmaceuticals, polymers and other valuable applications

Unfortunately however, the synthesis of hydrocarbon molecules, especially medium- to long-chain hydrocarbons (C6 to C18) with multiple hydroxy(—OH), oxo (=O), amino-(-NH2) or carboxyl (CO2H) groups in selective positions is extremely difficult and in many cases impractical starting from petrochemical feedstocks.

Thus, what is needed in the art are novel functionalized hydrocarbon compounds and methods for making them. In particular, a need exists for the production of medium- to long-chain hydrocarbons (C6 to C18) that have pre-designed, or “tailored,” specifications and properties. Further, there is a desire to source such compounds from renewable and sustainable resources. Fortunately, the instant disclosure provides for this and other needs.

II. Multifunctional Fatty Acid Derivative Molecules and Derivatives Thereof

A. General Methods

This disclosure utilizes routine techniques in the field of recombinant genetics. Basic texts disclosing the general methods and terms in molecular biology and genetics include e.g., Sambrook et al., Molecular Cloning, a Laboratory Manual, Cold Spring Harbor Press 4th edition (Cold Spring Harbor, N.Y. 2012); Current Protocols in Molecular Biology Volumes 1-3, John Wiley & Sons, Inc. (1994-1998). This disclosure also utilizes routine techniques in the field of biochemistry. Basic texts disclosing the general methods and terms in biochemistry include e.g., Lehninger Principles of Biochemistry sixth edition, David L. Nelson and Michael M. Cox eds. W.H. Freeman (2012). This disclosure also utilizes routine techniques in industrial fermentation. Basic texts disclosing the general methods and terms in fermentation include e.g., Principles of Fermentation Technology, 3rd Edition by Peter F. Stanbury, Allan Whitaker and Stephen J. Hall. Butterworth-Heinemann (2016); Fermentation Microbiology and Biotechnology, 2nd Edition, E. M. T. El-Mansi, C. F. A. Bryce, Arnold L. Demain and A. R. Allman eds. CRC Press (2007). This disclosure also utilizes routine techniques in the field of organic chemistry. Basic texts disclosing the general methods and terms in organic chemistry include e.g., Practical Synthetic Organic Chemistry: Reactions, Principles, and Techniques, Stephane Caron ed., John Wiley and Sons Inc. (2011); The Synthetic Organic Chemist's Companion, Michael C. Pirrung, John Wiley and Sons Inc. (2007); Organic Chemistry, 9th Edition—Francis Carey and Robert Giuliano, McGraw Hill (2013).

For nucleic acids, sizes are given in either kilobases (kb) or base pairs (bp). Estimates are typically derived from agarose or acrylamide gel electrophoresis, from sequenced nucleic acids, or from published DNA sequences. For proteins, sizes are given in kilodaltons (kDa) or amino acid residue numbers. Protein sizes may be estimated from gel electrophoresis, from sequenced proteins, from derived amino acid sequences, or from published protein sequences.

Oligonucleotides that are not commercially available can be chemically synthesized e.g., according to the solid phase phosphoramidite triester method first described by Beaucage & Caruthers, Tetrahedron Letts. 22:1859-1862 (1981), using an automated synthesizer, as described in Van Devanter et al., Nucleic Acids Res. 12:6159-6168 (1984). Purification of oligonucleotides is e.g., by either native acrylamide gel electrophoresis or by anion-exchange HPLC as described in Pearson & Reanier, J. Chrom. 255:137-149 (1983).

The sequence of cloned genes and synthetic oligonucleotides can be verified after cloning using, e.g., the chain termination method for sequencing double-stranded templates of Wallace et al., Gene 16:21-26 (1981).

B. Multifunctional Molecules

In an exemplary embodiment, the disclosure provides “multifunctional fatty acid derivatives” or equivalently “multifunctional molecules”. Typically, multifunctional fatty acid derivatives have a carbon chain length of between 6 and 16 carbons and have at least three functional groups which comprise a heteroatom. Exemplary functional groups which comprise a heteroatom include e.g. a hydroxyl or equivalently, hydroxyl (—OH), oxo (CHO), carboxyl (CO₂H), amino (CH₂NH₂), O-acetyl (CO₂C₂H₃), methoxy (COCH₃) or ester (CO₂CH₃, CO₂C₂H₅, CO₂C₃H₇, CO₂C₂H₃) group

In an exemplary embodiment, the present disclosure provides novel multifunctional fatty acid derivative molecules having a general formula according to Scheme 1.

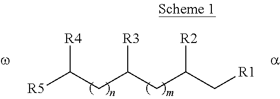

wherein
ω indicates the reduced end of the fatty acid derivative molecule and a indicates the carboxyl end of the fatty acid derivative molecule, and
   wherein
R1=-OH; —O₂H; —CH₂; —CH₂OH; —CHO; —CH₂NH₂; —CO₂H; —CO₂CH₃; —CO₂C₂H₅; —CO₂C₃H₇; —CO₂C₂H₃
R2=-H; —OH;
R3=-H; —OH;
R4=-H; —OH;
R5=-CH₃; —CH₂; —CH₂OH; —CHO; —CH₂NH₂; —CO₂H; —CO₂CH₃; —CO₂C₂H₅; —CO₂C₃H₇; —CO₂C₂H₃;

wherein
m=1-10 and n=0-9 and wherein
if R1 is OH or —O₂H then . . . 3<m+n≤10; and if R1 is other than OH or —O₂H, then 2<m+n≤10, and

wherein
the multifunctional molecule has at least three functional groups comprising a heteroatom, and

wherein
at least two of R2, R3 and R4 are OH; or when R5 is other than CH₃, CH₂ then at least one of R2, R3 and R4 are OH; and

wherein
when R5=CH₃ and R4=H then n=0 or 1; and

wherein
when R2=CH₃, then R4 does not=H; and

wherein
when R3=H, then n+m=p and p=3-10; unless R1=-OH or —O₂H in which case when R3-H then n+m=p and p=4-10; and

wherein
when the multifunctional molecule comprises a double bond that is not terminal, the double bond is in a position corresponding to an omega7 (ω-7) position; and

wherein
the multifunctional molecule is not 1,3,12-dodecane triol; is not 3,11-dihydroxy-tetradecanoic acid; is not 3,11-dihydroxy-tetradecanoic acid methyl ester and is not naturally occurring.

As will be apparent to one of skill in the art, when —OH groups are present at positions R2, R3 and/or R4, chiral center(s) are formed at the carbons to which the —OH groups are bound. Thus, the multifunctional molecules disclosed herein are useful as synthons for the production of chirally important compounds such as pharmaceuticals, nutraceuticals, pesticides, herbicides, flavors, fragrances, solvents, bioactive compounds, etc.

Double bonds, if present, can be either (Z) or (E). The presence of a double bond adds another layer of functionality to the molecules disclosed herein conferring on the molecules the ability to participate in chemical reactions involving a double bond including e.g., polymerization, alkylation, metathesis, etc. Chemical reactions utilizing the carbon-carbon double bond are known in the art (see e.g., Practical Synthetic Organic Chemistry: Reactions, Principles, and Techniques, Stephane Caron ed. (supra)).

Thus, the multifunctional molecules disclosed herein provide novel molecules with new functionalities that can be used to address old problems in an improved way and/or which can find new uses altogether.

(i) Molecules having R1=CH₂OH and R2=OH in Scheme 1

Referring to the general formula provided by Scheme 1 (above) when R1=CH₂OH and R2=OH the disclosure provides multifunctional polyol molecules having chemical structural formulas as follows:

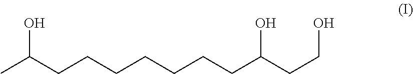

Formula I is referred to herein as 1,3,11-dodecane triol. With reference to Scheme 1, 1,3,11-dodecane triol, is described as R1=CH₂OH; R2=OH; R3=H; R4=OH; R5=CH₃; and m+n=p=6.

In another exemplary embodiment, the disclosure provides a multifunctional molecule having a chemical structural formula according to Formula II

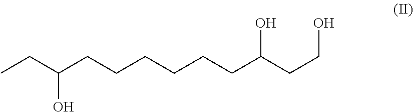

The molecule of Formula II is referred to herein as 1,3,10-dodecane triol. With reference to Scheme 1, 1,3,10-dodecane triol, is described as R1=CH₂OH; R2=OH; R3=OH; R4=H; R5=CH₃; n=0 and m=6.

In another exemplary embodiment, the disclosure provides a multifunctional molecule having a chemical structural formula according to Formula III

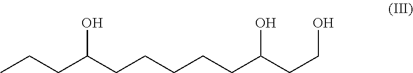

Formula III is referred to herein as 1,3,9-dodecane triol. With reference to Scheme 1, 1,3,9-dodecane triol, is described as R1=CH₂OH; R2=OH; R3=OH; R4=H; R5=CH₃; n=1 and m=5.

In another exemplary embodiment, the disclosure provides a multifunctional molecule having a chemical structural formula according to Formula IV.

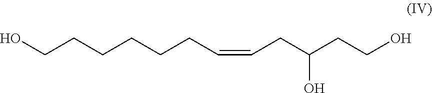 [SMILES: OCCCCCC/C=C\CC(O)CCO]

Formula IV is referred to herein as 1,3,12-dodecene triol. With reference to Scheme 1, the molecule of Formula IV, 1,3,12-dodecene triol, is described as R1=CH₂OH; R2=OH; R3=H; R4=H; R5=CH₂OH; n+m=6. In an exemplary embodiment, 1,3,12-dodecene triol, the double bond is in cis and therefore the molecule of Formula V is (z5)1,3,12-dodecene triol.

In another exemplary embodiment, the disclosure provides a multifunctional molecule having a chemical structural formula according to Formula V.

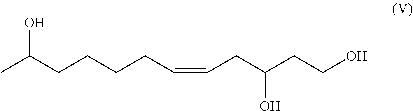

Formula V is referred to herein as 1,3,11-dodecene triol. With reference to Scheme 1, the molecule of Formula V, 1,3,11-dodecene triol, is described as R1=CH₂OH; R2=OH; R3=H; R4=OH; R5=CH₃; n+m=6. In an exemplary embodiment, 1,3,11-dodecene triol, the double bond is in cis and therefore the molecule of Formula V is (z5)1,3,11-dodecene triol.

In another exemplary embodiment, the disclosure provides a multifunctional molecule having a chemical structural formula according to Formula VI.

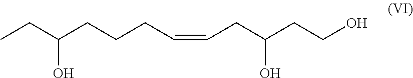

Formula VI is referred to herein as 1,3,10-dodecene triol. With reference to Scheme 1, the molecule of Formula V, 1,3,10-dodecene triol, is described as R1=CH₂OH; R2=OH; R3=OH; R4=H; R5=CH₃; n=0 m=6. In an exemplary embodiment, 1,3,10-dodecene triol, the double bond is in cis and therefore the molecule of Formula V is (z5)1,3,10-dodecene triol.

In another exemplary embodiment, the disclosure provides a multifunctional molecule having a chemical structural formula according to Formula VII.

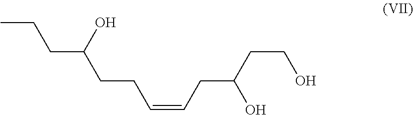

Formula VII is referred to herein as 1,3,9-dodecene triol. With reference to Scheme 1, the molecule of Formula V, 1,3,9-dodecene triol, is described as R1=CH₂OH; R2=OH; R3=OH; R4=H; R5=CH₃; n=1 and m=5. In an exemplary embodiment, 1,3,9-dodecene triol, the double bond is in cis and therefore the molecule of Formula V is (z5)1,3,9-dodecene triol.

In another exemplary embodiment, the disclosure provides a multifunctional molecule having a chemical structural formula according to Formula VIII.

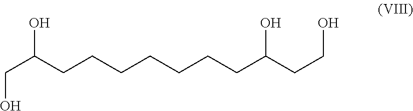 [SMILES: OCCC(O)CCCCCCCC(O)CO]

Formula VIII is referred to herein as 1,3,11,12-dodecane tetrol. With reference to Scheme 1, 1,3,11,12-dodecane tetrol is described as R1=CH₂OH; R2=OH; R3=H; R4=OH; R5=CH₂OH; n+m=6.

In another exemplary embodiment, the disclosure provides a multifunctional molecule having a chemical structural formula according to Formula IX.

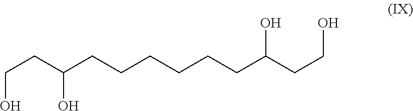 [SMILES: OCCC(O)CCCCCCC(O)CCO]

Formula IX is referred to herein as 1,3,10,12-dodecane tetrol. With reference to Scheme 1, 1,3,10,12-dodecane tetrol, is described as R1=CH₂OH; R2=O; R3=OH; R4=H; R5=CH₂OH; n=0 and m=6.

In another exemplary embodiment, the disclosure provides a multifunctional molecule having a chemical structural formula according to Formula X.

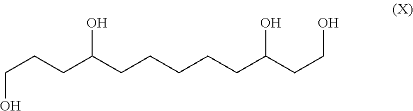 [SMILES: OCCCC(O)CCCCCC(O)CCO]

Formula X is referred to herein as 1,3,9,12-dodecane tetrol. With reference to Scheme 1, 1,3,9,12-dodecane tetrol, is described as R1=CH₂OH; R2=OH; R3=OH; R4=H; R5=CH₂OH; n=1 and m=5.

In another exemplary embodiment, the disclosure provides a multifunctional molecule having a chemical structural formula according to Formula XI.

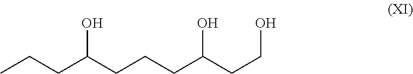

Formula XI is referred to herein as 1,3,7-decane triol. With reference to Scheme 1, 1,3,7-decane triol, is described as R1=CH₂OH; R2=OH; R3=OH; R4=H; R5=CH₃; n=1 and m=3.

In another exemplary embodiment, the disclosure provides a multifunctional molecule having a chemical structural formula according to Formula XII.

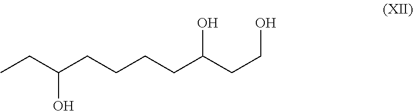

Formula XII is referred to herein as 1,3,8-decane triol. With reference to Scheme 1, 1,3,8-decane triol, is described as R1=CH₂OH; R2=OH; R3=OH; R4=H; R5=CH₃; n=0 and m=4.

In another exemplary embodiment, the disclosure provides a multifunctional molecule having a chemical structural formula according to Formula XIII.

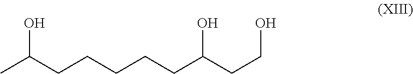

Formula XIII is referred to herein as 1,3,9-decane triol. With reference to Scheme 1, 1,3,9-decane triol, is described as R1=CH₂OH; R2=OH; R3=H; R4=OH; R5=CH₃; n+m=4.

In another exemplary embodiment, the disclosure provides a multifunctional molecule having a chemical structural formula according to Formula XIV.

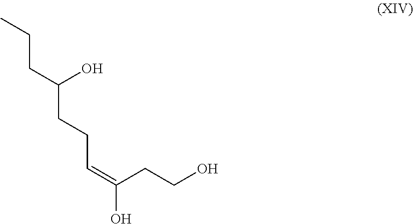

Formula XIV is referred to herein as 1,3,7-decene triol. 1,3,7-decene triol is the tautomer of keto-1,8-dihydroxydecane. With reference to Scheme 1, 1,3,7-decene triol, is described as R1=CH₂OH; R2=OH; R3=OH; R4=H; R5=CH₃; n=1 and m=3. In an exemplary embodiment, 1,3,7-decene triol, the double bond is in cis and therefore the molecule of Formula XIV is (z3)1,3,7-decene triol.

In another exemplary embodiment, the disclosure provides a multifunctional molecule having a chemical structural formula according to Formula XV.

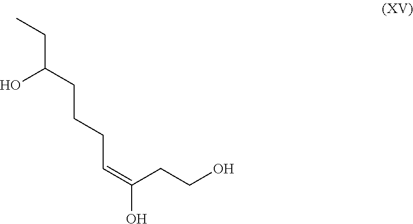

Formula XV is referred to herein as 1,3,8-decene triol. With reference to Scheme 1, 1,3,8-decene triol, is described as R1=CH₂OH; R2=OH; R3=OH; R4=H; R5=CH₃; n=0 and m=4. In an exemplary embodiment, 1,3,8-decene triol, the double bond is in cis and therefore the molecule of Formula XV is (z3)1,3,8-decene triol.

In another exemplary embodiment, the disclosure provides a multifunctional molecule having a chemical structural formula according to Formula XVI.

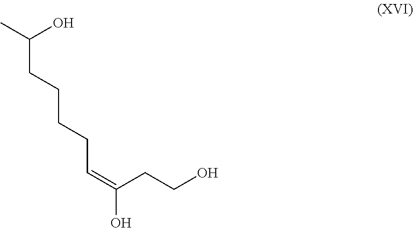

Formula XVI is referred to herein as 1,3,9-decene triol. With reference to Scheme 1, 1,3,9-decene triol, is described as R1=CH₂OH; R2=OH; R3=OH; R4=H; R5=CH₃; n=0 and m=4. In an exemplary embodiment, 1,3,9-decene triol, the double bond is in cis and therefore the molecule of Formula XV is (z3)1,3,9-decene triol.

(ii) Molecules Having R1=CO₂CH₃ or CO₂CH₂CH₃ and R2=OH in Scheme 1

The disclosure also provides multifunctional fatty acid methyl ester and ethyl esters.

### Methyl Esters

Thus, referring to the general formula provided by Scheme 1 (above) when R1=CO₂CH₃ and R2=OH the disclosure provides multifunctional molecules fatty acid methyl esters having chemical structural formulas as follows:

In one embodiment, the disclosure provides a multifunctional fatty acid derivative molecule having a chemical structural formula according to Formula XVII.

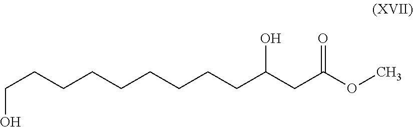 [SMILES: COC(=O)CC(O)CCCCCCCCCO]

Formula XVII is referred to herein as 3,12-dihydroxy dodecanoic acid methyl ester. With reference to Scheme 1, 3,12-dihydroxy dodecanoic acid methyl ester is described as R1=CO₂CH₃; R2=OH; R3=H; R4=H; R5=CH₂OH; n+m=6.

In another embodiment, the disclosure provides a multifunctional fatty acid derivative molecule having a chemical structural formula according to Formula XVIII.

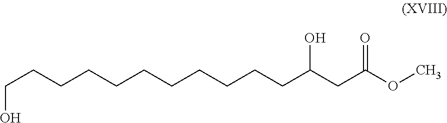 [SMILES: COC(=O)CC(O)CCCCCCCCCCCO]

Formula XVIII is referred to herein as 3,14-dihydroxy tetradecanoic acid methyl ester. With reference to Scheme 1, 3,14-dihydroxy tetradecanoic acid methyl ester is described as R1=CO₂CH₃; R2=OH; R3=H; R4=H; R5=CH₂OH; n+m=8.

In another embodiment, the disclosure provides a multifunctional fatty acid derivative molecule having a chemical structural formula according to Formula XIX.

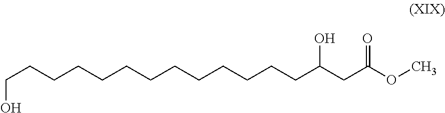 [SMILES: COC(=O)CC(O)CCCCCCCCCCCCCO]

Formula XIX is referred to herein as 3,16-dihydroxy hexadecanoic acid methyl ester. With reference to Scheme 1, 3,16-dihydroxy hexadecanoic acid methyl ester is described as R1=CO₂CH₃; R2=OH; R3=H; R4=H; R5=CH₂OH; n+m=10.

In another embodiment, the disclosure provides a multifunctional fatty acid derivative molecule having a chemical structural formula according to Formula XX.

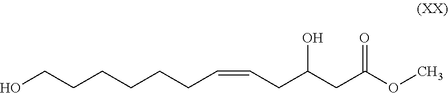 [SMILES: COC(=O)CC(O)C/C=C\CCCCCCO]

Formula XX is referred to herein as 3,12-dihydroxy dodecenoic acid methyl ester. With reference to Scheme 1, 3,12-dihydroxy dodecenoic acid methyl ester is described as R1=CO₂CH₃; R2=OH; R3=H; R4=H; R5=CH₂OH; n+m=6. In an exemplary embodiment, 3,12-dihydroxy dodecenoic acid methyl ester has the double bond is in cis and therefore the molecule of Formula XX is (z5) 3,12-dihydroxy dodecenoic acid methyl ester.

In another embodiment, the disclosure provides a multifunctional fatty acid derivative molecule having a chemical structural formula according to Formula XXI.

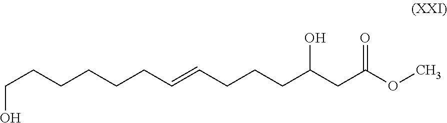 [SMILES: COC(=O)CC(O)CCC/C=C/CCCCCCO]

Formula XXI is referred to herein as 3,14-dihydroxy tetradecenoic acid methyl ester. With reference to Scheme 1, 3,14-dihydroxy tetradecenoic acid methyl ester is described as R1=CO₂CH₃; R2=OH; R3=H; R4=H; R5=CH₂OH; n+m=8. In an exemplary embodiment, 3,12-dihydroxy tetradecenoic acid methyl ester has the double bond is in cis and therefore the molecule of Formula XXI is (z7) 3,14-dihydroxy tetradecenoic acid methyl ester.

In another embodiment, the disclosure provides a multifunctional fatty acid derivative molecule having a chemical structural formula according to Formula XXII.

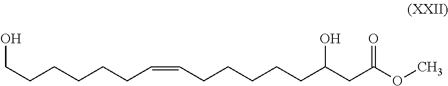 [SMILES: COC(=O)CC(O)CCCCC/C=C\CCCCCCO]

Formula XXII is referred to herein as 3,16-dihydroxy hexadecenoic acid methyl ester. With reference to Scheme 1, 3,16-dihydroxy hexadecenoic acid methyl ester is described as R1=CO₂CH₃; R2=OH; R3=H; R4=H; R5=CH₂OH; n+m=10. In an exemplary embodiment, 3,16-dihydroxy hexadecenoic acid methyl ester has the double bond is in cis and therefore the molecule of Formula XXII is (z9) 3,16-dihydroxy hexadecenoic acid methyl ester.

In another embodiment, the disclosure provides a multifunctional fatty acid derivative molecule having a chemical structural formula according to Formula XXIII.

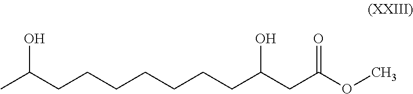 [SMILES: COC(=O)CC(O)CCCCCCCC(C)O]

Formula XXIII is referred to herein as 3,11-dihydroxy dodecanoic acid methyl ester. With reference to Scheme 1, 3,11-dihydroxy dodecanoic acid methyl ester is described as R1=CO₂CH₃; R2=OH; R3=H; R4=OH; R5=CH₃; n+m=6.

In another embodiment, the disclosure provides a multifunctional fatty acid derivative molecule having a chemical structural formula according to Formula XXIV.

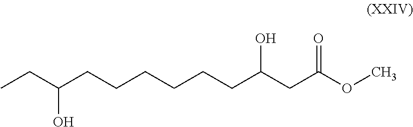 [SMILES: CCC(O)CCCCCCC(O)CC(=O)OC]

Formula XXIV is referred to herein as 3,10-dihydroxy dodecanoic acid methyl ester. With reference to Scheme 1, 3,10-dihydroxy dodecanoic acid methyl ester is described as R1=CO₂CH₃; R2=OH; R3=OH; R4=H; R5=CH₃; n=0 and m=6.

In another embodiment, the disclosure provides a multifunctional fatty acid derivative molecule having a chemical structural formula according to Formula XXV.

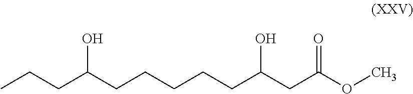 [SMILES: CCCC(O)CCCCCC(O)CC(=O)OC]

Formula XXV is referred to herein as 3,9-dihydroxy dodecanoic acid methyl ester. With reference to Scheme 1, 3,9-dihydroxy dodecanoic acid methyl ester is described as R1=CO₂CH₃; R2=OH; R3=OH; R4=H; R5=CH₃; n=O and m=6.

In another embodiment, the disclosure provides a multifunctional fatty acid derivative molecule having a chemical structural formula according to Formula XXVI.

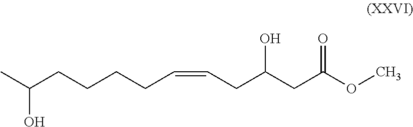 [SMILES: COC(=O)CC(O)C/C=C\CCCCC(C)O]

Formula XXVI is referred to herein as 3,11-dihydroxy dodecenoic acid methyl ester. With reference to Scheme 1, 3,11-dihydroxy dodecenoic acid methyl ester is described as R1=CO₂CH₃; R2=OH; R3=H; R4=OH; R5=CH₃; n+m=6. In an exemplary embodiment, 3,11-dihydroxy dodecenoic acid methyl ester has the double bond is in cis and therefore the molecule of Formula XXVI is (z5) 3,11-dihydroxy dodecenoic acid methyl ester.

In another embodiment, the disclosure provides a multifunctional fatty acid derivative molecule having a chemical structural formula according to Formula XXVII.

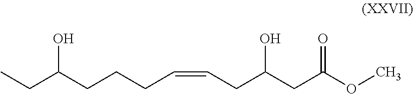 [SMILES: CCC(O)CCC/C=C\CC(O)CC(=O)OC]

Formula XXVII is referred to herein as 3,10-dihydroxy dodecenoic acid methyl ester. With reference to Scheme 1, 3,10-dihydroxy dodecenoic acid methyl ester is described as R1=CO₂CH₃; R2=OH; R3=H; R4=OH; R5=CH₃; n+m=6. In an exemplary embodiment, 3,10-dihydroxy dodecenoic acid methyl ester has the double bond is in cis and therefore the molecule of Formula XXVII is (z5) 3,10-dihydroxy dodecenoic acid methyl ester.

In another embodiment, the disclosure provides a multifunctional fatty acid derivative molecule having a chemical structural formula according to Formula XXVIII.

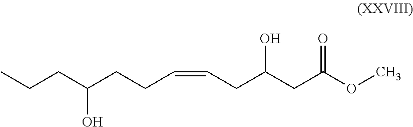 [SMILES: CCCC(O)CC/C=C\CC(O)CC(=O)OC]

Formula XXVIII is referred to herein as 3,9-dihydroxy dodecenoic acid methyl ester. With reference to Scheme 1, 3,9-dihydroxy dodecenoic acid methyl ester is described as R1=CO₂CH₃; R2=OH; R3=OH; R4=H; R5=CH₃; n=1 and m=5. In an exemplary embodiment, 3,9-dihydroxy dodecenoic acid methyl ester has the double bond is in cis and therefore the molecule of Formula XXVIII is (z5) 3,9-dihydroxy dodecenoic acid methyl ester.

In another embodiment, the disclosure provides a multifunctional fatty acid derivative molecule having a chemical structural formula according to Formula XXIX.

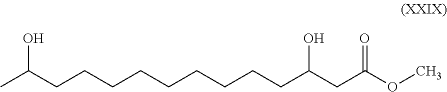 [SMILES: COC(=O)CC(O)CCCCCCCCCC(C)O]

Formula XXIX is referred to herein as 3,13-dihydroxy tetradecanoic acid methyl ester. With reference to Scheme 1, 3,13-dihydroxy tetradecanoic acid methyl ester is described as R1=CO₂CH₃; R2=OH; R3=H; R4=OH; R5=CH₃; n+m=8.

In another embodiment, the disclosure provides a multifunctional fatty acid derivative molecule having a chemical structural formula according to Formula XXX.

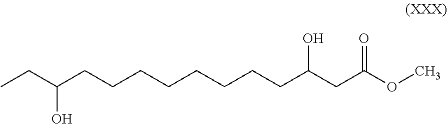 [SMILES: CCC(O)CCCCCCCCC(O)CC(=O)OC]

Formula XXX is referred to herein as 3,12-dihydroxy tetradecanoic acid methyl ester. With reference to Scheme 1, 3,12-dihydroxy tetradecanoic acid methyl ester is described as R1=CO₂CH₃; R2=OH; R3=OH; R4=H; R5=CH₃; n=0 and m=8.

In another embodiment, the disclosure provides a multifunctional fatty acid derivative molecule having a chemical structural formula according to Formula XXXI.

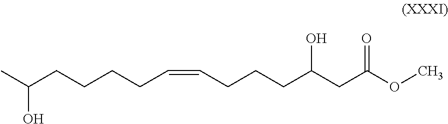 [SMILES: COC(=O)CC(O)CCC/C=C\CCCCC(C)O]

Formula XXXI is referred to herein as 3,13-dihydroxy tetradecenoic acid methyl ester. With reference to Scheme 1, 3,13-dihydroxy tetradecenoic acid methyl ester is described as R1=CO₂CH₃; R2=OH; R3=OH; R4=H; R5=CH₃; n+m=8. In an exemplary embodiment, 3,13-dihydroxy tetradecenoic acid methyl ester has the double bond is in cis and therefore the molecule of Formula XXXI is (z7) 3,13-dihydroxy tetradecenoic acid methyl ester.

In another embodiment, the disclosure provides a multifunctional fatty acid derivative molecule having a chemical structural formula according to Formula XXXII.

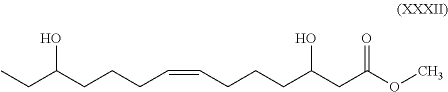 [SMILES: CCC(O)CCC/C=C\CCCC(O)CC(=O)OC]

Formula XXXII is referred to herein as 3,12-dihydroxy tetradecenoic acid methyl ester. With reference to Scheme 1, 3,12-dihydroxy tetradecenoic acid methyl ester is described as R1=CO₂CH₃; R2=OH; R3=OH; R4=H; R5=CH₃; n=0 and m=8. In an exemplary embodiment, 3,12-dihydroxy tetradecenoic acid methyl ester has the double bond is in cis and therefore the molecule of Formula XXXII is (z7) 3,12-dihydroxy tetradecenoic acid methyl ester.

In another embodiment, the disclosure provides a multifunctional fatty acid derivative molecule having a chemical structural formula according to Formula XXXIII.

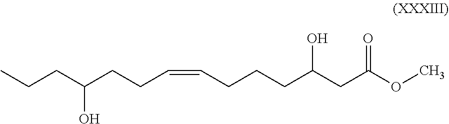 [SMILES: CCCC(O)CC/C=C\CCCC(O)CC(=O)OC]

Formula XXXIII is referred to herein as 3,11-dihydroxy tetradecenoic acid methyl ester. With reference to Scheme 1, 3,11-dihydroxy tetradecenoic acid methyl ester is described as R1=CO₂CH₃; R2=OH; R3=OH; R4=H; R5=CH₃; n=1 and m=7. In an exemplary embodiment, 3,11-dihydroxy tetradecenoic acid methyl ester has the double bond is in cis and therefore the molecule of Formula XXXIII is (z7) 3,11-dihydroxy tetradecenoic acid methyl ester.

In another embodiment, the disclosure provides a multifunctional fatty acid derivative molecule having a chemical structural formula according to Formula XXXIV.

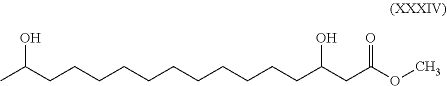 [SMILES: COC(=O)CC(O)CCCCCCCCCCCC(C)O]

Formula XXXIV is referred to herein as 3,15-dihydroxy hexadecanoic acid methyl ester. With reference to Scheme 1, 3,15-dihydroxy hexadecanoic acid methyl ester is described as R1=CO₂CH₃; R2=OH; R3=H; R4=OH; R5=CH₃; n+m=10.

In another embodiment, the disclosure provides a multifunctional fatty acid derivative molecule having a chemical structural formula according to Formula XXXV.

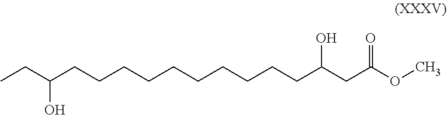 [SMILES: CCC(O)CCCCCCCCCCC(O)CC(=O)OC]

Formula XXXV is referred to herein as 3,14-dihydroxy hexadecanoic acid methyl ester. With reference to Scheme 1, 3,14-dihydroxy hexadecanoic acid methyl ester is described as R1=CO₂CH₃; R2=OH; R3=OH; R4=H; R5=CH₃; n=0 and m=10.

In another embodiment, the disclosure provides a multifunctional fatty acid derivative molecule having a chemical structural formula according to Formula XXXVI.

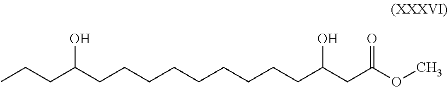 [SMILES: CCCC(O)CCCCCCCCCC(O)CC(=O)OC]

Formula XXXVI is referred to herein as 3,13-dihydroxy hexadecanoic acid methyl ester. With reference to Scheme 1, 3,13-dihydroxy hexadecanoic acid methyl ester is described as R1=CO₂CH₃; R2=OH; R3=OH; R4=H; R5=CH₃; n=1 and m=9.

In another embodiment, the disclosure provides a multifunctional fatty acid derivative molecule having a chemical structural formula according to Formula XXXVII.

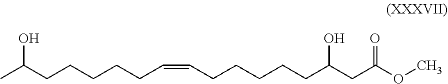 [SMILES: COC(=O)CC(O)CCCCC/C=C\CCCCCC(C)O]

Formula XXXVII is referred to herein as 3,15-dihydroxy hexadecenoic acid methyl ester. With reference to Scheme 1, 3,15-dihydroxy hexadecenoic acid methyl ester is described as R1=CO₂CH₃; R2=OH; R3=H; R4=OH; R5=CH₃; n+m=10. In an exemplary embodiment, 3,15-dihydroxy hexadecenoic acid methyl ester has the double bond is in cis and therefore the molecule of Formula XXXVIII is (z9) 3,15-dihydroxy hexadecenoic acid methyl ester.

In another embodiment, the disclosure provides a multifunctional fatty acid derivative molecule having a chemical structural formula according to Formula XXXVIII.

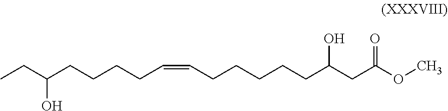 [SMILES: CCC(O)CCCC/C=C\CCCCCC(O)CC(=O)OC]

Formula XXXVIII is referred to herein as 3,14-dihydroxy hexadecanoic acid methyl ester. With reference to Scheme 1, 3,14-dihydroxy hexadecanoic acid methyl ester is described as R1=CO₂CH₃; R2=OH; R3=OH; R4=H; R5=CH₃; n=0 and m=10. In an exemplary embodiment, 3,13-dihydroxy hexadecenoic acid methyl ester has the double bond is in cis and therefore the molecule of Formula XXXVIII is (z9) 3,14-dihydroxy hexadecanoic acid methyl ester.

In another embodiment, the disclosure provides a multifunctional fatty acid derivative molecule having a chemical structural formula according to Formula XXXIX.

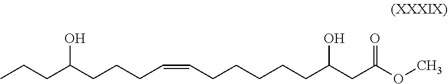 [SMILES: CCCC(O)CCC/C=C\CCCCCC(O)CC(=O)OC]

Formula XXXIX is referred to herein as 3,13-dihydroxy hexadecenoic acid methyl ester. With reference to Scheme 1, 3,13-dihydroxy hexadecenoic acid methyl ester is described as R1=CO₂CH₃; R2=OH; R3=OH; R4=H; R5=CH₃; n=1 and m=9. In an exemplary embodiment, 3,13-dihydroxy hexadecenoic acid methyl ester has the double bond is in cis and therefore the molecule of Formula XXXIX is (z9) 3,13-dihydroxy hexadecenoic acid methyl ester.

### Ethyl Esters

As noted above, the disclosure also provides multifunctional fatty acid methyl ester and ethyl esters. Exemplary methyl esters are disclosed above. Now, referring to the general formula provided by Scheme 1 (above) when R1=CO₂CH₂CH₃ and R2=OH the disclosure provides multifunctional fatty acid ethyl ester molecules having chemical structural formulas as follows:

In another embodiment, the disclosure provides a multifunctional fatty acid derivative molecule having a chemical structural formula according to Formula XL.

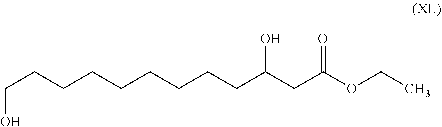 [SMILES: CCOC(=O)CC(O)CCCCCCCCCO]

Formula XL is referred to herein as 3,12-dihydroxy dodecanoic acid ethyl ester. With reference to Scheme 1, 3,12-dihydroxy dodecanoic acid ethyl ester is described as R1=CO₂CH₂CH₃; R2=OH; R3=H; R4=H; R5=CH₂OH; n+m=6.

In another embodiment, the disclosure provides a multifunctional fatty acid derivative molecule having a chemical structural formula according to Formula XLI.

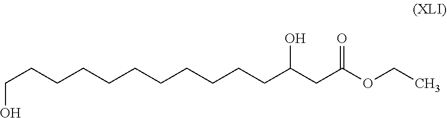 [SMILES: CCOC(=O)CC(O)CCCCCCCCCCCO]

Formula XLI is referred to herein as 3,14-dihydroxy tetradecanoic acid ethyl ester. With reference to Scheme 1, 3,14-dihydroxy tetradecanoic acid ethyl ester is described as R1=CO₂CH₂CH₃; R2=OH; R3=H; R4=H; R5=CH₂OH; n+m=8.

In another embodiment, the disclosure provides a multifunctional fatty acid derivative molecule having a chemical structural formula according to Formula XLII.

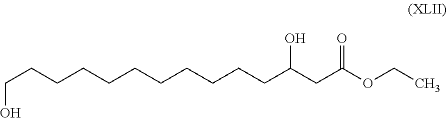 [SMILES: CCOC(=O)CC(O)CCCCCCCCCCCO]

Formula XLII is referred to herein as 3,16-dihydroxy hexadecanoic acid ethyl ester. With reference to Scheme 1, 3,16-dihydroxy hexadecanoic acid ethyl ester is described as R1=CO₂CH₂CH₃; R2=OH; R3=H; R4=H; R5=CH₂OH; n+m=10.

In another embodiment, the disclosure provides a multifunctional fatty acid derivative molecule having a chemical structural formula according to Formula XLIII.

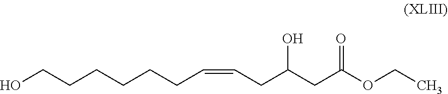 [SMILES: CCOC(=O)CC(O)C/C=C\CCCCCCO]

Formula XLIII is referred to herein as 3,12-dihydroxy dodecenoic acid ethyl ester. With reference to Scheme 1, 3,12-dihydroxy dodecenoic acid ethyl ester is described as R1=CO₂CH₂CH₃; R2=OH; R3=H; R4=H; R5=CH₂OH; n+m=6. In an exemplary embodiment, 3,12-dihydroxy dodecenoic acid ethyl ester has the double bond is in cis and therefore the molecule of Formula XLIII is (z5) 3,12-dihydroxy dodecenoic acid ethyl ester.

In another embodiment, the disclosure provides a multifunctional fatty acid derivative molecule having a chemical structural formula according to Formula XLIV.

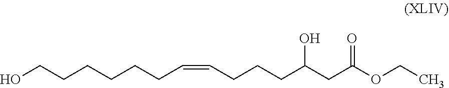 [SMILES: CCOC(=O)CC(O)CCC/C=C\CCCCCCO]

Formula XLIV is referred to herein as 3,14-dihydroxy tetradecenoic acid ethyl ester. With reference to Scheme 1, 3,14-dihydroxy tetradecenoic acid ethyl ester is described as R1=CO₂CH₂CH₃; R2=OH; R3=H; R4=H; R5=CH₂OH; n+m=8. In an exemplary embodiment, 3,14-dihydroxy tetradecenoic acid ethyl ester has the double bond is in cis and therefore the molecule of Formula XLIV is (z7) 3,14-dihydroxy tetradecenoic acid ethyl ester.

In another embodiment, the disclosure provides a multifunctional fatty acid derivative molecule having a chemical structural formula according to Formula XLV.

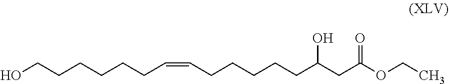 [SMILES: CCOC(=O)CC(O)CCCCC/C=C\CCCCCCO]

Formula XLV is referred to herein as 3,16-dihydroxy hexadecenoic acid ethyl ester. With reference to Scheme 1, 3,16-dihydroxy hexadecenoic acid ethyl ester is described as R1=CO₂CH₂CH₃; R2=OH; R3=H; R4=H; R5=CH₂OH; n+m=10. In an exemplary embodiment, 3,16-dihydroxy hexadecenoic acid ethyl ester has the double bond is in cis and therefore the molecule of Formula XLV is (z9) 3,16-dihydroxy hexadecenoic acid ethyl ester.

In another embodiment, the disclosure provides a multifunctional fatty acid derivative molecule having a chemical structural formula according to Formula XLVI.

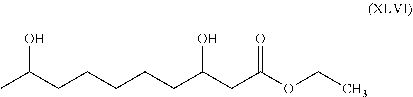 [SMILES: CCOC(=O)CC(O)CCCCCC(C)O]

Formula XLVI is referred to herein as 3,11-dihydroxy dodecanoic acid ethyl ester. With reference to Scheme 1, 3,11-dihydroxy dodecanoic acid ethyl ester is described as R1=CO₂CH₂CH₃; R2=OH; R3=H; R4=OH; R5=CH₃; n+m=6.

In another embodiment, the disclosure provides a multifunctional fatty acid derivative molecule having a chemical structural formula according to Formula XLVII.

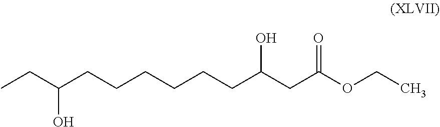 [SMILES: CCOC(=O)CC(O)CCCCCCC(O)CC]

Formula XLVII is referred to herein as 3,10-dihydroxy dodecanoic acid ethyl ester. With reference to Scheme 1, 3,10-dihydroxy dodecanoic acid ethyl ester is described as R1=CO₂CH₂CH₃; R2=OH; R3=OH; R4=H; R5=CH₃; n+m=6.

In another embodiment, the disclosure provides a multifunctional fatty acid derivative molecule having a chemical structural formula according to Formula XLVIII.

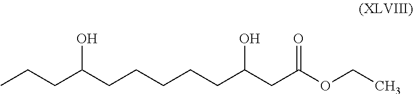 [SMILES: CCCC(O)CCCCCC(O)CC(=O)OCC]

Formula XLVIII is referred to herein as 3,9-dihydroxy dodecanoic acid ethyl ester. With reference to Scheme 1, 3,9-dihydroxy dodecanoic acid ethyl ester is described as R1=CO₂CH₂CH₃; R2=OH; R3=OH; R4=H; R5=CH₃; n=1 and m=5.

In another embodiment, the disclosure provides a multifunctional fatty acid derivative molecule having a chemical structural formula according to Formula XLIX.

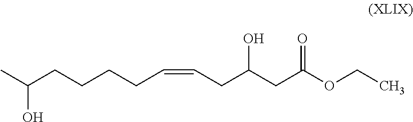 [SMILES: CCOC(=O)CC(O)C/C=C\CCCCC(C)O]

Formula XLIX is referred to herein as 3,11-dihydroxy dodecenoic acid ethyl ester. With reference to Scheme 1, 3,11-dihydroxy dodecenoic acid ethyl ester is described as R1=CO₂CH₂CH₃; R2=OH; R3=H; R4=OH; R5=CH₃; n+m=6. In an exemplary embodiment, 3,11-dihydroxy dodecenoic acid ethyl ester has the double bond is in cis and therefore the molecule of Formula XLIX is (z5) 3,11-dihydroxy dodecenoic acid ethyl ester.

In another embodiment, the disclosure provides a multifunctional fatty acid derivative molecule having a chemical structural formula according to Formula L.

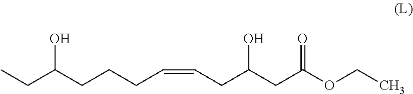 [SMILES: CCOC(=O)CC(O)C/C=C\CCCC(O)CC]

Formula L is referred to herein as 3,10-dihydroxy dodecenoic acid ethyl ester. With reference to Scheme 1, 3,10-dihydroxy dodecenoic acid ethyl ester is described as R1=CO₂CH₂CH₃; R2=OH; R3=OH; R4=H; R5=CH₃; n=0 and m=6. In an exemplary embodiment, 3,10-dihydroxy dodecenoic acid ethyl ester has the double bond is in cis and therefore the molecule of Formula L is (z5) 3,10-dihydroxy dodecenoic acid ethyl ester.

In another embodiment, the disclosure provides a multifunctional fatty acid derivative molecule having a chemical structural formula according to Formula LI.

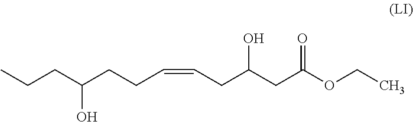 [SMILES: CCCC(O)CC/C=C\CC(O)CC(=O)OCC]

Formula LI is referred to herein as 3,9-dihydroxy dodecenoic acid ethyl ester. With reference to Scheme 1, 3,9-dihydroxy dodecenoic acid ethyl ester is described as R1=CO₂CH₂CH₃; R2=OH; R3=OH; R4=H; R5=CH₃; n=1 and m=5. In an exemplary embodiment, 3,9-dihydroxy dodecenoic acid ethyl ester has the double bond is in cis and therefore the molecule of Formula LI is (z5) 3,9-dihydroxy dodecenoic acid ethyl ester.

In another embodiment, the disclosure provides a multifunctional fatty acid derivative molecule having a chemical structural formula according to Formula LII.

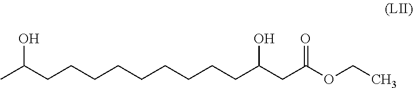 [SMILES: CCOC(=O)CC(O)CCCCCCCCCC(C)O]

Formula LII is referred to herein as 3,13-dihydroxy tetradecanoic acid ethyl ester. With reference to Scheme 1, 3,13-dihydroxy tetradecanoic acid ethyl ester is described as R1=CO₂CH₂CH₃; R2=OH; R3=H; R4=OH; R5=CH₃; n+m=8.

In another embodiment, the disclosure provides a multifunctional fatty acid derivative molecule having a chemical structural formula according to Formula LIII.

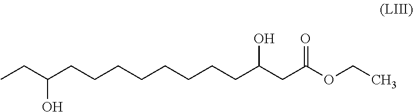 [SMILES: CCOC(=O)CC(O)CCCCCCCCC(O)CC]

Formula LIII is referred to herein as 3,12-dihydroxy tetradecanoic acid ethyl ester. With reference to Scheme 1, 3,12-dihydroxy tetradecanoic acid ethyl ester is described as R1=CO₂CH₂CH₃; R2=OH; R3=OH; R4=H; R5=CH₃; n=0 and m=8.

In another embodiment, the disclosure provides a multifunctional fatty acid derivative molecule having a chemical structural formula according to Formula LIV.

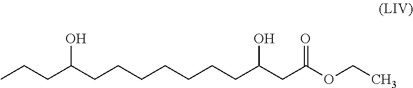 [SMILES: CCCC(O)CCCCCCCC(O)CC(=O)OCC]

Formula LIV is referred to herein as 3,11-dihydroxy tetradecanoic acid methyl ester. With reference to Scheme 1, 3,11-dihydroxy tetradecanoic acid methyl ester is described as R1=CO₂CH₂CH₃; R2=OH; R3=OH; R4=H; R5=CH₃; n=1 and m=7.

In another embodiment, the disclosure provides a multifunctional fatty acid derivative molecule having a chemical structural formula according to Formula LV.

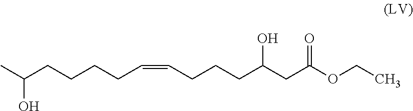 [SMILES: CCOC(=O)CC(O)CCC/C=C\CCCCC(C)O]

Formula LV is referred to herein as 3,13-dihydroxy tetradecenoic acid ethyl ester. With reference to Scheme 1, 3,13-dihydroxy tetradecenoic acid ethyl ester is described as R1=CO₂CH₂CH₃; R2=OH; R3=H; R4=OH; R5=CH₃; n+m=8. In an exemplary embodiment, 3,13-dihydroxy tetradecenoic acid ethyl ester has the double bond is in cis and therefore the molecule of Formula LV is (z7) 3,13-dihydroxy tetradecenoic acid ethyl ester.

In another embodiment, the disclosure provides a multifunctional fatty acid derivative molecule having a chemical structural formula according to Formula LVI.

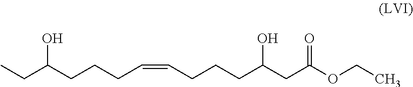 [SMILES: CCOC(=O)CC(O)CCC/C=C\CCCC(O)CC]

Formula LVI is referred to herein as 3,12-dihydroxy tetradecenoic acid ethyl ester. With reference to Scheme 1, 3,12-dihydroxy tetradecenoic acid ethyl ester is described as R1=CO₂CH₂CH₃; R2=OH; R3=OH; R4=H; R5=CH₃; n=0 and m=8. In an exemplary embodiment, 3,12-dihydroxy tetradecenoic acid ethyl ester has the double bond is in cis and therefore the molecule of Formula LVI is (z7) 3,12-dihydroxy tetradecenoic acid ethyl ester.

In another embodiment, the disclosure provides a multifunctional fatty acid derivative molecule having a chemical structural formula according to Formula LVII.

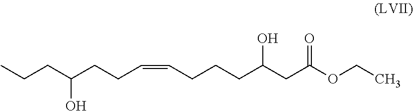 [SMILES: CCCC(O)CC/C=C\CCCC(O)CC(=O)OCC]

Formula LVII is referred to herein as 3,11-dihydroxy tetradecenoic acid ethyl ester. With reference to Scheme 1, 3,11-dihydroxy tetradecenoic acid ethyl ester is described as R1=CO₂CH₂CH₃; R2=OH; R3=OH; R4=H; R5=CH₃; n=1 and m=7. In an exemplary embodiment, 3,11-dihydroxy tetradecenoic acid ethyl ester has the double bond is in cis and therefore the molecule of Formula LVII is (z7) 3,11-dihydroxy tetradecenoic acid ethyl ester.

In another embodiment, the disclosure provides a multifunctional fatty acid derivative molecule having a chemical structural formula according to Formula LVIII.

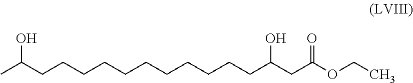 [SMILES: CCOC(=O)CC(O)CCCCCCCCCCCC(C)O]

Formula LVIII is referred to herein as 3,15-dihydroxy hexadecanoic acid ethyl ester. With reference to Scheme 1, 3,15-dihydroxy hexadecanoic acid ethyl ester is described as R1=CO₂CH₂CH₃; R2=OH; R3=H; R4=OH; R5=CH₃; n+m=10.

In another embodiment, the disclosure provides a multifunctional fatty acid derivative molecule having a chemical structural formula according to Formula LIX.

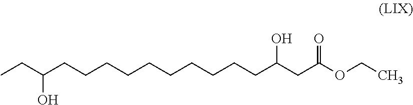 [SMILES: CCOC(=O)CC(O)CCCCCCCCCCC(O)CC]

Formula LIX is referred to herein as 3,14-dihydroxy hexadecanoic acid ethyl ester. With reference to Scheme 1, 3,14-dihydroxy hexadecanoic acid ethyl ester is described as R1=CO₂CH₂CH₃; R2=OH; R3=OH; R4=H; R5=CH₃; n=0 and m=10.

In another embodiment, the disclosure provides a multifunctional fatty acid derivative molecule having a chemical structural formula according to Formula LX.

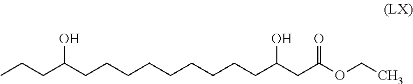 [SMILES: CCCC(O)CCCCCCCCCC(O)CC(=O)OCC]

Formula LX is referred to herein as 3,13-dihydroxy hexadecanoic acid ethyl ester. With reference to Scheme 1, 3,13-dihydroxy hexadecanoic acid ethyl ester is described as R1=CO₂CH₂CH₃; R2=OH; R3=OH; R4=H; R5=CH₃; n=1 and m=9.

In another embodiment, the disclosure provides a multifunctional fatty acid derivative molecule having a chemical structural formula according to Formula LXI.

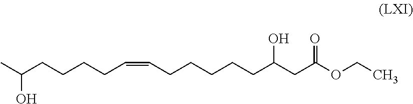 [SMILES: CCOC(=O)CC(O)CCCCC/C=C\CCCCC(C)O]

Formula LXI is referred to herein as 3,15-dihydroxy hexadecenoic acid ethyl ester. With reference to Scheme 1, 3,15-dihydroxy hexadecenoic acid ethyl ester is described as R1=CO₂CH₂CH₃; R2=OH; R3=H; R4=OH; R5=CH₃; n+m=7. In an exemplary embodiment, 3,15-dihydroxy hexadecenoic acid ethyl ester has the double bond is in cis and therefore the molecule of Formula LXI is (z9) 3,15-dihydroxy hexadecenoic acid ethyl ester.

In another embodiment, the disclosure provides a multifunctional fatty acid derivative molecule having a chemical structural formula according to Formula LXII.

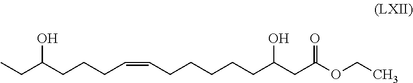 [SMILES: CCOC(=O)CC(O)CCCCC/C=C\CCCC(O)CC]

Formula LXII is referred to herein as 3,14-dihydroxy hexadecenoic acid ethyl ester. With reference to Scheme 1, 3,14-dihydroxy hexadecenoic acid ethyl ester is described as R1=CO₂CH₂CH₃; R2=OH; R3=OH; R4=H; R5=CH₃; n=0 and m=10. In an exemplary embodiment, 3,14-dihydroxy hexadecenoic acid ethyl ester has the double bond is in cis and therefore the molecule of Formula LXII is (z9) 3,14-dihydroxy hexadecenoic acid ethyl ester.

In another embodiment, the disclosure provides a multifunctional fatty acid derivative molecule having a chemical structural formula according to Formula LXIII.

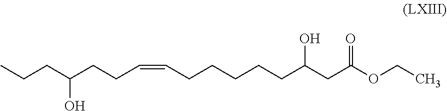 [SMILES: CCCC(O)CC/C=C\CCCCCC(O)CC(=O)OCC]

Formula LXIII is referred to herein as 3,13-dihydroxy hexadecenoic acid ethyl ester. With reference to Scheme 1, 3,13-dihydroxy hexadecenoic acid ethyl ester is described as R1=CO₂CH₂CH₃; R2=OH; R3=OH; R4=H; R5=CH₃; n=1 and m=9. In an exemplary embodiment, 3,13-dihydroxy hexadecenoic acid ethyl ester has the double bond is in cis and therefore the molecule of Formula LXIII is (z9) 3,13-dihydroxy hexadecenoic acid ethyl ester.

(iii) Molecules having R1=COOH and R2=H in Scheme 1

In an embodiment, the disclosure provides a multifunctional fatty acid derivative molecule having having R1=COOH and R2=H in Scheme 1, wherein n=0-3. Thus, in one embodiment R1=COOH and R2=H in Scheme 1, and n=0. In another embodiment, R1=COOH and R2=H in Scheme 1, and n=1. In another embodiment, R1=COOH and R2=H in Scheme 1, and n=2. In another embodiment, R1=COOH and R2=H in Scheme 1, and n=3.

In another embodiment, R1=COOH and R2=H in Scheme 1, and n≠4.

(iv) Novel Molecules Disclosed Herein not Described by Scheme 1

In addition to the molecules disclosed above which are conveniently described by Scheme 1, the disclosure further provides the following novel molecules which do not fit Scheme 1.

Thus, in one embodiment, the disclosure provides a multifunctional fatty acid derivative molecule having a chemical structural formula according to Formula LXVI.

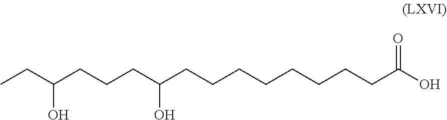 [SMILES: CCC(O)CCCC(O)CCCCCCCCC(=O)O]

Formula LXVI is referred to herein as 10,14-dihydroxy hexadecanoic acid. The molecule 10,14-dihydroxy hexadecanoic acid is not described with reference to Scheme 1.

In another embodiment, the disclosure provides a multifunctional fatty acid derivative molecule having a chemical structural formula according to Formula LXVII.

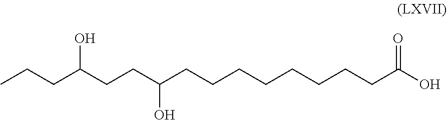 [SMILES: CCCC(O)CCC(O)CCCCCCCCC(=O)O]

Formula LXVII is referred to herein as 10,13-dihydroxy hexadecanoic acid. The molecule 10,13-dihydroxy hexadecanoic acid is not described with reference to Scheme 1.

In another embodiment, the disclosure provides a multifunctional fatty acid derivative molecule having a chemical structural formula according to Formula LXVIII.

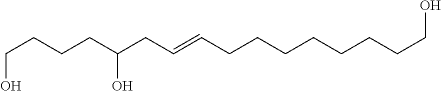 [SMILES: OCCCCCCCC/C=C/CC(O)CCCCO]

Formula LXVIII is referred to herein as 1,12,16-hexadecene triol. The molecule 1,12,16-hexadecene triol is not described with reference to Scheme 1.

In another embodiment, the disclosure provides a multifunctional fatty acid derivative molecule having a chemical structural formula according to Formula LXIX.

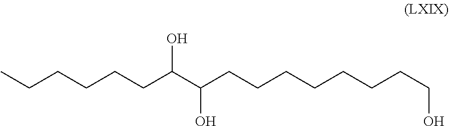 [SMILES: CCCCCCC(O)C(O)CCCCCCCCO]

Formula LXIX is referred to herein as 1,9,10-hexadecane triol. The molecule 1,9,10-hexadecane triol is not described with reference to Scheme 1.

In another embodiment, the disclosure provides a multifunctional fatty acid derivative molecule having a chemical structural formula according to Formula LXX.

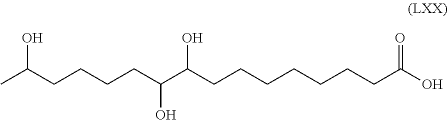 [SMILES: CC(O)CCCCC(O)C(O)CCCCCCCC(=O)O]

Formula LXX is referred to herein as 9,10,15 trihydroxy hexadecanoic acid. The molecule 9,10,15 trihydroxy hexadecanoic acid is not described with reference to Scheme 1.

In another embodiment, the disclosure provides a multifunctional fatty acid derivative molecule having a chemical structural formula according to Formula LXXI.

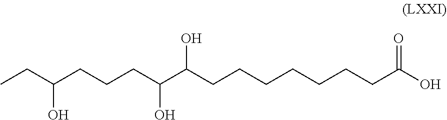 [SMILES: CCC(O)CCCC(O)C(O)CCCCCCCC(=O)O]

Formula LXXI is referred to herein as 9,10,14 trihydroxy hexadecanoic acid. The molecule 9,10,14 trihydroxy hexadecanoic acid is not described with reference to Scheme 1.

In another embodiment, the disclosure provides a multifunctional fatty acid derivative molecule having a chemical structural formula according to Formula LXXII.

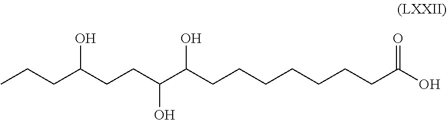 [SMILES: CCCC(O)CCC(O)C(O)CCCCCCCC(=O)O]

Formula LXXII is referred to herein as 9,10,13 trihydroxy hexadecanoic acid. The molecule 9,10,13 trihydroxy hexadecanoic acid is not described with reference to Scheme 1.

In another embodiment, the disclosure provides a multifunctional fatty acid derivative molecule having a chemical structural formula according to Formula LXXIII.

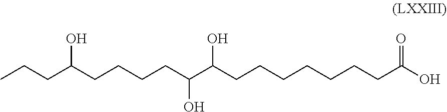 [SMILES: CCCC(O)CCCCC(O)C(O)CCCCCCCC(=O)O]

Formula LXXIII is referred to herein as 9,10,15 trihydroxy hexadecanoic acid. The molecule 9,10,15 trihydroxy hexadecanoic acid is not described with reference to Scheme 1.

In another embodiment, the disclosure provides a multifunctional fatty acid derivative molecule having a chemical structural formula according to Formula LXXIV.

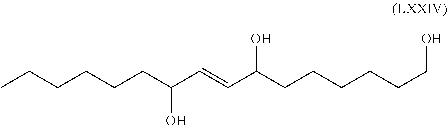 [SMILES: CCCCCCC(O)/C=C/C(O)CCCCCCO]

Formula LXXIV is referred to herein as 1,7,10-(8e)-hexadecene triol. The molecule 1,7,10-(8e)-hexadecene triol is not described with reference to Scheme 1.

In another embodiment, the disclosure provides a multifunctional fatty acid derivative molecule having a chemical structural formula according to Formula LXXV.

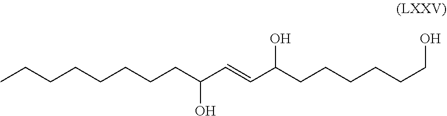 [SMILES: CCCCCCCCC(O)/C=C/C(O)CCCCCCO]

Formula LXXV is referred to herein as 1,7,10-(8e)-octadecene triol. The molecule 1,7,10-(8e)-octadecene triol is not described with reference to Scheme 1.

In another embodiment, the disclosure provides a multifunctional fatty acid derivative molecule having a chemical structural formula according to Formula LXXVI.

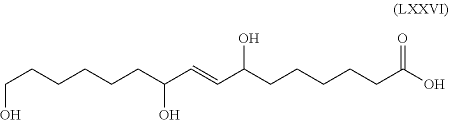 [SMILES: O=C(O)CCCCCC(O)/C=C/C(O)CCCCCCO]

Formula LXXVI is referred to herein as 7,10,16 trihydroxy-(8e)-hexadecenoic acid. The molecule 7,10,16 trihydroxy-(8e)-hexadecenoic acid is not described with reference to Scheme 1.

In another embodiment, the disclosure provides a multifunctional fatty acid derivative molecule having a chemical structural formula according to Formula LXXVII.

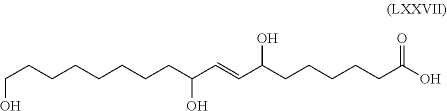 [SMILES: O=C(O)CCCCCC(O)/C=C/C(O)CCCCCCCCO]

Formula LXXVII is referred to herein as 7,10,18-trihydroxy-(8e)-octadecenoic acid. The molecule 7,10,18-trihydroxy-(8e)-octadecenoic acid is not described with reference to Scheme 1.

In another embodiment, the disclosure provides a multifunctional fatty acid derivative molecule having a chemical structural formula according to Formula LXXVIII.

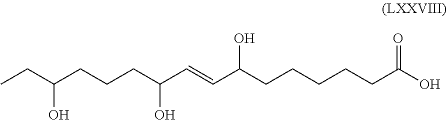 [SMILES: CCC(O)CCCC(O)/C=C/C(O)CCCCCC(=O)O]

Formula LXXVIII is referred to herein as 7,10,14-trihydroxy-(8e)-hexadecenoic acid. The molecule 7,10,14-trihydroxy-(8e)-hexadecenoic acid is not described with reference to Scheme 1.

In another embodiment, the disclosure provides a multifunctional fatty acid derivative molecule having a chemical structural formula according to Formula LXXIX.

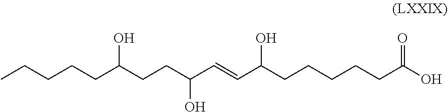 [SMILES: CCCCCC(O)CCC(O)/C=C/C(O)CCCCCC(=O)O]

Formula LXXIX is referred to herein as 7,10,13-trihydroxy-(8e)-octadecenoic acid. The molecule 7,10,13-trihydroxy-(8e)-octadecenoic acid is not described with reference to Scheme 1.

In another embodiment, the disclosure provides a multifunctional fatty acid derivative molecule having a chemical structural formula according to Formula LXXX.

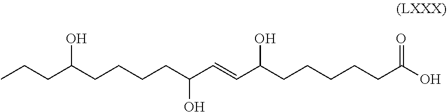 [SMILES: CCCC(O)CCCCC(O)/C=C/C(O)CCCCCC(=O)O]

Formula LXXX is referred to herein as 7,10,15-trihydroxy-(8e)-octadecenoic acid. The molecule 7,10,15-trihydroxy-(8e)-octadecenoic acid is not described with reference to Scheme 1.

In another embodiment, the disclosure provides a multifunctional fatty acid derivative molecule having a chemical structural formula according to Formula LXXXI.

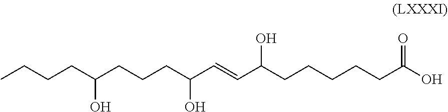 [SMILES: CCCCC(O)CCCC(O)/C=C/C(O)CCCCCC(=O)O]

Formula LXXXI is referred to herein as 7,10,14-trihydroxy-(8e)-octadecenoic acid. The molecule 7,10,14-trihydroxy-(8e)-octadecenoic acid is not described with reference to Scheme 1.

### Scheme 2

In another embodiment, the present disclosure provides novel multifunctional fatty acid derivative molecules having a general formula according to Scheme 2.

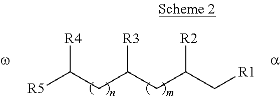

wherein
ω indicates the reduced end of the fatty acid derivative molecule and a indicates the carboxyl end of the fatty acid derivative molecule, and
   wherein
R1=-OH; —O₂H; —CH₂; —CH₂OH; —CHO; —CH₂NH₂; —CO₂H; —CO₂CH₃; —CO₂C₂H₅; —CO₂C₃H₇; —CO₂C₂H₃
R2=-H; —OH; —NH₂
R3=-H; —OH; —NH₂
R4=-H; —OH; —NH₂
R5=-CH₃; —CH₂; —CH₂OH; —CHO; —CH₂NH₂; —CO₂H; —CO₂CH₃; —CO₂C₂H₅; —CO₂C₃H₇; —CO₂C₂H₃;

wherein
m=1-10 and n=0-9 and wherein
if R1 is OH or —O₂H then . . . 3<m+n≤10; and if R1 is other than OH or —O₂H, then 2<m+n≤10, and

wherein
the multifunctional molecule has at least three functional groups comprising a heteroatom, and

wherein
when R5=CH₃ and R4=H then n=0 or 1; and

wherein
when R2=CH₃, then R4 does not=H; and

wherein
when R3=H, then n+m=p and p=3-10; unless R1=-OH or —O₂H in which case when R3-H then n+m=p and p=4-10; and

wherein
when the multifunctional molecule comprises a double bond that is not terminal, the double bond is in a position corresponding to an omega7 (ω-7) position; and

wherein
the multifunctional molecule is not 1,3,12-dodecane triol; is not 3,11-dihydroxy-tetradecanoic acid; is not 3,11-dihydroxy-tetradecanoic acid methyl ester and is not naturally occurring.

As will be apparent to one of skill in the art, when groups comprising a heteroatom are present at positions R2, R3 and/or R4, chiral center(s) are formed at the carbons to which the —OH groups are bound. Thus, the multifunctional molecules disclosed herein are useful as synthons for the production of chirally important compounds such as pharmaceuticals, nutraceuticals, pesticides, herbicides, flavors, fragrances, solvents, bioactive compounds, etc. Double bonds, if present, can be either (Z) or (E).

### (i) Molecules Having R1=COOH and R2=-NH₂ in Scheme 2

Referring to the general formula provided by Scheme 2 (above) when R1=COOH and R2=NH₂ the disclosure provides multifunctional molecules having chemical structural formulas as follows:

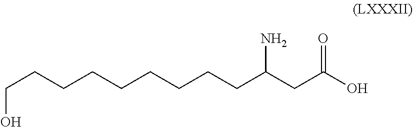

Formula LXXXII is referred to herein as 3-amino, 12-hydroxy-dodecanoic acid. With reference to Scheme 2, 3-amino, 12-hydroxy-dodecanoic acid is described as R1=CO₂H; R2=NH₂; R3=H; R4=H; R5=CH₂ OH; and m+n=p=6.

In another embodiment, the disclosure provides a multifunctional fatty acid derivative molecule having a chemical structural formula according to Formula LXXXIII.

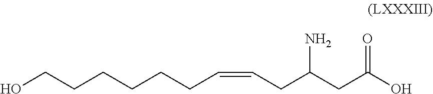 [SMILES: NC(C/C=C\CCCCCCO)CC(=O)O]

Formula LXXXIII is referred to herein as 3-amino, 12-hydroxy-dodecenoic acid. With reference to Scheme 2, 3-amino, 12-hydroxy dodecenoic acid is described as R1=CO₂H; R2=NH₂; R3=H; R4=H; R5=CH₂ OH; and m+n=p=6.

(ii) Molecules Having R1=CH₂OH and R2=-NH₂ in Scheme 2

Referring to the general formula provided by Scheme 2 (above) when R1=COOH and R2=NH₂ the disclosure provides multifunctional molecules having chemical structural formulas as follows:

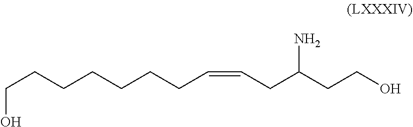

Formula LXXXIV is referred to herein as 3-amino dodecene 1,12-diol. With reference to Scheme 2, 3-amino dodecene 1,12-diol is described as R1=CH₂OH; R2=NH₂; R3=H; R4=H; R5=CH₂ OH; and m+n=p=6.

In another embodiment, the disclosure provides a multifunctional fatty acid derivative molecule having a chemical structural formula according to Formula LXXXV.

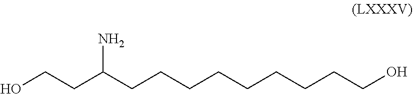 [SMILES: NC(CCO)CCCCCCCCCO]

Formula LXXXV is referred to herein as 3-amino dodecane 1,12-diol. With reference to Scheme 2, 3-amino dodecane 1,12-diol is described as R1=CO₂H; R2=NH₂; R3=H; R4=H; R5=CH₂ OH; and m+n=p=6.

(iii) Molecules Having R1=COOH and R5=-CH₂NH₂ in Scheme 2

Referring to the general formula provided by Scheme 2 (above) when R1=COOH and R5=CH₂NH₂ the disclosure provides multifunctional molecules having chemical structural formulas as follows:

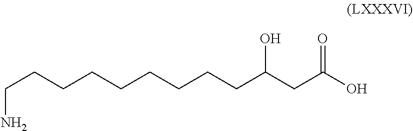

Formula LXXXVI is referred to herein as 3-hydroxy, 12-amino-dodecanoic acid. With reference to Scheme 2, 3-hydroxy, 12-amino-dodecanoic acid is described as R1=CO₂H; R2=OH; R3=H; R4=H; R5=CH₂NH₂; and m+n=p=6.

In another embodiment, the disclosure provides a multifunctional fatty acid derivative molecule having a chemical structural formula according to Formula LXXXVII.

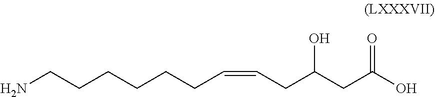 [SMILES: NCCCCCC/C=C\CC(O)CC(=O)O]

Formula LXXXV is referred to herein as 3-hydroxy, 12-amino dodecenoic acid. With reference to Scheme 2, 33-hydroxy, 12-amino dodecenoic acid is described as R1=CO₂H; R2=OH; R3=H; R4=H; R5=CH₂NH₂; and m+n=p=6.

(iv) Molecules Having R1=CH₂OH and R5=CH₂NH₂ in Scheme 2

Referring to the general formula provided by Scheme 2 (above) when R1=COOH and R5=CH₂NH₂ the disclosure provides multifunctional molecules having chemical structural formulas as follows:

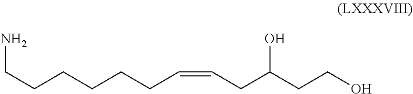

Formula LXXXVIII is referred to herein as 12-amino dodecene 1,3-diol. With reference to Scheme 2, 12-amino dodecene 1,3-diol is described as R1=CH₂OH; R2=OH; R3=H; R4=H; R5=CH₂NH₂; and m+n=p=6.

In another embodiment, the disclosure provides a multifunctional fatty acid derivative molecule having a chemical structural formula according to Formula LXXXIX.

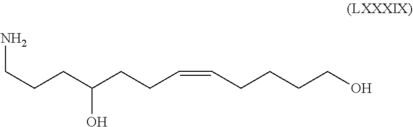 [SMILES: NCCCC(O)CC/C=C\CCCCO]

Formula LXXXIX is referred to herein as 12-amino dodecene 1,9-diol. With reference to Scheme 2, 12-amino dodecene 1,9-diol is described as R1=CH₂OH; R2=H; R3=OH; R4=H; R5=CH₂NH₂; and m=5 n=2.

In another embodiment, the disclosure provides a multifunctional fatty acid derivative molecule having a chemical structural formula according to Formula XC.

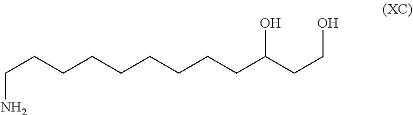 [SMILES: NCCCCCCCCCC(O)CCO]

Formula XC is referred to herein as 12-amino dodecane 1,3-diol. With reference to Scheme 2, 12-amino dodecane 1,3-diol is described as R1=CH₂OH; R2=OH; R3=H; R4=H; R5=CH₂NH₂; and m+n=p=6.

In another embodiment, the disclosure provides a multifunctional fatty acid derivative molecule having a chemical structural formula according to Formula XCI.

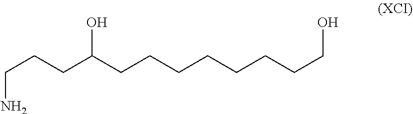 [SMILES: NCCCC(O)CCCCCCCCO]

Formula XCI is referred to herein as 12-amino-dodecane-1,9-diol. With reference to Scheme 2, 12-amino-dodecane-1,9-diol is described as R1=CH₂OH; R2=H; R3=OH; R4=H; R5=CH₂NH₂; and m=5 n=1.

1. Physical Properties Multifunctional fatty Acid Derivative Molecules

All of the multifunctional fatty acid derivative molecules (MFM) disclosed herein comprise a chiral center at R2, R3, and R4 when R2, R3 and/or R4 are hydroxyl groups. Additionally, the unsaturated MFM disclosed herein, also comprise a double bond. Thus, the MFM disclosed herein are able to undergo a wide array of chemical reactions to form a large variety of molecules. Thus, the MFM disclosed herein find use as unique chemicals which further provide for a number of unique and useful derivative molecules.

a. Hydroxyl Groups

In exemplary embodiments, the multifunctional molecules disclosed herein comprise hydroxyl functional groups. As is generally known in the art, the chemistry of polyols is much the same as that of alcohols. Thus, because of the polar nature of the —OH bond multifunctional molecules as disclosed herein readily form hydrogen bonds with other multifunctional molecules bearing hydroxyl groups or other hydrogen-bonding systems (e.g. water). Thus, multifunctional molecules bearing hydroxyl groups generally have relatively high melting and boiling points by comparison with analogous alkanes and relatively high solubility in aqueous media. (see e.g., Organic Chemistry ninth edition Francis Carey and Robert Giuliano (2013) supra).

The hydroxyl functional groups may participate in the large number of chemical reactions characteristic of hydroxyl groups. Thus, in one exemplary embodiment, the hydroxyl functional groups participate in nucleophilic substitution reactions wherein the hydroxyl acts as a leaving group or where —OH or —O— functions as a nucleophile e.g., substitution with a halide.

In other exemplary embodiments, the hydroxyl functional groups participate in nucleophilic addition reactions wherein the hydroxyl group acts as the nucleophile thereby forming acetals with aldehydes or ketones. Exemplary nucleophilic addition reactions include e.g., glycosylation reactions, which are discussed in more detail herein below.

In still other exemplary embodiments, the hydroxyl functional groups participate in nucleophilic acyl substitution reactions wherein the hydroxyl group acts as the nucleophile to form esters with carboxylic acids and carboxylic acid derivatives e.g., to form fatty esters.

In still other exemplary embodiments, the hydroxyl functional groups participate in elimination reactions wherein the hydroxyl group is removed as water and a carbon double bond (alkene) is formed.

In still other exemplary embodiments, the hydroxyl functional groups participate in oxidation reactions wherein the hydroxyl group is converted to a carbonyl group (C═O) thus producing a carbonyl compound. In oxidation reactions the resulting carbonyl compound may be an aldehyde, a ketone, or a carboxylic acid depending on the the oxidizing agent used (see e.g., Organic Chemistry 9th Edition, Francis Carey and Robert Giuliano (2013) supra).

Thus, the multiple hydroxyl functional groups of the multifunctional molecules disclosed herein make a wide variety of reactions possible and this in turn offers the possibility of numerous derivatives having unique and useful properties.

For example in some embodiments, a recombinant microbe that expresses a heterologous biochemical pathway comprising a thioesterase, at least one hydroxylating enzyme, an alcohol dehydrogenase or oxidase and a transaminase produces multifunctional fatty acid molecules with an amino group. Suitable thioesterases include any polypeptides that, when expressed in a microorganism in the presence of a carbon source, catalyze the production of fatty acids including 3-hydroxy fatty acids, e.g., enzymes having an Enzyme Commission number (EC 3.1.2.). Exemplary thioesterases include e.g., FatB1 from Umbellularia californica (Q41635) or PhaG from Pseudomonas putida (AAN67031).

Thus, in one embodiment, a recombinant microbe comprising a thioesterase such as FatB1 from Umbellularia californica, an alcohol oxidase such as AlkJ from Pseudomonas putida, a transaminase such as CV_2025 from Chromobacterium violaceum and a ω-hydroxylase such as cyp153A from Marinobacter aquaeolei produces the trifunctional molecules: 3-hydroxy, 12-amino dodecanoic acid; 3-amino, 12-hydroxy dodecanoic acid; (z5)3-hydroxy, 12-amino dodecenoic acid and (z5) 3-amino 12-hydroxy dodecenoic acid when the recombinant microbe is grown on a simple carbon source.

In another embodiment, a recombinant microbe comprising an thioesterase such as FatB1 from Umbellularia californica, an alcohol oxidase such as AlkJ from Pseudomonas putida, a transaminase such as CV_2025 from Chromobacterium violaceum, a carboxylic acid reductase such as CarB from Mycobacterium smegmatis and an ω-hydroxylase such as cyp153A from Marinobacter aquaeolei produces the trifunctional molecules 12-amino dodecane-1,3-diol; 3-amino dodecane-1,12-diol; 12-amino dodecane-1,9-diol; (z5)12-amino dodecene-1,3-diol; (z5)3-amino dodecene-1,12-diol and (z5)12-amino dodecene-1,9-diol.

Table 5 herein below discloses heterologous enzymes suitable for converting hydroxyl groups in multifunctional molecules into other functional groups. Further, Table 5 discloses the reactions catalyzed by the enzymes. Exemplary functional groups to which hydroxyl groups can be converted include e.g., oxo, carboxyl, amino, 0-acetyl, methoxy, ester, etc. Exemplary enzymes suitable for making these modifications includes dehydrogenases, oxidases, transaminases, acetyl-transferases, methyltransferases and ester synthases.

b. Chirality

Chiral molecules, such as multifunctional molecules disclosed herein, which may have a chiral center at R1, R2, R3 and/or R4, are building blocks for the synthesis of compounds e.g., pharmaceuticals, nutraceuticals, etc., which are affected by stereochemistry. Since most isomers of chiral drugs exhibit marked differences in biological activities such as e.g., pharmacology, toxicology, pharmacokinetics, biorecognition, metabolism, etc., chirality is an important property to consider e.g., in drug design. Indeed, selecting the appropriate enantiomer can have profound effect on the biological properties of a molecule. Thus, the novel multifunctional molecules disclosed herein provide building blocks for the synthesis of compounds such as e.g., pharmaceuticals, which are affected by stereochemistry.

The 3-hydroxy functionality of the multifunctional fatty acid derivative molecules disclosed herein may form a stereo center at the carbon bearing the hydroxy functionality, thereby providing a point of chirality for the molecule.

The stereoisomer of a multifunctional fatty acid derivative molecule that is produced by a microorganism depends on the selectivity of the fatty acid biosynthesis pathway (FAS) from which it is produced. By manipulating which FAS enzymes are responsible for synthesis of a multifunctional fatty acid derivative molecule the chirality of the resulting multifunctional fatty acid derivative molecule can be controlled.

For example, in an exemplary embodiment, the native E. coli FAS is exploited to produce the (R) enantiomer of multifunctional fatty acid derivative molecule. In this embodiment, the chiral center of the multifunctional fatty acid derivative molecule is created by the activity of by 3-ketoacyl-ACP reductase, an enzyme encoded by the FabG gene in E. coli. The activity of 3-ketoacyl-ACP reductase produces (R)-3-hydroxyl acyl ACP which can then enter engineered enzymatic pathway(s).

In other exemplary embodiments, the beta-oxidation pathway is exploited to produce the (S) enantiomer of a multifunctional fatty acid derivative molecule. In this embodiment, the (S) enantiomer of the multifunctional fatty acid derivative molecule is prepared by causing an accumulation of (S)-3-hydroxy acyl CoA which is an intermediate in the degradation of fatty acids through the beta-oxidation pathway. The excess (S)-3-hydroxy-acyl CoA is then converted to the (S) enantiomer of the multifunctional fatty acid derivative molecule through the action of fatty alcohol forming polypeptides, thioesterases or ester synthases.

Therefore, in an exemplary embodiment, to prepare the (S) enantiomer of an multifunctional fatty acid derivative molecule, available free fatty acids are first converted to acyl-CoAs by acyl-CoA synthase, a reaction catalyzed by FadD in E. coli (and homologs in other microorganisms). The resulting acyl-CoAs are then oxidized to trans-2-enoyl-CoA by fatty acyl-CoA dehydrogenase, a reaction catalyzed by FadE in E. coli (and homologs in other microorganisms). The resulting trans-2-enoyl-CoA is then hydrated to (S)-3-hydroxy-acyl-CoA by 2-trans-enoyl-CoA hydratase/(S)-3-hydroxy-acyl-CoA dehydratase, a reaction catalyzed by FadB in E. coli (and homologs in other microorganisms).

In the wild-type beta-oxidation pathway, (S)-3-hydroxy-acyl-CoA is then further oxidized to 3-keto-acyl-CoA by 3-keto-acyl-CoA dehydrogenase, a reaction also catalyzed by FadB in E. coli (and homologs in other microorganisms). The resulting 3-keto-acyl-CoA is thiolyzed to acyl-CoA and acetyl-CoA by 3-ketoacyl-CoA thiolase, a reaction catalyzed by FadA in E. coli (and homologs in other microorganisms).

In one exemplary embodiment, accumulation of (S)-3-hydroxy-acyl-CoA, is caused by selectively blocking the dehydrogenase activity of 3-keto-acyl-CoA dehydrogenase (FadB) to prevent the oxidation of (S)-3-hydroxy-acyl-CoA to 3-keto-acyl-CoA. In exemplary embodiments, selective blocking of the (S)-3-hydroxy-acyl-CoA dehydrogenase activity of FadB is achieved by mutation of Histidine 450 in the E. coli FadB gene (see e.g., He XY and Yang SY (1996) Biochemistry 35(29):9625-9630). (S)-3-hydroxy-acyl CoA accumulated in the cell is then converted to the (S) enantiomer of the multifunctional fatty acid derivative molecule, through the action of fatty alcohol forming polypeptides, thioesterases or ester synthases such as those disclosed e.g., in WO 2016/011430 A1.

Determination/confirmation of the resulting enantiomer configuration is achieved by any method known in the art e.g., by non-chromatographic techniques as polarimetry, by nuclear magnetic resonance, isotopic dilution, calorimetry, and enzyme techniques. These techniques require pure samples, and no separation of enantiomers is involved. Quantitation (which does not require pure samples) and separation of enantiomers can be done simultaneously by chiral chromatography such as gas chromatography (GC) or high performance liquid chromatography (HPLC) using chiral columns (see e.g., Stereochemistry of Organic Compounds, Ernest L. Elil and Sanuel H. Wilen, 1994, John Wiley & Sons, Inc.). The chiral purity of products can be identified using chiral chromatographic methods such as chiral HPLC or LC/MS (see e.g., US Patent Application Publication Nos. US2008/0248539A1 and US2013/0052699A1).

c. Metathesis

As discussed above, the double bond of an unsaturated multifunctional fatty acid derivative molecule, may be in either (E) configuration or (Z) configuration.

In general, unsaturated fatty acid derivative molecules produced utilizing microbes as disclosed hereinabove carry the double bond in (Z) configuration. However, as will be discussed herein below, methods are available to rearrange the (Z) double bond of an unsaturated fatty acid derivative molecule such that the double bond is produced in (E) configuration.

Multifunctional fatty acid derivative molecules produced as disclosed herein have a double bond predominantly in (Z) configuration. In some exemplary embodiments an unsaturated multifunctional fatty acid derivative molecule has a non-terminal double bond between the seventh and eighth carbons from the reduced end of the multifunctional fatty acid derivative molecule (in the co-7 position). In exemplary embodiments, the double bond in the co-7 position is in cis (Z) configuration.

U.S. Pat. No. 9,163,267 teaches methods for producing an olefin by contacting a composition comprising at least one omega-7-olefinic fatty acid or derivative thereof with a cross metathesis catalyst under conditions allowing a cross metathesis transformation, wherein the at least one omega-7-olefinic fatty acid or derivative thereof was produced in a genetically engineered microorganism. Thus, in exemplary embodiments, methods such as those disclosed in U.S. Pat. No. 9,163,267 are used to prepare a (E) isomer of an unsaturated (Z)-multifunctional fatty acid derivative e.g., (E) isomer of (z5) 1,3,12 dodecenetriol, made using engineered microbes as disclosed herein above. As is well known in the art, in cross metathesis reactions, the (Z)-(E) selectivity is typically biased towards the formation of the (E)-isomer (see e.g., Naeimeh Bahri-Laleh et al., (2011) Beilstein J. Org. Chem. 7:40-45).

2. Assaying for Multifunctional Fatty Acid Derivatives

In exemplary embodiments, multifunctional fatty acid derivatives are identified by assaying for the production of multifunctional fatty acid derivatives (e.g., 1,3,10 dodecanetriol, (z5) 1,3,12 dodecenetriol, 1,3,11 dodecane triol, etc.) by a recombinant microbial host strain. In exemplary embodiments, Gas-Chromatography with Flame-Ionization Detection (GC-FID) is used to assay the multifunctional acid derivative. GC-FID is known in the art (see e.g., Adlard, E. R.; Handley, Alan J. (2001). Gas chromatographic techniques and applications. London: Sheffield Academic). However, any appropriate method for quantitation and analysis may be used e.g., mass spectrometry (MS), Gas Chromatography-mass spectrometry (GC-MS), liquid chromatography-mass spectrometry (LC-MS), thin layer chromatography (TLC), etc.

3. Multifunctional Fatty Acid Derivative Molecules as Building Blocks for Polyols, Surfactants, Polyesters and Polyurethanes

Multifunctional fatty acid derivatives such as the multifunctional molecules disclosed herein, have applications as e.g., polyols, surfactants, and/or monomers in a variety of polymers, including but not limited to polyesters and polyurethanes.

In exemplary embodiments, the hydroxyl functional groups of multifunctional molecules are used to prepare polyurethanes in at least two different broad sets of chemistry: isocyanate-based polyurethanes and non-isocyanate polyurethanes.

In exemplary embodiments, multifunctional molecules act as polyols in standard isocyanate-based polyurethanes; or with mixed functionalities, available hydroxyl group(s) are reacted with isocyanates. As is known in the art, isocyanate reactions are promoted by ultraviolet light or by catalysts such as e.g., dibutyltin dilaurate or bismuth octanoate by methods known in the art (see e.g., Y. Li et al., Bio-based Polyols and Polyurethanes, Springer Briefs in Green Chemistry for Sustainability, DOI 10.1007/978-3-319-21539-6_2). Many different isocyanates, ranging from linear to aromatic, may be used; and techniques for preparing the polymer may or may not go through a pre-polymer phase, for instance prepping the available hydroxy groups, triol, or polyol with isocyanate groups (see e.g., U.S. Pat. No. 4,532,316). The available hydroxy groups of the multifunctional molecules disclosed herein may first be derivatized, for example by co-polymerizing with ethylene oxide by methods known in the art (see e.g., Anionic Polymerization: Principles, Practice, Strength, Consequences, Springer (2015) Nikos Hadjichristidis, Akira Hirao Eds.) thereby providing polyether polyols. The resulting polyether polyols may be used as-is in various applications, e.g., as building blocks of polyurethanes. Multifunctional molecules with mixed functionalities, for instance both hydroxyl and carboxylic groups, provide building blocks for copolymers, e.g., polyester polyurethanes.

In other embodiments certain specific arrangements of two or more hydroxyl groups in the multifunctional molecules disclosed herein provide chemical advantages in producing non-isocyanate polyurethanes. In particular, in exemplary embodiments a 1,3-hydroxy arrangement, for example as illustrated by 1,3,12-triol, is reacted with dimethyl carbonate or carbon dioxide to prepare a 6-membered cyclic carbonate ring to provide a molecule according to Formula XCII.

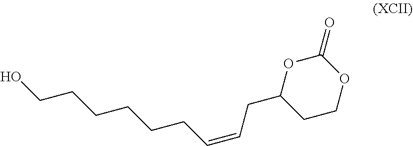 [SMILES: O=C1OCCC(C/C=C\CCCCCCO)O1]

The resulting 6-membered cyclic carbonate has a 30x reactivity versus a 5-membered cyclic carbonate from a 1,2-hydroxy moiety and is thus preferable in use (Maisonneuve et al, Chem. Rev., 2015). Catalysts useful for the preparation of carbonate derivatives on the hydroxyl groups of multifunctional molecules are readily selected by a person having ordinary skill in the art. Exemplary catalysts include e.g., 1,5,7-triazabicyclo[4.4.0]dec-5-ene with dimethyl carbonate (see e.g., Mutlu et al, Green Chem., 2012 pp. 1728-1735); various imidazolium or thiazolium carbene catalysts in the presence of cesium carbonate, dibromomethane, and CO₂ at atmospheric pressure (see e.g., Bobbink et al, Chem. Commun., 2016,52, 10787-10790); and CeO₂ with 2-cyanopyridine in the presence of CO₂ (see e.g., Honda et al, ACS Catal., 2014).

In exemplary embodiments, selective protection of the 1,3-hydroxy portion of the 1,3,12-triol allows for further chemistry on the C12 (terminal) hydroxyl group. Thus, for example, standard chemical conversion of the C12 OH group to an amine group in an alkylation with ammonia (see e.g., Bahn et al., ChemCatChem, 2011 pp. 1853-1864) is used. The resulting 1,3-carbonate and 12-amine provides a molecule according to Formula XCIII, which in exemplary embodiments, is self-polymerized as a non-isocyanate polyurethane.

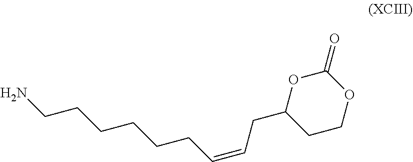 [SMILES: NCCCCCC/C=C\CC1CCOC(=O)O1]

Non-isocyanate polyurethanes are useful to the world because they allow the performance and properties of polyurethanes, used in such diverse applications from construction materials to medical devices, produced without the use of carcinogenic isocyanates. This enables safer working conditions for producers, commercial users, and even everyday consumers who may be exposed when using polyurethane products such as coatings and adhesives. It also has potential benefits of reducing environmental isocyanate exposure due to spills and waste removal.

The 1,3-hydroxy arrangement in the 1,3,12-triol has the advantage over 1,2-hydroxy arrangement in analogous structures in that there is less steric hindrance by the alcohol reaction centers. The derivatization of all three hydroxy groups on the 1,3,12-triol creates unusual branched structures—“three pointed stars”—that can form networks in solutions and polymer solids.

The 1,3,12-triol itself, or derivatives, may be useful in metal-ion chelation, useful in applications such as water treatment and catalyst development.

The arrangement of hydroxyl groups on the 1,3,12-triol molecule gives three places of hydrogen bonding, which in turn has implications for applied uses. For instance, it was observed that the 1,3,12-triol when mixed with 2-ethyl hexanol at a 60:40 triol:solvent ratio created a gel-like material that solidified. This thickening property is anticipated to act across a variety of solvents, at least with similar hydrogen bonding capability or dipole moment, and even in aqueous formulations. Thickening properties are useful in personal care formulations (such as lotions and shampoos), oil field applications (recovery methods), home and industrial cleaning products, and potential other fuel (semi-solid fuels) and industrial uses (low-volatiles cleaning, solid lubricants, etc.).

The 1,3,12-triol as-is or derivatized with polar groups may have further applications beyond thickening. In personal care applications, the 1,3,12-triol or its water-soluble derivatives may act as a humectant (retaining moisture on the skin) and as surface active ingredient for emulsifying and gentle cleaning. Gentle cleaning has the aim of removal of pollutive particles, external residues, excess oils, dead cell debris, and disruptive microbes without “stripping” the skin of protective oils and ceramides. In oil field recovery, surfactants with differentiated interfacial tension properties can help efficiently recover oil in conditions of high salinity and low temperatures (see e.g., Iglauer et al., Colloids and Surfaces A: Physiochemical and Engineering Aspects, 2009). Examples of derivatizing the 1,3,12-triol include water-soluble polyurethanes using standard polyurethane chemistries as described above; polyglycosides where mono-, di-, or polysaccharides are bound to one or more of the oxygens from the 1,3,12-triol; ethoxylation of the primary or all alcohols of the triol; or polyethylene glycol groups added to the alcohols of the 1,3,12-triol.

Chemistries for forming polyesters are well known in the art (see e.g., van der Ende, A. et al (2010) Macromolecules, 2010, 43 (13), pp 5665-5671; Modern Polyesters: Chemistry and Technology of Polyesters and Copolyesters, John Wiley & Sons, (2005) John Scheirs, Timothy E. Long Eds.) Exemplary chemistries include, but are not limited to, reactions catalyzed by heat and acid; lipase enzyme catalyzed polycondensation; the use of scandium triflates as catalysts, etc (see e.g., Diaz, A. et al., Macromolecules 2005, 38, 1048-1050).

In exemplary embodiments, 1,3,12-triol is reacted with diacids such as adipic acid to form “brush” polyesters (see e.g., W. Chen, et al. Macromolecules, 2017, 50 (11), pp 4089-4113). The resulting “brush” polyester from 1,3,12-triol have less crystallinity and, if highly networked, potentially more strength, rigidity, solvent-resistance, and scratch resistance than a polyester produced with alpha-omega diols.

In other exemplary embodiments, the 1,3,12-triol is a starting intermediate for differentiated performance properties in a wide variety of polymer applications. For example a multifunctional molecule e.g., 1,3,12-triol, is used in controlled mixed hydrophobic-hydrophilic copolymers for the creation of reverse micelles and dendrimer structures with highly specialized chelating and drug-delivery applications.

### II. Preparation of Multi-functional Fatty Acid Derivative Molecules

1. Introduction

Multi-functional molecules can be made by any method known in the art. Typically, multifunctional molecules are prepared from petrochemicals or oleochemicals, but such routes for preparation and synthesis of multifunctional molecules have many disadvantages and limitations. In particular, the synthesis of medium- to long-chain hydrocarbons (C₈ to C₁₆) with multiple hydroxy(—OH), oxo (=O), amino-(-NH₂) or carboxyl (CO₂H) groups in selective positions is extremely difficult and in many cases impractical starting from petrochemical feedstock.

Therefore, multifunctional molecules as disclosed herein are typically made using recombinant host cells e.g., using microbes e.g., bacterial cells, yeast cells, etc. that are engineered to produce multi-functional fatty acid derivative molecules. Accordingly, as disclosed herein, recombinant host cells are engineered and constructed to utilize nucleic acids and their corresponding polypeptides of enzymatic function in order to provide heterologous enzyme pathways for the in vivo production of the multifunctional fatty acid derivatives disclosed herein. Petrochemical or oleochemical feedstocks are not required, as the disclosed recombinant microbes use simple carbon sources to produce multifunctional fatty acid derivatives having desired carbon chain lengths and having specific functional groups placed in specific positions.

Therefore, in exemplary embodiments, the disclosed recombinant microbes use simple carbon sources to produce multifunctional fatty acid derivatives having specific functional groups placed in specific positions. In one exemplary embodiment, the disclosed recombinant microbes use simple carbon sources to produce multifunctional fatty acid derivatives having carbon chain lengths of ten (10) carbons to sixteen (16) carbons in length and having specific functional groups placed in specific positions (see e.g., Scheme 1, supra). In other exemplary embodiments, the disclosed recombinant microbes use simple carbon sources to produce multifunctional fatty acid derivatives having carbon chain lengths of between ten (10) carbons and sixteen (16) carbons in length and having functional groups placed in specific positions.

As will be discussed in detail below, the biosynthesis of multifunctional fatty acid derivatives takes advantage of the ability of the microbes fatty acid biosynthesis machinery to incorporate oxygen into medium to long carbon chains during fatty acid biosynthesis. In addition, oxygen molecules, e.g., hydroxyl groups, are incorporated into medium- to long-chain fatty acid derivatives by certain hydroxylases (also known as oxygenases) and hydratases. The hydroxylation reactions are usually regio- and stereo-selective thereby providing multifunctional fatty acid derivatives with chiral hydroxyl groups (R or S) in specific positions.

In exemplary embodiments, the incorporated hydroxyl groups are converted to other functional groups by employing additional enzymes to convert these hydroxyl groups into other functional groups, e.g. oxo (CHO), carboxyl (CO₂H), amino (CH₂NH₂), O-acetyl (CO₂C₂H₃), methoxy (COCH₃) or ester (CO₂C₂H₅, CO₂C₃H₇) groups.

Enzymes useful for making converting incorporated hydroxyl groups to other functional groups are disclosed herein below in Table 5.

Typically, the carbon chain length of the multifunctional molecules disclosed herein is between 8 and 16 carbons. In exemplary embodiments, the carbon chain (or equivalently, acyl chain) length of the multifunctional molecules disclosed herein is between 10 and 16 carbons. In exemplary embodiments, the multifunctional molecules disclosed herein comprise one double bond in either cis-(Z) or trans-(E) configuration. When the double bond is not terminal, the double bond is in the omega-7 (ω-7) position.

2. Host Cells and Host Cell Cultures

In view of the present disclosure, the person having ordinary skill in the art will appreciate that any of the embodiments contemplated herein may be practiced with any host cell or microorganism that can be genetically modified via the introduction of one or more nucleic acid sequences that code for the appropriate fatty acid biosynthetic enzymes. Accordingly, the recombinant microorganisms disclosed herein function as host cells and comprise one or more polynucleotide sequences that include an open reading frame that encode one or more fatty acid biosynthetic enzymes together with operably-linked regulatory sequences that facilitate expression of the fatty acid biosynthetic polypeptide(s) in the host cell.

Exemplary microorganisms that provide suitable host cells, include but are not limited to cells from the genus Escherichia, Bacillus, Lactobacillus, Zymomonas, Rhodococcus, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces. In some exemplary embodiments, the host cell is a Gram-positive bacterial cell. In other exemplary embodiments, the host cell is a Gram-negative bacterial cell. In some embodiments, the host cell is an E. coli cell. In other exemplary embodiments, the host cell is a Bacillus lentus cell, a Bacillus brevis cell, a Bacillus stearothermophilus cell, a Bacillus lichenoformis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, or a Bacillus amyloliquefaciens cell.

In still other exemplary embodiments, the host cell is a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhodococcus opacus cell, a Rhizomucor miehei cell, or a Mucor michei cell. In still other exemplary other embodiments, the host cell is a Streptomyces lividans cell or a Streptomyces murinus cell. In yet other embodiments, the host cell is an Actinomycetes cell. In some exemplary embodiments, the host cell is a Saccharomyces cerevisiae cell.

In still other exemplary embodiments, the host cell is a cell from a eukaryotic plant, algae, cyanobacterium, green-sulfur bacterium, green non-sulfur bacterium, purple sulfur bacterium, purple non-sulfur bacterium, extremophile, yeast, fungus, engineered organisms thereof, or a synthetic organism. In some exemplary embodiments, the host cell is a cell from Arabidopsis thaliana, Panicum virgatums, Miscanthus giganteus, Zea mays, Botryococcuse braunii, Chalamydomonas reinhardtii, Dunaliela salina, Thermosynechococcus elongatus, Synechococcus elongatus, Synechococcus sp., Synechocystis sp., Chlorobium tepidum, Chloroflexus auranticus, Chromatiumm vinosum, Rhodospirillum rubrum, Rhodobacter capsulatus, Rhodopseudomonas palusris, Clostridium ljungdahlii, Clostridiuthermocellum, or Pencillium chrysogenum. In some other exemplary embodiments, the host cell is from Pichia pastories, Saccharomyces cerevisiae, Yarrowia lipolytica, Schizosaccharomyces pombe, Pseudomonas fluorescens, Pseudomonas putida or Zymomonas mobilis. In still further exemplary embodiments, the host cell is a cell from Synechococcus sp. PCC 7002, Synechococcus sp. PCC 7942, or Synechocystis sp. PCC6803. In some exemplary embodiments, the host cell is a CHO cell, a COS cell, a VERO cell, a BHK cell, a HeLa cell, a Cvl cell, an MDCK cell, a 293 cell, a 3T3 cell, or a PC12 cell. In some exemplary embodiments, the host cell is an E. coli cell. In some exemplary embodiments, the E. coli cell is a strain B, a strain C, a strain K, or a strain W E. coli cell.

a. Expression of Heterologous Enzymatic Activities in Microorganisms

The expression of enzymatic activities in microorganisms and microbial cells for the production of fatty acid derivative molecules is taught e.g., in the following U.S. Pat. Nos. 9,133,406; 9,340,801; 9,200,299; 9,068,201; 8,999,686; 8,658,404; 8,597,922; 8,535,916; 8,530,221; 8,372,610; 8,323,924; 8,313,934; 8,283,143; 8,268,599; 8,183,028; 8,110,670; 8,110,093; and 8,097,439.

Therefore, in exemplary embodiments, the host cells or host microorganisms that are used to express polypeptides for biosynthesis of multifunctional fatty acid derivative molecules express heterologous thioesterase activity (E.C. 3.1.2.14, EC 3.1.2.20, etc.) for the production of fatty acids.

In other exemplary embodiments, the host cells or host microorganisms that are used to express polypeptides for biosynthesis of multifunctional fatty acid derivative molecules express ester synthase activity (E.C. 2.3.1.75) for the production of fatty esters. In another exemplary embodiment, the host cell has ester synthase activity (E.C. 2.3.1.75) and acyl-CoA synthase (FadD) (E.C. 6.2.1.3) activity for the production of fatty esters.

In another exemplary embodiment, the host cell has acyl-ACP reductase (AAR) (E.C. 1.2.1.80) activity and/or alcohol dehydrogenase activity (E.C. 1.1.1.1.) and/or fatty alcohol acyl-CoA reductase (FAR) (E.C. 1.1.1.-) activity and/or carboxylic acid reductase (CAR) (EC 1.2.99.6) activity for the production of fatty alcohols. In another exemplary embodiment, the host cell has acyl-CoA reductase (E.C. 1.2.1.50) activity, and acyl-CoA synthase (FadD) (E.C. 6.2.1.3) activity, for the production of fatty alcohols. In another exemplary embodiment, the host cell has acyl-ACP reductase (AAR) (E.C. 1.2.1.80) activity and alcohol dehydrogenase activity (E.C. 1.1.1.1.) for the production of fatty alcohols.

In another exemplary embodiment, the host cell has acyl-ACP reductase (AAR) (E.C. 1.2.1.80) activity for the production of fatty aldehydes. In another exemplary embodiment, the host cell has acyl-ACP reductase (AAR) (E.C. 1.2.1.80) activity and decarbonylase activity (aldehyde forming oxygenase) for the production of alkanes and alkenes.

In another exemplary embodiment, the host cell has OleA activity for the production of ketones. In another exemplary embodiment, the host cell has OleBCD activity for the production of internal olefins. In another exemplary embodiment, the host cell has decarboxylase activity for making terminal olefins.

In some exemplary embodiments, host cells or microorganisms that are used to express polypeptides for biosynthesis of multifunctional fatty acid derivative molecules comprise certain native enzyme activities that are upregulated or overexpressed in order to produce one or more particular fatty acid derivative(s) such as e.g., fatty esters, fatty alcohols, fatty amines, fatty aldehydes, bifunctional fatty acid derivatives, diacids, etc.

Typically, the multifunctional fatty acid derivatives disclosed herein are recovered from the culture medium and/or are isolated from the host cells. In one exemplary embodiment, the multifunctional fatty acid derivatives are recovered from the culture medium (extracellular). In another exemplary embodiment, the multifunctional fatty acid derivatives are isolated from the host cells (intracellular). In another exemplary embodiment, the multifunctional fatty acid derivatives are recovered from the culture medium and isolated from the host cells.

A fatty acid derivative composition produced by a host cell can be analyzed using methods known in the art, for example, Gas-Chromatography with Flame Ionization Detection (GC-FID) in order to determine the distribution of particular multifunctional fatty acid derivatives as well as chain lengths and degree of saturation of the components of the fatty acid derivative composition. Similarly, other compounds can be analyzed through methods well known in the art.

b. Genetic Alterations for Fine Tuning Recombinant Host Cells

In some exemplary embodiments, host cells comprise optional genetic manipulations and alterations can be used to enhance or otherwise fine tune the production of multifunctional fatty acid derivative molecules. As will be appreciated by a person having ordinary skill in the art, optional genetic manipulations can be used interchangeably from one host cell to another, depending on what other heterologous enzymes and what native enzymatic pathways are present in the host cell. Some optional genetic manipulations are discussed below.

### FadE

FadE (Acyl-CoA dehydrogenase) catalyzes the first step the first step in fatty acid utilization/degradation (p-oxidation cycle) which is the oxidation of acyl-CoA to 2-enoyl-CoA (see e.g., Campbell, J. W. and Cronan, J. E. Jr (2002) J. Bacteriol. 184(13): 3759-3764, Lennen, R. M. and Pfleger, B. F (2012) Trends Biotechnol. 30(12):659-667). Since fadE initiates the p-oxidation cycle, when E. coli lacks FadE, it cannot grow on fatty acids as a carbon source (see e.g., Campbell, J. W. and Cronan supra).

However, when E. coli is grown on a carbon source other than fatty acids e.g., grown on sugar, acetate, etc., fadE attenuation is optional because under such conditions fadE expression is repressed by FadR. Therefore, when cells are grown on a simple carbon source such as e.g., glucose, the fadE gene product is already attenuated. Accordingly, when grown on a carbon source other than fatty acids, a fadE mutation/deletion is optional.

fhuA

The gene fhuA codes for the TonA protein, which is an energy-coupled transporter and receptor in the outer membrane of E. coli (see e.g., V. Braun (2009) J Bacteriol. 191(11):3431-3436). The fhuA deletion allows the cell to become more resistant to phage attack. This phenotype can be beneficial in certain fermentation conditions. Its deletion is optional.

entD

For example, the entD gene codes for a phosphopantetheinyl transferase. Overexpression of native E. coli entD, a phosphopantetheinyl transferase, enables the activation of CarB from apo-CarB to holo-CarB, thereby allowing conversion of free fatty acids into fatty aldehydes, which can then be converted to fatty alcohols by a fatty aldehyde reductase see e.g., U.S. Pat. No. 9,340,801.

Overexpression of Non-Native and/or Native and/or Variants of Genes Involved in the Synthesis of Acyl-ACP

In some embodiments, the fatty acid biosynthetic pathway in the production host uses the precursors acetyl-CoA and malonyl-CoA. E. coli or other host organisms engineered to overproduce these components can serve as the starting point for subsequent genetic engineering steps to provide the specific output product (such as, fatty acids, fatty esters, hydrocarbons, fatty alcohols). Several different modifications can be made, either in combination or individually, to the host strain to obtain increased acetyl-CoA/malonyl-CoA/fatty acid and fatty acid derivative production see e.g., US Patent Application Publication 2010/0199548.

Other exemplary modifications of a host cell include e.g., overexpression of non-native and/or native and/or variants of genes involved in the synthesis of acyl-ACP. In general, by increasing acyl-ACP synthesis increases the amount of acyl-ACP, which is the substrate of thioesterases, estersynthases and acyl-ACP reductases. Exemplary enzymes that increase acyl-ACP production include e.g., enzymes that make up the “fatty acid synthase” (FAS). As is known in the art (see e.g., US 2010/0199548) FAS enzymes are a group of enzymes that catalyze the initiation and elongation of acyl chains. The acyl carrier protein (ACP) along with the enzymes in the FAS pathway control the length, degree of saturation, and branching of the fatty acids produced. Enzymes that comprise FAS include e.g., AccABCD, FabD, FabH, FabG, FabA, FabZ, FabI, FabK, FabL, FabM, FabQ, FabV, FabX,FabB, and FabF. Depending upon the desired product one or more of these genes can be attenuated or over-expressed.

Therefore, in exemplary embodiments a host strain may overexpress of one or more of the FAS genes. Exemplary FAS genes that may be overexpressed include e.g., fadR from Escherichia coli (NP_415705.1) fabA from Salmonella typhimurium (NP_460041), fabD from Salmonella typhimurium (NP_460164), fabG from Salmonella typhimurium (NP_460165), fabH from Salmonella typhimurium (NP_460163), fabV from Vibrio cholera (YP_001217283), and fabF from Clostridium acetobutylicum (NP_350156). In some exemplary embodiments, the overexpression of one or more of these genes, which code for enzymes and regulators in fatty acid biosynthesis, serves to further increase the titer of fatty-acid derivative compounds under particular culture conditions. In some exemplary embodiments, the wild-type E. coli strains MG1655 or W3110 (see e.g., Blattner, et al. (1997) 277(5331): 1453-1462; Jensen, K. F. (1993) J. Bact., 175(11): 3401-3407) are used as host strains.

3. Methods of Making Recombinant Host Cells and Cultures

Any method known in the art can be used to engineer host cells to produce fatty acid derivatives and/or fatty acid derivative compositions or other compounds. Methods for engineering host cells are well known in the art and are readily appreciated and accessible to the skilled practitioner. See e.g., Sambrook et al. (supra); Current Protocols in Molecular Biology (supra).

Generally, a polynucleotide (or gene) sequence is provided to the host cell by way of a recombinant vector that comprises a promoter operably linked to the fatty acid biosynthetic polynucleotide sequence of interest. Once a polynucleotide sequence(s) encoding fatty acid biosynthetic pathway polypeptide has been prepared and isolated, various methods may be used to construct expression cassettes, vectors and other DNA constructs. The skilled artisan is well aware of the genetic elements that must be present on an expression construct/vector in order to successfully transform, select and propagate the expression construct in host cells. Techniques for manipulation of nucleic acids such as subcloning nucleic acid sequences into expression vectors, labeling probes, DNA hybridization, and the like are described generally in e.g., Sambrook, et al., supra; Current Protocols in Molecular Biology, supra.

A number of recombinant vectors are available to those of skill in the art for use in the stable transformation/transfection of bacteria and other microorganisms (see e.g., Sambrook, et al., supra). Appropriate vectors are readily chosen by one of skill in the art.

Once an appropriate vector is identified and constructed, the appropriate transformation technique is readily chosen by the skilled practitioner. Exemplary transformation/transfection methods available to those skilled in the art include e.g., electroporation, calcium chloride transformation and etc., such methods being well known to the skilled artisan (see e.g., Sambrook, supra). In exemplary embodiments, polynucleotide sequences, comprising open reading frames encoding proteins and operably-linked regulatory sequences can be integrated into a chromosome of the recombinant host cells, incorporated in one or more plasmid expression system resident in the recombinant host cells, or both.

As will be appreciated by those skilled in the art, the design of the expression vector can depend on such factors as e.g., the choice of the host cell to be transformed, the level of expression of polypeptide desired, etc.

4. Recombinant Microbes Comprising a Heterologous Enzyme Pathway Capable of Producing Bifunctional Fatty Acid Derivative Molecules for the Production of Multifunctional Fatty Acid Derivative Molecules

As discussed above, a recombinant host cell comprising heterologous fatty acid biosynthetic polypeptides is used to produce particular types of multifunctional fatty acid derivatives. Thus, in some exemplary embodiments, the disclosure provides recombinant microbes that comprise heterologous enzyme pathway(s) capable of producing a bifunctional fatty acid derivative molecule and at least one heterologous hydroxylating enzyme. Therefore, in exemplary embodiments, a method for preparing a multifunctional molecule comprises: growing a recombinant microbe that comprises a heterologous enzyme pathway(s) capable of producing a bifunctional fatty acid derivative molecule and at least one heterologous hydroxylating enzyme in a culture medium that comprises a simple carbon source.

Exemplary disclosures that provide microbial strains that that comprise heterologous enzyme pathway(s) capable of producing a bifunctional fatty acid derivative molecule are known in the art see e.g., U.S. Patent Application Publication No. 2016/0130616 (LS48); U.S. Patent Application Publication No. 2017/0204436 (LS52); U.S. Patent Application Publication No. 2014/0215904 (LS35 β-OH esters), etc.

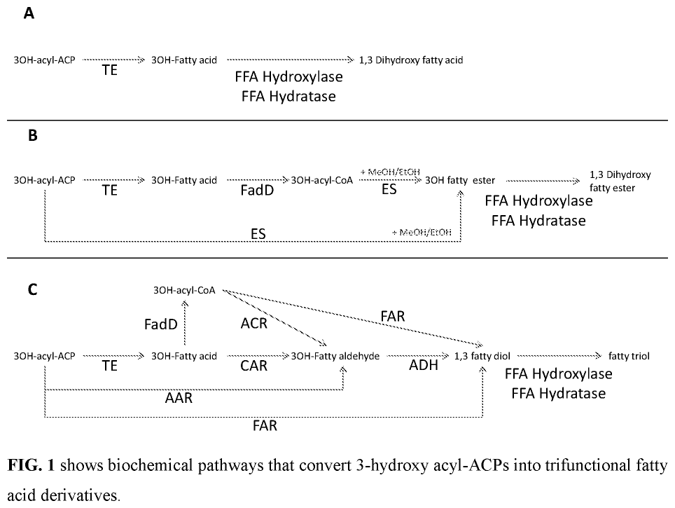

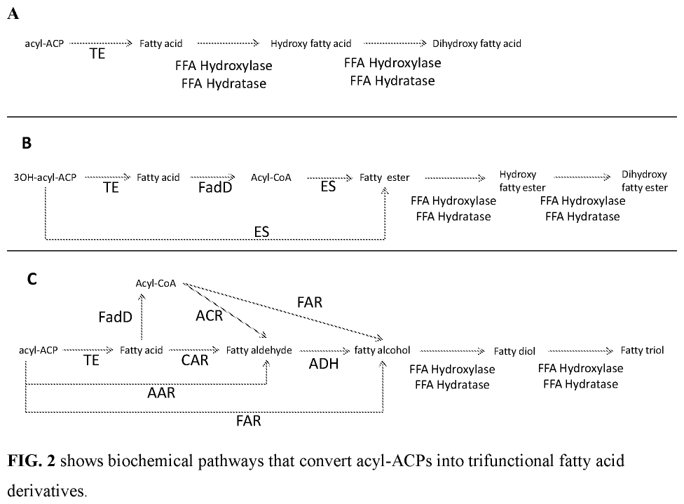

Some exemplary heterologous enzyme pathway(s) capable of producing a bifunctional fatty acid derivative molecule are illustrated in FIG. 1 and FIG. 2. In FIG. 1 and FIG. 2 one of skill in the art can see that if a pathway capable of producing a bifunctional fatty acid derivative molecule is combined with one additional hydroxylating enzyme, a trifunctional fatty acid derivative is produced. Furthermore, if a pathway capable of producing a bifunctional fatty acid derivative molecule is combined with further hydroxylating enzymes, then tetrafunctional fatty acid derivatives are produced. In some embodiments, tetrafunctional fatty acid derivatives are also produced if a pathway from FIG. 1 and/or FIG. 2 includes a hydroxylase that can hydroxylate in two different positions.

In particular, in exemplary embodiments, a recombinant host cell comprising heterologous enzyme pathway capable of producing a bifunctional fatty acid derivative molecules produces 3-hydroxy fatty acids from 3-hydroxy acyl-ACPs. See e.g., FIG. 1A. Addition of at least one heterologous hydroxylating enzyme to the recombinant host cell provides a host cell that, when grown on a simple carbon source produces multifunctional fatty acid derivative molecules.

In other exemplary embodiments, a recombinant host cell comprising heterologous enzyme pathway(s) capable of producing a bifunctional fatty acid derivative produces 3-hydroxy fatty esters from 3-hydroxy acyl-ACPs. See e.g., FIG. 1B. Addition of at least one heterologous hydroxylating enzyme to the recombinant host cell provides a host cell that, when grown on a simple carbon source produces multifunctional fatty acid derivative molecules.

In still other exemplary embodiments, a recombinant host cell comprising heterologous enzyme pathway(s) capable of producing a bifunctional fatty acid derivative produces 1,3-fatty diols from 3-hydroxy acyl-ACPs. See e.g., FIG. 1C. Addition of at least one heterologous hydroxylating enzyme to the recombinant host cell provides a host cell that, when grown on a simple carbon source produces multifunctional fatty acid derivative molecules.

In still other exemplary embodiments, a recombinant host cell comprising heterologous enzyme pathway(s) capable of producing a bifunctional fatty acid derivative produces hydroxy-fatty acids from fatty acids. See e.g., FIG. 2A. Addition of at least one heterologous hydroxylating enzyme to the recombinant host cell provides a host cell that, when grown on a simple carbon source produces multifunctional fatty acid derivative molecules.

In still other exemplary embodiments, a recombinant host cell comprising heterologous enzyme pathway(s) capable of producing a bifunctional fatty acid derivative produces hydroxy-fatty esters from a fatty esters. See e.g., FIG. 2B. Addition of at least one heterologous hydroxylating enzyme to the recombinant host cell provides a host cell that, when grown on a simple carbon source produces multifunctional fatty acid derivative molecules.

In still other exemplary embodiments, a recombinant host cell comprising heterologous enzyme pathway(s) capable of producing a bifunctional fatty acid derivative produces fatty diols from fatty alcohols. See e.g., FIG. 2C. Addition of at least one heterologous hydroxylating enzyme to the recombinant host cell provides a host cell that, when grown on a simple carbon source produces multifunctional fatty acid derivative molecules.

If a pathway from FIG. 1 or FIG. 2 is combined with an additional hydroxylating enzyme e.g., a hydroxylase or a hydratase, then tetrafunctional fatty acid derivatives with four functional groups are produced. Tetrafunctional fatty acid derivatives can also be produced if a pathway from FIG. 1 and FIG. 2 includes a hydroxylase that can hydroxylate in two different positions.

Although FIG. 1 and FIG. 2 depict the enzymatic hydroxylation as the last step of the biochemical pathway, hydroxylation can occur at an earlier step of the pathways, e.g. in FIG. 1C the 3OH fatty acid intermediate may be hydroxylated before it is converted to a fatty alcohol by carboxylacid reductase (CAR).

a. Multifunctional Fatty Acid Derivatives from 3-Hydroxy Fatty Acids

FIG. 1A illustrates and exemplary pathway for the production of multifunctional fatty acid derivative molecules from 3-hydroxy-acyl-ACPs (3OH-acyl-ACPs) via 3-hydroxy fatty acids. Thus, in addition to a heterologous biochemical pathway that generates 3-hydroxy fatty acids a recombinant cell need only comprise one additional hydroxylating enzyme to synthesize a trifunctional fatty acid derivative.

Thus, in exemplary embodiments, a recombinant microbe that expresses a heterologous biochemical pathway comprising a thioesterase, and at least one hydroxylating enzyme, produces multifunctional fatty acid molecules. Suitable thioesterases include any polypeptides that, when expressed in a microorganism in the presence of a carbon source, catalyze the production of fatty acids including 3-hydroxy fatty acids, e.g., enzymes having an Enzyme Commission number (EC 3.1.2.). Exemplary thioesterases include e.g., FatB1 from Umbellularia californica (Q41635) or PhaG from Pseudomonas putida (AAN67031).

Therefore in an exemplary embodiment, a recombinant microbe comprising a thioesterase such as FatB1 from Umbellularia californica and an ω-hydroxylase such as cyp153A from Marinobacter aquaeolei produces the trifunctional molecules 3,10-dihydoxy decanoic acid, 3,12-dihydoxy dodecanoic acid, 3,14-dihydoxy tetradecanoic acid, (z5)3,12-dihydoxy dodecenoic acid and (z7)3,14-dihydoxy tetradecanoic acid when the recombinant microbe is grown on a simple carbon source.

In another exemplary embodiment, a recombinant microbe expressing a heterologous biochemical pathway comprising an ester synthase such as FatB1 from Umbellularia californica and a “subterminal” hydroxylase such as cyp102A from a Bacillus produces the trifunctional molecules 3,9-dihydoxy dodecanoic acid; 3,8-dihydoxy dodecanoic acid; 3,7-dihydoxy decanoic acid; 3,11-dihydoxy dodecanoic acid; 3,10-dihydoxy dodecanoic acid; 3,9-dihydoxy dodecanoic acid; 3,13-dihydoxy tetradecanoic acid; 3,12-dihydoxy tetradecanoic acid; 3,11-dihydoxy tetradecanoic acid; (z5)3,11-dihydoxy dodecenoic acid; (z5)3,10-dihydoxy dodecenoic acid; (z5)3,9-dihydoxy dodecenoic acid; (z7)3,13-dihydoxy tetradecenoic acid; (z7)3,12-dihydoxy tetradecenoic acid; (z7)3,11-dihydoxy tetradecenoic acid, when the recombinant microbe is grown on a simple carbon source.

b. Multifunctional Fatty Acid Derivatives from 3-Hydroxy Fatty Esters

FIG. 1B shows biochemical pathways that convert 3-hydroxy-acyl-ACPs (3OH-acyl-ACPs) into trifunctional fatty acid derivatives via 3-hydroxy fatty methyl or ethyl esters. Thus, in addition to a heterologous biochemical pathway that generates 3-hydroxy fatty methyl or ethyl esters a recombinant cell need only comprise one additional hydroxylating enzyme to synthesize a trifunctional fatty acid derivative.

Thus, in exemplary embodiments, a recombinant microbe that expresses a heterologous biochemical pathway comprising an ester synthase and at least one hydroxylating enzyme, produces multifunctional fatty acid ester molecules. Suitable ester synthases include any polypeptides that, when expressed in a microorganism in the presence of a carbon source and an alcohol, catalyze the production of fatty esters, e.g., fatty acid methyl and ethyl esters, including 3-hydroxy esters e.g., enzymes having an Enzyme Commission number (EC 2.3.1.75). Exemplary ester synthases include e.g., ester synthase polypeptide, such as e.g., ES9, a wax ester synthase from Marinobacter hydrocarbonoclasticus DSM 8798 (UniProtKB A3RE51, GenBank AB021021, see e.g., U.S. Pat. No. 8,530,221, PCT Publication WO2011038132, U.S. Pat. No. 9,133,406), or ES376 (another wax ester synthase from Marinobacter hydrocarbonoclasticus DSM 8798).

Therefore in an exemplary embodiment, a recombinant microbe comprising an ester synthase such as ES9 and a ω-hydroxylase such as cyp153A from Marinobacter aquaeolei produces the trifunctional molecules 3,12-dihydoxy dodecanoic acid methyl ester, 3,14-dihydoxy tetradecanoic acid methyl ester, 3,16-dihydoxy hexadecanoic acid methyl ester, (z5)3,12-dihydoxy dodecenoic acid methyl ester, (z7)3,14-dihydoxy tetradecanoic acid methyl ester and (z9)3,16-dihydoxy hexadecanoic acid methyl ester when the recombinant microbe is grown on a simple carbon source with methanol added.

In another exemplary embodiment, a recombinant microbe expressing a heterologous biochemical pathway comprising an ester synthase such as ES9 from Marinobacter hydrocarbinoclasticus and an ω-hydroxylase such as cyp153A from Marinobacter aquaeolei produces the trifunctional molecules 3,12-dihydoxy dodecanoic acid ethyl ester, 3,14-dihydoxy tetradecanoic acid ethyl ester, 3,16-dihydoxy hexadecanoic acid ethyl ester, (z5)3,12-dihydoxy dodecenoic acid ethyl ester, (z7)3,14-dihydoxy tetradecanoic acid ethyl ester and (z9)3,16-dihydoxy hexadecanoic acid ethyl ester when the recombinant microbe is grown on a simple carbon source with ethanol added.

In another exemplary embodiment, a recombinant microbe expressing a heterologous biochemical pathway comprising an ester synthase such as ES9 from Marinobacter hydrocarbinoclasticus and a “subterminal” hydroxylase such as cyp102A from Bacillus licheniformis produces the trifunctional molecules 3,11-dihydoxy dodecanoic acid methyl ester, 3,10-dihydoxy dodecanoic acid methyl ester, 3,9-dihydoxy dodecanoic acid methyl ester, 3,13-dihydoxy tetradecanoic acid methyl ester, 3,12-dihydoxy tetradecanoic acid methyl ester, 3,11-dihydoxy tetradecanoic acid methyl ester, 3,15-dihydoxy hexadecanoic acid methyl ester, 3,14-dihydoxy hexadecanoic acid methyl ester, 3,13-dihydoxy hexadecanoic acid methyl ester, (z5)3,11-dihydoxy dodecenoic acid methyl ester, (z5)3,10-dihydoxy dodecenoic acid methyl ester, (z5)3,9-dihydoxy dodecenoic acid methyl ester, (z7)3,13-dihydoxy tetradecenoic acid methyl ester, (z7)3,12-dihydoxy tetradecenoic acid methyl ester, (z7)3,11-dihydoxy tetradecenoic acid methyl ester, (z9)3,15-dihydoxy hexadecenoic acid methyl ester, (z9)3,14-dihydoxy hexadecenoic acid methyl ester, (z9)3,13-dihydoxy hexadecenoic acid methyl ester when the recombinant microbe is grown on a simple carbon source and methanol is added.

For example, a recombinant microbe that expresses a heterologous biochemical pathway comprising an ester synthase such as ES9 from Marinobacter hydrocarbinoclasticus and a “subterminal” hydroxylase such as cyp102A from Bacillus lichenformis produces the trifunctional molecules 3,11-dihydoxy dodecanoic acid ethyl ester, 3,10-dihydoxy dodecanoic acid ethyl ester, 3,9-dihydoxy dodecanoic acid ethyl ester, 3,13-dihydoxy tetradecanoic acid ethyl ester, 3,12-dihydoxy tetradecanoic acid ethyl ester, 3,11-dihydoxy tetradecanoic acid ethyl ester, 3,15-dihydoxy hexadecanoic acid ethyl ester, 3,14-dihydoxy hexadecanoic acid ethyl ester, 3,13-dihydoxy hexadecanoic acid ethyl ester, (z5)3,11-dihydoxy dodecenoic acid ethyl ester, (z5)3,10-dihydoxy dodecenoic acid ethyl ester, (z5)3,9-dihydoxy dodecenoic acid ethyl ester, (z7)3,13-dihydoxy tetradecenoic acid ethyl ester, (z7)3,12-dihydoxy tetradecenoic acid ethyl ester, (z7)3,11-dihydoxy tetradecenoic acid ethyl ester, (z9)3,15-dihydoxy hexadecenoic acid ethyl ester, (z9)3,14-dihydoxy hexadecenoic acid ethyl ester, (z9)3,13-dihydoxy hexadecenoic acid ethyl ester when the recombinant microbe is grown on a simple carbon source and ethanol is added.

c. Multifunctional Fatty Acid Derivatives from 1,3-Fatty Diols

FIG. 1C illustrates biochemical pathways for the conversion of 3-hydroxy-acyl-ACPs into trifunctional fatty acid derivatives via 1,3 fatty diols.

Methods for the production of 1,3-fatty diols are known in the art (see e.g., US Patent Application Publication 2017/0204436). As will be shown below, the addition of only one additional hydroxylating enzyme provides for the synthesis of trifunctional fatty acid derivatives. Further combination with another hydroxylating enzyme e.g., a hydroxylase or hydratase, produces tetrafunctional fatty acid derivatives with four functional groups. Tetrafunctional fatty acid derivatives can also be produced if a pathway includes a hydroxylase that can hydroxylate in two different positions.

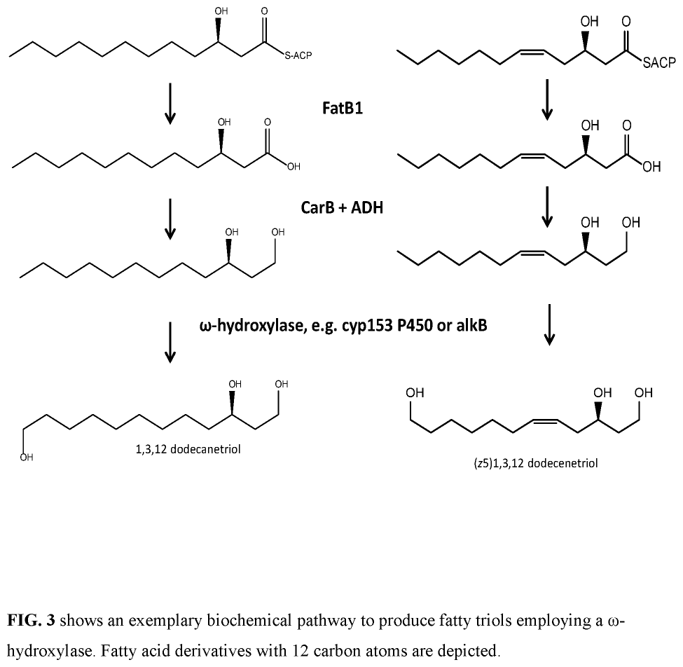

Thus, in an exemplary embodiment, a recombinant microbe expressing a heterologous a biochemical pathway that converts 3-hydroxy-acyl-ACPs into trifunctional fatty acid derivatives via 1,3 fatty diols comprises a thioesterase such as FatB1 from Umbellularia californica, a carboxylic acid reductase such as CarB from Mycobacterium smegmatis, an alcohol dehydrogenase such as AlrA from Acinetobacter baylyi and a w-hydroxylase such as cyp153A from Marinobacter aquaeolei. The recombinant microbe produces the trifunctional molecules 1,3,12 dodecanetriol and (z5)1,3,12 dodecenetriol when the recombinant microbe is grown on a simple carbon source (see e.g., FIG. 3). The heterologous expression of the alcohol dehydrogenase is optional, because most microbes express endogenous alcohol dehydrogenases.

In another exemplary embodiment, a recombinant microbe expressing a heterologous biochemical pathway comprising a thioesterase such as FatB1 from Umbellularia californica, a carboxylic acid reductase such as CarB from Mycobacterium smegmatis, an alcohol dehydrogenase such as AlrA from Acinetobacter baylyi and a ω-hydroxylase such as alkB from Pseudomonas putida produces the trifunctional molecules 1,3,12 dodecanetriol and (z5)1,3,12 dodecenetriol when the recombinant microbe is grown on a simple carbon source (see e.g., FIG. 3). Here again, the heterologous expression of the alcohol dehydrogenase is optional, because most microbes express endogenous alcohol dehydrogenases.

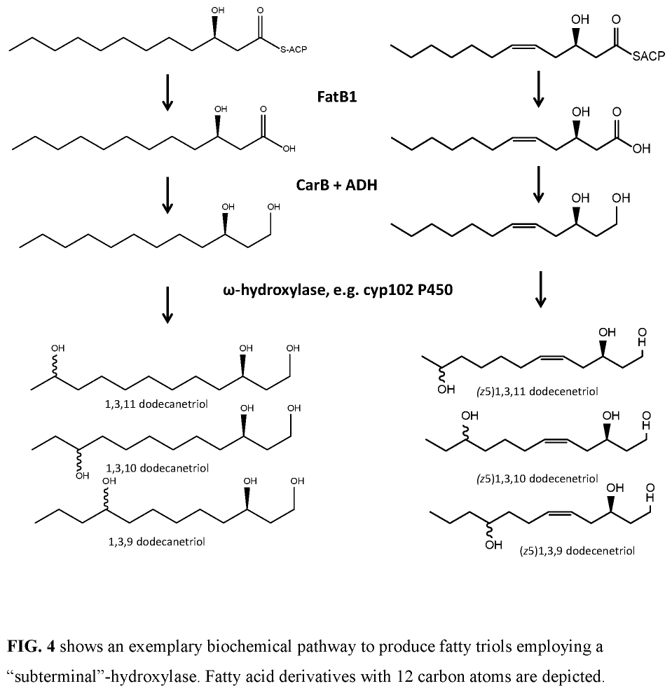

In another exemplary embodiment, a recombinant microbe that expresses a heterologous biochemical pathway comprising a thioesterase such as FatB1 from Umbellularia californica, a carboxylic acid reductase such as CarB from Mycobacterium smegmatis, an alcohol dehydrogenase such as AlrA from Acinetobacter baylyi and a “subterminal” hydroxylase such as cyp102A from a Bacillus (e.g., Bacillus licheniformis) produces the trifunctional molecules 1,3,11 dodecanetriol, 1,3,10 dodecanetriol, 1,3,9 dodecanetriol, (z5)1,3,11 dodecenetriol, (z5)1,3,10 dodecenetriol and (z5)1,3,9 dodecenetriol when the recombinant microbe is grown on a simple carbon source (see e.g., FIG. 4). The heterologous expression of the alcohol dehydrogenase is optional, because most microbes express endogenous alcohol dehydrogenases.

In another exemplary embodiment, a recombinant microbe that expresses a heterologous biochemical pathway comprising a thioesterase such as PhaG from Pseudomonas putida, a carboxylic acid reductase such as CarB from Mycobacterium smegmatis, an alcohol dehydrogenase such as AlrA from Acinetobacter baylyi and a ω-hydroxylase such as cyp153A from Marinobacter aquaeolei produces the trifunctional molecules 1,3,8 octanetriol, 1,3,10 decanetriol, 1,3,12 dodecanetriol, and (z5)1,3,12 dodecenetriol when the recombinant microbe is grown on a simple carbon source. The heterologous expression of the alcohol dehydrogenase is optional, because most microbes express endogenous alcohol dehydrogenases.

In another exemplary embodiment, a recombinant microbe that expresses a heterologous a biochemical pathway comprising of a thioesterase such as PhaG from Pseudomonas putida, a carboxylacid reductase such as CarB from Mycobacterium smegmatis, an alcohol dehydrogenase such as AlrA from Acinetobacter baylyi and a “subterminal” hydroxylase such as cyp102A from Bacillus lichenformis can produce from a simple carbon source the trifunctional molecules 1,3,7 octanetriol, 1,3,5 octanetriol, 1,3,5 octanetriol, 1,3,9 decanetriol, 1,3,8 decanetriol, 1,3,7 decanetriol, 1,3,11 dodecanetriol, 1,3,10 dodecanetriol, 1,3,9 dodecanetriol, (z5)1,3,11 dodecenetriol, (z5)1,3,10 dodecenetriol and (z5)1,3,9 dodecenetriol. The heterologous expression of the alcohol dehydrogenase is optional, because most microbes express endogenous alcohol dehydrogenases.

In another exemplary embodiment, a recombinant microbe that expresses a heterologous biochemical pathway comprising an acyl-ACP reductase such as AAR from Synechococcus elongatus, an alcohol dehydrogenase such as AlrA from Acinetobacter baylyi and a ω-hydroxylase such as cyp153A from Marinobacter aquaeolei produces the trifunctional molecules 1,3,14 tetradecanetriol, 1,3,16 hexadecanetriol, (z7)1,3,14 tetradecenetriol and (z9)1,3,16 hexadecenetriol when the recombinant microbe is grown on a simple carbon source. The heterologous expression of the alcohol dehydrogenase is optional, because most microbes express endogenous alcohol dehydrogenases.

In another exemplary embodiment, a recombinant microbe that expresses a heterologous biochemical pathway comprising an acyl-ACP reductase such as AAR from Synechococcus elongatus, an alcohol dehydrogenase such as AlrA from Acinetobacter baylyi and a “subterminal” hydroxylase such as cyp102A from a Bacillus produces the trifunctional molecules 1,3,13 tetradecanetriol, 1,3,12 tetradecanetriol, 1,3,11 tetradecanetriol, 1,3,15 hexadecanetriol, 1,3,14 hexadecanetriol, 1,3,13 hexadecanetriol, (z7)1,3,13 tetradecenetriol, (z7)1,3,12 tetradecenetriol, (z7)1,3,11 tetradecenetriol, (z9)1,3,15 hexadecenetriol, (z9)1,3,14 hexadecenetriol and (z9)1,3,13 hexadecenetriol when the recombinant microbe is grown on a simple carbon source. The heterologous expression of the alcohol dehydrogenase is optional, because most microbes express endogenous alcohol dehydrogenases.

Multifunctional fatty acid derivatives can also be derived from bifunctional 3-oxo fatty acids (R1: —COOH, R2: ═O), however 3-oxo fatty acids may spontaneously decarboxylate to form the corresponding methyl-ketone (R1: H, R2: ═O; carbon chain is one carbon shorter).

d. Multifunctional Fatty Acid Derivatives from Fatty Acids, Fatty Esters and Alcohols

It is not a requirement to incorporate hydroxylation at both R1 and R2 (scheme 1) for producing multifunctional fatty acid derivatives. Biochemical pathways towards multifunctional fatty acid derivatives without hydroxylation at R2, i.e., via acyl-ACPs and not 3-hydroxy acyl-ACPs, are disclosed in FIG. 2. Such pathways to the production of multifunctional fatty acid derivatives employ two hydroxylases or hydratases that hydroxylate at different positions of the carbon chain to produce trifunctional fatty acid derivatives. In some embodiments, only one hydroxylase that can hydroxylate two positions can be employed.

i. Multifunctional Fatty Acid Derivatives from Fatty Acids

Thus, in an exemplary embodiment, a recombinant microbe that expresses a heterologous biochemical pathway comprising a thioesterase such as FatA from Arabidopsis thaliana, a fatty acid hydratase such as OhyA1 or OhyA2 from Stenotrophomonas maltophilia and a ω-hydroxylase such as cyp153A from Marinobacter aquaeolei produces the trifunctional molecule 10,16-dihydroxyhexadecanoic acid when the microbe is grown on a simple carbon source.

In another exemplary embodiment, a recombinant microbe that expresses a heterologous biochemical pathway comprising a thioesterase such as FatA from Arabidopsis thaliana, a fatty acid hydratase such as OhyA1 or OhyA2 from Stenotrophomonas maltophilia and a ω-hydroxylase such as cyp102A from Bacilllus lichenformis produces the trifunctional molecules 10,15-dihydroxyhexadecanoic acid, 10,14-dihydroxyhexadecanoic acid and 10,13-dihydroxyhexadecanoic acid when the microbe is grown on a simple carbon source.

In another exemplary embodiment, a recombinant microbe that expresses a heterologous biochemical pathway comprising an epoxygenase such as delta 12 fatty acid epoxygenase from Stokasia laevis and epoxide hydrolase from Caenorhabditis elegans, and a thioesterase such as FatA from Arabidopsis thaliana produces the trifunctional molecules 9,10-dihydroxyhexadecanoic and 9,10-dihydroxyoctadecanoic acid when the recombinant microbe is grown on a simple carbon source.

In another exemplary embodiment, a recombinant microbe that expresses a heterologous biochemical pathway comprising an epoxygenase such as delta 12 fatty acid epoxygenase from Stokasia laevis and epoxide hydrolase from Caenorhabditis elegans, a thioesterase such as FatA from Arabidopsis thaliana and a ω-hydroxylase such as cyp153A from Marinobacter aquaeolei produces the tetrafunctional molecules 9,10,16-trihydroxyhexadecanoic acid and 9,10,18-trihydroxyoctadecanoic acid when the recombinant microbe is grown on a from a simple carbon source.

In another exemplary embodiment, a recombinant microbe that expresses a heterologous biochemical pathway comprising an epoxygenase such as delta 12 fatty acid epoxygenase from Stokasia laevis and epoxide hydrolase from Caenorhabditis elegans, a thioesterase such as FatA from Arabidopsis thaliana and a “subterminal” hydroxylase such as cyp102A from Bacillus lichenformis produces the tetrafunctional molecules 9,10,15-trihydroxyhexadecanoic acid; 9,10,14-trihydroxyhexadecanoic acid; 9,10,13-trihydroxyhexadecanoic acid; 9,10,15-trihydroxyoctadecanoic acid; 9,10,14-trihydroxyoctadecanoic acid; 9,10,13-trihydroxyoctadecanoic acid when the recombinant microbe is grown on a simple carbon source.

In another exemplary embodiment, a recombinant microbe that expresses a heterologous biochemical pathway comprising a 10S-Dioxygenase and 7,10-Diol Synthase such as PA2077 and PA2078 from Pseudomonas aeruginosa and a thioesterase such as FatA3 from Arabidopsis thaliana produces the trifunctional molecules 7,10-dihydroxy-(8e)-hexadecenoic acid and 7,10-dihydroxy-(8e)-octadecenoic acid when the recombinant microbe is grown on a simple carbon source.

In another exemplary embodiment, a recombinant microbe that expresses a heterologous biochemical pathway comprising a 10S-Dioxygenase and 7,10-Diol Synthase such as PA2077 and PA2078 from Pseudomonas aeruginosa, a thioesterase such as FatA3 from Arabidopsis thaliana and a ω-hydroxylase such as cyp153A from Marinobacter aquaeolei produces the tetrafunctional molecules 7,10,16-trihydroxy-(8e)-hexadecenoic acid and 7,10,18-trihydroxy-(8e)-octadecenoic acid when the recombinant microbe is grown on a simple carbon source.

In another exemplary embodiment, a recombinant microbe that expresses a heterologous biochemical pathway comprising a 10S-Dioxygenase and 7,10-Diol Synthase such as PA2077 and PA2078 from Pseudomonas aeruginosa, a thioesterase such as FatA3 from Arabidopsis thaliana and a “subterminal” hydroxylase such as cyp102A from Bacillus licheniformis produces the tetrafunctional molecules 7,10,15-trihydroxy-(8e)-hexadecenoic acid; 7,10, 14-trihydroxy-(8e)-hexadecenoic acid; 7,10,13-trihydroxy-(8e)-hexadecenoic acid; 7,10,15-trihydroxy-(8e)-octadecenoic acid; 7,10,14-trihydroxy-(8e)-octadecenoic acid and 7,10,13-trihydroxy-(8e)-octadecenoic acid when the recombinant microbe is grown on a simple carbon source.

ii. Multifunctional Fatty Acid Derivatives from Fatty Alcohols

In another exemplary embodiment, a recombinant microbe that expresses a heterologous a biochemical pathway comprising a thioesterase such as FatA3 from Arabidopsis thaliana, a fatty acid hydratase such as OhyA1 or OhyA2 from Stenotrophomonas maltophilia, a carboxylic acid reductase such as CarB from Mycobacterium smegmatis, an alcohol dehydrogenase such as AlrA from Acinetobacter baylyi and a ω-hydroxylase such as cyp153A from Marinobacter aquaeolei produces the trifunctional molecule 1,10,16-hexadecanetriol when the recombinant microbe is grown on a simple carbon source. The heterologous expression of the alcohol dehydrogenase is optional, because most microbes express endogenous alcohol dehydrogenases.

In another exemplary embodiment, a recombinant microbe that expresses a heterologous biochemical pathway comprising a thioesterase such as FatA3 from Arabidopsis thaliana, a fatty acid hydratase such as OhyA1 or OhyA2 from Stenotrophomonas maltophilia, a carboxylic acid reductase such as CarB from Mycobacterium smegmatis, an alcohol dehydrogenase such as AlrA from Acinetobacter baylyi and a ω-hydroxylase such as cyp102A from Bacilllus lichenformis produces the trifunctional molecules 1,10,15-hexadecanetriol, 1,10,14-hexadecanetriol and 1,10,13-hexadecanetriol when the recombinant microbe is grown on a simple carbon source. The heterologous expression of the alcohol dehydrogenase is optional, because most microbes express endogenous alcohol dehydrogenases.

In another exemplary embodiment, a recombinant microbe that expresses a heterologous biochemical pathway comprising an epoxygenase such as delta 12 fatty acid epoxygenase from Stokasia laevis and epoxide hydrolase from Caenorhabditis elegans, an acyl-ACP reductase such as AAR from Synechococcus elongatus and an alcohol dehydrogenase such as AlrA from Acinetobacter baylyi produces the trifunctional molecules 1,9,10-hexadecanetriol and 1,9,10-octadecanetriol when the recombinant microbe is grown on a simple carbon source.

In another exemplary embodiment, a recombinant microbe that expresses a heterologous biochemical pathway comprising a 10S-Dioxygenase and 7,10-Diol Synthase such as PA2077 and PA2078 from Pseudomonas aeruginosa, an acyl-ACP reductase such as AAR from Synechococcus elongatus and an alcohol dehydrogenase such as AlrA from Acinetobacter baylyi produces the trifunctional molecules 1,7,10-(8e)-hexadecenetriol acid and 1,7,10-(8e)-octadecenetriol when the recombinant microbe is grown on a simple carbon source.

Although FIG. 2 depicts the enzymatic hydroxylations as the last steps of the biochemical pathway, they can occur at earlier steps of the pathways, e.g. in FIG. 2C the fatty acid intermediate may be hydroxylated before it is converted to a fatty alcohol by carboxylic acid reductase (CAR).

5. Heterologous Hydroxylating Enzymes

In exemplary embodiments, ω-hydroxylases are used for hydroxylation at R5 in Scheme 1. Some exemplary ω-hydroxylases/o-oxygenases (EC 1.14.15.3) and their redox partners are provided in Tables 1A and 1B. In general, the ω-hydroxylases/o-oxygenases (EC 1.14.15.3) are non-heme di-iron oxygenases (e.g., alkB from Pseudomonas putida GPo1) or heme-type P450 oxygenases (e.g., cyp153A from Marinobacter aquaeolei) also known as cytochrome P450s.

Cytochromes P450s are proteins encoded by a superfamily of genes that convert a broad variety of substrates and catalyze a variety of chemical reactions.

An exemplary cytochrome P450 is cyp153A. Cyp153A is a sub-family of soluble bacterial cytochrome P450s that hydroxylate hydrocarbon chains with high selectivity for the ω-position (see e.g., van Beilen et al. (2006) Appl. Environ. Microbiol. 72:59-65; Funhoff et al. (2006) J. Bacteriol. 188:5220-5227; Scheps et al. (2011) Org. Biomol. Chem. 9:6727-6733; Honda-Malca et al. (2012) Chem. Commun. 48:5115-5117).

As with all cytochrome P450s, Cyp153A ω-hydroxylases require electrons for their catalytic activity, which are provided via specific redox proteins such as ferredoxin and ferredoxin reductase. Typically, the redox proteins are discrete proteins interacting with cyp153A.

A self-sufficient hybrid (chimeric) cyp153A oxygenase (i.e., an oxygenase that does not require discrete ferredoxin and ferredoxin reductase proteins for activity) has been created by fusing cyp153A from Alcanivorax borkumensis SK2 (see e.g., Kubota et al. (2005) Biosci. Biotechnol. Biochem. 69:2421-2430; Fujita et al. (2009) Biosci. Biotechnol. Biochem. 73:1825-1830) with the reductase domain from P450RhF, which includes flavin mononucleotide (FMN) and NADPH-binding sites and a [2FeS] ferredoxin center (see e.g., Hunter et al. (2005) FEBS Lett. 579:2215-2220). The resulting P450RhF belongs to the class-I P450-fused PFOR (see e.g., DeMot and Parret (2003) Trends Microbiol. 10: 502). Exemplary natural P450-Reductase fusion proteins are provided in Tables 1C and 1D.

Another CYP153A-reductase hybrid fusion proteins was prepared using a gene from Marinobacter aquaeoli coding for the CYP153A (G307A) P450 catalytic domain, where a glycine (G) was substituted for an alanine (A) at position 307, and a gene coding for the c-terminal FMN- and Fe/S-containing reductase domain of P450RhF from Rhodococcus sp. NCIMB9784 (see e.g. US Patent Application Publication 2016/0130616). The resulting polypeptides are CYP153A-RhF1 (SEQ ID NO:4) and CYP153A-RhF2 hybrid fusion polypeptide (SEQ ID NO:6). When this CYP153A-reductase hybrid fusion protein was expressed in E. coli cells with a simple carbon source such as glucose, fatty acid derivatives were efficiently converted to ω-hydroxy fatty acid derivatives source.

Other exemplary ω-hydroxylases (EC 1.14.15.3) and their redox partners that can be used to generate similar CYP153A-reductase hybrid fusion polypeptides are provided in Tables 1A, 1B and Table 7.

TABLE 1A
Examples of ω-Hydroxylase/ω-Oxygenase (EC 1.14.15.3)

| Gene | | | | Hydroxy- |
|---|---|---|---|---|
| Desig- | Source | Accession | Redox | lation |
| nation | Organism | No. | System | Position |
| cyp153A | Acineto- | BAE78452 | operon with | ω- |
| (aciA) | bacter | | ferredoxin and | hydroxylase |
| | sp. OC4 | | ferredoxin | |
| | | | reductase | |
| cyp153A16 | Myco- | YP_001851443 | operon with | ω- |
| | bacterium | | ferredoxin and | hydroxylase |
| | marinum M | | ferredoxin | |
| | | | reductase | |
| cyp153A6 | Myco- | AJ833989 | operon with | ω- |
| | bacterium | | ferredoxin and | hydroxylase |
| | sp. | | ferredoxin | |
| | HXN-1500 | | reductase | |
| cyp153A | Marino- | WP_011784142 | operon with | ω- |
| | bacter | | ferredoxin and | hydroxylase |
| | aquaeolei | | ferredoxin | |
| | VT8 | | reductase | |
| alkB | Pseudo- | CAB54050 | requires | ω- |
| | monas | | rubredoxin and | hydroxylase |
| | putida | | rubredoxin | |
| | GPo1 | | reductase | |
| alkB | Pseudo- | CAB51045 | requires | ω- |
| | monas | | rubredoxin and | hydroxylase |
| | fluorescens | | rubredoxin | |
| | CHA0 | | reductase | |
| alkM | Acineto- | YP_046098 | requires | ω- |
| | bacter | | rubredoxin and | hydroxylase |
| | baylyi | | rubredoxin | |
| | | | reductase | |
| alkB | Gordonia sp. | ADT82701 | requires | ω- |
| | SoGc | | rubredoxin and | hydroxylase |
| | | | rubredoxin | |
| | | | reductase | |
| alkW1 | Dietzia sp. | HQ850582 | c-terminal | ω- |
| | DQ12-45-1b | | rubredoxin | hydroxylase |
| | | | fusion, requires | |
| | | | rubredoxin | |
| | | | reductase | |
| alkB | Pseudo- | CAB54050 | requires | ω- |
| | monas | | rubredoxin and | hydroxylase |
| | putida | | rubredoxin | |
| | GPo1 | | reductase | |
| alkB | Pseudo- | CAB51045 | requires | ω- |
| | monas | | rubredoxin and | hydroxylase |
| | fluorescens | | rubredoxin | |
| | CHA0 | | reductase | |

TABLE 1B
Exemplary Redox Partners for ω-Hydroxylase/
ω-Oxygenase (EC 1.14.15.3)

| Designation/ | | |
|---|---|---|
| Name | Organism | Accession # |
| ferredoxin, | Acinetobacter sp. OC4 | BAE78451, |
| ferredoxin | | BAE78453 |
| reductase | | |
| Putidaredoxin, | Pseudomonas putida | BAA00414, |
| putidaredoxin | | BAA00413 |
| reductase | | |
| (camAB) | | |
| ferredoxin, | Mycobacterium | YP_001851444, |
| ferredoxin | marinum M | YP_001851442 |
| reductase | | |
| ferredoxin, | Marinobacter aquaeoli | YP_957887, |
| ferredoxin | VT8 | YP_957889 |
| reductase | | |
| alkG, alkT | Pseudomonas | CAB54052, |
| | putida GPo1 | CAB54063 |
| rubA, rubB | Acinetobacter | CAA86925, |
| | baylyi ADP1 | CAA86926 |

TABLE 1C
Examples of Self-Sufficient ω-1, ω-2, ω-3-Hydroxylase/
Oxygenase (EC 1.14.14.1) Fusion Proteins

| Gene | | | | Hydroxy- |
|---|---|---|---|---|
| Desig- | Source | Accession | Redox | lation |
| nation | Organism | No. | System | Position |
| cyp102A1 | Bacillus | AAA87602 | fusion protein | ω-1,-2,-3 |
| (P450- | megaterium | | with reductase | hydroxy- |
| BM3) | | | domain | lation |
| cyp102A3 | Bacillus | NP_390594 | fusion protein | ω-1,-2,-3 |
| | subtilis | | with reductase | hydroxy- |
| | | | domain | lation |
| cyp102A7 | Bacillus | AAU41718 | fusion protein | ω-1,-2,-3 |
| | licheniformis | | with reductase | hydroxy- |
| | | | domain | lation |
| Cyp102D1 | Streptomyces | WP_010982013 | fusion protein | ω-1,-2,-3 |
| | avermitilis | | with reductase | hydroxy- |
| | | | domain | lation |
| Cyp505A1 | Fusarium | AB030037 | fusion protein | ω-1,-2,-3 |
| | oxysporum | | with reductase | hydroxy- |
| | | | domain | lation |
| Cyp505E | Aspergillus | XP_001210151 | fusion protein | ω-1,-2, |
| | terreus | | with reductase | -3,-7 |
| | | | domain | hydroxy- |
| | | | | lation |

TABLE 1D
Examples of Self-Sufficient Class-I P450-Fused PFOR Fusion Proteins

| Designation/Name | Organism | Accession # |
|---|---|---|
| P450RhF | Rhodococcus sp. NCIMB 9784 | AAM67416 |
| REQ_44300 | Rhodococcus equi 103S | YP_004009071 |
| HMPREF0018_01193 | Acinetobacter radioresistens | ZP_06072406 |
| | SH164 | |
| BMAA1669 | Burkholderia mallei ATCC 23344 | YP_106239 |
| Rmet_4932 | Cupriavidus metallidurans CH34 | YP_587063 |
| H16_B1279 | Ralstonia eutropha H16 | YP_840799 |

In exemplary embodiments hydroxylation at R3 and R4 in Scheme 1 is achieved through the use of “subterminal” hydroxylases, “mid-chain” hydroxylases and/or oleate hydratases.

“Subterminal” hydroxylases incorporate one OH group at one or more of the omega-1 (ω-1) position, the omega-2 (ω-2) position, the omega-3 (ω-3) position, and/or the omega-4 (ω-4) position, etc. of a fatty acid or fatty acid derivative molecule. Typically, subterminal hydroxylases are cytochrome P450 oxygenases from the cyp102 or cyp505 family (see e.g., Whitehouse et al. (2012) Chem. Soc. Rev. 41: 1218; Kitazume et al. (2000) J. Biol. Chem. 2000, 275:39734-39740) which comprises self-sufficient natural P450-reductase fusion proteins. Cyp102 and Cyp505 family subterminal hydroxylases do not require additional redox partners.

Fatty acid hydroxylases incorporate one OH group at one or more positions close to the center of the hydrocarbon chain. Cytochrome P450 oxygenases can be “mid-chain” fatty acid hydroxylases. Another exemplary group of fatty acid hydroxylases are closely related to plant or fungal acyl-CoA desaturases (see e.g., Broun et al. 1998, Science vol. 282, pp. 1315) and belong to the non-heme diiron protein family. Exemplary “mid-chain” fatty acid hydroxylases include e.g., FAH12 from Ricinus communis (see e.g., Van De Loo et al. 1995, PNAS vol. 92, pp. 6743); CpFAH from Claviceps purpurea (see e.g., Meesapyodsuk and Xiao Qiu, Plant Physiol., vol. 147, pp and Table 2). They require redox partners similar to the ones listed in Table 1B.

Fatty acid hydratases act only on unsaturated carbon atoms, e.g. they can convert oleic acid into 10-hydroxy stearic acid. Exemplary fatty acid hydratases include e.g., ohyA1 and ohyA2 from Stenotrophomonas maltophilia (see e.g. Joo et al. 2012, J. Biotechnol. vol. 158, pp. 17; Kang et al. 2017, AEM vol. 83, pp. 1 and see Table 3). Although fatty acid hydratases contain FAD as a cofactor, cofactor regeneration during catalysis is not required (see e.g., Engleder et al. 2015, ChemBioChem vol. 16, pp. 1730). Additional redox partner as described above for the hydroxylases/oxygenases are not required for the ohyA-type hydratases.

In exemplary embodiments, hydroxylation at R2 and if R1=02H in scheme 1, occurs through the action of a-hydroxylases. Exemplary α-hydoxylases include P450 enzymes of the peroxygenase cyp152 family, for example cyp153A1 from Sphingomonas paucimobilis (see e.g., Table 4, Matsunaga et al. 1997, JBC, vol. 272, No. 38, pp. 23592, etc.). These enzymes can utilize hydrogen peroxide as electron donor, but they can also use redox partners as described in Table 1B.

TABLE 2
Examples of “mid-chain” Hydroxylase/Oxygenase (EC 1.14.19.6)

| Designation/Name | Organism | Accession # |
|---|---|---|
| CpFAH | Claviceps purpurea | CCE30184 |
| RcFAH12 | Ricinus communis | AAC49010 |
| FaFAD1 | Fragaria x ananassa | KF887973 |
| LFAH12 | Lesquerella fendleri | AAC32755 |

TABLE 3
Examples of Hydratases (EC 4.2.1.53)

| Designation/Name | Organism | Accession # |
|---|---|---|
| OhyA1 | Stenotrophomonas maltophilia | WP_012479975 |
| OhyA2 | Stenotrophomonas maltophilia | WP_017356052 |
| OhyA | Elizabethkingia meningoseptica | GQ144652 |
| LAH | Lactobacillus acidophilus | AAV42528 |
| FA-HY1 | Lactobacillus acidophilus | LC030242 |
| FA-HY2 | Lactobacillus acidophilus | LC030243 |
| Sn_OhyA2 | Stenotrophomonas nitritireducens | WP_062168826 |
| SPH | Sfreptococcus pyogenes | ACI60731 |

TABLE 4
Examples of α-Hydroxylases/Peroxygenases (EC 1.11.2.4)

| Designation/Name | Organism | Accession # |
|---|---|---|
| Cyp152B1 | Sphingomonas paucimobilis | BAA22987 |
| Cyp152A1 | Bacillus subtilis | NP_388092 |
| Cyp152A | Bacillus clausii | YP_176535 |

The combined activity of certain dioxygenases and diol synthases (see Table 11) convert fatty acids such as oleic acid into dihydroxy fatty acids via hydroperoxy fatty acid intermediates (Estupian et al. 2014, Biochimica et Biophysica Acta 1841:1360-1371). These enzymes belong to the class of Di-Heme cytochrome C peroxidases.

TABLE 11
Examples of bacterial diol synthases (EC 1.13.11.77)

| Designation/Name | Organism | Accession # |
|---|---|---|
| PA2077, PA2078 (10S- | P. aeruginosa PAO1 | NP_250767, |
| Dioxygenase; 7,10-Diol | | NP_250768 |
| Synthase) | | |
| azo2594, azo2595 | Azoarcus sp. BH72 | WP_011766321, |
| | | WP_011766322 |

The combined activity of certain epoxygenases (also known as peroxygenases or epoxidases) and epoxide hydrolases (see Table 12) convert fatty acids such as oleic acid into dihydroxy fatty acids via epoxy fatty acid intermediates (Kaprakkaden et al. 2017, Microb Cell Fact 16:85). Epoxygenases are heme-containing monooxygenases and catalyze hydroperoxide-dependent epoxidation of unsaturated fatty acids.

TABLE 12
Examples of epoxygenases (EC 1.11.2.3)
and epoxide hydrolases (EC 3.3.2.10)

| Designation/ | | Accession # |
|---|---|---|
| Name | Organism | or reference |
| AsPXG3 | Avena sativa | Benaragama, |
| | | Planta (2017) |
| | | 246:711-719 |
| delta 12 fatty acid | Stokasia laevis | AAR23815 |
| epoxygenase | | |
| AtPXG4 | Arabidopsis thaliana | Benaragama, |
| | | Planta (2017) |
| | | 246:711-719 |
| PpoC | Aspergillus nidulans | AY613780 |
| epoxide hydrolase | Caenorhabditis elegans | ABV45408 |
| epoxide hydrolase-2 | Mangifera idica | AQZ55557 |
| BmeH | Bacillus megaterium | ADV36302 |

6. Heterologous Enzymes that Modify Hydroxyl Groups of Multifunctional Fatty Acid Derivative Molecules

In exemplary embodiments, additional enzymes are employed to convert the hydroxyl groups of multifunctional fatty acid derivatives into other functional groups, e.g. oxo (CHO), carboxyl (CO₂H), amino (CH₂NH₂),O-acetyl (CO₂C₂H₃), methoxy (COCH₃) or ester (CO₂CH₃, CO₂C₂H₅, CO₂C₃H₇, CO₂C₂H₃) groups. Exemplary enzymes suitable for these modifications include dehydrogenases, oxidases, transaminases, acetyl-transferases, methyl transferases and ester synthases (see e.g., Table 5).

TABLE 5
Enzymes that convert hydroxyl groups in multifunctional
fatty acid derivatives into other functional groups.

| Designation/Name | Organism | Accession # | Enzyme Function |
|---|---|---|---|
| Alcohol | | | C—OH ↔ C═O |
| dehydrogenase | | | |
| ADH | Micrococcus luteus | ADD83022 | |
| cddC | Rhodococcus ruber | AAL14237 | |
| AlrA | Acinetobacter baylyi | CAG70252 | |
| yjgB | Escherichia coli | NP_418690 | |
| yahK | Escherichia coli | NP_414859 | |
| ybbO | Escherichia coli | NP_415026 | |
| adhP | Escherichia coli | P39451 | |
| Alcohol Oxidase | | | C—OH → C═O |
| alkJ | Pseudomonas putida GPo1 | CAB54054 | |
| alkJ | Alcanivorax borkumensis AP1 | CAC38030 | |
| FAO1 | Yarrowia lipolytica | YALI0B14014g | |
| Aldehyde | | | C—OH → C═O → COOH |
| dehydrogenase | | | |
| alkH | Pseudomonas putida GPo1 | CAB51050 | |
| alkH | Alcanivorax borkumensis AP1 | CAC38029 | |
| cddD | Rhodococcus ruber SC1 | AAL14238 | |
| FAldDH | Acinetobacter baylyi | WP_004927398 | |
| Maqu_3410 | Marinobacter aquaeolei VT8 | WP_011786822 | |
| feaB | Escherichia coli | P80668 | |
| patD | Escherichia coli | P77674 | |
| paaZ | Escherichia coli | P77455 | |
| Transaminase/Amine | | | C—OH → C═O↔C—NH₂ |
| dehydrogenase | | | |
| CV_2025 | Chromobacterium violaceum | Q7NWG4 | |
| gabT | Pseudomonas sp.AAC | KES23458 | |
| ygjG | Escherichia coli | NP_417544 | |
| GabT | Pseudomonas aeruginosa | WP_003156103 | |
| GabT | Pseudomonas aeruginosa | AAG03655 | |
| PRK08297 | Mycobacterium tuberculosis | NP_217807 | |
| Ald | Bacillus subtilis | BAI86717 | |
| gdhA | Escherichia coli | NP_416275 | |
| gdh | Peptoniphilus | AAA25611 | |
| | asaccharolyticus | | |
| Lysine DH | Achromobacter denitrificans | AAZ94428 | |
| Acetyl-O-transferase | 2.3.1.84 | | C—OH → C—O₂C₂H₃ |
| atf1 | Saccharomyces cerevisiae | CAA99708 | |
| SAAT | Fragaria ananassa | AF193789 | |
| Methyl-O-transferase | | | C—OH → C—OCH₃ |
| FAMT | Mycobacterium marinum | NC010612 | |
| DmJHAMT | Drosophila melanogaster | Q9VJK8 | |

III. Fermentation and Production of Multifunctional Fatty Acid Derivatives

As used herein, fermentation broadly refers to the conversion of organic materials into target substances by recombinant host cells. For example, this includes the conversion of a carbon source by recombinant host cells into multifunctional fatty acid derivative molecules as disclosed herein by propagating a culture of the recombinant host cells in a media comprising a carbon source. Conditions permissive for the production of target substances such as e.g., multifunctional fatty acid derivative molecules as disclosed herein, are any conditions that allow a host cell to produce a desired product, such as a multifunctional fatty acid derivative composition. Suitable conditions include, for example, typical fermentation conditions see e.g., Principles of Fermentation Technology, 3rd Edition (2016) supra; Fermentation Microbiology and Biotechnology, 2nd Edition, (2007) supra.

Fermentation conditions can include many parameters, well known in the art, including but not limited to temperature ranges, pH levels, levels of aeration, feed rates and media composition. Each of these conditions, individually and in combination, allows the host cell to grow. Fermentation can be aerobic, anaerobic, or variations thereof (such as micro-aerobic). Exemplary culture media include broths (liquid) or gels (solid). Generally, the medium includes a carbon source (e.g., a simple carbon source derived from a renewable feedstock) that can be metabolized by a host cell directly. In addition, enzymes can be used in the medium to facilitate the mobilization (e.g., the depolymerization of starch or cellulose to fermentable sugars) and subsequent metabolism of the carbon source to produce multifunctional fatty acid derivatives.

For small scale production, the host cells engineered to produce multifunctional fatty acid derivative compositions are typically grown in batches of, for example, about 100 μL, 200 μL, 300 μL, 400 μL, 500 μL, 1 mL, 5 mL, 10 mL, 15 mL, 25 mL, 50 mL, 75 mL, 100 mL, 500 mL, 1 L, 2 L, 5 L, or 10 L.

For large scale production, the engineered host cells can be grown in cultures having a volume batches of about 10 L, 100 L, 1000 L, 10,000 L, 100,000 L, 1,000,000 L or larger; fermented; and induced to express any desired polynucleotide sequence.

The multifunctional fatty acid derivative compositions disclosed herein can be found in the extracellular environment of the recombinant host cell culture and can be readily isolated from the culture medium by methods known in the art. A multifunctional fatty acid derivative may be secreted by the recombinant host cell, transported into the extracellular environment or passively transferred into the extracellular environment of the recombinant host cell culture.

Exemplary microorganisms suitable for use as production host cells include e.g., bacteria, cyanobacteria, yeast, algae, filamentous fungi, etc. To produce fatty acid derivative compositions production host cells (or equivalently, host cells) are engineered to comprise fatty acid biosynthesis pathways that are modified relative to non-engineered or native host cells e.g., engineered as discussed above and as disclosed e.g., in U.S. Patent Application Publication 2015/0064782. Production hosts engineered to comprise modified fatty acid biosynthesis pathways are able to efficiently convert glucose or other renewable feedstocks into fatty acid derivatives. Protocols and procedures for high density fermentations for the production of various compounds have been established (see, e.g., U.S. Pat. Nos. 8,372,610; 8,323,924; 8,313,934; 8,283,143; 8,268,599; 8,183,028; 8,110,670; 8,110,093; and 8,097,439).

In some exemplary embodiments, a production host cell is cultured in a culture medium (e.g., fermentation medium) comprising an initial concentration of a carbon source (e.g., a simple carbon source) of about 20 g/L to about 900 g/L. In other embodiments, the culture medium comprises an initial concentration of a carbon source of about 2 g/L to about 10 g/L; of about 10 g/L to about 20 g/L; of about 20 g/L to about 30 g/L; of about 30 g/L to about 40 g/L; or of about 40 g/L to about 50 g/L. In some embodiments, the level of available carbon source in the culture medium can be monitored during the fermentation proceeding. In some embodiments, the method further includes adding a supplemental carbon source to the culture medium when the level of the initial carbon source in the medium is less than about 0.5 g/L.

In some exemplary embodiments, a supplemental carbon source is added to the culture medium when the level of the carbon source in the medium is less than about 0.4 g/L, less than about 0.3 g/L, less than about 0.2 g/L, or less than about 0.1 g/L. In some embodiments, the supplemental carbon source is added to maintain a carbon source level of about 1 g/L to about 25 g/L. In some embodiments, the supplemental carbon source is added to maintain a carbon source level of about 2 g/L or more (e.g., about 2 g/L or more, about 3 g/L or more, about 4 g/L or more). In certain embodiments, the supplemental carbon source is added to maintain a carbon source level of about 5 g/L or less (e.g., about 5 g/L or less, about 4 g/L or less, about 3 g/L or less). In some embodiments, the supplemental carbon source is added to maintain a carbon source level of about 2 g/L to about 5 g/L, of about 5 g/L to about 10 g/L, or of about 10 g/L to about 25 g/L.

In one exemplary embodiment the carbon source for the fermentation is derived from a renewable feedstock. In some embodiments, the carbon source is glucose. In other embodiments, the carbon source is glycerol. Other possible carbon sources include, but are not limited to, fructose, mannose, galactose, xylose, arabinose, starch, cellulose, pectin, xylan, sucrose, maltose, cellobiose, and turanose; cellulosic material and variants such as hemicelluloses, methyl cellulose and sodium carboxymethyl cellulose; saturated or unsaturated fatty acids, succinate, lactate, and acetate; alcohols, such as ethanol, methanol, and glycerol, or mixtures thereof. In one embodiment, the carbon source is derived from corn, sugar cane, sorghum, beet, switch grass, ensilage, straw, lumber, pulp, sewage, garbage, cellulosic urban waste, flu-gas, syn-gas, or carbon dioxide. The simple carbon source can also be a product of photosynthesis, such as glucose or sucrose. In one embodiment, the carbon source is derived from a waste product such as glycerol, flu-gas, or syn-gas; or from the reformation of organic materials such as biomass; or from natural gas or from methane, or from the reformation of these materials to syn-gas; or from carbon dioxide that is fixed photosynthetically, for example multifunctional fatty acid derivatives may be produced by recombinant cyanobacteria growing photosynthetically and using CO₂ as carbon source. In some exemplary embodiments, the carbon source is derived from biomass. An exemplary source of biomass is plant matter or vegetation, such as corn, sugar cane, or switchgrass. Another exemplary source of biomass is metabolic waste products, such as animal matter (e.g., cow manure). Further exemplary sources of biomass include algae and other marine plants. Biomass also includes waste products from industry, agriculture, forestry, and households, including, but not limited to, fermentation waste, ensilage, straw, lumber, sewage, garbage, cellulosic urban waste, municipal solid waste, and food leftovers.

In some exemplary embodiments, a multifunctional fatty acid derivative is produced at a concentration of about 0.5 g/L to about 40 g/L. In some embodiments, a fatty acid derivative is produced at a concentration of about 1 g/L or more (e.g., about 1 g/L or more, about 10 g/L or more, about 20 g/L or more, about 50 g/L or more, about 100 g/L or more). In some embodiments, a fatty acid derivative is produced at a concentration of about 1 g/L to about 170 g/L, of about 1 g/L to about 10 g/L, of about 40 g/L to about 170 g/L, of about 100 g/L to about 170 g/L, of about 10 g/L to about 100 g/L, of about 1 g/L to about 40 g/L, of about 40 g/L to about 100 g/L, or of about 1 g/L to about 100 g/L.

In other exemplary embodiments, a multifunctional fatty acid derivative is produced at a titer of about 25 mg/L, about 50 mg/L, about 75 mg/L, about 100 mg/L, about 125 mg/L, about 150 mg/L, about 175 mg/L, about 200 mg/L, about 225 mg/L, about 250 mg/L, about 275 mg/L, about 300 mg/L, about 325 mg/L, about 350 mg/L, about 375 mg/L, about 400 mg/L, about 425 mg/L, about 450 mg/L, about 475 mg/L, about 500 mg/L, about 525 mg/L, about 550 mg/L, about 575 mg/L, about 600 mg/L, about 625 mg/L, about 650 mg/L, about 675 mg/L, about 700 mg/L, about 725 mg/L, about 750 mg/L, about 775 mg/L, about 800 mg/L, about 825 mg/L, about 850 mg/L, about 875 mg/L, about 900 mg/L, about 925 mg/L, about 950 mg/L, about 975 mg/L, about 1000 mg/L, about 1050 mg/L, about 1075 mg/L, about 1100 mg/L, about 1125 mg/L, about 1150 mg/L, about 1175 mg/L, about 1200 mg/L, about 1225 mg/L, about 1250 mg/L, about 1275 mg/L, about 1300 mg/L, about 1325 mg/L, about 1350 mg/L, about 1375 mg/L, about 1400 mg/L, about 1425 mg/L, about 1450 mg/L, about 1475 mg/L, about 1500 mg/L, about 1525 mg/L, about 1550 mg/L, about 1575 mg/L, about 1600 mg/L, about 1625 mg/L, about 1650 mg/L, about 1675 mg/L, about 1700 mg/L, about 1725 mg/L, about 1750 mg/L, about 1775 mg/L, about 1800 mg/L, about 1825 mg/L, about 1850 mg/L, about 1875 mg/L, about 1900 mg/L, about 1925 mg/L, about 1950 mg/L, about 1975 mg/L, about 2000 mg/L (2 g/L), 3 g/L, 5 g/L, 10 g/L, 20 g/L, 30 g/L, 40 g/L, 50 g/L, 60 g/L, 70 g/L, 80 g/L, 90 g/L, 100 g/L or a range bounded by any two of the foregoing values. In other embodiments, a fatty acid derivative or other compound is produced at a titer of more than 100 g/L, more than 200 g/L, or more than 300 g/L. In exemplary embodiments, the titer of fatty acid derivative or other compound produced by a recombinant host cell according to the methods disclosed herein is from 5 g/L to 200 g/L, 10 g/L to 150 g/L, 20 g/L to 120 g/L and 30 g/L to 100 g/L. The titer may refer to a particular fatty acid derivative or a combination of fatty acid derivatives or another compound or a combination of other compounds produced by a given recombinant host cell culture. In exemplary embodiments, the expression of ChFatB2 thioesterase variant in a recombinant host cell such as E. coli results in the production of a higher titer as compared to a recombinant host cell expressing the corresponding wild type polypeptide. In one embodiment, the higher titer ranges from at least about 5 g/L to about 200 g/L.

In other exemplary embodiments, the host cells engineered to produce a multifunctional fatty acid derivative according to the methods of the disclosure have a yield of at least 1%, at least 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, or at least about 30% or a range bounded by any two of the foregoing values. In other embodiments, a fatty acid derivative or derivatives or other compound(s) are produced at a yield of more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, more than 100%, more than 200%, more than 250%, more than 300%, more than 350%, more than 400%, more than 450%, more than 500%, more than 550%, more than 600%, more than 650%, more than 700%, more than 750%, or more. Alternatively, or in addition, the yield is about 30% or less, about 27% or less, about 25% or less, or about 22% or less. In another embodiment, the yield is about 50% or less, about 45% or less, or about 35% or less. In another embodiment, the yield is about 95% or less, or 90% or less, or 85% or less, or 80% or less, or 75% or less, or 70% or less, or 65% or less, or 60% or less, or 55% or less, or 50% or less. Thus, the yield can be bounded by any two of the above endpoints. For example, the yield of a multifunctional fatty acid derivative e.g., a 6, 7, 8, 9, 10, 11, 12, 13, 14, 15, 16, etc. carbon multifunctional fatty acid derivative produced by the recombinant host cell according to the methods disclosed herein can be about 5% to about 15%, about 10% to about 25%, about 10% to about 22%, about 15% to about 27%, about 18% to about 22%, about 20% to about 28%, about 20% to about 30%, about 30% to about 40%, about 40% to about 50%, about 50% to about 60%, about 60% to about 70%, about 70% to about 80%, about 80% to about 90%, about 90% to about 100%, about 100% to about 200%, about 200% to about 300%, about 300% to about 400%, about 400% to about 500%, about 500% to about 600%, about 600% to about 700%, or about 700% to about 800%. The yield may refer to a particular multifunctional fatty acid derivative or a combination of fatty acid derivatives. In one embodiment, the higher yield ranges from about 10% to about 800% of theoretical yield. In addition, the yield will also be dependent on the feedstock used.

In some exemplary embodiments, the productivity of the host cells engineered to produce a multifunctional fatty acid derivative according to the methods of the disclosure is at least 100 mg/L/hour, at least 200 mg/L/hour, at least 300 mg/L/hour, at least 400 mg/L/hour, at least 500 mg/L/hour, at least 600 mg/L/hour, at least 700 mg/L/hour, at least 800 mg/L/hour, at least 900 mg/L/hour, at least 1000 mg/L/hour, at least 1100 mg/L/hour, at least 1200 mg/L/hour, at least 1300 mg/L/hour, at least 1400 mg/L/hour, at least 1500 mg/L/hour, at least 1600 mg/L/hour, at least 1700 mg/L/hour, at least 1800 mg/L/hour, at least 1900 mg/L/hour, at least 2000 mg/L/hour, at least 2100 mg/L/hour, at least 2200 mg/L/hour, at least 2300 mg/L/hour, at least 2400 mg/L/hour, 2500 mg/L/hour, or as high as 10 g/L/hour (dependent upon cell mass). For example, the productivity of a malonyl-CoA derived compound including a fatty acid derivative or derivatives or other compound(s) produced by a recombinant host cell according to the methods of the disclosure may be from 500 mg/L/hour to 2500 mg/L/hour, or from 700 mg/L/hour to 2000 mg/L/hour. The productivity may refer to a particular 8 and/or 10 carbon fatty acid derivative or a combination of fatty acid derivatives or other compound(s) produced by a given host cell culture. For example, the expression of a ChFatB2 thioesterase variant in a recombinant host cell such as E. coli results in increased productivity of an 8 and/or 10 carbon fatty acid derivatives or other compounds as compared to a recombinant host cell expressing the corresponding wild type polypeptide. In exemplary embodiments, higher productivity ranges from about 0.3 g/L/h to about 3 g/L/h to about 10 g/L/h to about 100 g/L/h to about a 1000 g/L/h.

As disclosed supra, in some exemplary embodiments, the host cell used in the fermentation procedures discussed herein (supra) is a mammalian cell, plant cell, insect cell, yeast cell, fungus cell, filamentous fungi cell, an algal cell, a cyanobacterial cell, and bacterial cell.

IV. Isolation

Bioproducts e.g., compositions comprising multifunctional fatty acid derivatives as disclosed herein which are produced utilizing recombinant host cells as discussed above are typically isolated from the fermentation broth by methods known in the art.

V. Compositions and Formulations of Multifunctional Fatty Acid Derivative Molecules

Bioproducts e.g., compositions comprising multifunctional fatty acid derivative molecules produced utilizing engineered microbes as discussed herein, are produced from renewable sources (e.g., from a simple carbon source derived from renewable feedstocks) and, as such, are new compositions of matter. These new bioproducts can be distinguished from organic compounds derived from petrochemical carbon on the basis of dual carbon-isotopic fingerprinting or ¹⁴C dating. Additionally, the specific source of biosourced carbon (e.g., glucose vs. glycerol) can be determined by dual carbon-isotopic fingerprinting by methods known in the art (see, e.g., U.S. Pat. No. 7,169,588, WO 2016/011430 A1, etc.).

The following examples are offered to illustrate, but not to limit the invention.

### EXAMPLES

### Example 1

The following Example illustrates materials and methods for Examples 2-9 disclosed herein below.

### Methods

### Small Scale Fermentation Protocol:

40 μL LB culture (from an LB culture growing in a 96 well plate) was used to inoculate 360 μL LB media, which was then incubated for approximately 4 hours at 32° C. shaking. 80 μL of the LB seed was used to inoculate 320 μL Nlim media (Table 6). After growing at 32° C. for 2 hours, the cultures were induced with IPTG (final concentration 1 mM). The cultures were then incubated at 32° C. with shaking for 20 hours if not noted otherwise, after which they were extracted following the standard extraction protocol detailed below.

TABLE 6
N-lim Media Formulation
N-lini Media Formulation

| | | |
|---|---|---|
| 1 | × | 5 × Salt Soln. with NH4Cl |
| 1 | × | 1000 × Trace Vitamins |
| 1 | mg/L | 10 mg/mL Thiamine |
| 1 | mM | 1M MgSO4 |
| 0.1 | mM | 1M CaCl2 |
| 40 | g/L | 500 g/L glucose |
| 1 | × | 1000 × Trace minerals |
| 10 | mg/L | 10 g/L Fe Citrate |
| 100 | μg/mL | 100 mg/ml spectinomycin |
| 100 | mM | 2M BisTris (pH7.0) |
| 0.5 | mM | Aminolevulinic acid |

### Fatty Acid Species Standard Extraction and Analytical Protocol

To each well to be extracted, 80 μL of 1M HCl, followed by 400 μL of butyl acetate containing 500 mg/L 1-undecanol or 500 mg/L undecanoic acid as internal standard (IS) was added as internal standard (IS) was added. The 96 well plates were then heat-sealed using a plate sealer (ALPS-300 heater; Abgene, ThermoScientific, Rockford, Ill.), and shaken for 15 minutes at 2000 rpm using MIXMATE mixer (Eppendorf, Hamburg, Germany). After shaking, the plates were centrifuged for 10 minutes at 4500 rpm at room temperature (Allegra X-15R, rotor SX4750A, Beckman Coulter, Brea, Calif.) to separate the aqueous and organic layers. 50 μL of the organic layer was transferred to a 96 well plate (polypropylene, Corning, Amsterdam, The Netherlands) and derivatized with 50 uL of trimethylsiloxy/N,O-Bis(trimethylsilyl)trifluoroacetamide (TMS/BSTFA). The plate was subsequently heat sealed and stored at −20° C. until evaluated by either Gas Chromatography with Flame Ionization Detection (GC-FID) or Gas Chromatography-Mass Spectrometry (GC-MS).

The GC-MS parameters used to generate chromatograms and mass spectra for compounds identification were as follows: 1 μl sample was injected into analytical Column: DB-1HT, 15m×250 μm×0.1 μm, available from Agilent with cat #J&W 122-1111E, Oven temperature: initial at 50° C., hold for 5 minutes, increase to 300° C. at 25° C./min, and hold for 5.24 minutes for a total run time of 24 minutes. Column flow: 1.2 mL/min, Inlet temperature: 300° C., Split ratio: 20:1, Software: ChemStation E.02.01.1177. MS parameters: Transfer line temperature: 300° C., MS source: 230° C., MS Quad: 150° C. Auto sampler: Combi PAL (CTC analytics) distributed by LEAP Technologies. The GC-FID parameters used to quantify each compound were carried out as follows: 1 μL of sample was injected onto an analytical column (UFC Rtx-1, 5 M×0.1 mm×0.1 μM) in a Thermo Fisher UltraFast TRACE GC (Thermo Fisher Scientific, West Palm Beach, Fla.). Oven temperature: initial at 100° C., hold for 0.2 minutes, increase to 320° C. at 100° C./min, and hold for 0.5 minutes for a total run time of 2.5 minutes using column flow of 0.5 ml/min, Inlet temperature: 300° C. and flame ionization detector temperature: 300° C.

The protocol detailed above represents standard conditions, which may be modified as necessary to optimize the analytical results.

### Example 2

The following Example illustrates the conversion of exogenously added 1,3 dodeca(e)nediols to 1,3,12 dodeca(e)netriols by recombinant E. coli strains expressing various ω-hydroxylases. Most of the ω-hydroxylases are from the cyp153A family and were expressed either (i) as free standing catalytic cyp153 P450 enzymes or (ii) as chimeric hybrid fusion proteins (cyp153A P450 enzyme fused with a reductase domain). One ω-hydroxylases was an alkB-type ω-hydroxylase, which does not belong to the P450 family.

The genes for the cyp153 ω-hydroxylases were either amplified from genomic DNA or obtained by gene synthesis and cloned into a pACYC-derivative vector (p15A replicon, kanamycin resistance marker) such that they were under the control of the IPTG-inducible Ptrc promoter and in an operon with the CamA (putraredoxin reductase) and CamB (putredoxin) genes from Pseudomonas. Chimeric P450 fusion proteins did not require CamAB coexpression. The alkB-type ω-hydroxylase from Pseudomonas putida was coexpressed in an operon with its cognate redox proteins alkG and alkT. All ω-hydroxylase plasmids were then transformed into an E. coli MG1655 derivative strain. The small scale fermentation protocol (see above) was followed and at the time of induction a mixture of 1,3 dodecanediol and (z5)1,3-dodecenediol (˜65/35%) was added to the cultures at a final concentration of 1 g/L.

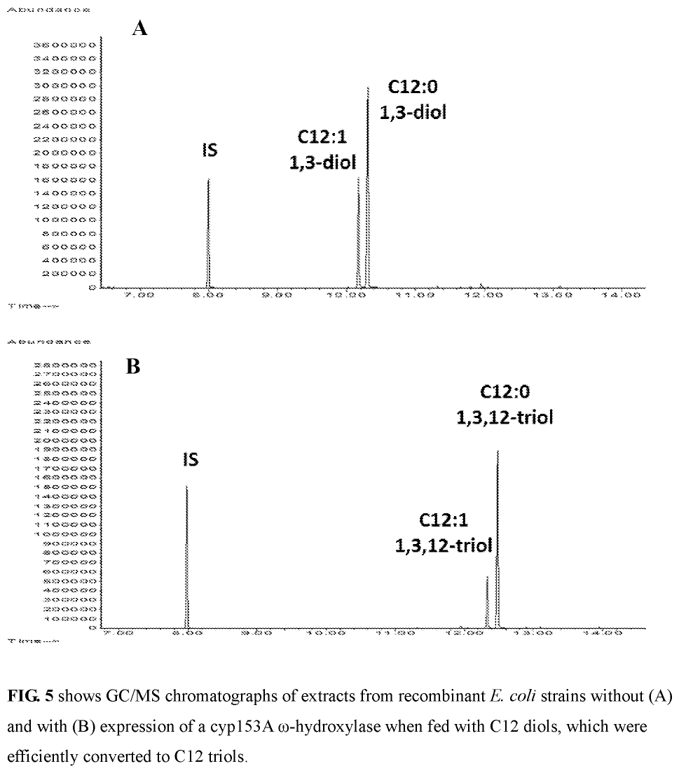

Surprisingly, most strains with the ω-hydroxylases completely or partially converted 1,3-dodeca(e)nediols to 1,3,12-dodeca(e)netriols. In comparison to the control strain without expression of an ω-hydroxylases, two new peaks at RT 12.33 and RT 12.48 min (after TMS/BSTFA derivatization) appeared, while the two peaks corresponding to derivatized 1,3-dodeca(e)nediols disappeared or were significantly reduced (see FIG. 5).

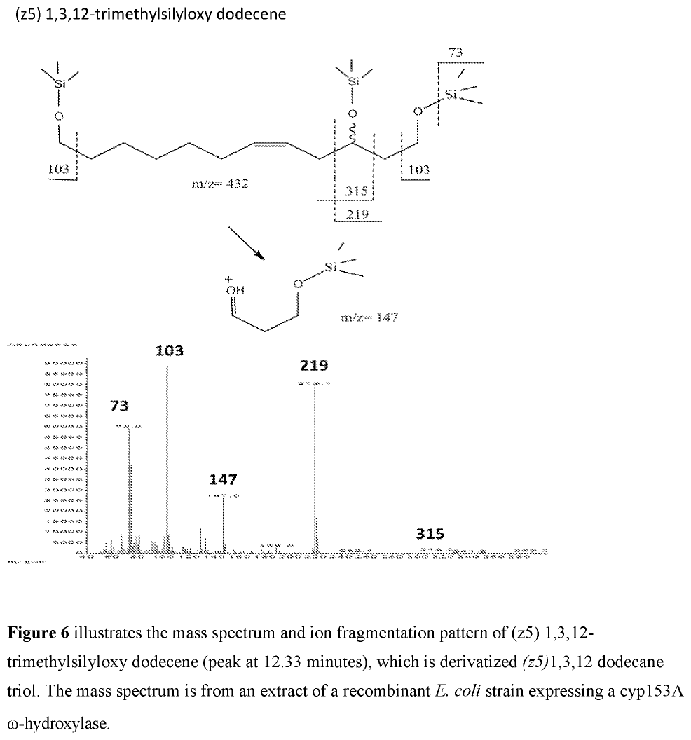

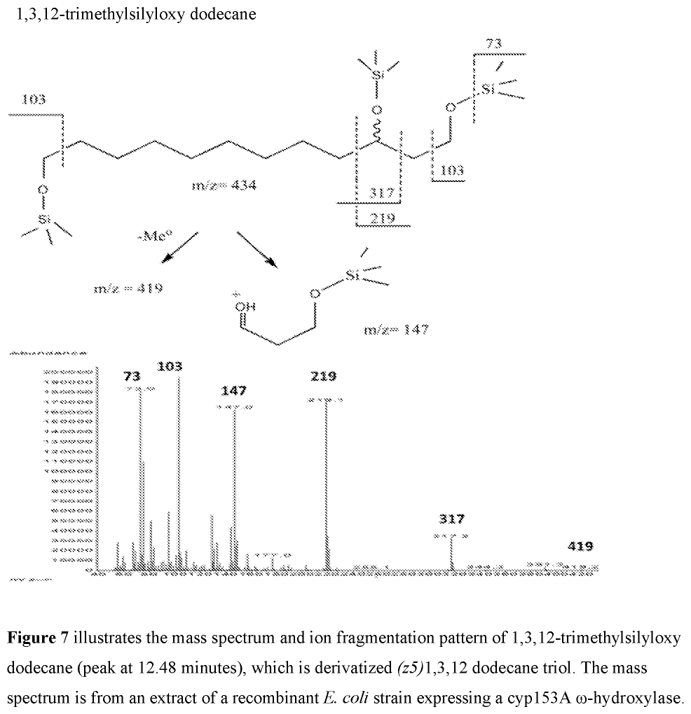

The mass spectrum of the peak at RT 12.33 min is shown in FIG. 6 The fragmentation pattern indicated that this peak was (z5) 1,3,12-trimethylsilyloxy dodecene, which is the derivatized form of (z5) 1,3,12-decenetriol. Characteristic ion fragments are shown in FIG. 6. Ions at m/z=315, 219, 103 are useful diagnostic markers for this compound. Analogously, the peak at RT 12.48 min was identified as 1,3,12-trimethylsilyloxy dodecane, which is the derivatized form of 1,3,12-decanetriol. Characteristic ion fragments are shown in FIG. 7. Ions at m/z=419, 317, 103 are useful diagnostic markers for this compound.

As shown in table 7, the cyp153 P450 enzymes from Marinobacter aquaeolei, Congregibacter litoralis, Limnobacter sp. MED105, Gordonia paraffinivorans, Blastomonas sp. CACIA14H2 and Caulobacter sp. K31 showed the highest conversion to 1,3,12 dodeca(e)netriols. Table 7 also shows that cyp153A P450 from M. aquaeolei efficiently converted 1,3-dodeca(e)nediols to 1,3,12-dodeca(e)netriols as free standing catalytic P450 domain (CYP153A_Maqu) with discrete redox proteins or as chimeric fusion enzymes with either a PFROR-type reductase domain from Rhodococcus (CYP153A_RhF1/2) or a BM3-type reductase domain from Bacillus (CYP153A-BM3). In addition, the alkB-type ω-hydroxylase from Pseudomonas putida (alkBGT) also efficiently converted 1,3-dodeca(e)nediols to 1,3,12-dodeca(e)netriols.

TABLE 7
Conversion of 1,3-dodecanediol to 1,3,12-dodecanetriol using various ω-hydroxylases

| | | | % Conversion |
|---|---|---|---|
| | | Accession Number | 1,3-C12 diol* to |
| ω-hydroxylase | Microorganism | or SEQ ID NO. | 1,3,12-C12 triol** |
| CYP153_mgp_HTCC2143 | marine gamma | WP_007223979 | 1.8 |
| | proteobacterium | | |
| | HTCC2143 | | |
| CYP153_mgp_HTCC2080 | marine gamma | WP_007235225 | 15.8 |
| | proteobacterium | | |
| | HTCC2080 | | |
| CYP153_Clit | Congregibacter | WP_008292970 | 97.0 |
| | litoralis | | |
| CYP153_Lim | Limnobacter sp. | WP_008247244 | 93.9 |
| | MED105 | | |
| CYP153_Caul | Caulobacter sp. K31 | ABZ74416 | 44.3 |
| CYP153_Mmar | Mycobacterium | ACC41588 | 1.7 |
| | marinum str. M | | |
| CYP153_mgp_HTCC2148 | Marine gamma | EEB77967 | 6.6 |
| | proteobacterium | | |
| | HTCC2148 | | |
| CYP153_Pmed | Patulibacter | EHN09160 | 6.0 |
| | medicamentivorans | | |
| CYP153_Ppsy | Paraglaciecola | AGH45156 | 1.7 |
| | psychrophila 170 | | |
| CYP153_Abro | Afipia broomeae | WP_006021902 | 5.5 |
| | ATCC 49717 | | |
| CYP153_Rrub | Rhodococcus ruber | WP_003937314 | 16.1 |
| | BKS 20-38 | | |
| CYP153_Gpar | Gordonia | WP_006901781 | 70.8 |
| | paraffinivorans | | |
| CYP153_A_OC4 | Acinetobacter sp. | AB221118 | 0.0 |
| | OC4 | | |
| CYP153_Smac | Sphingopyxis | AJ850057 | 0.0 |
| | macrogoltabida | | |
| CYP153_M_HXN1500 | Mycobacterium sp. | AJ783967 | 26.4 |
| | HXN-1500 | | |
| CYP153_Cmic | Candidatus | WP_012228788 | 0.9 |
| | Microthrix parvicella | | |
| | RN1 | | |
| CYP153_A_P52 | Afipia sp. P52-10 | WP_034469539 | 0.0 |
| CYP153_B_CACIA14H2 | Blastomonas sp. | ESZ86026 | 47.2 |
| | CACIA14H2 | | |
| CYP153_CPha1 | Candidatus | WP_043950289 | 2.0 |
| | Phaeomarinobacter | | |
| | ectocarpi | | |
| CYP153_CPha2 | Candidatus | WP_043950063 | 15.5 |
| | Phaeomarinobacter | | |
| | ectocarpi | | |
| CYP153A_Maqu | Marinobacter | WP_011784142 | 100.0 |
| | aquaeolei | | |
| CYP153A_RhF1 | Marinobacter | SEQ ID NO: 3 and | 74.9 |
| | aquaeolei | SEQ ID NO: 4 | |
| CYP153A_RhF2 | Marinobacter | SEQ ID NO: 5 and | 97.4 |
| | aquaeolei | SEQ ID NO: 6 | |
| CYP153A-BM3 | Marinobacter | SEQ ID NO: 7 and | 90.2 |
| | aquaeolei | SEQ ID NO: 8 | |
| AlkB | Pseudomonas putida | CAB54050 | 72.7 |

*C12 diol substrate is ~65% 1,3 dodecanediol and ~35% (z5)1,3 dodecenediol
**C12 triol product is 1,3, 12 dodecanetriol and (5z)1,3,12 dodecenetriol

### Example 3

The following Example illustrates the conversion of exogenously added 3-hydroxy dodecanoic acid to 3,12-dihydroxy dodecanoic acid by recombinant E. coli strains expressing various ω-hydroxylases.

The E. coli strains, ω-hydroxylases and experimental design are identical to Example 2, except that in this Example, 3-hydroxy dodecanoic acid was added at induction at a final concentration of 1 g/L (instead of 1,3 dodeca(e)nediol).

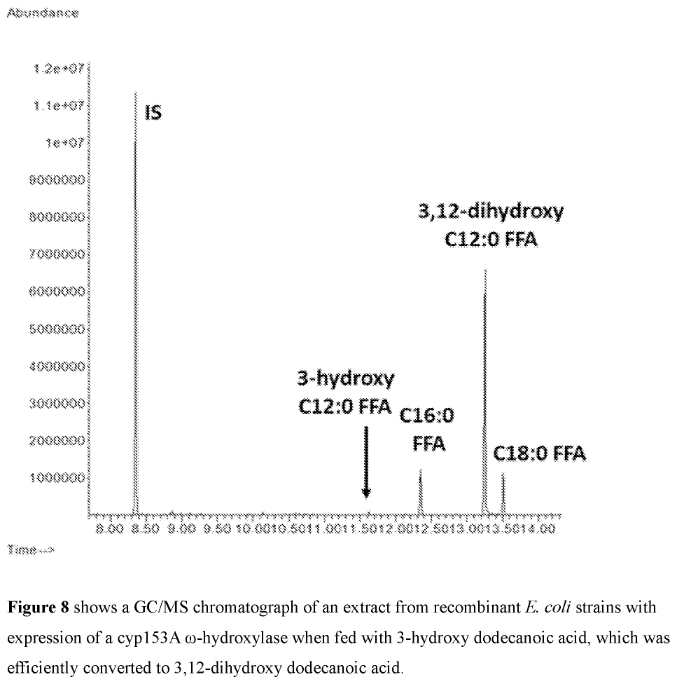

Surprisingly, most strains with the ω-hydroxylases completely or partially converted 3-hydroxy dodecanoic acid to 3,12-dihydroxy dodecanoic acid. In comparison to the control strain without expression of an ω-hydroxylases, one new peak at RT 13.25 minutes (after BSTFA derivatization) appeared, while the 3-hydroxy dodecanoic acid peak disappeared or was reduced (see FIG. 8).

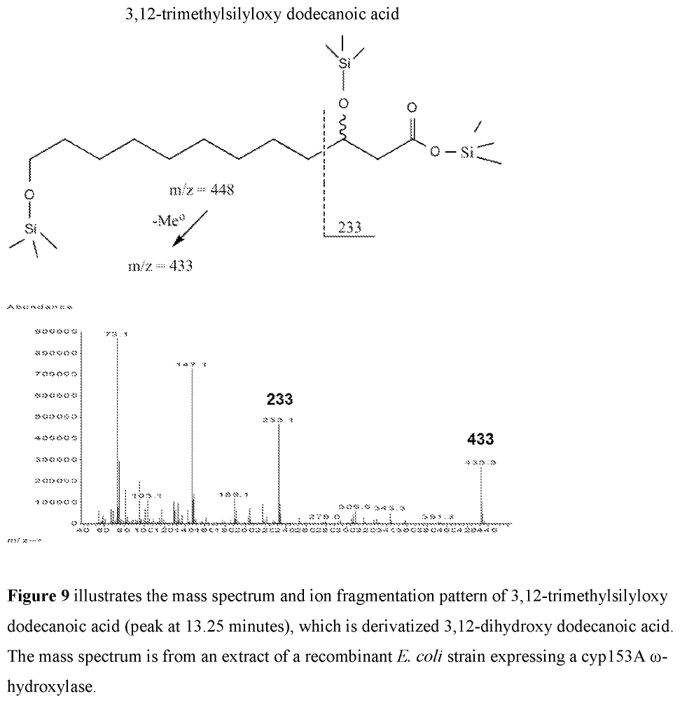

The mass spectrum of the peak at RT 13.25 min is shown in FIG. 9. The fragmentation pattern indicated that this peak was 3,12-trimethylsilyloxy-dodecanoic acid trimethylsilyl ester, which is the derivatized form of 3,12-dihydroxy dodecanoic acid. Characteristic ion fragments used to identify 3,12-trimethylsilyloxy-dodecanoic acid trimethylsilyl ester are shown in FIG. 9. Ions at m/z=433 and 233 are useful diagnostic markers for these compounds. The ion at m/z 433 was used to determine the chain length after loss of CH₃ (m/z=448-15) and ion at 233 was used to determine the fragment ion of —(CH3SiO)CHCH2COOSi(CH3)3 (FIG. 9).

As shown in Table 8, the cyp153A P450 enzymes from Marinobacter aquaeolei, Congregibacter litoralis, Limnobacter sp. MED105, Blastomonas sp. CACIA14H2 and Caulobacter sp. K31 showed the highest conversion to 3,12-dihydroxy dodecanoic acid. Table 8 also shows that cyp153A P450 from M aquaeolei efficiently converted 3-hydroxy dodecanoic acid to 3,12-dihydroxy dodecanoic acid as free standing catalytic P450 domain (CYP153A_Maqu) with discrete redox proteins and as chimeric fusion enzyme with either a PFROR-type reductase domain from Rhodococcus (CYP153A_RhF01/2) or a BM3-type reductase domain from Bacillus (CYP153A-BM3). In addition, the alKB-type ω-hydroxylase from Pseudomonas putida (alkBGT) also converted 3-hydroxy dodecanoic acid to 3,12-dihydroxy dodecanoic acid.

TABLE 8
Conversion of 3-hydroxy dodecanoic acid to 3,12-dihydroxy
dodecanoic acid using various ω-hydroxylases

| | | | % Conversion |
|---|---|---|---|
| | | | 3-OH dodecanoic acid |
| | | | to 3,12 diOH |
| ω-hydroxylase | Microorganism | Accession Number | dodecanoic acid |
| CYP153_mgp_HTCC2143 | marine gamma | WP_007223979 | 0.0 |
| | proteobactenum | | |
| | HTCC2143 | | |
| CYP153_mgp_HTCC2080 | marine gamma | WP_007235225 | 12.4 |
| | proteobacterium | | |
| | HTCC2080 | | |
| CYP153_Clit | Congregibacter | WP_008292970 | 100.0 |
| | litoralis | | |
| CYP153_Lim | Limnobacter sp. | WP_008247244 | 94.3 |
| | MED105 | | |
| CYP153_Caul | Caulobacter sp. K31 | ABZ74416 | 42.0 |
| CYP153_Mmar | Mycobacterium | ACC41588 | 0.0 |
| | marinum str. M | | |
| CYP153_mgp_HTCC2148 | Marine gamma | EEB77967 | 21.5 |
| | proteobacterium | | |
| | HTCC2148 | | |
| CYP153_Pmed | Patulibacter | EHN09160 | 0.0 |
| | medicamentivorans | | |
| CYP153_Ppsy | Paraglaciecola | AGH45156 | 1.3 |
| | psychrophila 170 | | |
| CYP153_Abro | Afipia broomeae | WP_006021902 | 12.5 |
| | ATCC 49717 | | |
| CYP153_Rrub | Rhodococcus ruber | WP_003937314 | 8.3 |
| | BKS 20-38 | | |
| CYP153_Gpar | Gordonia | WP_006901781 | 2.0 |
| | paraffinivorans | | |
| CYP153_A_OC4 | Acinetobacter sp. | AB221118 | 0.0 |
| | OC4 | | |
| CYP153_Smac | Sphingopyxis | AJ850057 | 1.0 |
| | macrogoltabida | | |
| CYP153_M_HXN1500 | Mycobacterium sp. | AJ783967 | 0.5 |
| | HXN-1500 | | |
| CYP153_Cmic | Candidatus | WP_012228788 | 2.0 |
| | Microthrix | | |
| | parvicella RN1 | | |
| CYP153_A_P52 | Afipia sp. P52-10 | WP_034469539 | 0.0 |
| CYP153_B_CACIA14H2 | Blastomonas sp. | ESZ86026 | 92.0 |
| | CACIA14H2 | | |
| CYP153_CPha1 | Candidatus | WP_043950289 | 0.0 |
| | Phaeomarinobacter | | |
| | ectocarpi | | |
| CYP153_CPha2 | Candidatus | WP_043950063 | 20.7 |
| | Phaeomarinobacter | | |
| | ectocarpi | | |
| CYP153A_Maqu | Marinobacter | WP_011784142 | 100.0 |
| | aquaeolei | | |
| CYP153A_RhF1 | Marinobacter | SEQ ID NO: 3 and | 86.1 |
| | aquaeolei | SEQ ID NO: 4 | |
| CYP153A_RhF2 | Marinobacter | SEQ ID NO: 5 and | 29.4 |
| | aquaeolei | SEQ ID NO: 6 | |
| CYP153A-BM3 | Marinobacter | SEQ ID NO: 7 and | 86.8 |
| | aquaeolei | SEQ ID NO: 8 | |
| AlkB | Pseudomonas putida | CAB54050 | 23.2 |

### Example 4

The following Example illustrates the conversion of exogenously added 1,3-dodeca(e)nediols to various dodeca(e)netriols by a recombinant E. coli strain expressing a “subterminal” co-hydroxylase from Bacillus lichenformis (cyp102A1_Blic).

The gene for cyp102A1_Blic was amplified from genomic DNA and cloned into a pCL-derivative vector (SC101 replicon, spectinomycin resistance marker) such that it was under the control of the IPTG-inducible Ptrc. The resulting plasmid, pKM.046, was then transformed into an E. coli MG1655 derivative strain. The small scale fermentation protocol (see Example 1 above) was followed and at the time of induction a mixture of 1,3 dodecanediol and (z5)1,3-dodecenediol (˜65/35%) was added to the cultures at a final concentration of 1 g/L.

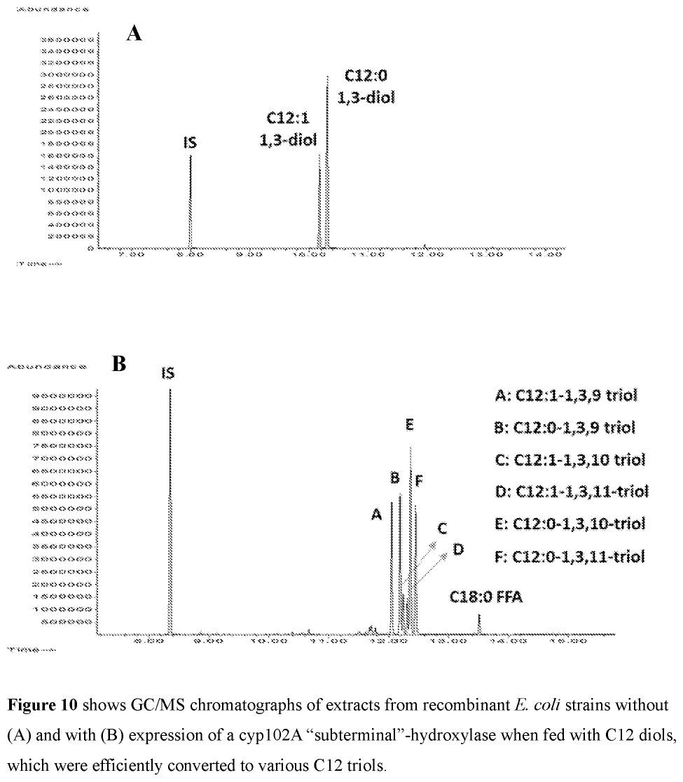

Surprisingly, the strain with cyp102A7_Blic almost completely converted 1,3-dodeca(e)nediols to various dodeca(e)netriols. In comparison to the control strain without expression of an ω-hydroxylases, six new peaks between RT 12.0 and 12.5 min (after TMS/BSTFA derivatization) appeared, while the two 1,3-dodeca(e)nediols derivatized peaks almost completely disappeared (see FIG. 10).

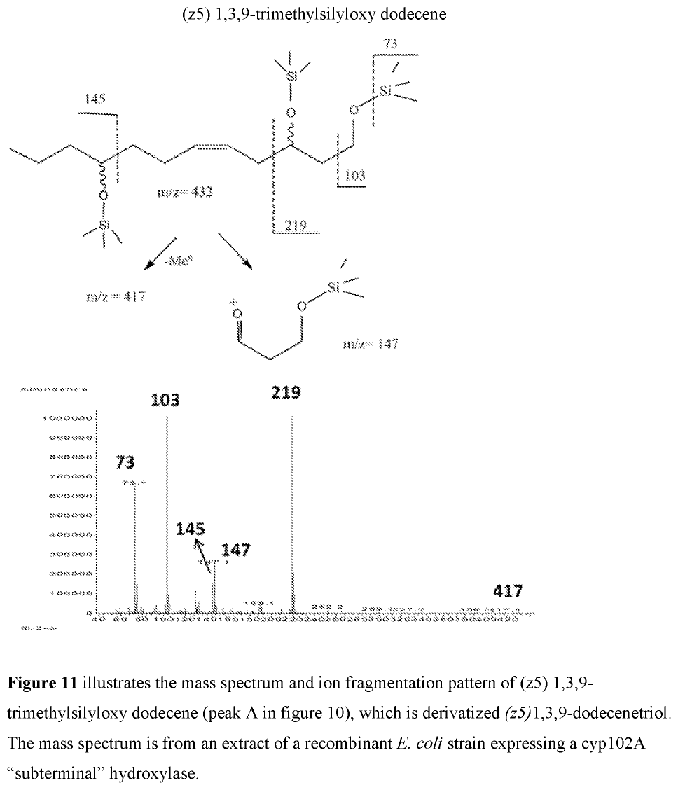

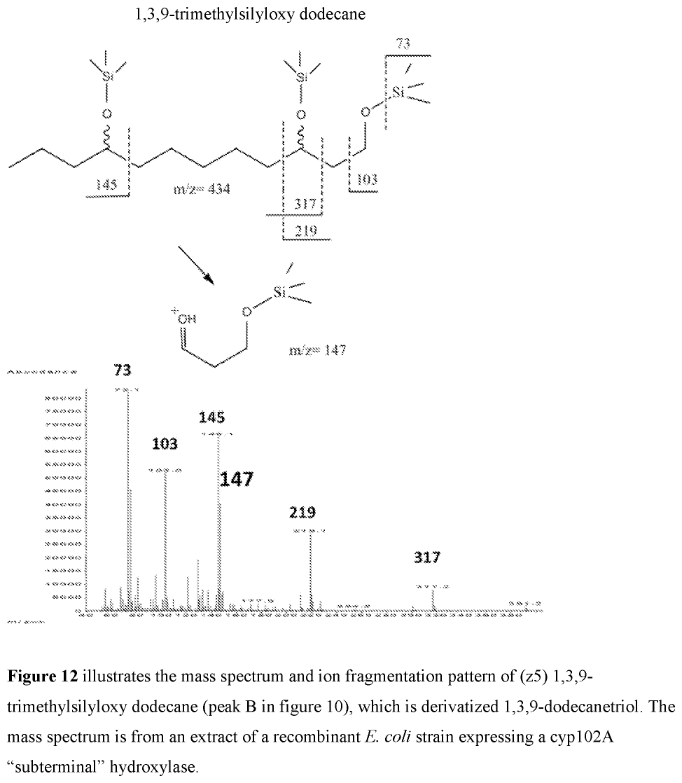

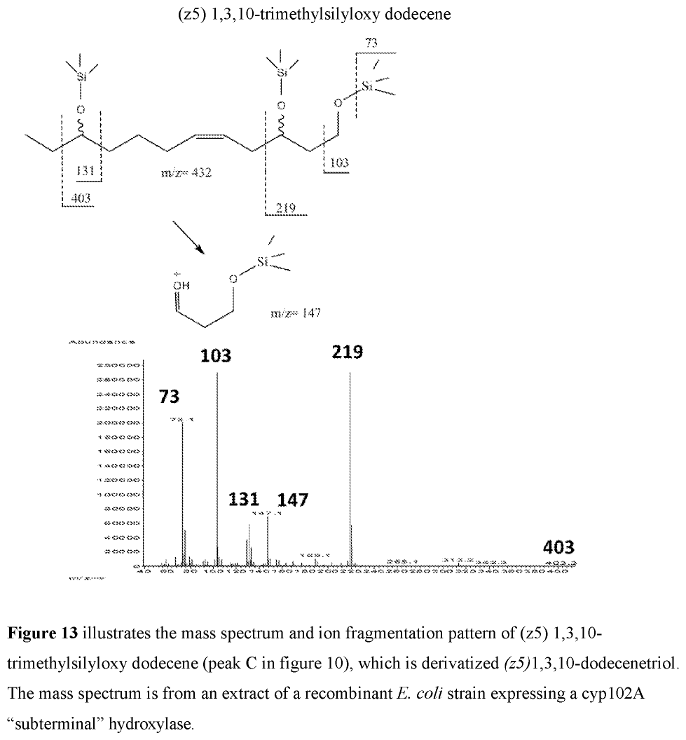

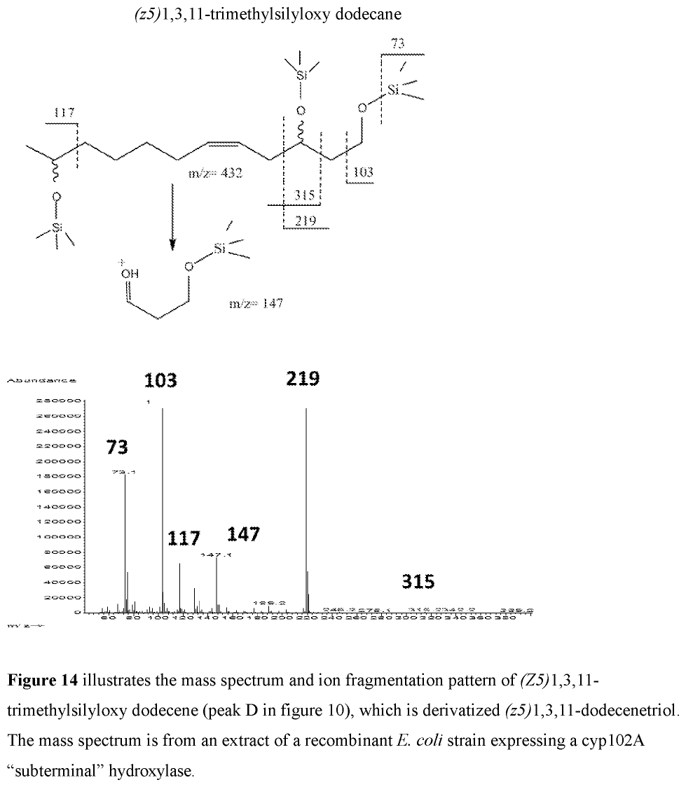

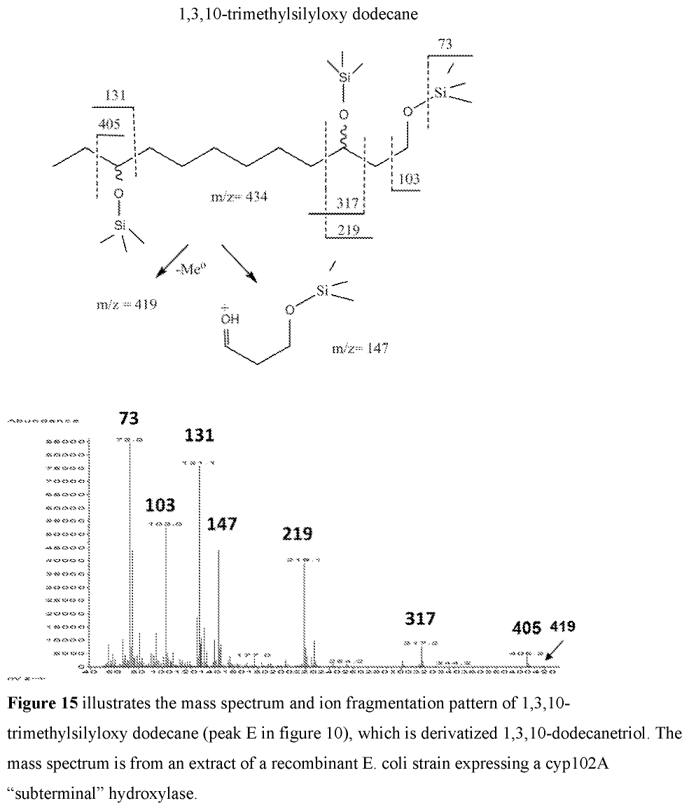

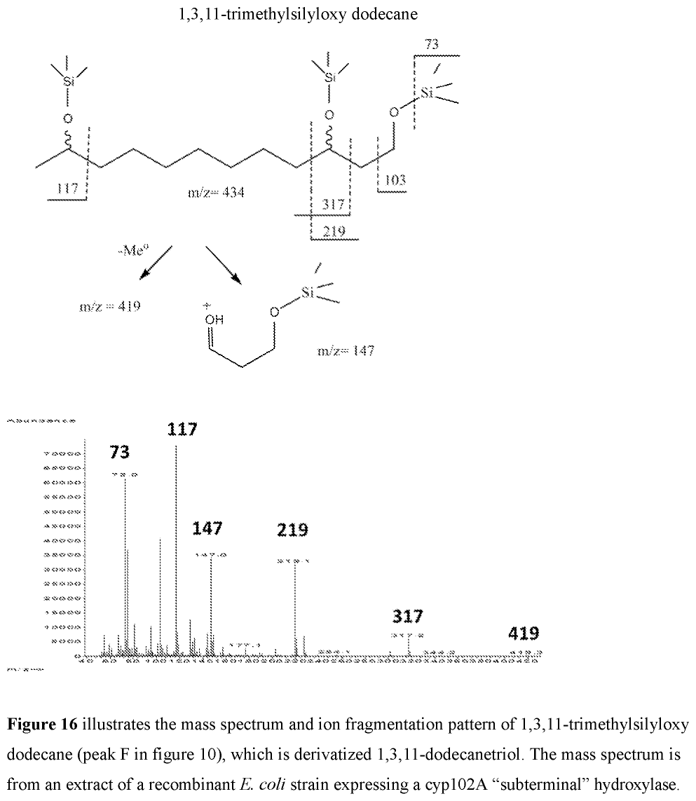

After TMS/BSTFA derivatization, the six peaks were identified from shortest to longest RT as (z5) 1,3,9-trimethylsilyloxy dodecene, 1,3,9-trimethylsilyloxy dodecane, (z5) 1,3,10-trimethylsilyloxy dodecene, (z5) 1,3,11-trimethylsilyloxy dodecene, 1,3,10-trimethylsilyloxy dodecane and 1,3,11-trimethylsilyloxy dodecane which are the derivatized form of (z5) 1,3,9-dodecenetriol, 1,3,9-dodecanetriol, (z5) 1,3,10-dodecenetriol, (z5) 1,3,11-dodecenetriol, 1,3,10-dodecanetriol and 1,3,11-dodecanetriol, respectively (see FIG. 10). The mass spectrum of the derivatized compounds and characteristic fragment ions used to identify these structures are shown in FIGS. 11-16: FIG. 11: (z5) 1,3,9-trimethylsilyloxy dodecene, FIG. 12: 1,3,9-trimethylsilyloxy dodecane, FIG. 13: (z5) 1,3,10-trimethylsilyloxy dodecene, FIG. 14: (z5) 1,3,11-trimethylsilyloxy dodecene, FIG. 15: 1,3,10-trimethylsilyloxy dodecane and FIG. 16: 1,3,11-trimethylsilyloxy dodecane.

### Example 5

The following Example illustrates production of 1,3,12-dodecanetriol from a renewable carbohydrate feedstock such as glucose, by recombinant E. coli strains expressing pathway genes for the production of 1,3 diols and either a P450 ω-hydroxylase, cyp153A(G307A) from M. aquaeolei, or a chimeric hybrid-protein in which a CYP153A P450 hydroxylase is fused with a reductase domain, cyp153A-RhF2.

The gene for the cyp153A(G307A) was amplified from genomic DNA and cloned into a pACYC-derivative vector (p15A replicon, kanamycin resistance marker) such that it was under the control of the IPTG-inducible Ptrc promoter and in an operon with the CamA (putraredoxin reductase) and CamB (putredoxin) genes resulting in plasmid pZR.395 (Table 9). The gene for cyp153A-RhF2 was amplified from a plasmid and cloned into a pACYC-derivative vector (p15A replicon, kanamycin resistance marker) resulting in plasmid pIR.092 (Table 9).

Plasmid pNH308 (Table 9), a pCL1920-derivative vector (SC101 replicon, spectinomycin resistance marker), contained the following operon controlled by the IPTG-inducible Ptrc promoter: a fatty acid reductase variant, carB8 from Mycobacterium smegmatis, a thioesterase, fatB1 from Umbellularia californica, an alcohol dehydrogenase, AlrA from Acinetobacter baylyi, and variants of β-ketoacyl-ACP synthase, fabB, and of a transcriptional regulator, fadR, both from E.coli.

The genome of base strain stNH1525 (Table 10) was engineered as follows: the acyl-CoA dehydrogenase (fadE) gene was attenuated. A phosphopantetheinyl transferase (entD) and a synthetic fatty acid biosynthesis operon (consisting of an enoyl-ACP reductase, an ACP-malonyltransferase, a β-ketoacyl-ACP reductase and a 3-hydroxyacyl-ACP dehydratase and two β-ketoacyl-ACP synthases) were overexpressed. Plasmids pZR.395 and pIR.092 were cotransformed with plasmid pNH.308 into stHN1525 resulting in strains sAS.548 and sZR519, respectively (Table 9). Both strains were subjected to small scale fermentation and product analysis as described in the methods (see above).

Both strains produced triols, which were identified as described in example 1. Strain sAS.548 produced 104 mg/L 1,3,12 dodecanetriol from glucose and strain sZR.519 produced 62 mg/L 1,3,12 dodecanetriol from glucose. Besides triols, sAS.548 and sZR.519 produced various fatty alcohols (533 and 898 mg/L, respectively) and diols (477 and 377 mg/L, respectively).

This example showed that E. coli strains engineered for producing 1,3 diols when combined with the expression of aCYP153 P450 ω-hydroxylase or achimeric hybrid-protein CYP153 P450 ω-hydroxylase produced fatty triols from glucose.

TABLE 9
Plasmids used for triol or dihydroxy fatty acid production from glucose

| Plasmid Name | Pathway Genes |
|---|---|
| pZR.395 | pACYC-trc-cyp153A(G307A)-camA-camB |
| pIR.092 | pACYC-trc-cyp153A(G307A)-RhF2 |
| pZR.468 | pACYC-trc-cyp102A7 |
| pKM.046 | pCL-trc-cyp102A7 |
| pNH.308 | pCL-trc-carB-fatB1-alrA-fabB-fadR |
| pNH.328 | pCL-trc-carB-phaG-alrA |
| pKEV199 | pCL-trc-fatB1 |
| pZR.427 | pCL-trc-fatA3-fabB-T5-cyp153A-RhF2 |
| pKM.080 | pCL-trc-fatA3-fabB-T5-cyp102A7 |
| pAL.001 | pACYC-ohyA1_Smal |
| pAL.002 | pACYC-ohyA2_Smal |

TABLE 10
Strains used for triol or dihydroxy
fatty acid production from glucose

| Strain Name | Description |
|---|---|
| stNH1525 | Base strain, no plasmids |
| AA.207 | Base strain, no plasmids |
| TLC2 | Base strains, no plasmids |
| sAS.548 | stNH1525 with plasmids pNH308 and pZR.395 |
| sZR.519 | stNH1525 with plasmids pNH308 and pIR.092 |
| sZR.521 | stNH1525 with plasmids pNH308 and pZR.468 |
| sZR.525 | AA.207 with plasmids pKEV199 and pIR.092 |
| sAL.131 | TLC2 with plasmids AA.233 with plasmids |
| | pAL.001 and pZR.427 |
| sAL.132 | TLC2 with plasmids pAL.002 and pZR.427 |
| sAL.134 | TLC2 with plasmids pAL.001 and pKM.080 |
| sAL.135 | TLC2 with plasmids pAL.002 and pKM.080 |

### Example 6

The following Example illustrates production of subterminally-hydroxylated triols from a renewable carbohydrate feedstock such as glucose, by a recombinant E. coli strain expressing pathway genes for the production of 1,3 diols and a cyp102A7 P450 hydroxylase from B. licheniformis, cyp102A7 Blic.

The gene for the cyp102A7_Blic was amplified from genomic DNA and cloned into a pACYC-derivative vector (p15A replicon, kanamycin resistance marker) such that it was under the control of the IPTG-inducible Ptrc promoter resulting in plasmid pZR.468 (Table 9).

Plasmids pZR.468 was cotransformed with plasmid pNH.308 into stNH1525 (see Example 5) resulting in strain sZR.521 (Table 10). The strain was subjected to small scale fermentation and product analysis as described in the methods (Example 1).

The strain produced triols, which were identified as described in Example 4. sZR.521 produced 122 mg/L 1,3,10-dodecanetriol and 24 mg/L 1,3,11-dodecanetriol from glucose. Besides triols, sZR.521 produced various fatty alcohols (614 mg/L) and diols (318 mg/L, respectively).

This example showed that E. coli strains engineered for producing 1,3 diols when combined with the expression of a CYP102A7 P450 hydroxylase produced fatty triols from glucose.

### Example 7

The following Example illustrates production of 3,12-dihydroxy dodecanoic acid from a renewable carbohydrate feedstock such as glucose, by recombinant E. coli strains expressing pathway genes for the production of 3-hydroxy fatty acids and a cyp153A P450 w-hydroxylase.

The gene for cyp153A-RhF2 was amplified from a plasmid and cloned into a pACYC-derivative vector (p15A replicon, kanamycin resistance marker) such that it was under the control of the IPTG-inducible Ptrc promoter resulting in plasmid pIR.092 (Table 9).

Plasmid pKEV199 (Table 9), a pCL1920-derivative vector (SC101 replicon, spectinomycin resistance marker), contained a thioesterase, fatB1 from Umbellularia californica, controlled by the IPTG-inducible Ptrc promoter.

The genome of base strain AA.207 (Table 9) was engineered as follows: the acyl-CoA dehydrogenase (fadE) gene was attenuated and a synthetic fatty acid biosynthesis operon (consisting of an enoyl-ACP reductase, an ACP-malonyltransferase, a β-ketoacyl-ACP reductase and a 3-hydroxyacyl-ACP dehydratase and two β-ketoacyl-ACP synthases) and a variant of the transcriptional regulator fadR were overexpressed.

Plasmid pIR.092 (see Example 5 and Table 9) was cotransformed with plasmid pKEV199 into stHN1525 resulting in strain sZR525 (Table 9). The strain was subjected to small scale fermentation and product analysis as described in the methods (see above).

The strains produced dihydroxy-fatty acids, which were identified as described in Example 3. Strain sZR.525 produced 6 mg/L 3,12-dihydroxy dodecanoic acid from glucose. Besides dihydroxy fatty acids, sZR.525 produced various fatty acids (411 mg/L) and 3-hydroxy fatty acids (1089 mg/L, respectively).

This example showed that an E. coli strain engineered for producing 3-hydroxy fatty acids when combined with the expression of a CYP153 P450 w-hydroxylase produced dihydroxy fatty acids from glucose.

### Example 8

Production of 10,16-dihydroxy hexadecanoic acid by a recombinant E. coli strain from a simple carbon source.

This example shows the production of 10,16-dihydroxy hexadecanoic acid from a renewable carbohydrate feedstock such as glucose, by recombinant E. coli strains expressing pathway genes for two fatty acid-hydroxylating enzymes, the chimeric hybrid-protein cyp153A-RhF2 from M. aquaeolei, and OhyA1 or OhyA2 from Stenotrophomonas maltophilia.

Plasmid pAL.001 and pAL.002 (Table 9), pACYC-derivative vectors (p15A replicon, kanamycin resistance marker), contained the fatty acid hydroxylases ohyA1 and ohyA2 from S. maltophilia, respectively, controlled by the IPTG-inducible Ptrc promoter. Plasmid pZR.427 (Table 9), a pCL1920-derivative vector (SC101 replicon, spectinomycin resistance marker), contained the following two operons: (i) the IPTG-inducible Ptrc promoter controlling thioesterase FatA from A. thaliana and p-ketoacyl-ACP synthase, fabB, and (ii) a IPTG-inducible PT5 promoter controlling cyp153A-RhF2 from M. aquaeolei.

The genome of base strain TLC2 (Table 10) was a derivative of E. coli MG1655 with an attenuated acyl-CoA dehydrogenase (fadE) gene.

Plasmid pZR.427 was cotransformed with plasmids pAL.001 or pAL.002 into TLC2 resulting in strains sAL.131 and sAL.132, respectively (Table 9). The strains were subjected to small scale fermentation and product analysis as described in the methods (see above).

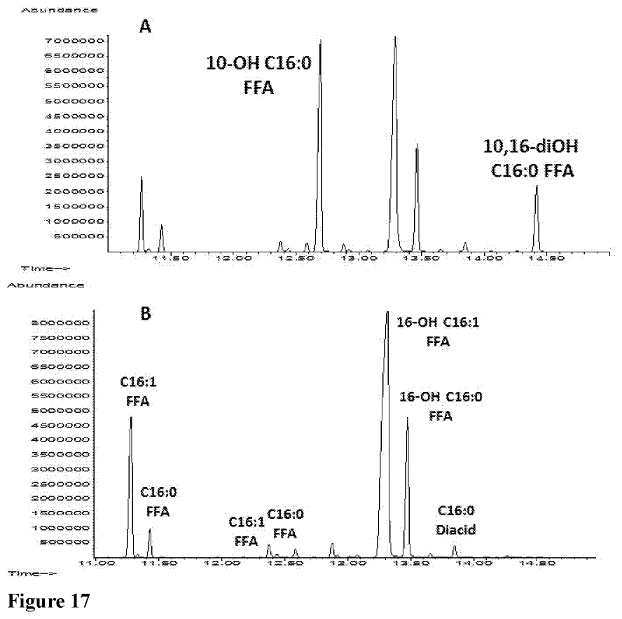

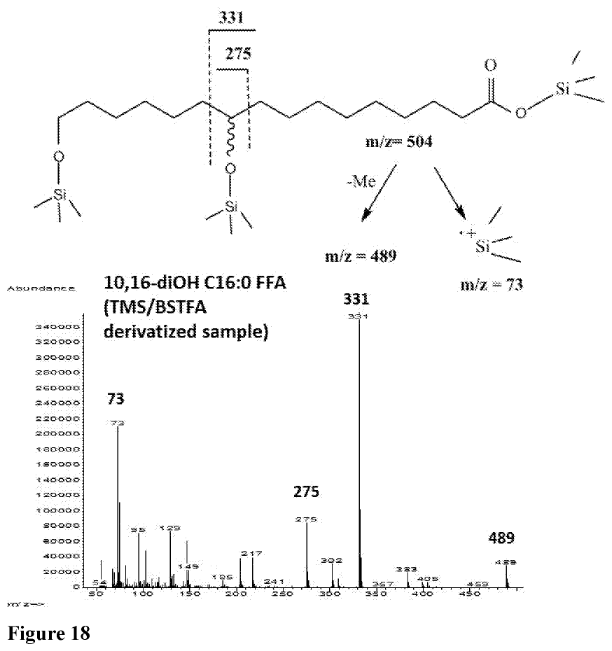

The extracts from strains sAL.131 (FIG. 17A) and sAL.132 showed two new peaks at RT 12.692 and 14.415 min (after TMS/BSTFA derivatization), that were not observed in the control strain without expressing ohyA1 or ohyA2 (FIG. 17B). The mass spectrum and the retention time of the peak at RT 12.692 min in comparison with authentic standard sample indicated that this peak was trimethylsilyl derivative of 10-hydroxy hexadecanoic acid (10-OH C16:0 FFA). The mass spectrum of the peak at 14.415 min is shown in FIG. 18. The fragmentation pattern indicated that this peak was trimethylsilyl derivative of 10,16-dihydroxy hexadecanoic acid (10,16-diOH C16:0 FFA) which is derived from 10-hydroxy hexadecanoic acid (10-OH C16:0 FFA). Characteristic ion fragments are shown in FIG. 18. Ions at m/z=489, 331 and 275 are useful diagnostic markers for this compound.

Strain sAL.131 and sAL.132 produced 14 mg/L and 9 mg/L 10,16-hexadecanoic acid from glucose, respectively. Besides 10,16-hexadecanoic acid both strains also produced 10-hydroxy hexadecanoic acid, 16-hydroxy hexadecanoic acid, hexadecanoic acid, (z9)-hexadecenoic acid and small amounts of other fatty acid derivatives.

This example showed that E. coli strains engineered for producing fatty acids when combined with the expression of a CYP153A P450 ω-hydroxylase and a fatty acid hydratase produced dihydroxy fatty acids from glucose.

### Example 9

Production of 10,13-, 10,14- and 10,15-dihydroxy hexadecanoic acid by a recombinant E. coli strain from a simple carbon source.

This example shows the production of 10,13-, 10,14- and 10,15-dihydroxy hexadecanoic acid from a renewable carbohydrate feedstock such as glucose, by recombinant E. coli strains expressing pathway genes for two fatty acid-hydroxylating enzymes, cyp102A7 from B. licheniformis, and OhyA1 or OhyA2 from Stenotrophomonas maltophilia.

Plasmid pAL.001 and pAL.002 (Table 9), pACYC-derivative vectors (p15A replicon, kanamycin resistance marker), contained the fatty acid hydroxylases ohyA1 and ohyA2 from S. maltophilia, respectively, controlled by the IPTG-inducible Ptrc promoter. Plasmid pKM.080 (Table 9), a pCL1920-derivative vector (SC101 replicon, spectinomycin resistance marker), contained the following two operons: (i) the IPTG-inducible Ptrc promoter controlling thioesterase fatA from A. thaliana and p-ketoacyl-ACP synthase, fabB, and (ii) a IPTG-inducible PT5 promoter controlling cyp102A7 from B. licheniformis.

Plasmid pKM.080 was cotransformed with plasmids pAL.001 or pAL.002 into TLC2 (see Example 8 and Table 10) resulting in strains sAL.134 and sAL.135, respectively (Table 9). The strains were subjected to small scale fermentation and product analysis as described in the methods (see above).

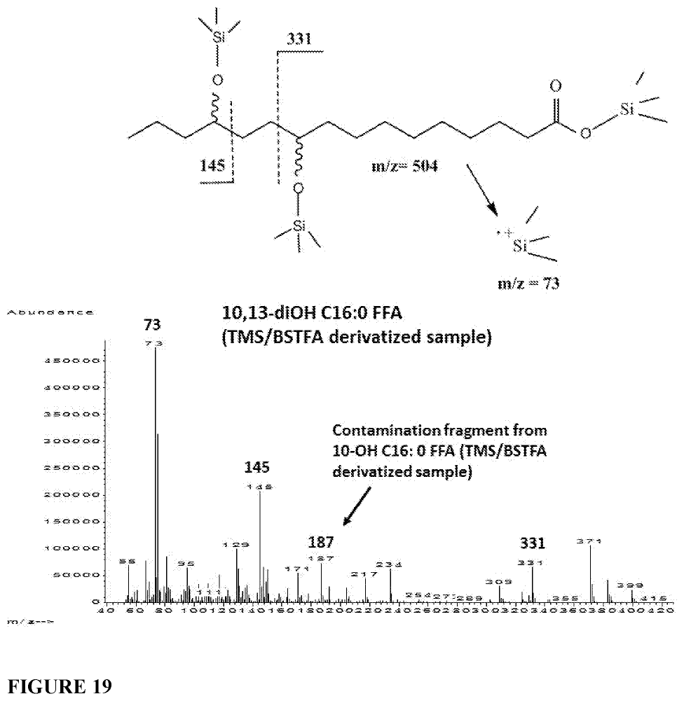

In comparison to a control strain without expression of ohyA1 or ohyA2, three major new peaks at RT 12.692, 13.948 and 14.062 min (after TMS/BSTFA derivatization) appeared in extracts from strains sAL.134 and sAL.135. (data not shown). The mass spectrum scan through the peak at RT 12.692 indicated that this peak was a mixture of two products. The fragmentation patterns indicated that the major product was the trimethylsilyl derivative of 10-hydroxy hexadecanoic acid (10-OH C16:0 FFA) as confirmed using authentic standard sample and the minor product was trimethylsilyl derivative of 10,13-dihydroxy hexadecanoic acid (10,13-diOH C16:0) (FIG. 19). Characteristic ions of trimethylsilyl derivative of 10,13-dihydroxy hexadecanoic acid (10,13-diOH C16:0) obtained from this scan is shown in FIG. 19. Characteristic ions at 331 and 145 are useful diagnostic markers for this compound.

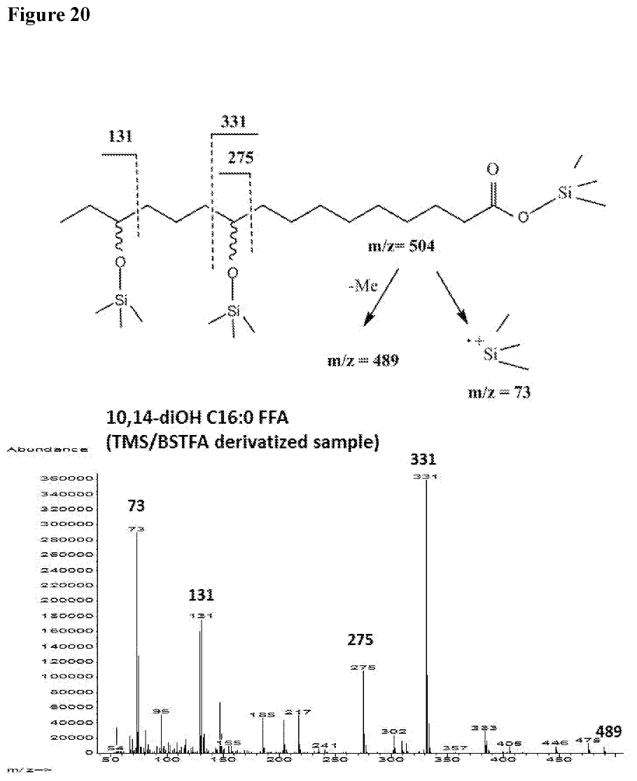

The mass spectrum of the peak at 13.948 min is shown in FIG. 20. The fragmentation patterns indicated that this peak was the tetramethylsilyl derivative of 10,14-dihydroxy hexadecanoic acid (10,14-diOH C16:0 FFA). Characteristic ion fragments are shown in FIG. 20. Ions at m/z=489, 331, 275 and 131 are useful diagnostic markers for this compound.

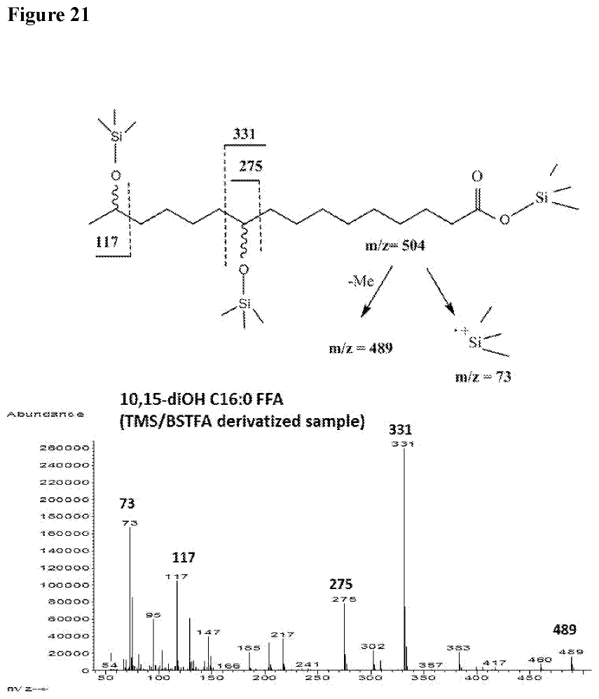

The mass spectrum of the peak at RT 14.062 min is shown in FIG. 21. The fragmentation pattern indicated that this peak was the trimethylsilyl derivative of 10,15-dihydroxy hexadecanoic acid (10,15-diOH C16:0 FFA). Characteristic ion fragments are shown in FIG. 21. Ions at m/z=489, 331, 275, 117 are useful diagnostic markers for this compound.

Strain sAL.134 produced from glucose 17 mg/L 10,14-dihydroxy hexadecanoic acid and 28 mg/L 10,15-dihydroxy hexadecanoic acid, the amount of 10,13-dihydroxy hexadecanoic acid could not be quantified as the GC peak overlapped with 10-hydroxy hexadecanoic acid. Strain sAL.135 produced from glucose 13 mg/L 10,14-dihydroxy hexadecanoic acid and 10,15-dihydroxy hexadecanoic acid produced was under the quantitation limit, the amount of 10,13-dihydroxy hexadecanoic acid could not be quantified as the GC peak overlapped with 10-hydroxy hexadecanoic acid.

Both strains also produced 10-hydroxy hexadecanoic acid, 15-hydroxy hexadecanoic acid, 14-hydroxy hexadecanoic acid, 13-hydroxy hexadecanoic acid, hexadecanoic acid, (z9)-hexadecenoic acid and small amounts of other fatty acid derivatives.

This example showed that E. coli strains engineered for producing fatty acids when combined with the expression of a CYP1012A subterminal-hydroxylase and a fatty acid hydratase produced dihydroxy fatty acids from glucose.

As is apparent to one of skill in the art, various modifications and variations of the above aspects and embodiments can be made without departing from the spirit and scope of this disclosure. Such modifications and variations are thus within the scope of this disclosure.

APPENDIX 1
SEQUENCES

Cyp153A (G307A) from Marinobacter aquaeolei (DNA)
SEQ ID NO: 1
ATGCCAACACTGCCCAGAACATTTGACGACATTCAGTCCCGACTGATTAACGCCACC
TCCAGGGTGGTGCCGATGCAGAGGCAAATTCAGGGACTGAAATTCTTAATGAGCGC
CAAGAGGAAGACCTTCGGCCCACGCCGACCGATGCCCGAATTCGTTGAAACACCCA
TCCCGGACGTTAACACGCTGGCCCTTGAGGACATCGATGTCAGCAATCCGTTTTTAT
ACCGGCAGGGTCAGTGGCGCGCCTATTTCAAACGGTTGCGTGATGAGGCGCCGGTC
CATTACCAGAAGAACAGCCCTTTCGGCCCCTTCTGGTCGGTAACTCGGTTTGAAGAC
ATCCTGTTCGTGGATAAGAGTCACGACCTGTTTTCCGCCGAGCCGCAAATCATTCTC
GGTGACCCTCCGGAGGGGCTGTCGGTGGAAATGTTCATAGCGATGGATCCGCCGAA
ACACGATGTGCAGCGCAGCTCGGTGCAGGGAGTAGTGGCACCGAAAAACCTGAAGG
AGATGGAGGGGCTGATCCGATCACGCACCGGCGATGTGCTTGACAGCCTGCCTACA
GACAAACCCTTTAACTGGGTACCTGCTGTTTCCAAGGAACTCACAGGCCGCATGCTG
GCGACGCTTCTGGATTTTCCTTACGAGGAACGCCACAAGCTGGTTGAGTGGTCGGAC
AGAATGGCAGGTGCAGCATCGGCCACCGGCGGGGAGTTTGCCGATGAAAATGCCAT
GTTTGACGACGCGGCAGACATGGCCCGGTCTTTCTCCAGGCTTTGGCGGGACAAGGA
GGCGCGCCGCGCAGCAGGCGAGGAGCCCGGTTTCGATTTGATCAGCCTGTTGCAGA
GCAACAAAGAAACGAAAGACCTGATCAATCGGCCGATGGAGTTTATCGGTAATTTG
ACGCTGCTCATAGTCGcCGGCAACGATACGACGCGCAACTCGATGAGTGGTGGCCTG
GTGGCCATGAACGAATTCCCCAGGGAATTTGAAAAATTGAAGGCAAAACCGGAGTT
GATTCCGAACATGGTGTCGGAAATCATCCGCTGGCAAACGCCGCTGGCCTATATGCG
CCGAATCGCCAAGCAGGATGTCGAACTGGGCGGCCAGACCATCAAGAAGGGTGATC
GAGTTGTCATGTGGTACGCGTCGGGTAACCGGGACGAGCGCAAATTTGACAACCCC
GATCAGTTCATCATTGATCGCAAGGACGCACGAAACCACATGTCGTTCGGCTATGGG
GTTCACCGTTGCATGGGCAACCGTCTGGCTGAACTGCAACTGCGCATCCTCTGGGAA
GAAATACTCAAGCGTTTTGACAACATCGAAGTCGTCGAAGAGCCCGAGCGGGTGCA
GTCCAACTTCGTGCGGGGCTATTCCAGGTTGATGGTCAAACTGACACCGAACAGTTA
A
Cyp153A (G307A) from Marinobacter aquaeolei (protein)
SEQ ID NO: 2
MPTLPRTFDDIQSRLINATSRVVPMQRQIQGLKFLMSAKRKTFGPRRPMPEFVETPIPDV
NTLALEDIDVSNPFLYRQGQWRAYFKRLRDEAPVHYQKNSPFGPFWSVTRFEDILFVDK
SHDLFSAEPQIILGDPPEGLSVEMFIAMDPPKHDVQRSSVQGVVAPKNLKEMEGLIRSRT
GDVLDSLPTDKPFNWVPAVSKELTGRMLATLLDFPYEERHKLVEWSDRMAGAASATG
GEFADENAMFDDAADMARSFSRLWRDKEARRAAGEEPGFDLISLLQSNKETKDLINRP
MEFIGNLTLLIVAGNDTTRNSMSGGLVAMNEFPREFEKLKAKPELIPNMVSEIIRWQTPL
AYMRRIAKQDVELGGQTIKKGDRVVMWYASGNRDERKFDNPDQFIIDRKDARNHMSF
GYGVHRCMGNRLAELQLRILWEEILKRFDNIEVVEEPERVQSNFVRGYSRLMVKLTPNS.
Cyp153A-RhF1, chimeric hybrid-fusion protein (DNA)
SEQ ID NO: 3
ATGCCAACACTGCCCAGAACATTTGACGACATTCAGTCCCGACTGATTAACGCCACC
TCCAGGGTGGTGCCGATGCAGAGGCAAATTCAGGGACTGAAATTCTTAATGAGCGC
CAAGAGGAAGACCTTCGGCCCACGCCGACCGATGCCCGAATTCGTTGAAACACCCA
TCCCGGACGTTAACACGCTGGCCCTTGAGGACATCGATGTCAGCAATCCGTTTTTAT
ACCGGCAGGGTCAGTGGCGCGCCTATTTCAAACGGTTGCGTGATGAGGCGCCGGTC
CATTACCAGAAGAACAGCCCTTTCGGCCCCTTCTGGTCGGTAACTCGGTTTGAAGAC
ATCCTGTTCGTGGATAAGAGTCACGACCTGTTTTCCGCCGAGCCGCAAATCATTCTC
GGTGACCCTCCGGAGGGGCTGTCGGTGGAAATGTTCATAGCGATGGATCCGCCGAA
ACACGATGTGCAGCGCAGCTCGGTGCAGGGAGTAGTGGCACCGAAAAACCTGAAGG
AGATGGAGGGGCTGATCCGATCACGCACCGGCGATGTGCTTGACAGCCTGCCTACA
GACAAACCCTTTAACTGGGTACCTGCTGTTTCCAAGGAACTCACAGGCCGCATGCTG
GCGACGCTTCTGGATTTTCCTTACGAGGAACGCCACAAGCTGGTTGAGTGGTCGGAC
AGAATGGCAGGTGCAGCATCGGCCACCGGCGGGGAGTTTGCCGATGAAAATGCCAT
GTTTGACGACGCGGCAGACATGGCCCGGTCTTTCTCCAGGCTTTGGCGGGACAAGGA
GGCGCGCCGCGCAGCAGGCGAGGAGCCCGGTTTCGATTTGATCAGCCTGTTGCAGA
GCAACAAAGAAACGAAAGACCTGATCAATCGGCCGATGGAGTTTATCGGTAATTTG
ACGCTGCTCATAGTCGcCGGCAACGATACGACGCGCAACTCGATGAGTGGTGGCCTG
GTGGCCATGAACGAATTCCCCAGGGAATTTGAAAAATTGAAGGCAAAACCGGAGTT
GATTCCGAACATGGTGTCGGAAATCATCCGCTGGCAAACGCCGCTGGCCTATATGCG
CCGAATCGCCAAGCAGGATGTCGAACTGGGCGGCCAGACCATCAAGAAGGGTGATC
GAGTTGTCATGTGGTACGCGTCGGGTAACCGGGACGAGCGCAAATTTGACAACCCC
GATCAGTTCATCATTGATCGCAAGGACGCACGAAACCACATGTCGTTCGGCTATGGG
GTTCACCGTTGCATGGGCAACCGTCTGGCTGAACTGCAACTGCGCATCCTCTGGGAA
GAAATACTCAAGCGTTTTGACAACATCGAAGTCGTCGAAGAGCCCGAGCGGGTGCA
GTCCAACTTCGTGCGGGGCTATTCCAGGTTGATGGTCAAACTGACACCGAACAGTGT
ACTCCATCGTCATCAACCTGTCACCATCGGCGAGCCGGCCGCTCGTGCTGTGAGCCG
CACGGTGACCGTTGAGCGTCTTGATCGCATTGCCGACGATGTCCTTCGCCTGGTCCTT
CGCGATGCTGGAGGTAAAACCCTCCCGACGTGGACGCCTGGCGCTCACATCGACCT
GGATCTGGGtGCTCTGAGCCGTCAGTATTCGCTCTGCGGCGCTCCGGATGCTCCGTCG
TACGAAATCGCCGTGCACTTAGATCCgGAAAGCCGTGGTGGAAGCCGCTATATTCAT
GAACAGCTGGAAGTTGGAAGTCCGCTGCGTATGCGtGGCCCACGCAACCATTTCGCC
CTGGATCCGGGtGCGGAACATTACGTGTTTGTTGCCGGGGGTATCGGCATCACGCCG
GTGCTGGCAATGGCGGATCATGCCCGTGCGCGTGGTTGGTCGTACGAACTGCATTAT
TGTGGTCGTAATCGTAGCGGTATGGCTTACCTGGAACGCGTCGCGGGACATGGTGAC
CGCGCtGCCTTGCACGTATCTGAAGAAGGCACCCGCATTGATCTGGCGGCATTACTT
GCTGAACCGGCGCCGGGCGTGCAAATCTACGCCTGCGGTCCGGGCCGTTTATTAGCG
GGTCTTGAAGACGCGTCTCGTAATTGGCCGGATGGCGCGCTTCATGTGGAGCATTTC
ACTTCGAGTTTAGCCGCTTTGGATCCGGATGTCGAACATGCCTTTGATTTGGAGCTG
CGTGACTCTGGCCTTACCGTTCGCGTCGAGCCAACTCAGACCGTTTTAGACGCTTTG
CGTGCGAACAATATCGACGTCCCGTCGGATTGCGAAGAGGGGCTGTGTGGTTCTTGC
GAAGTAGCCGTTCTGGATGGCGAGGTTGATCACCGTGATACCGTTCTGACTAAGGCC
GAGCGCGCCGCGAATCGTCAGATGATGACTTGCTGCAGTCGTGCATGCGGTGATCGT
CTGGCGCTGCGCCTCTAA
Cyp153A-RhF1, chimeric hybrid-fusion protein (protein)
SEQ ID NO: 4
MPTLPRTFDDIQSRLINATSRVVPMQRQIQGLKFLMSAKRKTFGPRRPMPEFVETPIPDV
NTLALEDIDVSNPFLYRQGQWRAYFKRLRDEAPVHYQKNSPFGPFWSVTRFEDILFVDK
SHDLFSAEPQIILGDPPEGLSVEMFIAMDPPKHDVQRSSVQGVVAPKNLKEMEGLIRSRT
GDVLDSLPTDKPFNWVPAVSKELTGRMLATLLDFPYEERHKLVEWSDRMAGAASATG
GEFADENAMFDDAADMARSFSRLWRDKEARRAAGEEPGFDLISLLQSNKETKDLINRP
MEFIGNLTLLIVAGNDTTRNSMSGGLVAMNEFPREFEKLKAKPELIPNMVSEIIRWQTPL
AYMRRIAKQDVELGGQTIKKGDRVVMWYASGNRDERKFDNPDQFIIDRKDARNHMSF
GYGVHRCMGNRLAELQLRILWEEILKRFDNIEVVEEPERVQSNFVRGYSRLMVKLTPNS
VLHRHQPVTIGEPAARAVSRTVTVERLDRIADDVLRLVLRDAGGKTLPTWTPGAHIDLD
LGALSRQYSLCGAPDAPSYEIAVHLDPESRGGSRYIHEQLEVGSPLRMRGPRNHFALDPG
AEHYVFVAGGIGITPVLAMADHARARGWSYELHYCGRNRSGMAYLERVAGHGDRAAL
HVSEEGTRIDLAALLAEPAPGVQIYACGPGRLLAGLEDASRNWPDGALHVEHFTSSLAA
LDPDVEHAFDLELRDSGLTVRVEPTQTVLDALRANNIDVPSDCEEGLCGSCEVAVLDGE
VDHRDTVLTKAERAANRQMMTCCSRACGDRLALRL.
Cyp153A-RhF2, chimeric hybrid-fusion protein (DNA)
SEQ ID NO: 5
ATGCCAACACTGCCCAGAACATTTGACGACATTCAGTCCCGACTGATTAACGCCACC
TCCAGGGTGGTGCCGATGCAGAGGCAAATTCAGGGACTGAAATTCTTAATGAGCGC
CAAGAGGAAGACCTTCGGCCCACGCCGACCGATGCCCGAATTCGTTGAAACACCCA
TCCCGGACGTTAACACGCTGGCCCTTGAGGACATCGATGTCAGCAATCCGTTTTTAT
ACCGGCAGGGTCAGTGGCGCGCCTATTTCAAACGGTTGCGTGATGAGGCGCCGGTC
CATTACCAGAAGAACAGCCCTTTCGGCCCCTTCTGGTCGGTAACTCGGTTTGAAGAC
ATCCTGTTCGTGGATAAGAGTCACGACCTGTTTTCCGCCGAGCCGCAAATCATTCTC
GGTGACCCTCCGGAGGGGCTGTCGGTGGAAATGTTCATAGCGATGGATCCGCCGAA
ACACGATGTGCAGCGCAGCTCGGTGCAGGGAGTAGTGGCACCGAAAAACCTGAAGG
AGATGGAGGGGCTGATCCGATCACGCACCGGCGATGTGCTTGACAGCCTGCCTACA
GACAAACCCTTTAACTGGGTACCTGCTGTTTCCAAGGAACTCACAGGCCGCATGCTG
GCGACGCTTCTGGATTTTCCTTACGAGGAACGCCACAAGCTGGTTGAGTGGTCGGAC
AGAATGGCAGGTGCAGCATCGGCCACCGGCGGGGAGTTTGCCGATGAAAATGCCAT
GTTTGACGACGCGGCAGACATGGCCCGGTCTTTCTCCAGGCTTTGGCGGGACAAGGA
GGCGCGCCGCGCAGCAGGCGAGGAGCCCGGTTTCGATTTGATCAGCCTGTTGCAGA
GCAACAAAGAAACGAAAGACCTGATCAATCGGCCGATGGAGTTTATCGGTAATTTG
ACGCTGCTCATAGTCGcCGGCAACGATACGACGCGCAACTCGATGAGTGGTGGCCTG
GTGGCCATGAACGAATTCCCCAGGGAATTTGAAAAATTGAAGGCAAAACCGGAGTT
GATTCCGAACATGGTGTCGGAAATCATCCGCTGGCAAACGCCGCTGGCCTATATGCG
CCGAATCGCCAAGCAGGATGTCGAACTGGGCGGCCAGACCATCAAGAAGGGTGATC
GAGTTGTCATGTGGTACGCGTCGGGTAACCGGGACGAGCGCAAATTTGACAACCCC
GATCAGTTCATCATTGATCGCAAGGACGCACGAAACCACATGTCGTTCGGCTATGGG
GTTCACCGTTGCATGGGCAACCGTCTGGCTGAACTGCAACTGCGCATCCTCTGGGAA
GAAATACTCAAGCGTTTTGACAACATCGAAGTCGTCGAAGAGCCCGAGCGGGTGCA
GTCCAACTTCGTGCGGGGCTATTCCAGGTTGATGGTCAAACTGACACCGAACAGTGT
ACTCCATCGTCATCAACCTGTCACCATCGGCGAGCCGGCCGCTCGTGCTGTGAGCCG
CACGGTGACCGTTGAGCGTCTTGATCGCATTGCCGACGATGTCCTTCGCCTGGTCCTT
CGCGATGCTGGAGGTAAAACCCTCCCGACGTGGACGCCTGGCGCTCACATCGACCT
GGATCTGGGtGCTCTGAGCCGTCAGTATTCGCTCTGCGGCGCTCCGGATGCTCCGTCG
TACGAAATCGCCGTGCACTTAGATCCgGAAAGCCGTGGTGGAAGCCGCTATATTCAT
GAACAGCTGGAAGTTGGAAGTCCGCTGCGTATGCGtGGCCCACGCAACCATTTCGCC
CTGGATCCGGGtGCGGAACATTACGTGTTTGTTGCCGGGGGTATCGGCATCACGCCG
GTGCTGGCAATGGCGGATCATGCCCGTGCGCGTGGTTGGTCGTACGAACTGCATTAT
TGTGGTCGTAATCGTAGCGGTATGGCTTACCTGGAACGCGTCGCGGGACATGGTGAC
CGCGCtGCCTTGCACGTATCTGAAGAAGGCACCCGCATTGATCTGGCGGCATTACTT
GCTGAACCGGCGCCGGGCGTGCAAATCTACGCCTGCGGTCCGGGCCGTTTATTAGCG
GGTCTTGAAGACGCGTCTCGTAATTGGCCGGATGGCGCGCTTCATGTGGAGCATTTC
ACTTCGAGTTTAGCCGCTTTGGATCCGGATGTCGAACATGCCTTTGATTTGGAGCTG
CGTGACTCTGGCCTTACCGTTCGCGTCGAGCCAACTCAGACCGTTTTAGACGCTTTG
CGTGCGAACAATATCGACGTCCCGTCGGATTGCgaaGAGGGGCTGTGTGGTTCTTGCG
AAGTAGCCGTTCTGGATGGCGAGGTTGATCACCGTGATACCGTTCTGACTAAGGCCG
AGCGCGCCGCGAATCGTCAGATGATGACTTGCTGCAGTCGTGCATGCGGTGATCGTC
TGGtcCTGCGCCTCTAA
Cyp153A-RhF2, chimeric hybrid-fusion protein (protein)
SEQ ID NO: 6
MPTLPRTFDDIQSRLINATSRVVPMQRQIQGLKFLMSAKRKTFGPRRPMPEFVETPIPDV
NTLALEDIDVSNPFLYRQGQWRAYFKRLRDEAPVHYQKNSPFGPFWSVTRFEDILFVDK
SHDLFSAEPQIILGDPPEGLSVEMFIAMDPPKHDVQRSSVQGVVAPKNLKEMEGLIRSRT
GDVLDSLPTDKPFNWVPAVSKELTGRMLATLLDFPYEERHKLVEWSDRMAGAASATG
GEFADENAMFDDAADMARSFSRLWRDKEARRAAGEEPGFDLISLLQSNKETKDLINRP
MEFIGNLTLLIVAGNDTTRNSMSGGLVAMNEFPREFEKLKAKPELIPNMVSEIIRWQTPL
AYMRRIAKQDVELGGQTIKKGDRVVMWYASGNRDERKFDNPDQFIIDRKDARNHMSF
GYGVHRCMGNRLAELQLRILWEEILKRFDNIEVVEEPERVQSNFVRGYSRLMVKLTPNS
VLHRHQPVTIGEPAARAVSRTVTVERLDRIADDVLRLVLRDAGGKTLPTWTPGAHIDLD
LGALSRQYSLCGAPDAPSYEIAVHLDPESRGGSRYIHEQLEVGSPLRMRGPRNHFALDPG
AEHYVFVAGGIGITPVLAMADHARARGWSYELHYCGRNRSGMAYLERVAGHGDRAAL
HVSEEGTRIDLAALLAEPAPGVQIYACGPGRLLAGLEDASRNWPDGALHVEHFTSSLAA
LDPDVEHAFDLELRDSGLTVRVEPTQTVLDALRANNIDVPSDCEEGLCGSCEVAVLDGE
VDHRDTVLTKAERAANRQMMTCCSRACGDRLVLRL.
Cyp153A-BM3 chimeric hybrid-fusion protein (DNA)
SEQ ID NO: 7
ATGCCAACACTGCCCAGAACATTTGACGACATTCAGTCCCGACTGATTAACGCCACC
TCCAGGGTGGTGCCGATGCAGAGGCAAATTCAGGGACTGAAATTCTTAATGAGCGC
CAAGAGGAAGACCTTCGGCCCACGCCGACCGATGCCCGAATTCGTTGAAACACCCA
TCCCGGACGTTAACACGCTGGCCCTTGAGGACATCGATGTCAGCAATCCGTTTTTAT
ACCGGCAGGGTCAGTGGCGCGCCTATTTCAAACGGTTGCGTGATGAGGCGCCGGTC
CATTACCAGAAGAACAGCCCTTTCGGCCCCTTCTGGTCGGTAACTCGGTTTGAAGAC
ATCCTGTTCGTGGATAAGAGTCACGACCTGTTTTCCGCCGAGCCGCAAATCATTCTC
GGTGACCCTCCGGAGGGGCTGTCGGTGGAAATGTTCATAGCGATGGATCCGCCGAA
ACACGATGTGCAGCGCAGCTCGGTGCAGGGAGTAGTGGCACCGAAAAACCTGAAGG
AGATGGAGGGGCTGATCCGATCACGCACCGGCGATGTGCTTGACAGCCTGCCTACA
GACAAACCCTTTAACTGGGTACCTGCTGTTTCCAAGGAACTCACAGGCCGCATGCTG
GCGACGCTTCTGGATTTTCCTTACGAGGAACGCCACAAGCTGGTTGAGTGGTCGGAC
AGAATGGCAGGTGCAGCATCGGCCACCGGCGGGGAGTTTGCCGATGAAAATGCCAT
GTTTGACGACGCGGCAGACATGGCCCGGTCTTTCTCCAGGCTTTGGCGGGACAAGGA
GGCGCGCCGCGCAGCAGGCGAGGAGCCCGGTTTCGATTTGATCAGCCTGTTGCAGA
GCAACAAAGAAACGAAAGACCTGATCAATCGGCCGATGGAGTTTATCGGTAATTTG
ACGCTGCTCATAGTCGcCGGCAACGATACGACGCGCAACTCGATGAGTGGTGGCCTG
GTGGCCATGAACGAATTCCCCAGGGAATTTGAAAAATTGAAGGCAAAACCGGAGTT
GATTCCGAACATGGTGTCGGAAATCATCCGCTGGCAAACGCCGCTGGCCTATATGCG
CCGAATCGCCAAGCAGGATGTCGAACTGGGCGGCCAGACCATCAAGAAGGGTGATC
GAGTTGTCATGTGGTACGCGTCGGGTAACCGGGACGAGCGCAAATTTGACAACCCC
GATCAGTTCATCATTGATCGCAAGGACGCACGAAACCACATGTCGTTCGGCTATGGG
GTTCACCGTTGCATGGGCAACCGTCTGGCTGAACTGCAACTGCGCATCCTCTGGGAA
GAAATACTCAAGCGTTTTGACAACATCGAAGTCGTCGAAGAGCCCGAGCGGGTGCA
GTCCAACTTCGTGCGGGGCTATTCCAGGTTGATGGTCAAACTGACACCGAACAGTTC
ACCTAGCACTGAACAGTCTGCTAAAAAAGTACGCAAAAAGGCAGAAAACGCTCATA
ATACGCCGCTGCTTGTGCTATACGGTTCAAATATGGGAACAGCTGAAGGAACGGCG
CGTGATTTAGCAGATATTGCAATGAGCAAAGGATTTGCACCGCAGGTCGCAACGCTT
GATTCACACGCCGGAAATCTTCCGCGCGAAGGAGCTGTATTAATTGTAACGGCGTCT
TATAACGGTCATCCGCCTGATAACGCAAAGCAATTTGTCGACTGGTTAGACCAAGCG
TCTGCTGATGAAGTAAAAGGCGTTCGCTACTCCGTATTTGGATGCGGCGATAAAAAC
TGGGCTACTACGTATCAAAAAGTGCCTGCTTTTATCGATGAAACGCTTGCCGCTAAA
GGGGCAGAAAACATCGCTGACCGCGGTGAAGCAGATGCAAGCGACGACTTTGAAGG
CACATATGAAGAATGGCGTGAACATATGTGGAGTGACGTAGCAGCCTACTTTAACCT
CGACATTGAAAACAGTGAAGATAATAAATCTACTCTTTCACTTCAATTTGTCGACAG
CGCCGCGGATATGCCGCTTGCGAAAATGCACGGTGCGTTTTCAACGAACGTCGTAGC
AAGCAAAGAACTTCAACAGCCAGGCAGTGCACGAAGCACGCGACATCTTGAAATTG
AACTTCCAAAAGAAGCTTCTTATCAAGAAGGAGATCATTTAGGTGTTATTCCTCGCA
ACTATGAAGGAATAGTAAACCGTGTAACAGCAAGGTTCGGCCTAGATGCATCACAG
CAAATCCGTCTGGAAGCAGAAGAAGAAAAATTAGCTCATTTGCCACTCGCTAAAAC
AGTATCCGTAGAAGAGCTTCTGCAATACGTGGAGCTTCAAGATCCTGTTACGCGCAC
GCAGCTTCGCGCAATGGCTGCTAAAACGGTCTGCCCGCCGCATAAAGTAGAGCTTG
AAGCCTTGCTTGAAAAGCAAGCCTACAAAGAACAAGTGCTGGCAAAACGTTTAACA
ATGCTTGAACTGCTTGAAAAATACCCGGCGTGTGAAATGAAATTCAGCGAATTTATC
GCCCTTCTGCCAAGCATACGCCCGCGCTATTACTCGATTTCTTCATCACCTCGTGTCG
ATGAAAAACAAGCAAGCATCACGGTCAGCGTTGTCTCAGGAGAAGCGTGGAGCGGA
TATGGAGAATATAAAGGAATTGCGTCGAACTATCTTGCCGAGCTGCAAGAAGGAGA
TACGATTACGTGCTTTATTTCCACACCGCAGTCAGAATTTACGCTGCCAAAAGACCC
TGAAACGCCGCTTATCATGGTCGGACCGGGAACAGGCGTCGCGCCGTTTAGAGGCTT
TGTGCAGGCGCGCAAACAGCTAAAAGAACAAGGACAGTCACTTGGAGAAGCACATT
TATACTTCGGCTGCCGTTCACCTCATGAAGACTATCTGTATCAAGAAGAGCTTGAAA
ACGCCCAAAGCGAAGGCATCATTACGCTTCATACCGCTTTTTCTCGCATGCCAAATC
AGCCGAAAACATACGTTCAGCACGTAATGGAACAAGACGGCAAGAAATTGATTGAA
CTTCTTGATCAAGGAGCGCACTTCTATATTTGCGGAGACGGAAGCCAAATGGCACCT
GCCGTTGAAGCAACGCTTATGAAAAGCTATGCTGACGTTCACCAAGTGAGTGAAGC
AGACGCTCGCTTATGGCTGCAGCAGCTAGAAGAAAAAGGCCGATACGCAAAAGACG
TGTGGGCTGGGTAA
Cyp153A-BM3 chimeric hybrid-fusion protein (protein)
SEQ ID NO: 8
MPTLPRTFDDIQSRLINATSRVVPMQRQIQGLKFLMSAKRKTFGPRRPMPEFVETPIPDV
NTLALEDIDVSNPFLYRQGQWRAYFKRLRDEAPVHYQKNSPEGPFWSVTRFEDILFVDK
SHDLFSAEPQIILGDPPEGLSVEMFIAMDPPKHDVQRSSVQGVVAPKNLKEMEGLIRSRT
GDVLDSLPTDKPFNWVPAVSKELTGRMLATLLDFPYEERHKLVEWSDRMAGAASATG
GEFADENAMFDDAADMARSFSRLWRDKEARRAAGEEPGFDLISLLQSNKETKDLINRP
MEFIGNLTLLIVAGNDTTRNSMSGGLVAMNEFPREFEKLKAKPELIPNMVSEIIRWQTPL
AYMRRIAKQDVELGGQTIKKGDRVVMWYASGNRDERKFDNPDQFIIDRKDARNHMSF
GYGVHRCMGNRLAELQLRILWEEILKRFDNIEVVEEPERVQSNFVRGYSRLMVKLTPNS
SPSTEQSAKKVRKKAENAHNTPLLVLYGSNMGTAEGTARDLADIAMSKGFAPQVATLD
SHAGNLPREGAVLIVTASYNGHPPDNAKQFVDWLDQASADEVKGVRYSVFGCGDKNW
ATTYQKVPAFIDETLAAKGAENIADRGEADASDDFEGTYEEWREHMWSDVAAYFNLDI
ENSEDNKSTLSLQFVDSAADMPLAKMHGAFSTNVVASKELQQPGSARSTRHLEIELPKE
ASYQEGDHLGVIPRNYEGIVNRVTARFGLDASQQIRLEAEEEKLAHLPLAKTVSVEELLQ
YVELQDPVTRTQLRAMAAKTVCPPHKVELEALLEKQAYKEQVLAKRLTMLELLEKYPA
CEMKFSEFIALLPSIRPRYYSISSSPRVDEKQASITVSVVSGEAWSGYGEYKGIASNYLAE
LQEGDTITCFISTPQSEETLPKDPETPLEVIVGPGTGVAPERGEVQARKQLKEQGQSLGEAH
LYFGCRSPHEDYLYQEELENAQSEGIITLHTAFSRMPNQPKTYVQHVMEQDGKKLIELL
DQGAHFYICGDGSQMAPAVEATLMKSYADVHQVSEADARLWLQQLEEKGRYAKDVW
AG.
CYP153_mgp_HTCC2143 from marine gamma proteobacterium
HTCC2143 (DNA) SEQ ID NO: 9
ATGGGAAGCTTGGAGCGTATCTCCATGTTGGATTACGATCCTGCAACGATGCCATTA
GAGGATATTGATGTATCTGACGTAGACTTATGGATCAACGACGCCAAGTGGGATTAT
TTGACGCGCTTGCGTAATGACGCTCCCGTCCATTACTGTAAGAGTTCCGAATTCGGC
CCTTACTGGTCCATCACGCGCTTCGATGACATCATGAAAGTAGAGAAGAATTGGGA
GGTTTTCTCCTCTTTCCCTAGTATCACAATTAGTGATCCTGCTGAAGATTCGGACTTC
ACTGCGCCAACCTTCATTGCCATGGACCCACCAAAGCACGACGACCAGCGTCGCGC
TGTGCAAAACGTAGTGGCACCTCCAAACCTTAAAGAGTTAGAATCGACTATTCGTTC
ACGCGCAGTCAACATCCTGGATTCACTTCCTATCGGAGAGACTTTTAATTGGGTTGA
TCGCGTTTCAATTGAACTGACTACACAGATGTTAGCTACCTTGTTTGATTTTCCTTTT
GAGGACCGTTATAAGCTGACGCATTGGAGCGATGTCACGTTTGCTCGCCTGGGAGCG
GGTCTGATCGAAACAGAGGAGGAGCGTCAGCAAGAACTGATTGAGTGTTTGAACTA
TTTCTTACAACTTTGGAAGGAACGTGAAGAGAACCCGGTGGGAAACGACCTTATCA
GTATGATGATTCGTGGAGATGCGACTAAAAACTTAAGTCCTCAAGAGTACCTTGGGA
ACATCTTACTTCTTATTGTAGGAGGGAATGACACAACCCGTAATAGCATTACAGGAG
GACTGTTGGCTCTGAACGAAAATCCCGCTGAATATGAGAAACTGCGTTCCCATCCTG
AACACATCCCGAACATGGTATCGGAAATCATCCGCTGGGTAACGCCTTTGGCTCACA
TGCGTCGTACAGCTACTCGTGATTATGAGTTCGAAGGGCAACAGATTAAGAAGGGG
GATAAAATTATCATGTGGTATGCAAGTGGCAACCGTGATGAGCGCACAATCCCCAA
TCCCAACAAGTTTTTGATCGATCGCGAGCGCGCCCGTAACCACATCAGTTTCGGCTT
CGGTTTGCACCGCTGCATGGGGAATCGTTTAGCTGAAATGCAACTGCGTGTCTTGTG
GGAGGAAATCCAAAAACGCTTTAAGACAATTGAGGTTGTCGGAGCACCAGAACGCA
TTCCTTCGTGTTTTGTCCACGGGATTTCTTCCCTGCCAGTGCGTTTACACTCTTATTAC
TAA
CYP153_mgp_HTCC2143 from marine gamma proteobacterium
HTCC2143 (protein) SEQ ID NO: 10
MGSLERISMLDYDPATMPLEDIDVSDVDLWINDAKWDYLTRLRNDAPVHYCKSSEFGP
YWSITRFDDIMKVEKNWEVFSSFPSITISDPAEDSDFTAPTFIAMDPPKHDDQRRAVQNV
VAPPNLKELESTIRSRAVNILDSLPIGETFNWVDRVSIELTTQMLATLFDFPFEDRYKLTH
WSDVTFARLGAGLIETEEERQQELIECLNYFLQLWKEREENPVGNDLISMMIRGDATKN
LSPQEYLGNILLLIVGGNDTTRNSITGGLLALNENPAEYEKLRSHPEHIPNMVSEIIRWVT
PLAHMRRTATRDYEFEGQQIKKGDKIIMWYASGNRDERTIPNPNKFLIDRERARNHISFG
FGLHRCMGNRLAEMQLRVLWEEIQKRFKTIEVVGAPERIPSCFVHGISSLPVRLHSYY.
CYP153_mgp_HTCC2080 from marine gamma proteobacterium
HTCC2080 (DNA) SEQ ID NO: 11
ATGAACCAGGCAGTAACGCGTAAAGATGGTTTGCCAGACCCCCTGTCCACTCCTCTG
GACCAATTGGACATTGCTGACCCACGTCGTTTCGAATTCGACACCTGGCAGCCATTG
TTTGAGCGTCTGCGCTCCGAGGCTCCGGTCCACTACCAAGCACAGGGACCTGCAGGT
CCCGCTCCCCACGGCGACTTCTGGTCTGTAACTCGTTTCGAAGATATTGTCGAGGTT
GAAAAAAATTGGGAGGCGTTTAGTTCCGAGCCAAGCATCGCCATCCTTGACCCGGA
ACCAGACATGTCCGTACAGATGTTCATCGCAACAGATCCACCCCTTCATGATGACCA
ACGCCGTGCGGTACAAGGTGCGGTCGCACCAAAAAATTTACAGGAATTTGAGGCCC
TGATTCGTCAGCGCACGCAGGAAACTCTGGATGGGCTGCCACTTGGGGAGACTTTCA
ATTGGGTCGACCGTGTAAGCATCGATTTGACTACCAAAATGCTTGCGACATTATTTG
ATTTCCCCCAGGAGCAGCGCCAATTATTGACTCACTGGTCCGACGCCGCGACTTCAG
ATGCGCGCATCACCGCAGGGCGCGGGCTGTCGATTGATGAACGTCGTACCATCATG
GGGGAGATGCTGCAAAGCTTTACCCAGCTGTGGCACGACCACAAAGCGAATGACCG
TGAGTCCTTCGATTTAATCCGTATGTTACAGCGTGATCCAAAGACCCAAAATATGGT
AGACGAGCCATTTTCTTACCTGGGTAACCTTATGTTACTTATCGTTGGGGGGAACGA
CACAACTCGTAATTCCATGACGGGAGGTGTATTGCATTTGCATCAAAACCCCAGCGA
AATGGCGAAACTGAAGGCCAATCACGGGTTGATTCCCTCGATGGTTTCGGAAATCAT
CCGTTATCAGACGCCATTGCCACACATGCGCCGTACAGCCACACGCGATGTCGAATT
AAACGGAACCGTTATCCCGAAAGGCGGTCGTGTGGTATTGTGGTATGTTAGCGGAA
ATCGTGACTCTAATGCAATTCCCGAGGCAGACAAGTTCATCATCGATCGCCCGAATG
TACGTAACCATTTGTCATTCGGGATGGGTATTCATCGCTGCATGGGGAATCGTTTGG
CTGAAATGCAATTACGCATTGTTTGGGAAGAAATCCTTGAGCGTTTTGAGCGCATTG
AAGTTGTAGGACCAGCAGTTCGCACTTGTAATTCCTTTGTTCGTGGCTATCACGAAC
TTCCGGTTCGTCTTCACCCTAAGGTTTACTAA
CYP153_mgp_HTCC2080 from marine gamma proteobacterium
HTCC2080 (protein) SEQ ID NO: 12
MNQAVTRKDGLPDPLSTPLDQLDIADPRRFEFDTWQPLFERLRSEAPVHYQAQGPAGPA
PHGDFWSVTRFEDIVEVEKNWEAFSSEPSIAILDPEPDMSVQMFIATDPPLHDDQRRAVQ
GAVAPKNLQEFEALIRQRTQETLDGLPLGETFNWVDRVSIDLTTKMLATLFDFPQEQRQ
LLTHWSDAATSDARITAGRGLSIDERRTIMGEMLQSFTQLWHDHKANDRESFDLIRMLQ
RDPKTQNMVDEPFSYLGNLMLLIVGGNDTTRNSMTGGVLHLHQNPSEMAKLKANHGLI
PSMVSEIIRYQTPLPHMRRTATRDVELNGTVIPKGGRVVLWYVSGNRDSNAIPEADKFII
DRPNVRNHLSFGMGIHRCMGNRLAEMQLRIVWEEILERFERIEVVGPAVRTCNSFVRGY
HELPVRLHPKVY.
CYP153_Clit from Congregibacter litoralis (DNA)
SEQ ID NO: 13
ATGAACGTCGCTCAAGATCTGCCTCATCCATCGGATTTAGCTTTGGAAGACATCGAC
GTAAGCGATTCGCGCATCTACCAACAGGATGCATGGCGCCCCTACTTTGAACGTCTG
CGCAAGGAGGACCCTGTTCACTACGTAGCAGACTCACAGTTCGGGCCTTTCTGGTCC
ATCACCCGCTGGGAGGATATTGTAGCGGTGGATTCCAACTTCGAGGATTTTTCTAGT
GAACCGGCCATCGTCATTGGAGACAACAGCGAAGAGCTGCCCATTGCCAATTTCATT
AGCATGGACCCTCCCAAGCATGACGTTCAACGCCGCGCCGTCCAGGGCGTAGTGGC
CCCAAAAAACTTGGCGGAGATGGAGGCGCTGATCCGCTCCCGCGTGGTAGAAATTT
TGGATGGCTTACCTGTGGGAGAGACATTCAACTGGGTCGACCGCGTCTCGATCAATC
TTACAACGCAAATGCTGGCTACACTTTTCGACTTTCCATTCGAAGAACGTTTTAAACT
GACCTACTGGAGCGATTTGGGCGCCGGCAGCCCTGAAATTGCGGGTGGCGATGTCG
ATCCTGAGGAACGTTTAGCTGGTTTACATGACTGCTTAGAAACATTCACTCGCATCT
GGCACGAACGCAAAGACGACGGTGTCGAGACTTTGGACCTTATTCGCATGTTGCAA
CGTGACCCGAATACGAAGGACATGGTCGATGATCCAATGGAATATCTTGGAAATCT
GCTGCTTCTGATCATTGGTGGAAATGACACGACCCGTAATAGCGCTAGCGCTGGCGT
TCTGGCGTTGAATCAAAACCCTGCCGAATATGAAAAATTGCGTGCTAACCCCGCACT
TATCCCGAGTATGGTATCAGAGGTGATTCGTTGGCAGACGCCCTTAATGCACATGCG
TCGCACTGCTAACAAGGATGTTGAATTTCAGGGCAAGCAGATTCGCAAAGGTGACA
AGGTCGTTATGTGGTATCTTAGTGGCAACCATGATGAAACAGCTATCGAAAACCCCA
ATTCCTTCATCATTGATCGTGAAAATCCTCGTAAACATATGTCTTTTGGGTTCGGGGT
TCACCGCTGCATGGGTAACCGCCTTGCCGAGATGCAACTTCGTGTTCTTTGGGAGGA
AATTATGCAACGTTTTTCAAAGGTAGAAGTCGTAGGGGAACCAGAGCGTGTTAAGT
CCAACTTTGTGCGTGGGATTAGCAATTTGCCAGTCCGTGTGCACCCTCTTTACTAA
CYP153_Clit from Congregibacter litoralis (protein)
SEQ ID NO: 14
MNVAQDLPHPSDLALEDIDVSDSRIYQQDAWRPYFERLRKEDPVHYVADSQFGPFWSIT
RWEDIVAVDSNFEDFSSEPAIVIGDNSEELPIANFISMDPPKHDVQRRAVQGVVAPKNLA
EMEALIRSRVVEILDGLPVGETFNWVDRVSINLTTQMLATLFDFPFEERFKLTYWSDLGA
GSPEIAGGDVDPEERLAGLHDCLETFTRIWHERKDDGVETLDLIRMLQRDPNTKDMVD
DPMEYLGNLLLLIIGGNDTTRNSASAGVLALNQNPAEYEKLRANPALIPSMVSEVIRWQ
TPLMHMRRTANKDVEFQGKQIRKGDKVVMWYLSGNHDETAIENPNSFIIDRENPRKHM
SFGFGVHRCMGNRLAEMQLRVLWEEIMQRFSKVEVVGEPERVKSNFVRGISNLPVRVH
PLY.
CYP153_Lim from Limnobacter sp. MED105 (DNA) SEQ ID NO: 15
ATGTCAACGCAATCAAAGACATTCGACCAGATTCAAACACGCGTAATCAATGCGAC
AGCAAAGGTTATTCCAATGCACTTGCAGATTCAAGGTTTAAAATTCCTTATGCGCGC
GAAGAAAAAAACCATTGGGGCCCGCCGCCCAACTCCCTCCTTTGTGGAGTACCCACT
GCCAGATGTCGGGACTTTACGCATTGACGATATTGATGTCTCAAACCCTTTCTTATAC
CGCCAGGGTCAATGGCGCGCATATTTCAAGCGCCTTCGTGATGAAGCACCCGTTCAT
TTCCAAAAGAATTCACCCTTCGGTCCGTTCTGGAGCGTTACCCGTTATGAGGATATC
CTTTTCGTAGACAAACATCACGATTTATTTTCGGCAGAGCCCGTTATCATCTTAGGA
GACCCACCGGAGGGTCTTTCGGTAGAGATGTTTATCGCAATGGATCCTCCTAAGCAC
GATGCTCAACGTAGTTCAGTACAAGGGGTGGTGGCCCCCCAAAACTTAAAGGAGAT
GGAGAGCCTGATTCGTTCACGCACGGGAGATGTGTTAGATTCCCTTCCCGTCGGCCA
GCCTTTTGACTGGGTTCCCACAGTTTCGAAGGAGCTGACTGGACGCATGCTTGCGAC
TCTGTTAGATTTTCCGTACGACGAGCGTGACAAGCTGATTTATTGGTCGGATCTTCTG
GCTGGTGCGGCGTCCGCCACCGGGGGAGAGTTTACTGATGAGGAAGCGATGTTTGA
TGCAGCGGCAGATATGGCACGCGACTTTAGCCGTTTATGGCGCGACAAACAGGCGC
GCCGCGCAGCTGGGGAAGCTCCCGGCTTCGATTTAATTAGCCTGCTTCAGAGCAGCG
AAGATACGCGTGACCTGATCAATCGCCCGATGGAGTTTATCGGCAACTTGGCATTGT
TAATTGTTGGGGGAAACGATACAACACGTAACTCCATGAGTGGTGGCCTTCTGGCAA
TGAATCAATTTCCTAAGGAGTTTAAAAAGCTGAAGGCAAACCCAGCATTGATCCCTA
ATATGGTGTCGGAAATTATTCGCTGGCAGACACCTCTGGCCTACATGCGTCGTATTG
CTAAACAAGATGTTGAACTTGGCGGACGCACTATCAAGAAAGGAGATCGCGTAGTC
ATGTGGTATGCCTCCGGTAACCGCGACGAACGTAAGTTCACCGACCCCGATCAATTT
TTGATTGATCGCAGTGGGGCCCGCAACCACTTGTCCTTCGGATATGGAGTACACCGT
TGTATGGGTAATCGTCTTGCGGAGCTTCAACTGCGTATTTTGTGGGAAGAATTGTTA
AAGCGTTTCGAAAATATTGAAGTTGTGGGCGAGCCTGAGCGTGTTCAATCAAACTTT
GTTCGCGGATATTCAGAGATGATGGTGAAATTAACTCCACTGCGTTACTAA
CYP153_Lim from Limnobacter sp. MED105 (protein)
SEQ ID NO: 16
MSTQSKTFDQIQTRVINATAKVIPMHLQIQGLKFLMRAKKKTIGARRPTPSFVEYPLPDV
GTLRIDDIDVSNPFLYRQGQWRAYFKRLRDEAPVHFQKNSPFGPFWSVTRYEDILFVDK
HHDLFSAEPVIILGDPPEGLSVEMFIAMDPPKHDAQRSSVQGVVAPQNLKEMESLIRSRT
GDVLDSLPVGQPFDWVPTVSKELTGRMLATLLDFPYDERDKLIYWSDLLAGAASATGG
EFTDEEAMFDAAADMARDFSRLWRDKQARRAAGEAPGFDLISLLQSSEDTRDLINRPM
EFIGNLALLIVGGNDTTRNSMSGGLLAMNQFPKEFKKLKANPALIPNMVSEIIRWQTPLA
YMRRIAKQDVELGGRTIKKGDRVVMWYASGNRDERKFTDPDQFLIDRSGARNHLSFGY
GVHRCMGNRLAELQLRILWEELLKRFENIEVVGEPERVQSNFVRGYSEMMVKLTPLRY
CYP153_Caul from Caulobacter sp. K31 (DNA) SEQ ID NO: 17
ATGAGCGATGGTGCAATTGATTTACGCGCGGAGGCGCGCGCCAAGGCGTATGCGAT
GCCCTTAGAGGATATCAACCTTGCAGATACCGAATTGTGGCGCACAGATACGGTGTG
GCCGTATCTGGAGCGCTTGCGTAAGGAAGACCCCGTTCATTTACACCCTGCTCATCA
CCATCCCGACGGAGCGTTTTGGAGTATTACTAAATATGCAGATATCATGGCCGTCGA
CATTAATCATGAGGTATTTTCAAGTGAACCGTCGATCACCATCTTTGACCCCAAAGA
GGATTTTACCCTTCCTATGTTCATTGCGATGGACCCGCCAAAGCATGATGTGCAACG
CAAGACTGTCAGTCCGATTGTGAGTCCGGCAAACTTACACTTGATGGAACCTCTTAT
CCGCAGTCGCATCACCAAAACGCTTGACGAACTTCCTATTGGGGAGCCATTTGATTG
GGTGGATCGCGTCTCTATCGAGCTTACAACTCAGATGTTGGCGACCCTTTTCGACTTC
CCATGGGACGAGCGCCGCAAACTGACACGCTGGTCCGATATTGCAACAGCAGTACC
CGAAAGCGGCTTATTTGTTACGGACGATTATGAAACCGAGCGCCGCATGGAATTATT
CGGGTGCGTCGATTATTTCACTCGTCTTTGGAACGAACGTGTGAATGCCCCACCTAA
GGGTGATCTGATCTCTATGTTAGCCCATGGGGAGGCCACCCGTAATATGGACCGTAT
GGAGTACCTTGGGAACTTATTACTTCTTATTATTGGAGGGAACGACACAACTCGTAA
CACTATGACAGGCTCGATCCTGGCGATGAATCAAAACCCCGACCAATTACGCAAAT
TACGTGAAAACCCGAGTCTGATTCCGTCCATGGTCAGTGAAACCATTCGCTGGCAGA
CTCCACTGTCAAACATGCGCCGCACAGCCACCCAAGACTTTGAGTTAGGTGGGAAA
TTGATTAAGAAAGGTGATAAAGTGCTGATCTGGTATGCCTCCGGTAATCGTGATGAA
GAAGCTATCGAAAACCCAGAAGCGTACATTATTGACCGTGAGCGTCCTCGTAATCAT
CTTTCGTTTGGTTTCGGTATCCACCGTTGCGTCGGGAATCGCCTGGCAGAATTGCAAT
TGCGTATTCTGTGGGAAGAAATTTTACCCCGTTTTCCAGAGATTCAGGTACTGGAGG
ATCCGCAGCGTGTTCCGTCAGTTCTGATTCGTGGGTACTCCTCCATGCCCGTCATGAT
CCCTGCACGCTCATACTAA
CYP153_Caul from Caulobacter sp. K31 (protein)
SEQ ID NO: 18
MSDGAIDLRAEARAKAYAMPLEDINLADTELWRTDTVWPYLERLRKEDPVHLHPAHH
HPDGAFWSITKYADIMAVDINHEVFSSEPSITIFDPKEDFTLPMFIAMDPPKHDVQRKTVS
PIVSPANLHLMEPLIRSRITKTLDELPIGEPFDWVDRVSIELTTQMLATLFDFPWDERRKL
TRWSDIATAVPESGLFVTDDYETERRMELFGCVDYFTRLWNERVNAPPKGDLISMLAH
GEATRNMDRMEYLGNLLLLIIGGNDTTRNTMTGSILAMNQNPDQLRKLRENPSLIPSMV
SETIRWQTPLSNMIRRTATQDFELGGKLIKKGDKVLIWYASGNRDEEAIENPEAYIIDRER
PRNHLSFGFGIHRCVGNRLAELQLRILWEEILPRFPEIQVLEDPQRVPSVLIRGYSSMPVMI
PARSY
CYP153_Mmar from Mycobacterium marinum str. M (DNA)
SEQ ID NO: 19
ATGTCGAACATTCGTGAAGCAGTAACGGCAAAGGCGCAAGCCACTATTCCAATGGA
TCGTATTATCCAGGGAGCGCACTTGTATGATCGTACACGCCGCTGGGTAACCGGCAC
GAATGGTGAGAAAATTTTCATTGAACGCCCGATCCCGCCTGCCGACGAAGTCGAGCT
TACAGACATCGACTTAAGTAATCCCTTCCTTTACCGCCAGGGCCGCTGGAAGTCTTA
TTACGAGCGCCTTCGTAACGAAGCGCCTGTCCACTACCAAGCGCATTCGGCCTTCGG
GCCTTTCTGGTCTGTTACTCGTCACGCAGATATTGTGGCAGTCGATAAGAACCATGA
AGTTTTTAGCAGTGAACCGTTTATCGTGATTGGGTCCCCACCACGCTTTTTGGATATC
GCGATGTTCATCGCTATGGATCCACCCAAACATGATCGCCAACGCCAGGCAGTTCAG
GGCGTCGTGGCACCAAAGAATTTACGTGAAATGGAAGGATTAATTCGCGAGCGCGT
CGTGGACGTGTTGGATGCCCTGCCCCTTGGCGAACCTTTCAATTGGGTGCAGCACGT
CTCAATTGAATTGACTGCGCGCATGTTAGCTACATTATTGGACTTTCCTTTTGAACAG
CGTCGCAAGTTGGTCCAGTGGTCCGACTTGGCGACGAGCATGGAACAAGCAAATGG
CGGTCCTAGCGATAATGACGAAATCTTCCGCGGTATGGTGGACATGGCGCGTGGGTT
ATCTGCCCATTGGCGCGACAAAGCAGCACGTACTGCGGCAGGAGAACTTCCAGGAT
TCGACCTTATCACTATGTTACAGAGCGACGAATCCACGAAGGACCTGATTGACCGCC
CGATGGAGTTTTTAGGTAATCTTGTTCTTCTTATCGTGGGAGGTAATGATACTACCCG
TAATTCTATGAGTGGTGGCGTATTGGCTCTGAACGAGTTCCCAGATCAGTTTGAGAA
ACTGAAGGCTAACCCAGAACTGATTCCTAATATGGTATCTGAGATTATTCGCTGGCA
AACACCGTTGGCTCACATGCGTCGCATTGCCAAAGCCGATACGGTGCTGAATGGGC
AATTTATTCGCAAGGGAGACAAAGTTTTGATGTGGTATGCTTCAGGTAATCGCGATG
AGCGTGTATTTGACCGCCCCGATGACTTGATTATCGACCGCGCAAATGCCCGTAACC
ATATTTCGTTCGGGTTCGGAGTTCATCGCTGTATGGGAAATCGTCTTGCTGAGATGC
AGTTACGCATCTTATGGGAGGAACTTTTACCACGCTTTGAGAACATTGAAGTAGTGG
GGGAACCTGAGTACGTCCAATCTAATTTCGTGCGTGGGATCAGCAAACTGATGGTCC
GTTTGACCCCCAAGGGCGGTGCCTACTAA
CYP153_Mmar from Mycobacterium marinum str. M (protein)
SEQ ID NO: 20
MSNIREAVTAKAQATIPMDRIIQGAHLYDRTRRWVTGTNGEKIFIERPIPPADEVELTDID
LSNPFLYRQGRWKSYYERLRNEAPVHYQAHSAFGPFWSVTRHADIVAVDKNHEVFSSE
PFIVIGSPPRFLDIAMFIAMDPPKHDRQRQAVQGVVAPKNLREMEGLIRERVVDVLDALP
LGEPFNWVQHVSIELTARMLATLLDFPFEQRRKLVQWSDLATSMEQANGGPSDNDEIFR
GMVDMARGLSAHWRDKAARTAAGELPGFDLITMLQSDESTKDLIDRPMEFLGNLVLLI
VGGNDTTRNSMSGGVLALNEFPDQFEKLKANPELIPNMVSEIIRWQTPLAHMRRIAKAD
TVLNGQFIRKGDKVLMWYASGNRDERVFDRPDDLIIDRANARNHISFGFGVHRCMGNR
LAEMQLRILWEELLPRFENIEVVGEPEYVQSNFVRGISKLMVRLTPKGGAY.
CYP153_mgp_HTCC2148 from marine gamma proteobacterium
HTCC2148 (DNA) SEQ ID NO: 21
ATGCCGGAAGTTGCATTGGACAACTCCCAGGATAAACTTCAGCAGGATCGTATCGA
GAACTACGACGCGACGGTCGTAGACAAGGACCCTTATGACGTTGACATCGCGGACT
TCAACATGATTAACCCCCACCTTTTCTCTTCTAACACCCTTTGGCCCTGGTTCAAACG
CCTGCGTGAGGAGGAGCCAGTCCATTATTGTCGCGACTCCGTATTTGGTCCGTATTG
GAGCGTTACGAGTTATGATGCTATCATGAAAGTAGACACGTCACATGACATCTTTAG
CTCGGAGCCGAACATTACCGTAGGCGATTTACAAGAGGACTTTCCTTTAGCAACTTT
TATCGCCATGGATCGTCCGAAGCACGACGAACAACGCGCCGTAGTAAATCCTGCTG
TGCGCGGACAGCAGTTGCGCGACTTTGAGCCCCTTATTCGCGAGCGCACTCAACGCG
TACTGGATTCTTTACCGGTGGGCGAAGAATTCAATTGGGTGGAAAAGGTCTCTATTG
AATTAACTACCTGTATGCTGGCTACCTTGTTTGACTTTCCGTTCGAGGACCGTGCCAA
ATTAACCCGTTGGTCTGATGTGACGTTCGCAATCCCTGGACTGGGTGTAGTGGATAG
TGAAGAACAGCGTCGTGAGGAACTTTTGGAGTGCCTTGCGGCCTTCACCGAACTTTG
GAACCAACGTGTCAACGAAGAACCGCGCGGGGACTTCATTAGTCTGCTGGCTCATG
GCAGCGCAACGCAGGAAATGGATCCCTTTGAATATCTGGGCAACATCCTTCTTTTGA
TCATTGGTGGTAATGACACAACACGTAATTCGATGAGCGGAAGCGTGTTGTGTCAAA
ACCTGTACCCAGAGCAATTTGATAAGTTAAAGGCACAACCGGAGCTGATTCCATCTA
TGGTTAGTGAGACGATTCGTTGGCAAACTCCCCTGGCCTATATGCGTCGCACCGCAA
ATCAAGACACAATCTTAGGGGATAAGCAAATCAAGAAAGGTGATAAGGTTTTGATG
TGGTATGTGAGCGGCAATCGCGATGAATCTCACTTTGAAAATCCTGACGACTATATC
ATCGATCGCCCTAACGTCCGCAGCCACCTTTCATTTGGTTTTGGGATCCATCGTTGTA
TGGGCAATGCGGTCGGTGAGATGCAGTTGCGTGTCCTGTGGGAGGAGATCCTGCAA
CGTTTCGACCGTATTGAGGTAGTTGGTGATGTTGAACGCGTGCCTTCCTCATTCGTAA
AGGGGTATAGCAGCTTACCTGTGATTCTGCACCCTAAAAAGTACTAA
CYP153_mgp_HTCC2148 from marine gamma proteobacterium
HTCC2148 (protein) SEQ ID NO: 22
MPEVALDNSQDKLQQDRIENYDATVVDKDPYDVDIADFNMINPHLFSSNTLWPWFKRL
REEEPVHYCRDSVFGPYWSVTSYDAIMKVDTSHDIFSSEPNITVGDLQEDFPLATFIAMD
RPKHDEQRAVVNPAVRGQQLRDFEPLIRERTQRVLDSLPVGEEFNWVEKVSIELTTCML
ATLFDFPFEDRAKLTRWSDVTFAIPGLGVVDSEEQRREELLECLAAFTELWNQRVNEEP
RGDFISLLAHGSATQEMDPFEYLGNILLLIIGGNDTTRNSMSGSVLCQNLYPEQFDKLKA
QPELIPSMVSETIRWQTPLAYMRRTANQDTILGDKQIKKGDKVLMWYVSGNRDESHFE
NPDDYIIDRPNVRSHLSFGFGIHRCMGNAVGEMQLRVLWEEILQRFDRIEVVGDVERVP
SSFVKGYSSLPVILHPKKY.
CYP153_Pmed from Patulibacter medicamentivorans (DNA)
SEQ ID NO :23
ATGTTTGAACAAACAACCACGAAGCGCGAGACCATGACAACGAACAGCACGTTATT
CCAGCGTACAAAGGTCCGCGTGACTGACACGGTTCAAGCTACTGTCCCGGTAGACC
GTGTAATCCAGTCTGTGGCCCTGACGTTGAAAGCAAAACGCTTGGCTGGGATGATGA
AAGCATTGCGTTTCGAAGAACGTCCCATTCCTGATCCGGCAGATGTTCCACTGGAGG
AAATCGACGTCTCTAATCCTTTTATGAACCGCCAAGGACAATGGTATCCGTATTTTG
CGCGTTTACGCGAGGAGGCGCCTGTGCATTATCAACCCAAGTCGCCTTTTGGGCCGT
TCTGGTCGGTTACGCGTTACGCGGACATTCAAGCGGTTGATTCAAACGCAGAGGTCT
TTAGTGCAGAACCGTATATCGTCTTAGGCCCTCCACCGTTCAACGCCGAAATGTTCA
TCGCCATGGACCCTCCCAAACACGACGTCCAGCGTCGTGCAGTCCAAGGTGTGGTCG
CGCCGAAGAACTTAAAAGAAATGGAGAGTTTAATCCGCGAACGCGTACAGGATGTA
TTAGATAATTTACCGACAGACCGCCCCTTTGATTGGGTCGAACGTGTCAGTAAAGAA
ATCACAGGGCGCATGTTAGCCACCTTATTGGATTTCCCATTCGAGGAGCGCCATAAG
TTGACGTATTGGAGTGACACGATCAGTGGCTCAGCAGACGCAACTGGTGGCACGAC
CCATCAAGACGAGTTGTTCCCTGCCGCAGTTGATTTGGCACGCAATTTCACCGCCCT
GTGGCACGATAAGGCAGCTCGTCGCGCTGCGGGCGAACCAGATGGGTTTGACCTTA
TTACACTTATGCAGACATCGGAGGACACAAAAGACCTTATCAAAAAGCCGTTTGAG
TTTCTGGGGAATTTGACTTTATTGATTGTTGGTGGAAATGACACAACTCGCAACTCG
ATGTCAGGAGGCGTTCTGGCACTTAACCGCTTTCCAGATGAATTTGAGAAGTTGAAA
GCGGACCCAAAACTGATTCCGAACATGGTATCCGAGATCATCCGTTGGCAGACTCCC
TTGGCATATATGCGCCGCGTAGCTAAAAAAGACGTTCATTTTGGCGGGCAATTTATT
CGCAAAGGTGACGTTGTATTAATGTGGTACGCCTCTGGGAATCGTGACGAGCGCAA
GTTTGAAAACGCCGACGACTTCATCATCGACCGTAAGAATGCACGCAACCACATGT
CTTTCGGATTCGGAGTCCACCGTTGCATGGGTAACCGCTTAGCTGAGATGCAGCTTC
GTATCCTTTGGGAAGAGCTTTTGGAACGTTTTGACCGCATCGACGTAATTGGCGAAC
CAGGGTATGTACAGTCAAACTTTGTCAAAGGGTACTCCGACATGCAGGTCCAATTAA
CGCCTAAGCCCGCTCGCGAACAAACACCTGTCCGCGTTGAGGCCACTTCGGGTGCAT
ACTAA
CYP153_Pmed from Patulibacter medicamentivorans (protein)
SEQ ID NO: 24
MFEQTTTKRETMTTNSTLFQRTKVRVTDTVQATVPVDRVIQSVALTLKAKRLAGMMK
ALRFEERPIPDPADVPLEEIDVSNPFMNRQGQWYPYFARLREEAPVHYQPKSPFGPFWSV
TRYADIQAVDSNAEVFSAEPYIVLGPPPFNAEMFIAMDPPKHDVQRRAVQGVVAPKNLK
EMESLIRERVQDVLDNLPTDRPFDWVERVSKEITGRMLATLLDFPFEERHKLTYWSDTIS
GSADATGGTTHQDELFPAAVDLARNFTALWHDKAARRAAGEPDGFDLITLMQTSEDTK
DLIKKPFEFLGNLTLLIVGGNDTTRNSMSGGVLALNRFPDEFEKLKADPKLIPNMVSEIIR
WQTPLAYMRRVAKKDVHFGGQFIRKGDVVLMWYASGNRDERKFENADDFIIDRKNAR
NHMSFGFGVHRCMGNRLAEMQLRILWEELLERFDRIDVIGEPGYVQSNFVKGYSDMQV
QLTPKPAREQTPVRVEATSGAY.
CYP153_Ppsy from Paraglaciecola psychrophila 170 (DNA)
SEQ ID NO: 25
ATGAACTCGCTGACGAACACCACGGCTGAGCCTGTCGCGACAAGTTCTATCACACC
GACCCCTGCTGTGAAATTCATCGAACAACCTATTGCCGATGTTTCTACGGTGGCTCT
GGAGGACATCGATGTGTCGAATCCATTCATGTTCCGTCAGAACAAGTGGCAATCGTA
CTTTAAACGTTTGCGCGATGAGTGCCCGGTTCATTATCAAAAAAATTCACCTTTCGG
GGCATTCTGGTCGGTAACCCGTTTCGAGGATATTATGTTTGTGGATAAGAATCACAC
GCTGTTTAGTTCCGAACCTGCCATCGTGATCGGAGACCGCCCAGCTGACTATATGCT
TGACATGTTTATTGCAATGGACCCGCCTAAACATGATGCTCAGCGTCAGGCTGTGCA
GAGCGCGGTTGCCCCCAAAAACCTGGCGGAGATGGAGGAACTGATTCGCGAGCGTA
CAGTAGATGTCCTGAACGATCTTCCGGTAGGTGAATCGTTCGACTGGGTTGAGAAAG
TAAGTGTGGAACTGACTACTCGCATGCTGGCGACACTGTTTGACTTCCCGTACGAAA
AGCGCCATAAGCTGCCTTACTGGTCGGATCTTGCGTCCGGCTCCCCCGAAATGACTG
GTGGCTTAGTGCAAGACGATGAACGCGTGGCAGGCATCACGGACCTTATGACAGAG
TTCTCCCAGCTTTGGCACATCAAGGCGGCACAGAAAGCAGCGGGTGAACAGGGAGG
CTTTGATCTTATTTCATTGATGCAGGCCAATGACAACACAAAAAACATGGTCGACAC
CCCGTTAGAGTTCCTGGGTAACCTTGTGCTGCTGATTGTTGGTGGCAACGACACCAC
TCGCAACAGTATGACTGGCGGCGTCGATGCGCTGAACGAGTTTCCTCAAGAGTTCAT
TAAGCTGAAAAATGACCCGAGCTTAATTCCCAATATGGTATCTGAAATCATCCGCTG
GCAGACTCCCCTTGCTCACATGCGTCGCATCGCGACAGAAGACGTGGAATTAAATG
GGAAGACGATTAAGAAGGGAGACAAGGTTGTTATGTGGTACGTGTCCGGAAATCGT
GATGAACGTGTAATTAATAATCCTGATCAATTCGTCATTGACCGCGACAAAGCCCGT
AATCATCTGTCGTTTGGATTTGGGATTCATCGTTGCATGGGTAACCGCTTAGCGGAA
ATGCAACTGCGCATCCTTTGGGAGGAAATCTTACAACGTTTTGAGAATATCGAAGTG
ATTAACAAGCCCAAATACGTTCAGTCTAACTTCGTCAAGGGATATACGGAACTTCGT
GTTAAATTGACAGCCAAAGTGTACTAA
CYP153_Ppsy from Paraglaciecola psychrophila 170
(protein) SEQ ID NO: 26
MNSLTNTTAEPVATSSITPTPAVKFIEQPIADVSTVALEDIDVSNPFMFRQNKWQSYFKR
LRDECPVHYQKNSPFGAFWSVTRFEDIMFVDKNHTLFSSEPAIVIGDRPADYMLDMFIA
MDPPKHDAQRQAVQSAVAPKNLAEMEELIRERTVDVLNDLPVGESFDWVEKVSVELTT
RMLATLFDFPYEKRUKLPYWSDLASGSPEMTGGLVQDDERVAGITDLMTEFSQLWHIK
AAQKAAGEQGGFDLISLMQANDNTKNIVIVDTPLEFLGNLVLLIVGGNDTTRNSMTGGV
DALNEFPQEFIKLKNDPSLIPNMVSEIIRWQTPLAHMRRIATEDVELNGKTIKKGDKVVM
WYVSGNRDERVINNPDQFVIDRDKARNHLSFGFGIHRCMGNRLAEMQLRILWEEILQRF
ENIEVINKPKYVQSNFVKGYTELRVKLTAKVY.
CYP153_Abro from Afipia broomeae ATCC 49717 (DNA)
SEQ ID NO: 27
ATGCACGGTACATTAGACCCCGCCCGCCATTCAGAACTTAATGCCGCACGCGAGAA
AGCCTACAGTACGCCGATCGAGCAGTTTAATCCCGGCGACCCAGACCTTTTCCGCAA
CGATACATTCTGGCCGTACTTCGAACGTTTGCGTAAGGAGGACCCAGTGCACTATTG
TAAAGATTCTCAATTCGGGGCCTATTGGTCCGTGATGAAATATAACGATATTATGCA
TGTTGAGACGAATCATCAAATTTACAGCAGTGAAGCCAGCCTTGGTGGCATTACTAT
TCGCGACGCTCCTCCGGAATTCCGTCGTCCAATGTTTATTGCAATGGACCAACCAAA
ACACAGCGCCCAGCGCAAGACGGTCGCTCCGATGTTTACACCCACACATTTGGATG
AACTGGCCGGAACAATCCGCCAGCGTGCTGCACAATGCTTAGACGCACTTCCTCGTA
ATGCAACTTTCGACTGGGTAGATCGCGTAAGCATCGAACTTACGACTCAGATGCTGG
CGACTCTTTTTGATTTTCCCTGGGAGGACCGTCGCAAATTGACGCGCTGGTCCGATG
TAGCTACCGCCTTGCCTGGTAACACTGACATCATCGCGTCTGAGGATGCCCGTCAAG
CGGAGCTTCAGGAGTGTGCTGCATATTTCTCTCGTTTGTGGAACGAACGCATTAACG
CGCCCCCCAAATCAGATCTTCTTTCAATGATGGCCCATGCCGAAGCAACTCGCGACA
TGGACCCCAAGACGTTTCTTGGCAACCTGATCTTGTTGATCGTGGGAGGGAACGACA
CGACACGTAATTCACTGTCCGGAGGATTATACGCTTTGAACGAAAACCCAGCCGAAT
ACCAAAAGTTACGTAGTAATCACGCCTTAGTGGACTCGATGGTGCCAGAGGTGATCC
GCTGGCAGACACCGTTGGCACATATGCGTCGCACAGCCCTGCAAGACACAGAGTTG
CGCGGCAAGCACATTAAAAAGGGAGATAAGGTTGTCATGTGGTACGTATCTGGCAA
TCGCGACGATGAAGTGATTGAAAATCCAGACAGTTTCATTATCGATCGCGCACGTCC
CCGCCAACACCTGTCGTTCGGCTTTGGAATCCACCGCTGTGTCGGCATCCGTCTTGCT
GAGTTACAGTTGAAGATCGTCTGGGAAGAGATCCTTAAACGCTTCGATAACATCGA
GGTAGTAGAACCCCCACACCGCGTTTACTCCTCCTTTGTGAAAGGGTACGAAACACT
GCCCGTGCGTTTGGCATCATACTAA
CYP153_Abro from Afipia broomeae ATCC 49717 (protein)
SEQ ID NO: 28
MHGTLDPARHSELNAAREKAYSTPIEQFNPGDPDLFRNDTFWPYFERLRKEDPVHYCKD
SQFGAYWSVMKYNDIMHVETNHQIYSSEASLGGITIRDAPPEFRRPMFIAMDQPKHSAQ
RKTVAPMFTPTHLDELAGTIRQRAAQCLDALPRNATFDWVDRVSIELTTQMLATLFDFP
WEDRRKLTRWSDVATALPGNTDIIASEDARQAELQECAAYFSRLWNERINAPPKSDLLS
MMAHAEATRDMDPKTFLGNLILLIVGGNDTTRNSLSGGLYALNENPAEYQKLRSNHAL
VDSMVPEVIRWQTPLAHMRRTALQDTELRGKHIKKGDKVVMWYVSGNRDDEVIENPD
SFIIDRARPRQHLSFGFGIHRCVGIRLAELQLKIVWEEILKRFDNIEVVEPPHRVYSSFVKG
YETLPVRLASY.
CYP153_Rrub from Rhodococcus ruber BKS 20-38 (DNA)
SEQ ID NO: 29
ATGAAGATTCCCGAGGCTATTACAGCCAAAGTGCAATCTACGATTCCCATGGATCTG
CAGATTCAGGGTGCACATTTGTACGATAAAACACGTCGCTGGGTGACGGGGACAAA
TGGTGAAAAATTGTTTGTGGAGAGCCCTATCCCTCCCGTTGAGGACGTTGAGCTTGC
AGATATTGATTTATCGAATCCTTTTCTTTATCGTCAAGGACGCTGGCAGTCCTACTTC
GAACGCTTGCGCAACGAAGCTCCTGTCCACTATCAGCCTAACTCGGCCTTCGGTCCG
TTTTGGTCCGTAACCCGTCACGCAGACATCGTCGCAGTGGATAAAAATCACGAGCTG
TTCTCGGCCGAACCCTTTATCGTGATTGGAGCCCCACCGCGTTTCCTTGACATTGCCA
TGTTTATCGCAATGGACCCGCCGCGCCACGATGCACAACGTGCCGCAGTACAAGGG
GTGGTTGCCCCCAAGAACTTACGCGAAATGGAAGGTTTGATCCGCTCCCGTGTGCAA
GAAGTTTTGGATGACCTGCCGGTAGACCAACCCTTTGATTGGGTCCAAAATGTTAGT
ATCGAGTTAACGGCTCGCATGTTAGCCACATTATTGGACTTTCCGTATGAACAACGC
CGTAAGTTGGTGTACTGGTCAGACCTGGCGACCTCTCTGGAGCAGGCGAACGGGGG
CCCCAGCGATAATGACGAAGTGTTTCCCGGAATGCGCGATATGGCTCGTGGGTTATC
AGCATTATGGCATGACAAAGCCGCTCGCATGGCTGCGGGGGAAGAACCAGGCTTTG
ATCTGATCACTATGTTGCAGAGCAATGAGAACACGAAGGATCTTATCGATCGTCCTA
TGGAATTTCTGGGCAATCTGGTGTTACTGATTGTAGGAGGAAACGACACGACGCGC
AACTCTATGTCCGGAGGAGTCCTTGCGTTGAACCGCTTCCCAGATCAGTTCGAGAAG
CTTAAGGCTAACCCAGATTTGATTCCAAATATGGGTTCAGAAATTATTCGTTGGCAA
ACACCTTTGGCTTACATGCGTCGTGTTGCGAAAGCCGATACCATTTTGAACGGTCAG
TTTATCCGTAAGGGGGATAAGGTTGTGATGTGGTATGCAAGTGGTAATCGTGATGAG
CGCGTGTTCGAACGCCCCGATGAATTTATTATTGACCGTGCTAACGCCCGTAACCAC
ATCTCTTTTGGCTTCGGGGTCCACCGTTGTATGGGTAATCGTTTGGCCGAATTGCAAT
TACGTATCCTTTGGGAAGAATTATTGCCTCGCTTTGACAATATCGAGGTAGTAGGGG
AGCCTGAGTACGTTCAGTCGAATTTTGTACGCGGTATTAGTAAAATGATGGTACGTC
TGACCCCCAAATCCAGTGCCTACTAA
CYP153_Rrub from Rhodococcus ruber BKS 20-38 (protein)
SEQ ID NO: 30
MKIPEAITAKVQSTIPMDLQIQGAHLYDKTRRWVTGTNGEKLFVESPIPPVEDVELADID
LSNPFLYRQGRWQSYFERLRNEAPVHYQPNSAFGPFWSVTRHADIVAVDKNHELFSAEP
FIVIGAPPRFLDIAMFIAMDPPRHDAQRAAVQGVVAPKNLREMEGLIRSRVQEVLDDLP
VDQPFDWVQNVSIELTARMLATLLDFPYEQRRKLVYWSDLATSLEQANGGPSDNDEVF
PGMRDMARGLSALWHDKAARMAAGEEPGFDLITMLQSNENTKDLIDRPMEFLGNLVL
LIVGGNDTTRNSMSGGVLALNRFPDQFEKLKANPDLIPNMGSEIIRWQTPLAYMRRVAK
ADTILNGQFIRKGDKVVMWYASGNRDERVFERPDEFIIDRANARNHISFGFGVHRCMGN
RLAELQLRILWEELLPRFDNIEVVGEPEYVQSNFVRGISKMMVRLTPKSSAY.
CYP153_Gpar from Gordonia paraffinivorans (DNA)
SEQ ID NO: 31
ATGCAGATCCTTGACCGTGTCGTCGAGACGGTGCAGGCCAATATTCCGGTCGACCGC
CAGGTGCAGGCGTTACAACTGTTTCACAAAGCACGTGGTCGCCTGGTAGGGGAATC
ACGTCCAGAGCCGTTTGTAGAGAAGCCCATCCCCCCAGTTGATGAAGTATCGCTGGA
TGCCATTGACATGTCTAATCCCTTCATGTATCGCCAAGGCCAGTGGGCGCCATACTT
CGCGCGCTTGCGTGAAGAAGCGCCAGTTCATTACCAGCCCAATTCCCGTTTTGGGCC
GTTTTGGTCGGTGACCCGTTACGAAGACATCCTGACCGTCGATAAAGACCACGAGAC
CTTCAGTGCAGAACCATTTATCGTAATTGGGACGCCGCCCCCTGGATTGGATGTAGA
GATGTTTATTGCTATGGACCCGCCGCGCCACGATGTGCAACGCCGCGCGGTCCAGGG
AGTCGTAGCCCCAAAAAACTTAAAAGAAATGGAAGGACTGATCCGCGAGCGTGTTT
GTGAAGTGTTAGATAATCTTCCCGTAGGTCAGCCATTTAACTGGGTCGATCGCGTTA
GTGTAGAGATCACTGCTCGCACCTTAGCGACGATCTTAGATTTCCCCTATGAGCAGC
GCCGTAATTTAGTTCGCTGGAGTGACCTGGCCGCAGGATCCGAAGAAGCCACAGGT
GGTGCCAGCGATCCAGACGTCGTCTACCAGGCGGCGCTTGAGATGGTACGTGCTTTC
TCAGAGTTATGGCACGATAAGGCCGCACGTCGTGCCGCAGGCGAGGAACCAGGCTT
TGATTTGATCTCAATGTTACAATCCAATGAGGACACCAAGGATTTGGTTAAACGTCC
GATGGAATTCATTGGGAATCTTACATTACTGATTGTCGGTGGAAACGACACCACTCG
CAATAGCATGTCGGGTGGAGTGTACGCGCTGAATAAGTTTCCTGGAGAGTTCGAGA
AACTTAAGGCAGACCCGTCTTTGATTCCGAATATGGTTTCTGAGATCATTCGTTGGC
AGACGCCCTTGGCGTACATGCGTCGCGTCGCGACTAAAGACACCATCCTTAATGGCC
AATTCATTCGTAAAGGCGACAAGTTGGTGATGTGGTATGCATCCGGAAACCGCGAT
GAGTCTAAGTTCGAGCGTGCGGATGAGTTGATTATCGACCGCCCCAACGCTCGTAAT
CACATGGCGTTTGGGTATGGGGTGCACCGCTGCATGGGCAATCGTCTTGCCGAGCTG
CAACTTCGCATCTTATGGGAAGAATTACTGGAACGCTTCGACGACATCAAGGTCTTG
GAGGAACCAGAGTATGTTCAGTCTAACTTTGTTCGCGGTTACTCTAAGTTGATGGTG
GAGCTGACGGCGAAGAAGACGTCTTACTAA
CYP153_Gpar from Gordonia paraffinivorans (protein)
SEQ ID NO: 32
MQILDRVVETVQANIPVDRQVQALQLFHKARGRLVGESRPEPFVEKPIPPVDEVSLDAID
MSNPFMYRQGQWAPYFARLREEAPVHYQPNSRFGPFWSVTRYEDILTVDKDHETFSAE
PFIVIGTPPPGLDVEMFIAMDPPRHDVQRRAVQGVVAPKNLKEMEGLIRERVCEVLDNL
PVGQPFNWVDRVSVEITARTLATILDFPYEQRRNLVRWSDLAAGSEEATGGASDPDVV
YQAALEMVRAFSELWHDKAARRAAGEEPGFDLISMLQSNEDTKDLVKRPMEFIGNLTL
LIVGGNDTTRNSMSGGVYALNKFPGEFEKLKADPSLIPNMVSEIIRWQTPLAYMRRVAT
KDTILNGQFIRKGDKLVMWYASGNRDESKFERADELIIDRPNARNHMAFGYGVHRCMG
NRLAELQLRILWEELLERFDDIKVLEEPEYVQSNFVRGYSKLMVELTAKKTSY.
CYP153_A_OC4 from Acinetobacter sp. OC4 (DNA)
SEQ ID NO: 33
ATGAACTCGGTGGCAGAGATTTTTGAGAAGATTACTCAAACGGTCACTAGTACAGC
AGCAGACGTTGCCACAACAGTGACTGACAAGGTGAAATCGAACGAACAGTTTCAGA
CGGGAAAGCAATTCTTACACGGACAAGTTACCCGCTTCGTACCTTTGCACACACAGG
TCCGCGGTATTCAGTGGATGCAAAAGGCTAAGTTCCGCGTGTTTAACGTGCAAGAGT
TTCCCGCATTTATTGAGCAACCTATTCCTGAGGTCGCCACGCTTGCTCTTGCGGAAAT
CGACGTTTCCAACCCCTTTTTGTACAAACAGAAGAAATGGCAGTCTTATTTCAAGCG
CTTGCGTGATGAAGCGCCCGTGCATTATCAGGCGAACTCTCCCTTCGGCGCATTCTG
GTCAGTCACCCGTTATGACGACATTGTCTATGTCGACAAGAATCACGAGATCTTTTC
GGCTGAACCAGTAATTGCCATTGGGAATACGCCGCCCGGCTTAGGTGCAGAGATGTT
TATCGCGATGGACCCACCGAAGCACGATGTCCAACGCCAGGCTGTCCAGGATGTAG
TCGCACCGAAAAACTTGAAAGAATTAGAGGGCTTGATTCGCCTTCGTGTTCAGGAAG
TACTGGACCAATTGCCCACGGATCAACCCTTTGATTGGGTGCAAAATGTATCCATCG
AATTGACGGCGCGTATGTTGGCAACCTTGTTTGATTTTCCATACGAAAAGCGTCACA
AGTTAGTAGAGTGGTCAGATCTGATGGCCGGGACTGCGGAGGCAACAGGGGGTACA
GTGACTAACCTGGATGAGATTTTCGACGCTGCTGTCGACGCGGCTAAACACTTTGCT
GAATTGTGGCATCGCAAAGCCGCTCAGAAGTCTGCAGGAGCGGAGATGGGTTATGA
TTTGATTTCTCTTATGCAGAGTAATGAGGCAACCAAAGATTTGATCTATCGCCCCAT
GGAATTTATGGGAAATCTGGTGTTATTAATCGTAGGTGGTAATGACACTACACGTAA
TAGTATGACGGGTGGTGTTTATGCGTTGAATCTTTTTCCAAATGAGTTTGTAAAGTTG
AAGAATAATCCCTCACTGATTCCGAATATGGTCAGCGAAATTATCCGCTGGCAAACA
CCTCTGGCATATATGCGCCGCATCGCCAAGCAGGACGTCGAGTTAAATGGGCAAAC
AATTAAAAAAGGTGACAAAGTGGTAATGTGGTATGTTTCGGGGAACCGTGATGAAC
GTGTGATTGAGCGCCCAGATGAACTTATCATTGACCGTAAGGGTGCTCGTAATCACC
TTTCTTTCGGTTTCGGAGTGCATCGCTGCATGGGAAACCGTTTAGCGGAGATGCAGC
TTCGTATCTTGTGGGAAGAGCTGCTTCAACGCTTTGAAAATATCGAAGTTCTGGGGG
AGCCGGAAATCGTGCAATCGAACTTTGTCCGCGGTTACGCCAAAATGATGGTGAAA
CTGACCGCGAAGGCCTACTAA
CYP153_A_OC4 from Acinetobacter sp. OC4 (protein)
SEQ ID NO: 34
MNSVAEIFEKITQTVTSTAADVATTVTDKVKSNEQFQTGKQFLHGQVTRFVPLHTQVRG
IQWMQKAKFRVFNVQEFPAFIEQPIPEVATLALAEIDVSNPFLYKQKKWQSYFKRLRDE
APVHYQANSPFGAFWSVTRYDDIVYVDKNHEIFSAEPVIAIGNTPPGLGAEMFIAMDPPK
HDVQRQAVQDVVAPKNLKELEGLIRLRVQEVLDQLPTDQPFDWVQNVSIELTARMLAT
LFDFPYEKRHKLVEWSDLMAGTAEATGGTVTNLDEIFDAAVDAAKHFAELWHRKAAQ
KSAGAEMGYDLISLMQSNEATKDLIYRPMEFMGNLVLLIVGGNDTTRNSMTGGVYALN
LFPNEFVKLKNNPSLIPNMVSEIIRWQTPLAYMRRIAKQDVELNGQTIKKGDKVVMWYV
SGNRDERVIERPDELIIDRKGARNHLSFGFGVHRCMGNRLAEMQLRILWEELLQRFENIE
VLGEPEIVQSNFVRGYAKMMVKLTAKAY.
CYP153_Smac from Sphingopyxis macrogoltabida (DNA)
SEQ ID NO: 35
ATGGAGCACACCGGTCAATCTGCGGCAGCCACAATGCCACTGGATAGCATCGATGT
GTCCATCCCAGAACTTTTTTATAACGATTCGGTAGGAGAGTATTTCAAGCGTTTACG
CAAGGATGACCCCGTGCACTATTGTGCAGATTCCGCGTTCGGCCCATATTGGTCCAT
CACTAAGTATAACGATATCATGCACGTGGACACAAACCATGACATTTTTTCTAGCGA
CGCCGGATACGGGGGTATTATTATTGACGACGGCATTCAAAAGGGTGGGGACGGTG
GGTTAGATTTACCGAATTTCATCGCTATGGATCGTCCCCGTCATGACGAGCAACGTA
AGGCTGTGAGCCCAATCGTGGCTCCTGCGAACTTGGCTGCCCTTGAGGGAACGATCC
GCGAGCGTGTCAGCAAGACTCTGGACGGTCTGCCGGTTGGTGAGGAGTTCGACTGG
GTTGACCGTGTATCAATCGAGATTACTACGCAGATGTTAGCGACTTTGTTCGACTTTC
CGTTCGAAGAGCGCCGTAAACTGACCCGCTGGAGTGATGTAACGACCGCCGCACCC
GGAGGCGGAGTCGTGGAGAGTTGGGACCAACGTAAGACCGAATTACTTGAGTGCGC
GGCCTATTTTCAGGTTCTTTGGAATGAGCGTGTGAACAAAGACCCCGGCAACGATCT
TATTAGTATGCTTGCACATAGTCCTGCTACTCGTAACATGACACCTGAAGAATACTT
GGGTAATGTTCTGCTTTTGATCGTAGGCGGGAACGACACCACGCGTAACTCAATGAC
TGGTGGCGTATTGGCTCTGCACAAGAACCCGGATCAATTTGCAAAGCTGAAAGCTA
ACCCCGCCCTTGTTGAAACGATGGTCCCCGAGATCATCCGTTGGCAAACGCCTTTAG
CGCATATGCGTCGTACGGCAATTGCGGACTCGGAGCTGGGTGGTAAGACTATTCGCA
AGGGTGATAAAGTCGTGATGTGGTACTATTCAGGTAACCGCGACGATGAAGTTATTG
ATCGTCCCGAAGAATTCATTATTGACCGCCCTCGCCCCCGTCAACACCTTTCCTTCGG
TTTCGGCATCCATCGCTGTGTTGGGAATCGCTTAGCTGAAATGCAACTTCGTATTCTG
TGGGAAGAAATTCTTACGCGCTTCAGTCGCATTGAGGTGATGGCGGAACCTGAGCGT
GTGCGCTCGAATTTTGTTCGCGGTTACGCGAAAATGATGGTCCGTGTTCATGCTTACT
AA
CYP153_Smac from Sphingopyxis macrogoltabida (protein)
SEQ ID NO: 36
MEHTGQSAAATMPLDSIDVSIPELFYNDSVGEYFKRLRKDDPVHYCADSAFGPYWSITK
YNDIMHVDTNHDIFSSDAGYGGIIIDDGIQKGGDGGLDLPNFIAMDRPRHDEQRKAVSPI
VAPANLAALEGTIRERVSKTLDGLPVGEEFDWVDRVSIEITTQMLATLFDFPFEERRKLT
RWSDVTTAAPGGGVVESWDQRKTELLECAAYFQVLWNERVNKDPGNDLISMLAHSPA
TRNMTPEEYLGNVLLLIVGGNDTTRNSMTGGVLALHKNPDQFAKLKANPALVETMVPE
IIRWQTPLAHMRRTAIADSELGGKTIRKGDKVVMWYYSGNRDDEVIDRPEEFIIDRPRPR
QHLSFGFGIHRCVGNRLAEMQLRILWEEILTRFSRIEVMAEPERVRSNFVRGYAKMMVR
VHAY.
CYP153_M_HXN1500 from Mycobacterium sp. HXN-1500
(DNA) SEQ ID NO: 37
ATGACGGAAATGACAGTCGCAGCCTCCGACGCGACTAATGCGGCTTATGGTATGGC
CCTGGAAGATATCGATGTCAGTAACCCTGTGCTGTTCCGTGACAACACATGGCACCC
TTACTTTAAACGCTTACGCGAGGAGGACCCAGTTCATTATTGCAAATCCAGTATGTT
CGGGCCCTATTGGTCAGTGACAAAATATCGTGATATTATGGCCGTAGAAACCAATCC
GAAGGTCTTTAGCTCTGAGGCGAAATCTGGCGGAATCACAATTATGGACGACAACG
CGGCTGCTAGTCTGCCAATGTTTATTGCGATGGATCCTCCGAAACATGACGTACAAC
GTAAAACGGTTTCGCCGATCGTCGCGCCAGAGAACTTGGCCACAATGGAGTCAGTG
ATTCGTCAGCGCACGGCTGATTTACTTGACGGGTTACCCATTAACGAGGAGTTCGAC
TGGGTCCATCGTGTCTCTATCGAGCTGACCACGAAGATGCTGGCCACGCTTTTCGAT
TTCCCTTGGGACGATCGCGCCAAACTGACACGCTGGTCCGACGTTACAACTGCGCTT
CCCGGAGGGGGCATTATTGATAGTGAGGAACAGCGTATGGCGGAGTTGATGGAGTG
TGCGACATATTTCACTGAATTGTGGAATCAGCGTGTGAATGCCGAGCCAAAGAACG
ATTTAATTTCCATGATGGCTCACAGCGAGTCAACCCGTCATATGGCTCCGGAGGAGT
ACTTAGGGAATATCGTACTGCTGATTGTTGGTGGAAACGATACTACACGCAATTCGA
TGACAGGGGGGGTGCTTGCGTTAAACGAATTTCCGGATGAATATCGTAAGTTATCCG
CCAACCCTGCCTTGATTAGTTCGATGGTATCAGAAATCATTCGTTGGCAAACCCCAC
TTAGTCACATGCGTCGTACAGCCCTGGAAGATATCGAATTTGGTGGTAAACATATCC
GCCAGGGTGACAAAGTTGTGATGTGGTACGTCTCTGGTAACCGTGACCCGGAAGCT
ATCGATAATCCCGACACATTCATTATTGACCGCGCTAAGCCGCGCCAGCATTTATCC
TTCGGGTTCGGGATTCATCGTTGTGTAGGCAACCGTTTAGCTGAATTGCAGTTAAAT
ATCCTTTGGGAGGAGATTTTAAAACGTTGGCCGGATCCACTGCAGATCCAAGTGTTA
CAGGAACCCACCCGTGTTCTGAGCCCCTTCGTTAAGGGCTACGAGAGTCTGCCGGTA
CGTATCAATGCGTACTAA
CYP153_M_HXN1500 from Mycobacterium sp. HXN-1500
(protein) SEQ ID NO: 38
MTEMTVAASDATNAAYGMALEDIDVSNPVLFRDNTWHPYFKRLREEDPVHYCKSSMF
GPYWSVTKYRDIMAVETNPKVFSSEAKSGGITIMDDNAAASLPMFIAMDPPKHDVQRK
TVSPIVAPENLATMESVIRQRTADLLDGLPINEEFDWVHRVSIELTTKMLATLFDFPWDD
RAKLTRWSDVTTALPGGGIIDSEEQRMAELMECATYFTELWNQRVNAEPKNDLISMMA
HSESTRHMAPEEYLGNIVLLIVGGNDTTRNSMTGGVLALNEFPDEYRKLSANPALISSM
VSEIIRWQTPLSHMRRTALEDIEFGGKHIRQGDKVVMWYVSGNRDPEAIDNPDTFIIDRA
KPRQHLSFGFGIHRCVGNRLAELQLNILWEEILKRWPDPLQIQVLQEPTRVLSPFVKGYE
SLPVRINAY.
CYP153_Cmic from Candidatus Microthrix parvicella RN1
(DNA) SEQ ID NO: 39
ATGACCGATGACACGAAGCCGCGCATTGATTTCGACCCATCGATTCGTACGCCAGA
GATGGAAATGGCCGAAGTTGGGGCGGGGGTTCCAGATGCTGCTGACTTAAAACTTA
CAGACTTAAATCCCGCTAATCCACATTTGTTTAAGGAGGACCGCTGGCACGATCATT
TCGCTCGCTTACGTGCGGAGGACCCGGTCCATCTTAATGAAATCGAGACTGCGGGTC
GCTACTGGTCTATCACGAAGTATGACGATGTGCGCGCCGTCGACGGCGATTGGCAA
ACTTTCTCGTCGGCACAAGGTATGACGTTGGGGCTGCGCCCTGATCCCGACCGCCCG
AATCCGCTGGTACAAATCACCCCTTTCATTGCGATGGACCCGCCGGAGCACACAGCA
CAACGTAAAACTGTTCGCAGCGTGTCTGCTCCGTCCAACCTTCGTAACTTAGAACCC
TTGATCCGCGAACGTACTGTTGCAGTGCTTGATTCTTTACCCGAAGGAGAAACCTTC
GACTGGGTCGATACGGTCAGTATTGAGCTGACAACCCTTTTACTTGCCACATTATTT
GACTTTCCGCTTGAGGATCGTCGCAAGCTTACCCGCTGGTCCGACATTGTTTTCGCCG
TGCCCGGGTCGGGTGGAGTTGTAGAAACGCAGCAACAGAAAATCGAAGAGCTTATG
GAGTGCGTCACTTACTTTGAGGGGTTATGGGAAGAGCGTCGTGGTGGTGACGGGAC
AGACTTAGTTTCGATGTTGGCCAACGGGGAAGCAACTAAAGACATGCCCACCATTTC
GCATTTGGGTAATCTGTTGCTGTTAATCGTGGGAGGGAACGATACAACCCGCAATAC
CATGACAGGTTCGGTGTACGGATTGAACAAATATCCAGATCAATATGACAAATTAA
CTGCGGACCCTGGCTTGATTAGCACCTTTGTTCCGGAGATTATTCGCTGGCAGACAC
CACTGTCTTATATGCGTCGCACAGCCACGAAAGATTGTACAATCCGTGATAAACAGA
TCCTTGAAGGCGACCAAGTGCTTATGTGGTATATTTCTGCTAATCGTGATGAGGACG
TTTTTGTAGACGCTGAGCGCATCGACTTAGACCGCTCTAATGCGGACCGCCAACTGG
CGTTCGGCTACGGAATCCACTTCTGCATGGGGTCGCGTCTTGCTGAATTGCAACTGC
GCATTTTATGGGAAGAGGTACTTGCACGTTTCGAACGTATTGAGCTTCAAGCCGAGC
CTGAGCGCACACTTAGTTCCTTTGTTCATGGGTACACGAAACTGCCTGTAACTGTTA
CTCGCCGTTACTAA
CYP153_Cmic from Candidatus Microthrix parvicella RN1
(protein) SEQ ID NO: 40
MTDDTKPRIDFDPSIRTPEMEMAEVGAGVPDAADLKLTDLNPANPHLFKEDRWHDHFA
RLRAEDPVHLNEIETAGRYWSITKYDDVRAVDGDWQTFSSAQGMTLGLRPDPDRPNPL
VQITPFIAMDPPEHTAQRKTVRSVSAPSNLRNLEPLIRERTVAVLDSLPEGETFDWVDTV
SIELTTLLLATLFDFPLEDRRKLTRWSDIVFAVPGSGGVVETQQQKIEELMECVTYFEGL
WEERRGGDGTDLVSMLANGEATKDMPTISHLGNLLLLIVGGNDTTRNTMTGSVYGLNK
YPDQYDKLTADPGLISTFVPEIIRWQTPLSYMRRTATKDCTIRDKQILEGDQVLMWYISA
NRDEDVFVDAERIDLDRSNADRQLAFGYGIHFCMGSRLAELQLRILWEEVLARFERIELQ
AEPERTLSSFVHGYTKLPVTVTRRY.
CYP153_A_P52 from Afipia sp. P52-10 (DNA) SEQ ID NO: 41
ATGCAATCGACGCAACGTGGGGCCCGTGATTTTGCGACACGTCTGCCATTGGACGCG
ATTGACGTATCCGATCCACAACTGTATCAGGACGATACCTGGCGTCCTCTTTTCGCG
CGCCTTCGCGCGGAAGACCCGGTCCACTATTGTCGCGACTCGGCGTTTGGTCCCTAC
TGGAGTGTAACGACGTACGACGATATCTTGAAAGTGGAACTTGATCATTCAACGTAC
TCATCCAGCTCGGAACTGGGAGGCATTCAGGTGACCGATCAACCGAAGGGAAAGGA
AACTATTTCTTTCATTCGTATGGACCCACCTGGCCACACTGCGCAACGCCGTATCGTT
GCTCCTATCGTCGCCCCCACACACTTGGCAAACTTTGAGCCAGTGATCCGTGAGCGC
ACAGCCCGTGTTCTTGACGGCCTGCCTCGTAATGAAACCTTCGATTGGGCTGACCGC
GTCTCCGTCGAACTTACCGCAATGATGTTAGCAACTCTTTTCGACTTTCCCATGGAAG
AACGTCGCAAGTTAACTTACTGGTCCGACGTAGCGATTGCTAACATTAACTCGCCGG
AGAGCCCTATCACATCTGAGGATGAGCGCTCGGAGAAATTAGGGGAGATGGCCGCG
TGCTTCAAGGCTTTGTGGGATCGCCGTGCAGCAGTCGAGCCCAAGTTCGACCTTGTA
TCAATGTTGGCGCATGGGGCAGCGACTCGTGATATGGGGGTCCGTGAATTAACAGG
TACTATCGGTTTGCTGATCGTGGGGGGTAATGATACAACCCGTAATTCCATGACGGG
TGGAGTACTGGCGCTTCACGACTATCCCGAGGAGGCAGAAAAGTTGCGCGGCAATC
CAGCCTTAATCCCGTCGTTAGTGTCCGAAATTATTCGTTATCAATCCCCGGTTTTGCA
CATGCGTCGTACGGCCCGTGTTGATGCTGAGATTGGAGGAAAGACCATCCGCGCTG
GGGACAAGGTCGTCATGTGGTACATTTCCGGGAACCGTGATGAGAAAAAGATCGAG
CACGCAGATCGTTTTGTGATCGACCGTGCCAAGCCCCGTCAACATTTGGCTTTCGGG
GCCGGCGTTCACCGTTGTGTTGGAGATCGCCTTGCCGAATTGCAGCTGCGCATTCTTT
GGGAAGCAATCCTGGAACGCGGCTTTGTTATTGACGTAGTTGGGGAACCAAAGCGT
CTTTATTCCAATTTCATCCGTGGCTTTCGCTCTCTTCCCGTGCGCATTCGCACTGCCTA
CTAA
CYP153_A_P52 from Afipia sp. P52-10 (protein) SEQ ID NO: 42
MQSTQRGARDFATRLPLDAIDVSDPQLYQDDTWRPLFARLRAEDPVHYCRDSAFGPYW
SVTTYDDILKVELDHSTYSSSSELGGIQVTDQPKGKETISFIRMDPPGHTAQRRIVAPIVAP
THLANFEPVIRERTARVLDGLPRNETFDWADRVSVELTAMMLATLFDFPMEERRKLTY
WSDVAIANINSPESPITSEDERSEKLGEMAACFKALWDRRAAVEPKFDLVSMLAHGAAT
RDMGVRELTGTIGLLIVGGNDTTRNSMTGGVLALHDYPEEAEKLRGNPALIPSLVSEIIR
YQSPVLHMRRTARVDAEIGGKTIRAGDKVVMWYISGNRDEKKIEHADRFVIDRAKPRQ
HLAFGAGVHRCVGDRLAELQLRILWEAILERGFVIDVVGEPKRLYSNFIRGFRSLPVRIR
TAY.
CYP153_B_CACIA14H2 from Blastomonas sp. CACI414H2
(DNA) SEQ ID NO: 43
ATGGCAACAGTGCTGAAGGAGCCCGGCGCGGCGTTGAACTACGATATGAGTGATGC
GTCCTGGTATGTGGAGGATCGCTGGCAAGAACCATTCCGTCAAATGCGTGAGCAAG
ACCCGATTCACTGGACTGAAAATGGCATGTTCGGTAGTTTCTGGAATGTAACTAATC
ACAAAGCGATCCAACACGTCGAAGCATTGCCAGAAATTTTCAGTTCCTCCTACGAAC
ATGGAGGGATTACACTGGCAGACCGTATCGACGACGGGACGGAACTGGTTATGCCA
ATGTTCATTGCCATGGACCGTCCGAAACACACAGGACAACGTCGTACTGTAGCTCCG
GCCTTCACCCCTACAGAAATGAAGCGTATGTCTGACGATATCCGCCGTCGCACTGCC
GAAATTTTAGACGGCCTTCCATGGGACCAGCCGTTCGATTGGGTTGATCGTGTCAGT
ATCGAGCTTACGACACAGATGCTGGCTATTTTGTTCGACTTCCCATGGGAGGATCGT
CGTAAACTGACCGAATGGAGTGATTGGGCCGGAGATATTGAATTGATTCATTCGGA
GGAAATGCGTCAAGAGCGCTTAAAACATCTGTACGATATGGGCGCGTACTTTAAAA
AGCTTTGGGATGCCAAAATCAACGCAGAACCAACCCCAGATTTAATCTCTATGATGA
TCCATTCCGACGCCATGAGCGAGATGGATGAATTTGAGTTTATGGGTAACTTAATCC
TGTTAATCGTCGGTGGCAACGATACGACGCGTAATTCAATGTCGGGTCTTGTGTATG
GCCTGCAGCAGTTCCCGGATCAACGCGAGAAGCTTGAACAAAATCCGGCGCTTATC
CCTAATGCGGTGCAAGAGATCATTCGCTGGCAGACACCCTTAGCCCATATGCGCCGC
ACAGCACTGGAAGATTATGACTTATTCGGCAAGACTATTCGCAAAGGAGATAAACT
GGCACTTTGGTACATTTCAGGGAACCGTGACGAGAGTGTGTTTGAGGATGCGGATA
AAATCATCGTAGATCGTGAAAATGCTCGCCGCCACCTTGCGTTCGGATATGGTATCC
ACCGTTGTGTCGGCGCTCGTTTAGCCGAATTGCAGATTGCGATTCTGTTAGAAGAGA
TGGCGAAACGTCGTATGCGTGTTAATGTCCTTGAAGAGCCAGTTCGCGTTCGCGCGT
GTTTTGTGCATGGGTATCGCTCCATGCAAGTTTCCTTATCAAAGTACTACTAA
CYP153_B_CACIA14H2 from Blastomonas sp. CACI414H2
(protein) SEQ ID NO: 44
MATVLKEPGAALNYDMSDASWYVEDRWQEPFRQMREQDPIHWTENGMFGSFWNVTN
HKAIQHVEALPEIFSSSYEHGGITLADRIDDGTELVMPMFIAMDRPKHTGQRRTVAPAFT
PTEMKRMSDDIRRRTAEILDGLPWDQPFDWVDRVSIELTTQMLAILFDFPWEDRRKLTE
WSDWAGDIELIHSEEMRQERLKHLYDMGAYFKKLWDAKINAEPTPDLISMMIHSDAMS
EMDEFEFMGNLILLIVGGNDTTRNSMSGLVYGLQQFPDQREKLEQNPALIPNAVQEIIRW
QTPLAHMRRTALEDYDLFGKTIRKGDKLALWYISGNRDESVFEDADKIIVDRENARRHL
AFGYGIHRCVGARLAELQIAILLEEMAKRRIVIRVNVLEEPVRVRACFVHGYRSMQVSLS
KYY.
CYP153_CPha1 from Candidatus Phaeomarinobacter ectocarpi
(DNA) SEQ ID NO: 45
ATGACGACCGCCAATCAAACTAGCCCAAATGGAGCCATTGACGTGAACGATATCCC
TTTGGCAGAGTTAGATGTGAGCCAACCTCATCTGTTTAAGAACGACACCTGGCGCCC
ATGGTTCGCACGCCTGCGTGCTGAGGCGCCCGTCCATTATCTTGCCGATAGCGAAAA
CGGACCTTTCTGGTCGGTCACGTCACACGATATGACTAAAGCGGTCGACGCAAACC
ATAAGGTCTTCTCATCCGAGGAGGGCGGCATTGCCATCGTCGACCCACAGCCTTTGG
ACGGTGAGCAATTAATGCGTGACCCTTCGTTTATCTCAATGGATGAGCCAAAGCATG
CTACACAACGCAAGGCCGTGTCGCCGGCTGTAGCTCCCAAAAACCTTGCAGAGCTG
GAACCTTTGATTCGCGAGCGTGCCGCTGACATCCTTGATAACCTGCCAGTCGGGGAA
ACCTTTAACTGGGTCGACCGTGTTTCAGTTGAACTTACAGCGCGCATGCTTGCGACG
TTGTTCGACTTCCCTTATGAACGCCGTCGTGACTTGATTCGCTGGAGTGATGTGGCCA
CCGCTGTACCTAAAGTGACTGGGGAGGCAAATGACATGGGTGCCCGCCGCGATGCG
CTTATTGAATGCGCTACTACATTCTACCAATTATGGCAGGAACGCGCGGCACAACCT
CCGAAGTTCGACTTCGTCAGCATGTTAGCCCATGGGGAAGCGACAAAACACCTTTCA
GAAGACCCATTATTGATGTTGGGGAACATCATCTTATTGATCGTGGGTGGCAATGAC
ACTACACGTAATTCGATTTCCGGGGGTGTTGTTGCTTTGAACCAGTATCCAGAAGAG
TATCAGAAGTTGCGTGATACTCCCGCCCTGATCCCCAATATGGTGGCGGAGACGGTA
CGCTGGCAAACCCCTGTTATCCACATGCGCCGTACCGCCCTTGAGGACGTTGAGCTG
GGCGGTAAGACGATCCGTAAAGGAGACAAAGTCGTGATGTGGTACCTTAGTGGTAA
CCGTGATGAGGCAGTCTTCCCAGACGCAGACCGCTTGATCATTGACCGCCCAAATGC
ACGCCAGCATGTATCGTTTGGATTCGGCGTGCACCGTTGCATGGGCAACCGTTTAGC
TGAAATGCAACTTCGTGTCTTGTGGGAGGAGATCATGAAACGCTTTCATACAGTCGA
AGTTGTGGGGGAAGTTGAGCGCCTTAGCAACAACTTTATCCGTGGGATCGCGTCAGT
CCCTGTGCGTTTGCACCCGTTATACTAA
CYP153_CPha1 from Candidatus Phaeomarinobacter ectocarpi
(protein) SEQ ID NO: 46
MTTANQTSPNGAIDVNDIPLAELDVSQPHLFKNDTWRPWFARLRAEAPVHYLADSENG
PFWSVTSHDMTKAVDANHKVFSSEEGGIAIVDPQPLDGEQLMRDPSFISMDEPKHATQR
KAVSPAVAPKNLAELEPLIRERAADILDNLPVGETFNWVDRVSVELTARMLATLFDFPY
ERRRDLIRWSDVATAVPKVTGEANDMGARRDALIECATTFYQLWQERAAQPPKFDFVS
MLAHGEATKHLSEDPLLMLGNIILLIVGGNDTTRNSISGGVVALNQYPEEYQKLRDTPAL
IPNMVAETVRWQTPVIHMRRTALEDVELGGKTIRKGDKVVMWYLSGNRDEAVFPDAD
RLIIDRPNARQHVSFGFGVHRCMGNRLAEMQLRVLWEEIMKRFHTVEVVGEVERLSNN
FIRGIASVPVRLHPLY.
CYP153_CPha2 from Candidatus Phaeomarinobacter ectocarpi
(DNA) SEQ ID NO: 47
ATGTCGCAAGCTGCGGCAGAGACCCCTAGCACAGTCGATCATCAGGAGCGTGCATG
GTCTATGCCTCTGGAAGATATCAACGTGGCTGACGGTGCACTTTTCCAAGACGATGC
TATTTGGCCCTACTTTGAACGTCTTCGCAAGGAAGCACCGGTTCATAAGGGACATAG
CGACGAGTTCGGTGACTATTGGAGTGTGACTCGTTATGAAGACATTATGGCGGTGGA
CACCAATCATCATGTTTTCTCCTCGGAGGGGGCCATTACCCTTGCAGATCCGTTGGA
AGATTTCCGTGCTCCAATGTTCATCGCAATGGATCCCCCGAAGCACGACAAACAGCG
TATTACTGTCCAACCGATCGTCGCCCCAAAAAATCTGCAAAACTGGGAGGGCTTGAT
CCGTGAGCGTACCGGCTTAATTCTGGATCAACTGCCCCGCAACGAGACGTTTGATTG
GGTCGATAAAGTAAGCATTGAACTTACCACTATGATGCTTGCCACGTTATTCGATTT
CCCTTTCGAGGAGCGCCGTCGCTTGACCCGTTGGTCTGATGTCGCCACAGGGCGTGA
TAATCCCGAAATTTATAAAAGCGAAGAACAGTGGCGTGGGGAGCTTATGGAGTGTT
TAGAGGCGTTCACTGGGTTATGGAACGACCGCGTTAACTCAGACACGCCCGGAAAC
GATTTAATTTCGATGCTGGCGAGCGGGGAATCTACTAAGAATATGGATCCCATGGAG
TATTTAGGCAACATTATTCTGCTTATTGTCGGGGGAAATGATACTACTCGCAACTCA
ATGACGGGATCTGTTTACGCCTTAAACAAGTTTGCGGGCGAATATGATAAGTTAATT
GCTGATCCTTCACTGATTCCCAATCTTTCGAGCGAGATCATTCGCTGGCAAACACCA
TTGGCACATATGCGTCGTACTGCACTTGAAGACATTGAGCTGAACGGCCAGATGATC
AAAAAAGGAGACAAAGTGGCCATGTGGTACGTATCCGGAAACCGCGACACGGCTGT
GTTCGAGAACGCTGATGACGTAATTATTGACCGCCCCAATGCACGTCGCCAGATGTC
CTTCGGGTATGGGATTCACCGCTGCGTTGGAAATCGCCTTGGTGAATTACAGATTAA
GATCCTGTGGGAGGAACTGCTGAAGCGTTTTCCCAAAATCGAGGTCATGGAAGAAC
CAACGCGCACACGTTCACCATTCGTCAAGGGATACACGTATATGCCCGTTCGTATTC
CGGCCTACTAA
CYP153_CPha2 from Candidatus Phaeomarinobacter ectocarpi
(protein) SEQ ID NO: 48
MSQAAAETPSTVDHQERAWSMPLEDINVADGALFQDDAIWPYFERLRKEAPVHKGHSD
EFGDYWSVTRYEDIMAVDTNHHVFSSEGAITLADPLEDFRAPMFIAMDPPKHDKQRITV
QPIVAPKNLQNWEGLIRERTGLILDQLPRNETFDWVDKVSIELTTMMLATLFDFPFEERR
RLTRWSDVATGRDNPEIYKSEEQWRGELMECLEAFTGLWNDRVNSDTPGNDLISMLAS
GESTKNMDPMEYLGNIILLIVGGNDTTRNSMTGSVYALNKFAGEYDKLIADPSLIPNLSS
EIIRWQTPLAHMRRTALEDIELNGQMIKKGDKVAMWYVSGNRDTAVFENADDVIIDRP
NARRQMSFGYGIHRCVGNRLGELQIKILWEELLKRFPKIEVMEEPTRTRSPFVKGYTYM
PVRIPAY.
CamA from Pseudomonas putida ATCC 17453 (DNA)
SEQ ID NO: 49
ATGAACGCAAATGATAACGTCGTAATCGTCGGGACGGGACTTGCGGGGGTAGAAGT
TGCATTTGGCTTACGCGCCAGTGGATGGGAGGGTAATATTCGCCTGGTTGGAGATGC
GACTGTTATTCCACACCACTTGCCCCCGTTGAGCAAAGCGTACTTAGCCGGTAAGGC
GACTGCAGAGTCATTATATTTACGTACTCCGGACGCCTATGCAGCACAAAACATCCA
ATTGCTTGGAGGTACGCAGGTTACAGCGATTAATCGTGACCGTCAACAAGTCATCCT
GTCTGATGGCCGTGCGTTGGACTATGACCGCTTAGTGCTGGCAACTGGTGGCCGCCC
GCGCCCGCTTCCTGTGGCAAGTGGTGCTGTCGGGAAAGCGAATAATTTCCGCTATCT
GCGTACTCTTGAAGACGCCGAGTGCATCCGCCGCCAGTTAATCGCCGATAATCGTCT
GGTGGTTATTGGTGGAGGTTACATTGGTCTGGAAGTAGCCGCGACCGCAATCAAGG
CGAACATGCATGTTACATTATTGGATACGGCCGCACGTGTGCTTGAACGCGTAACCG
CCCCGCCAGTATCCGCATTCTATGAACACTTACATCGCGAGGCAGGGGTTGATATCC
GTACTGGGACCCAGGTCTGTGGATTTGAAATGTCGACAGATCAGCAGAAAGTTACT
GCGGTCTTGTGCGAAGACGGGACCCGCCTTCCGGCGGATCTTGTGATCGCAGGAATT
GGTCTGATCCCCAATTGCGAATTAGCATCCGCAGCTGGCTTACAAGTGGATAACGGC
ATCGTGATTAACGAACACATGCAGACGAGTGACCCTCTTATCATGGCGGTTGGCGAC
TGTGCGCGCTTTCATAGCCAACTTTACGACCGTTGGGTGCGTATTGAGTCCGTACCC
AACGCGCTGGAACAAGCTCGTAAGATCGCGGCTATTTTGTGCGGTAAAGTGCCTCGT
GATGAAGCAGCTCCTTGGTTCTGGAGTGACCAATACGAGATTGGATTGAAGATGGT
AGGATTATCAGAAGGCTACGATCGCATTATTGTCCGCGGCTCACTTGCTCAGCCCGA
TTTCTCCGTCTTCTATCTTCAGGGAGATCGCGTGTTGGCTGTCGATACAGTTAATCGT
CCTGTGGAATTCAATCAAAGCAAACAGATTATCACGGATCGTTTACCCGTAGAGCCT
AACTTACTGGGCGACGAAAGCGTCCCACTTAAGGAGATCATTGCAGCCGCGAAGGC
TGAGTTATCGAGTGCATAA
CamA from Pseudomonas putida ATCC 17453 (protein)
SEQ ID NO: 50
MNANDNVVIVGTGLAGVEVAFGLRASGWEGNIRLVGDATVIPHHLPPLSKAYLAGKAT
AESLYLRTPDAYAAQNIQLLGGTQVTAINRDRQQVILSDGRALDYDRLVLATGGRPRPL
PVASGAVGKANNFRYLRTLEDAECIRRQLIADNRLVVIGGGYIGLEVAATAIKANMHVT
LLDTAARVLERVTAPPVSAFYEHLHREAGVDIRTGTQVCGFEMSTDQQKVTAVLCEDG
TRLPADLVIAGIGLIPNCELASAAGLQVDNGIVINEHMQTSDPLIMAVGDCARFHSQLYD
RWVRIESVPNALEQARKIAAILCGKVPRDEAAPWFWSDQYEIGLKMVGLSEGYDRIIVR
GSLAQPDFSVFYLQGDRVLAVDTVNRPVEFNQSKQIITDRLPVEPNLLGDESVPLKEIIAA
AKAELSSA.
CamB from Pseudomonas putida ATCC 17453 (DNA)
SEQ ID NO: 51
ATGTCTAAAGTTGTCTATGTCTCCCATGACGGAACCCGTCGTGAGTTAGACGTTGCT
GATGGTGTGTCACTGATGCAAGCAGCGGTCAGCAATGGAATTTATGATATTGTGGGC
GACTGTGGAGGTTCTGCCTCTTGCGCCACTTGTCATGTTTACGTAAACGAAGCGTTT
ACGGATAAGGTCCCTGCCGCCAACGAACGTGAGATTGGTATGTTAGAGTGCGTTACC
GCCGAGTTAAAGCCGAATTCGCGTTTGTGTTGTCAGATCATTATGACACCGGAGTTA
GATGGCATTGTTGTCGATGTCCCTGACCGTCAATGGTAA
CamB from Pseudomonas putida ATCC 17453 (protein)
SEQ ID NO: 52
MSKVVYVSHDGTRRELDVADGVSLMQAAVSNGIYDIVGDCGGSASCATCHVYVNEAF
TDKVPAANEREIGMLECVTAELKPNSRLCCQIIMTPELDGIVVDVPDRQW.
AlkB from Pseudomonas putida (DNA) SEQ ID NO: 53
ATGCTTGAGAAACACAGAGTTCTGGATTCCGCTCCAGAGTACGTAGATAAAAAGAA
ATATCTCTGGATACTATCAACTTTGTGGCCGGCTACTCCGATGATCGGAATCTGGCTT
GCAAATGAAACTGGTTGGGGGATTTTTTATGGGCTGGTATTGCTCGTATGGTACGGC
GCACTTCCATTGCTTGATGCGATGTTTGGTGAGGACTTTAATAATCCGCCTGAAGAA
GTGGTGCCGAAACTAGAGAAGGAGCGGTACTATCGAGTTTTGACATATCTAACAGTT
CCTATGCATTACGCTGCATTAATTGTGTCAGCATGGTGGGTCGGAACTCAGCCAATG
TCTTGGCTTGAAATTGGTGCGCTTGCCTTGTCACTGGGTATCGTGAACGGACTAGCG
CTCAATACAGGACACGAACTCGGTCACAAGAAGGAGACTTTTGATCGTTGGATGGC
CAAAATTGTGTTGGCTGTCGTAGGGTACGGTCACTTCTTTATTGAGCATAATAAGGG
TCATCACCGTGATGTCGCTACACCGATGGATCCTGCAACATCCCGGATGGGAGAAA
GCATTTATAAGTTTTCAATCCGTGAGATCCCAGGAGCATTTATTCGTGCTTGGGGGC
TTGAGGAACAACGCCTTTCGCGCCGTGGCCAAAGCGTTTGGAGTTTCGATAATGAAA
TCCTCCAACCAATGATCATCACAGTTATTCTTTACGCCGTTCTCCTTGCCTTGTTTGG
ACCTAAGATGCTGGTGTTCCTGCCGATTCAAATGGCTTTCGGTTGGTGGCAGCTGAC
CAGTGCGAACTATATTGAACATTACGGCTTGCTCCGTCAAAAAATGGAGGACGGTC
GATATGAGCATCAAAAGCCGCACCATTCTTGGAATAGTAATCACATCGTCTCTAATC
TAGTGCTGTTCCACCTTCAGCGGCACTCGGATCACCACGCGCATCCAACACGTTCTT
ATCAGTCACTTCGGGATTTTCCCGGCCTGCCGGCTCTTCCGACGGGTTACCCTGGTG
CATTTTTGATGGCGATGATTCCTCAGTGGTTTAGATCAGTTATGGATCCCAAGGTAG
TAGATTGGGCTGGTGGTGACCTTAATAAGATCCAAATTGATGATTCGATGCGAGAAA
CCTATTTGAAAAAATTTGGCACTAGTAGTGCTGGTCATAGTTCGAGTACCTCTGCGG
TAGCATCGTAG
AlkB from Pseudomonas putida (Protein) SEQ ID NO: 54
MLEKHRVLDSAPEYVDKKKYLWILSTLWPATPMIGIWLANETGWGIFYGLVLLVWYG
ALPLLDAMFGEDFNNPPEEVVPKLEKERYYRVLTYLTVPMHYAALIVSAWWVGTQPM
SWLEIGALALSLGIVNGLALNTGHELGHKKETFDRWMAKIVLAVVGYGHFFIEHNKGH
HRDVATPMDPATSRMGESIYKF SIREIPGAFIRAWGLEEQRLSRRGQSVWSFDNEILQPM
IITVILYAVLLALFGPKMLVFLPIQMAFGWWQLTSANYIEHYGLLRQKMEDGRYEHQKP
HHSWNSNHIVSNLVLFHLQRHSDHHAEIPTRSYQSLRDFPGLPALPTGYPGAFLMAMIPQ
WFRSVMDPKVVDWAGGDLNKIQIDDSMRETYLKKFGTSSAGHSSSTSAVAS.
AlkG from Pseudomonas putida (DNA) SEQ ID NO: 55
ATGGCTAGCTATAAATGCCCGGATTGTAATTATGTTTATGATGAGAGTGCGGGTAAT
GTGCATGAGGGGTTTTCTCCAGGTACGCCTTGGCACCTTATTCCTGAGGATTGGTGC
TGCCCCGATTGCGCCGTTCGAGACAAGCTTGACTTCATGTTAATTGAGAGCGGCGTA
GGTGAAAAGGGCGTCACCTCAACCCATACTTCGCCAAATTTATCCGAGGTTAGTGGC
ACAAGTTTAACTGCTGAAGCAGTGGTTGCGCCGACAAGCTTAGAGAAATTGCCTAGT
GCCGACGTTAAAGGCCAAGATCTATATAAAACTCAACCTCCAAGGTCTGATGCCCA
AGGCGGGAAAGCATACTTGAAGTGGATATGTATTACTTGTGGCCATATATATGATGA
GGCGTTGGGCGATGAGGCCGAGGGTTTTACTCCAGGTACTCGCTTTGAGGATATTCC
TGATGACTGGTGCTGTCCGGATTGCGGGGCTACGAAAGAAGACTATGTGCTCTACGA
GGAAAAGTGA
AlkG from Pseudomonas putida (Protein) SEQ ID NO: 56
MASYKCPDCNYVYDESAGNVHEGFSPGTPWHLIPEDWCCPDCAVRDKLDFMLIESGVG
EKGVTSTHTSPNLSEVSGTSLTAEAVVAPTSLEKLPSADVKGQDLYKTQPPRSDAQGGK
AYLKWICITCGHIYDEALGDEAEGFTPGTRFEDIPDDWCCPDCGATKEDYVLYEEK.
AlkT from Pseudomonas putida (DNA) SEQ ID NO: 57
ATGGCAATCGTTGTTGTTGGCGCTGGTACAGCTGGAGTAAATGCTGCGTTCTGGCTT
CGTCAATATGGTTATAAAGGGGAAATTAGGATTTTTAGCAGGGAGTCTGTGGCGCCT
TATCAGCGGCCTCCTCTATCCAAGGCTTTTCTGACAAGTGAGATTGCAGAATCCGCA
GTGCCATTAAAGCCAGAAGGTTTTTATACGAATAACAATATTACCATTTCGTTAAAT
ACACCGATTGTATCAATCGACGTGGGGCGTAAGATAGTTTCTTCTAAAGATGGAAAA
GAATACGCGTATGAAAAATTGATTCTTGCAACACCTGCTAGCGCACGTAGGTTAACC
TGCGAGGGGTCTGAACTGTCTGGGGTCTGCTATTTACGCAGTATGGAAGACGCCAAA
AATTTACGTAGGAAACTTGTGGAGAGTGCGTCTGTTGTTGTGTTGGGCGGCGGAGTA
ATCGGGCTTGAAGTCGCCTCAGCTGCGGTGGGCTTAGGGAAGAGGGTCACAGTGAT
AGAAGCCACCCCGCGTGTAATGGCGCGCGTGGTTACGCCGGCAGCAGCAAACTTAG
TCAGAGCCCGCCTGGAGGCTGAAGGAATTGAGTTCAAGCTGAATGCGAAATTAACG
TCTATAAAGGGCAGGAATGGCCATGTTGAACAATGCGTACTTGAAAGTGGAGAAGA
AATTCAGGCGGATCTGATTGTAGTTGGAATCGGTGCTATCCCAGAGCTAGAGCTGGC
AACTGAGGCGGCCCTTGAAGTGAGTAATGGTGTTGTGGTCGATGATCAGATGTGTAC
ATCGGATACAAGTATATATGCAATCGGCGACTGCGCAATGGCTAGAAATCCTTTTTG
GGGAACGATGGTACGTTTAGAGACAATTCATAATGCGGTTACACACGCTCAAATTGT
CGCAAGTAGCATCTGTGGCACATCAACACCAGCACCAACCCCACCACGGTTCTGGTC
TGATCTTAAAGGGATGGCGCTGCAAGGACTTGGTGCTCTAAAGGACTACGATAAAC
TCGTTGTTGCAATTAATAACGAAACTCTTGAACTAGAAGTCCTTGCGTACAAGCAGG
AGCGACTGATTGCAACTGAGACAATAAATTTGCCTAAACGTCAAGGTGCGCTTGCA
GGGAGTATAAAATTACCTGATTAG
AlkT from Pseudomonas putida (Protein) SEQ ID NO: 58
MAIVVVGAGTAGVNAAFWLRQYGYKGEIRIFSRESVAPYQRPPLSKAFLTSEIAESAVPL
KPEGFYTNNNITISLNTPIVSIDVGRKIVSSKDGKEYAYEKLILATPASARRLTCEGSELSG
VCYLRSMEDAKNLRRKLVESASVVVLGGGVIGLEVASAAVGLGKRVTVIEATPRVMAR
VVTPAAANLVRARLEAEGIEFKLNAKLTSIKGRNGHVEQCVLESGEEIQADLIVVGIGAIP
ELELATEAALEVSNGVVVDDQMCTSDTSIYAIGDCAMARNPFWGTMVRLETIHNAVTH
AQIVASSICGTSTPAPTPPRFWSDLKGMALQGLGALKDYDKLVVAINNETLELEVLAYK
QERLIATETINLPKRQGALAGSIKLPD.
Cyp102A7 from Bacillus licheniformis (DNA) SEQ ID NO: 59
ATGAACAAGTTAGATGGAATTCCAATCCCTAAAACTTACGGGCCGCTCGGCAACCTG
CCTTTGCTTGACAAAAACAGGGTCTCCCAGTCACTTTGGAAAATCGCGGATGAGATG
GGGCCTATCTTTCAATTTAAGTTTGCGGATGCGATTGGGGTTTTTGTGTCCAGCCATG
AACTGGTTAAAGAAGTCTCTGAAGAATCCCGTTTTGACAAAAACATGGGGAAGGGG
CTATTGAAAGTTCGCGAGTTCAGCGGAGACGGGCTCTTTACAAGCTGGACGGAAGA
ACCCAATTGGCGGAAAGCCCACAACATCCTTCTGCCGAGCTTCAGCCAGAAAGCGA
TGAAGGGATACCATCCCATGATGCAGGATATCGCCGTCCAGCTCATTCAAAAGTGGT
CCCGTCTCAATCAGGATGAAAGCATTGATGTGCCGGACGATATGACGCGGCTGACG
CTGGACACGATCGGCTTATGCGGGTTTAACTACCGCTTTAACAGCTTCTACCGTGAA
GGGCAGCATCCGTTTATTGAGAGCATGGTCCGGGGTTTGAGCGAAGCGATGAGACA
GACGAAGCGCTTCCCGCTGCAGGATAAGCTGATGATTCAAACGAAGCGCCGGTTTA
ACAGCGATGTCGAGTCGATGTTTTCTCTTGTTGACCGGATCATCGCTGACCGGAAGC
AGGCCGAGAGTGAAAGCGGAAATGACCTCTTGTCGCTTATGCTTCATGCGAAAGAC
CCTGAGACCGGCGAAAAACTGGATGATGAGAATATCCGCTATCAAATTATTACATTT
TTGATTGCCGGACACGAGACGACGAGCGGTTTATTATCGTTTGCAATCTATCTGCTC
CTGAAGCATCCGGATAAGCTTAAGAAAGCGTATGAAGAAGCAGACCGCGTGCTGAC
CGATCCCGTCCCATCCTACAAACAGGTTCAGCAGCTGAAATACATCCGAATGATTTT
GAATGAATCGATAAGGCTTTGGCCGACGGCACCGGCTTTCTCTCTTTATGCAAAAGA
AGAAACGGTTATCGGGGGAAAATATTTGATTCCAAAAGGACAGAGCGTTACAGTGC
TCATCCCAAAACTGCACAGAGATCAAAGCGTCTGGGGAGAAGATGCCGAGGCATTC
CGGCCTGAACGGTTCGAGCAGATGGACAGCATTCCGGCGCACGCATACAAACCGTT
TGGCAACGGCCAAAGGGCATGCATCGGCATGCAGTTCGCCCTTCATGAAGCGACGC
TTGTGCTCGGCATGATTCTTCAGTACTTTGATCTTGAAGATCATGCAAACTACCAATT
GAAGATCAAAGAATCGCTGACATTAAAACCGGATGGTTTCACAATCCGGGTGAGGC
CGAGGAAAAAAGAAGCAATGACGGCGATGCCGGGCGCTCAGCCTGAAGAGAACGG
ACGGCAGGAAGAACGGCCTTCCGCACCGGCGGCGGAAAATACGCACGGAACCCCTC
TTCTTGTGCTCTACGGTTCAAATCTCGGCACAGCCGAAGAGATTGCGAAGGAGCTTG
CTGAAGAAGCGCGTGAGCAAGGGTTTCACAGCCGGACGGCGGAGCTTGATCAATAC
GCAGGCGCCATCCCGGCAGAAGGGGCTGTTATCATTGTGACGGCTTCCTATAACGGA
AACCCGCCCGATTGCGCAAAGGAATTTGTCAATTGGCTTGAGCATGATCAGACAGA
CGATTTGCGTGGTGTCAAATATGCGGTATTCGGCTGCGGTAACCGCAGCTGGGCCAG
CACCTACCAGCGGATTCCGCGCCTGATTGACAGCGTATTGGAAAAAAAAGGCGCCC
AAAGGCTGCACAAGCTTGGAGAAGGGGATGCAGGCGATGATTTTGAAGGACAGTTT
GAGTCATGGAAATATGATCTGTGGCCGCTTTTAAGAACCGAATTTTCATTGGCCGAA
CCCGAGCCGAATCAAACAGAAACAGACAGGCAAGCCTTATCTGTCGAGTTCGTAAA
CGCACCTGCGGCTTCGCCGCTGGCTAAAGCTTATCAGGTGTTCACAGCGAAGATATC
GGCAAACCGAGAACTGCAGTGTGAAAAGAGCGGGAGAAGCACAAGGCATATTGAA
ATATCGCTTCCTGAAGGCGCCGCATATCAGGAGGGAGACCATCTCGGTGTGCTACCG
CAAAAtAGCGAAGTGCTGATTGGCCGCGTTTTTCAGCGGTTTGGGCTGAACGGAAAT
GAACAAATTCTGATTAGCGGCCGGAATCAAGCATCACATTTGCCTTTGGAGAGGCCC
GTTCATGTCAAAGACCTTTTTCAACATTGCGTCGAGCTCCAGGAACCGGCCACAAGG
GCCCAGATACGCGAGCTGGCGGCTCATACTGTTTGTCCGCCTCATCAGCGCGAGCTT
GAAGACCTGCTGAAAGATGACGTCTATAAGGATCAAGTGTTGAATAAGCGGCTGAC
AATGCTTGACCTGCTTGAGCAATACCCGGCCTGTGAACTGCCGTTCGCCCGTTTTCTG
GCGCTTCTTCCTCCGCTAAAACCGAGGTACTATTCGATTTCCAGTTCGCCGCAGCTTA
ACCCGCGGCAAACAAGCATCACCGTCTCTGTCGTAAGTGGCCCGGCGTTGAGCGGC
CGCGGGCATTATAAGGGAGTTGCATCGAACTATCTCGCCGGCCTTGAGCCGGGAGA
CGCGATTTCGTGTTTCATCAGAGAGCCTCAGTCAGGCTTCCGGCTTCCCGAAGATCC
TGAAACACCGGTGATCATGGTCGGGCCGGGCACCGGAATCGCCCCTTACCGCGGAT
TTCTTCAGGCGCGCCGCATCCAGCGCGATGCCGGTGTGAAGCTCGGTGAAGCGCATT
TGTACTTCGGCTGCCGCCGTCCGAACGAAGATTTTCTGTATCGAGACGAGTTGGAGC
AAGCGGAAAAGGACGGAATCGTCCATCTGCATACAGCGTTTTCCCGGCTTGAGGGC
CGGCCGAAAACATATGTGCAAGATTTGCTCAGAGAGGATGCAGCCTTGCTGATTCAC
TTGTTGAACGAAGGCGGCCGCCTGTATGTGTGCGGAGACGGAAGCCGCATGGCTCC
AGCTGTTGAACAAGCTTTGTGCGAGGCGTATCGCATAGTACAGGGTGCGAGTCGGG
AAGAGTCGCAAAGCTGGCTGTCCGCACTTTTAGAAGAAGGGCGCTATGCAAAGGAT
GTATGGGACGGCGGCGTTTCCCAACATAATGTGAAGGCGGACTGCATTGCAAGAAC
GTAA
Cyp102A7 from Bacillus licheniformis (Protein) SEQ ID NO: 60
MNKLDGIPIPKTYGPLGNLPLLDKNRVSQSLWKIADEMGPIFQFKFADAIGVFVSSHELV
KEVSEESRFDKNMGKGLLKVREFSGDGLFTSWTEEPNWRKAHNILLPSFSQKAMKGYH
PMMQDIAVQLIQKWSRLNQDESIDVPDDMTRLTLDTIGLCGFNYRFNSFYREGQHPFIES
MVRGLSEAMRQTKRFPLQDKLMIQTKRRFNSDVESMFSLVDRIIADRKQAESESGNDLL
SLMLHAKDPETGEKLDDENIRYQIITFLIAGHETTSGLLSFAIYLLLKHPDKLKKAYEEAD
RVLTDPVPSYKQVQQLKYIRMILNESIRLWPTAPAFSLYAKEETVIGGKYLIPKGQSVTV
LIPKLHRDQSVWGEDAEAFRPERFEQMDSIPAHAYKPFGNGQRACIGMQFALHEATLVL
GMILQYFDLEDHANYQLKIKESLTLKPDGFTIRVRPRKKEAMTAMPGAQPEENGRQEER
PSAPAAENTHGTPLLVLYGSNLGTAEEIAKELAEEAREQGFHSRTAELDQYAGAIPAEGA
VIIVTASYNGNPPDCAKEFVNWLEHDQTDDLRGVKYAVFGCGNRSWASTYQRIPRLIDS
VLEKKGAQRLHKLGEGDAGDDFEGQFESWKYDLWPLLRTEFSLAEPEPNQTETDRQAL
SVEFVNAPAASPLAKAYQVFTAKISANRELQCEKSGRSTRHIEISLPEGAAYQEGDHLGV
LPQNSEVLIGRVFQRFGLNGNEQILISGRNQASHLPLERPVHVKDLFQHCVELQEPATRA
QIRELAAHTVCPPHQRELEDLLKDDVYKDQVLNKRLTMLDLLEQYPACELPFARFLALL
PPLKPRYYSISSSPQLNPRQTSITVSVVSGPALSGRGHYKGVASNYLAGLEPGDAISCFIRE
PQSGFRLPEDPETPVIMVGPGTGIAPYRGFLQARRIQRDAGVKLGEAHLYFGCRRPNEDF
LYRDELEQAEKDGIVHLHTAFSRLEGRPKTYVQDLLREDAALLIHLLNEGGRLYVCGDG
SRMAPAVEQALCEAYRIVQGASREESQSWLSALLEEGRYAKDVWDGGVSQHNVKADC
IART.
OhyA1 from Stenotrophomonas maltophilia ATCC17679 (DNA)
SEQ ID NO: 61
ATGTACTACAGCAGTGGCAACTACGAAGCCTTTGCGCGCCCGCGCAAGCCCGCCGG
TGTCGATGACAAGCGCGCATGGTTCGTCGGTTCGGGCCTGGCCTCGCTGGCCGGCGC
CGCGTTCCTGGTGCGCGACGGCCGCATGGCCGGTGAGCGCATCACCATTCTCGAGCA
GCAGCGGATTGCCGGAGGCGCGCTGGATGGCCTGAAGGTGCCTGAAAAGGGCTTCG
TGATCCGCGGTGGACGCGAGATGGAAGACCACTTCGAGTGTCTGTGGGATCTGTTCC
GCTCGATTCCGTCGCTGGAGATTGAAGATGCCAGCGTGCTGGACGAGTTCTACTGGC
TGAACAAGGACGACCCCAACTATTCGCTGCAGCGTGCCACGATCAATCGCGGTGAG
GATGCGCACACCGACGGCCTGTTCACCCTGACCGAGCAGGCGCAGAAGGACATCAT
CGCGCTGTTCCTGGCCACCCGGCAGGAGATGGAGAACAAGCGCATCGACGAGGTGC
TGGGCCGCGACTTCCTGGACAGCAACTTCTGGCTGTACTGGCGCACCATGTTCGCCT
TCGAGGAATGGCATTCGGCGCTGGAGATGAAGCTGTACCTGCATCGCTTCATCCACC
ATATCGGCGGCCTGCCGGATTTCTCGGCGCTGAAGTTCACCAAGTACAACCAGTACG
AATCGCTGGTGCTGCCGCTGGTGAAGTGGCTGCAGGACCACGGCGTGGTGTTCCAGT
ACGGCACCGAGGTGACCGACGTCGACTTCGATCTGGCGGCCGGCCGCAAGCAGGCC
ACGCGCATCCACTGGACGCGTGACGGTGTAGCCGGTGGCGTGGATCTGAGCGCGGA
TGACCTTGTGTTCATGACGATCGGTTCGTTGACCGAGAACTCGGACAACGGCGACCA
CCGCACGGCTGCGCGTTTGAATGAAGGCGCGGCGCCGGCCTGGGACCTGTGGCGAC
GTATTGCCGCCAGGGATCCTGCGTTCGGGCGCCCGGATGTGTTTGGCGCGCATATCC
CGCAGACCAAGTGGGAATCGGCGACGGTGACCACGCTGGATGCACGCATTCCGGCC
TACATCCAGAAGATCGCCAAGCGCGACCCGTTCAGTGGCAAGGTGGTGACCGGCGG
CATCGTCAGTGTGCGTGACTCGCGCTGGTTGATGAGCTGGACGGTGAACCGCCAGCC
GCATTTCAAGAACCAGCCCAAGGATCAGATCGTGGTCTGGGTGTATTCGCTGTTCGT
GGATACGCCTGGAGACTACGTGAAGAAGCCGATGCAGGATTGCACCGGCGAGGAGA
TCACCCGTGAGTGGCTGTACCACCTGGGCGTGCCGGTGGAAGAAATCGACGAATTG
GCCGCGACCGGCGCGAAAACGGTGCCGGTGATGATGCCGTACATCACCGCGTTCTT
CATGCCACGCCAGGCCGGTGATCGCCCGGACGTGGTGCCGGAGGGTGCGGTGAACT
TCGCCTTCATCGGCCAGTTTGCCGAATCGAAGCAGCGCGACTGCATCTTCACCACCG
AGTATTCGGTGCGCACACCGATGGAAGCGGTGTATACCCTGCTGGGCATCGAGCGC
GGCGTGCCGGAGGTGTTCAATTCCACCTATGACGTGCGCTCGTTGCTGGCGGCGACC
GGTCGCCTGCGCGATGGCAGGGAACTGGATATTCCCGGCCCGGCGTTCCTGCGCAA
CCTGCTGATGAACAAGCTGGACAAGACCCAGATCGGTGGCCTCCTGCGCGAGTTCA
AGCTGGTGCAGGAGGACTGA
OhyA1 from Stenotrophomonas maltophilia ATCC17679 (Protein)
SEQ ID NO: 62
MYYSSGNYEAFARPRKPAGVDDKRAWFVGSGLASLAGAAFLVRDGRMAGERITILEQQ
RIAGGALDGLKVPEKGFVIRGGREMEDHFECLWDLFRSIPSLEIEDASVLDEFYWLNKD
DPNYSLQRATINRGEDAHTDGLFTLTEQAQKDIIALFLATRQEMENKRIDEVLGRDFLDS
NFWLYWRTMFAFEEWHSALEMKLYLHRFIHHIGGLPDFSALKFTKYNQYESLVLPLVK
WLQDHGVVFQYGTEVTDVDFDLAAGRKQATRIHWTRDGVAGGVDLSADDLVFMTIGS
LTENSDNGDHRTAARLNEGAAPAWDLWRRIAARDPAFGRPDVFGAHIPQTKWESATVT
TLDARIPAYIQKIAKRDPFSGKVVTGGIVSVRDSRWLMSWTVNRQPHFKNQPKDQIVV
WVYSLFVDTPGDYVKKPMQDCTGEEITREWLYHLGVPVEEIDELAATGAKTVPVMMP
YITAFFMPRQAGDRPDVVPEGAVNFAFIGQFAESKQRDCIFTTEYSVRTPMEAVYTLLGI
ERGVPEVFNSTYDVRSLLAATGRLRDGRELDIPGPAFLRNLLMNKLDKTQIGGLLREFK
LVQED.
OhyA2 from Stenotrophomonas maltophilia S208 (DNA)
SEQ ID NO: 63
ATGAGCCAGCCCACCGCACCGGGACGCAACGCAGGGGCCACGCCGGCCTTCGAGCA
CGAGCCGGACAGCACCGGCGGCTACTGGTCCAACCGGCCGGAGAACACACTGCCAC
CGCCGGACATGATGGGGGCCTACATGCGCAACCGGCCGCTGCCGCCGGAGGACGTG
GCGCAGCGCAAGGCCTACATCATCGGCACCGGCATCGCCGGGTTGGCGGCGGCGTT
CTACCTGATCCGCGACGGCGGCATGCCGCCGGCCAACATCACGCTGCTGGACAGCC
TGGAGATCGAAGGCGGTTCGCTGGATGGCGCGGGGGATGCCGAGCAGGGCTACCTG
ATCCGCGGCGGCCGCGAGATGAACTGGAACTACGACAATTTCTGGGACCTGTTCCA
GGATGTGCCGGCACTCGAACTGCCGGCCGGCTTCAGCGTGCTCGACGAGTACCGCG
CCGTCAACGACAACGATCCGAACTGGTCCAAGGCGCGGCTGCTGCACCAGCAGGGC
AAGGTCAAGGATTTCGCCACGTTCGGGTTGAGCCGCGGCCAGCAATGGGAGCTGGT
CAAGCTGCTGCTCAAGCGCAAGGAAGACCTGGACGACGTCACCATCGAGGACTACT
TCAGCGAAGGCTTTCTGCAGAGCAACTTCTGGTTCTTCTGGCGCTCGATGTTCGCCTT
CGAGAACTGGCAGAGCCTGCTCGAGATGAAGCTGTACATGCATCGGTTCCTGGATG
CCATCGACGGTCTGAACGACATGTCCGCGCTGGTGTTTCCCAAGTACAACCAATACG
AGAGTTTTGTGGTGCCGCTGTCGCGGATGCTGCGCGCGCAGGGCGTCAACGTGCAGT
TCGATACCCGCGTCCACGACCTGGAGATGGCGGTGGACGGGCAGTCACGCACCGTC
ACCGCGCTGCGCTGCCGGGTGGCCGGCAACGAGACCACGCTGCCGGTTGCGGCGGG
CGACCTGGTGTTCGCGCTCACCGGTTCGATGACCGAAGGCACGGCGTACGGCGACA
TGGACACCGTGCCGCCGCTGGCGCGCGACCGCCGGGACCCGGGCGAGGACAGTGAC
TGGGCGCTGTGGCGCAATCTCGCGCGGCAGTCGCCGATCTTCGGCAAGCCGGAGAA
GTTCTACGGCGACGTGGACCGCTCGATGTGGGAGTCGGCCACGTTGACCTGCCGCCC
CTCGCCGCTGGTGGACAAGATCCGCACGTTGTCGGTCAACGATCCGTACTCCGGGCG
CACCGTGACCGGTGGGGTCATCACCATCACCGATTCCAACTGGGTGCTCAGCTTCAC
CGTCAACCGCCAGCCGCATTTCGTGGACCAGCCCAAGGACGTGCTGGTGGTATGGG
TCTATGCCCTGTTGATGGATCAGGACGGCAACCACATCAAAAAGCCGATGCCGGCG
TGTACCGGACGCGAGGTGCTGGCCGAACTGTGCCACCACCTGGGCATCGGCGACCA
GATCGATGCGGTGGCCGCCGCGACCAGGGTGCGGCTGGCGTTGATGCCGTACATCA
CCGCGCAGTTCATGCCGCGTGCCGCTGGCGACCGTCCGCACGTGGTACCGGCCGGCT
GCACCAACCTGGGCCTGCTCGGCCAGTTCGTGGAAACGCGCAACGATGTGATCTTCA
CGATGGAAAGCTCGATCCGCACGGCGCGTGTGGCGGTGTACACCCTGCTGGGGCTG
CGCAAGCAGGTACCGGACCTGAGCCCGACCCAGTACGACATCCGCAATCTGATCAA
AGCGGCACGGGCGTTGAACAACAACGCGCCGTTCCCCGGCGAACGGCTGCTGCACC
GTCTGCTCGGCAACAGTTATTACGCCCACATCCTGCCGCCGCTGCCACAGCCTGAAA
AGGGCCGGGAGGCCTTCCTTGAAGAGGAGCTGTCGTGGTTGTCAGGCAAGGGCAGC
GTGGTGCTGAAGGACCTGTCTGCACGGCTGGATCGGCTTGGCGAAACGCTGGGCAG
GCGGCGCGCGCCCTGA
OhyA2 from Stenotrophomonas maltophilia S208 (Protein)
SEQ ID NO: 64
MSQPTAPGRNAGATPAFEHEPDSTGGYWSNRPENTLPPPDMMGAYMRNRPLPPEDVAQ
RKAYIIGTGIAGLAAAFYLIRDGGMPPANITLLDSLEIEGGSLDGAGDAEQGYLIRGGRE
MNWNYDNFWDLFQDVPALELPAGFSVLDEYRAVNDNDPNWSKARLLHQQGKVKDFA
TFGLSRGQQWELVKLLLKRKEDLDDVTIEDYFSEGFLQSNFWFFWRSMFAFENWQSLL
EMKLYMHRFLDAIDGLNDMSALVFPKYNQYESFVVPLSRMLRAQGVNVQFDTRVHDL
EMAVDGQSRTVTALRCRVAGNETTLPVAAGDLVFALTGSMTEGTAYGDMDTVPPLAR
DRRDPGEDSDWALWRNLARQSPIFGKPEKFYGDVDRSMWESATLTCRPSPLVDKIRTLS
VNDPYSGRTVTGGVITITDSNWVLSFTVNRQPHFVDQPKDVLVVWVYALLMDQDGNHI
KKPMPACTGREVLAELCHHLGIGDQIDAVAAATRVRLALMPYITAQFMPRAAGDRPHV
VPAGCTNLGLLGQFVETRNDVIFTMESSIRTARVAVYTLLGLRKQVPDLSPTQYDIRNLI
KAARALNNNAPFPGERLLHRLLGNSYYAHILPPLPQPEKGREAFLEEELSWLSGKGSVV
LKDLSARLDRLGETLGRRRAP.

1. A multifunctional molecule having a chemical formula according to 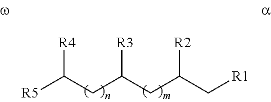
wherein
R1=-OH; —O₂H; —CH₂; —CH₂OH; —CHO; —CH₂NH₂; —CO₂H; —CO₂CH₃; —CO₂C₂H₅; —CO₂C₃H₇; —CO₂C₂H₃
R2=-H; —OH; —NH₂
R3=-H; —OH;
R4=-H; —OH;
R5=-CH₃; —CH₂; —CH₂OH; —CHO; —CH₂NH₂; —CO₂H; —CO₂CH₃; —CO₂C₂H₅; —CO₂C₃H₇; —CO₂C₂H₃;
wherein
m=1-10 and n=0-9 and
wherein
if R1 is OH or —O₂H then 3<m+n≤10; and if R1 is other than OH or —O₂H, then 2<m+n≤10, and
wherein
the multifunctional molecule has at least three functional groups comprising a heteroatom, and
wherein
at least two of R2, R3 and R4 are OH; or when R5 is other than CH₃, CH₂ or C₃H₇ CH₂H₅ then at least one of R2, R3 and R4 are OH; and
wherein
when R5=CH₃ and R4=H then n=0 or 1; and
wherein
when R2=H, then R4 does not=H; and
wherein
when R3=H, then n+m=p and p=3-10; unless R1=-OH or —O₂H in which case when R3=-H then n+m=p and p=4-10; and
wherein
when the multifunctional molecule comprises a double bond that is not terminal, the double bond is in a position corresponding to an omega7 (ω-7) position; and
wherein
the multifunctional molecule is not 1,3,12-dodecane triol; is not 3,11-dihydroxy-tetradecanoic acid; is not 3,11-dihydroxy-tetradecanoic acid methyl ester and is not naturally occurring.

2. The multifunctional fatty acid derivative molecule of claim 1, wherein the multifunctional fatty acid derivative molecule is a multifunctional alcohol.

3. The multifunctional fatty acid derivative molecule of claim 2,
wherein
R1=CH₂OH and R2=OH.

4. The multifunctional fatty acid derivative molecule of claim 3, wherein the multifunctional fatty acid derivative molecule is a member selected from the group consisting of 1,3,11-dodecane triol, 1,3,-10-dodecane triol, 1,3,9-dodecane triol, 1,3,12-dodecene triol, 1,3,11-dodecene triol, 1,3,10-dodecene triol, 1,3,9-dodecene triol, 1,3,11,12-dodecane tetrol, 1,3,10,12-dodecane tetrol, 1,3,9,12 dodecane tetrol, 1,3,7-decane triol, 1,3,8-decane triol, and 1,3,9-decane triol.

5. The multifunctional fatty acid derivative molecule of claim 1, wherein the multifunctional fatty acid derivative molecule is a multifunctional fatty acid ester.

6. The multifunctional fatty acid derivative molecule of claim 5, wherein
R1=CO₂CH₃ and R2=OH.

7. The multifunctional fatty acid derivative molecule of claim 6, wherein the multifunctional fatty acid derivative molecule is a member selected from the group consisting of 3,12-dihydroxy dodecanoic acid methyl ester, 3,14-dihydroxy tetradecanoic acid methyl ester, 3,16-dihydroxy hexadecanoic acid methyl ester, 3,12-dihydroxy dodecenoic acid methyl ester, 3,14-dihydroxy tetradecenoic acid methyl ester, 3,16-dihydroxy hexadecenoic acid methyl ester, 3,11-dihydroxy dodecanoic acid methyl ester, 3,10-dihydroxy dodecanoic acid methyl ester, 3,9-dihydroxy dodecanoic acid methyl ester, 3,11-dihydroxy dodecenoic acid methyl ester, 3,10-dihydroxy dodecenoic acid methyl ester, 3,9-dihydroxy dodecenoic acid methyl ester, 3,13-dihydroxy tetradecanoic acid methyl ester, 3,12-dihydroxy tetradecanoic acid methyl ester, 3,13-dihydroxy tetradecenoic acid methyl ester, 3,12-dihydroxy tetradecenoic acid methyl ester, 3,11-dihydroxy tetradecenoic acid methyl ester, 3,15-dihydroxy hexadecanoic acid methyl ester, 3,14-dihydroxy hexadecanoic acid methyl ester, 3,13-dihydroxy hexadecanoic acid methyl ester, 3,15-dihydroxy hexadecenoic acid methyl ester, 3,14-dihydroxy hexadecenoic acid methyl ester, and 3,13-dihydroxy hexadecenoic acid methyl ester.

8. The multifunctional fatty acid derivative molecule of claim 5, wherein
R1=CO₂CH₂CH₃ and R2=OH.

9. The multifunctional fatty acid derivative molecule of claim 8, wherein the multifunctional fatty acid derivative molecule is a member selected from the group consisting of 3,12-dihydroxy dodecanoic acid ethyl ester, 3,14-dihydroxy tetradecanoic acid ethyl ester, 3,16-dihydroxy hexadecanoic acid ethyl ester, 3,12-dihydroxy dodecenoic acid ethyl ester, 3,14-dihydroxy tetradecenoic acid ethyl ester, 3,16-dihydroxy hexadecenoic acid ethyl ester, 3,11-dihydroxy dodecanoic acid ethyl ester, 3,10-dihydroxy dodecanoic acid ethyl ester, 3,9-dihydroxy dodecanoic acid ethyl ester, 3,11-dihydroxy dodecenoic acid ethyl ester, 3,10-dihydroxy dodecenoic acid ethyl ester, 3,9-dihydroxy dodecenoic acid ethyl ester, 3,13-dihydroxy tetradecanoic acid ethyl ester, 3,12-dihydroxy tetradecanoic acid ethyl ester, 3,11-dihydroxy tetradecanoic acid ethyl ester, 3,13-dihydroxy tetradecenoic acid ethyl ester, 3,12-dihydroxy tetradecenoic acid ethyl ester, 3,11-dihydroxy tetradecenoic acid ethyl ester, 3,15-dihydroxy hexadecanoic acid ethyl ester, 3,14-dihydroxy hexadecanoic acid ethyl ester, 3,13-dihydroxy hexadecanoic acid ethyl ester, 3,15-dihydroxy hexadecenoic acid ethyl ester, 3,14-dihydroxy hexadecenoic acid ethyl ester, and 3,13-dihydroxy hexadecenoic acid ethyl ester.

10. The multifunctional fatty acid derivative molecule of claim 1, wherein the multifunctional fatty acid derivative molecule is a multifunctional fatty acid.

11. The multifunctional fatty acid derivative molecule of claim 10, wherein n≠4.

12. The multifunctional fatty acid derivative molecule of claim 11, wherein
R2=H.

13.-36. (canceled)

37. A method for making a multifunctional fatty acid derivative molecule having a chemical formula according to: 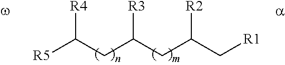
wherein
R1=-OH; —O₂H; —CH₂; —CH₂OH; —CHO; —CH₂NH₂; —CO₂H; —CO₂CH₃; —CO₂C₂H₅; —CO₂C₃H₇; —CO₂C₂H₃
R2=-H; —OH; —NH₂
R3=-H; —OH;
R4=-H; —OH;
R5=-CH₃; —CH₂; —CH₂OH; —CHO; —CH₂NH₂; —CO₂H; —CO₂CH₃; —CO₂C₂H₅; —CO₂C₃H₇; —CO₂C₂H₃;
wherein
m=1-10 and n=0-9 and
wherein
if R1 is OH or —O₂H then 3<m+n≤10; and if R1 is other than OH or —O₂H, then 2<m+n≤10, and
wherein
the multifunctional molecule has at least three functional groups comprising a heteroatom, and
wherein
at least two of R2, R3 and R4 are OH; or when R5 is other than CH₃, CH₂ or C₃H₇ CH₂H₅ then at least one of R2, R3 and R4 are OH; and
wherein
when R5=CH₃ and R4=H then n=0 or 1; and
wherein
when R2=H, then R4 does not=H; and
wherein
when R3=H, then n+m=p and p=3-10; unless R1=-OH or —O₂H in which case when R3=-H then n+m=p and p=4-10; and
wherein
when the multifunctional molecule comprises a double bond that is not terminal, the double bond is in a position corresponding to an omega7 (ω-7) position; and
wherein
the multifunctional molecule is not 1,3,12-dodecane triol; is not 3,11-dihydroxy-tetradecanoic acid; is not 3,11-dihydroxy-tetradecanoic acid methyl ester and is not naturally occurring,
the method comprising:
culturing
a recombinant microbe that comprises a heterologous enzyme pathway capable of producing a bifunctional fatty acid derivative molecule, and at least one heterologous hydroxylating enzyme,
in a culture medium comprising a simple carbon source,
wherein
the heterologous enzyme pathway capable of producing bifunctional fatty acid derivative molecule is selected from:
(i) a heterologous enzyme pathway capable of producing a 3-hydroxy fatty acid;
(ii) a heterologous enzyme pathway capable of producing a 3-hydroxy fatty ester;
(iii) a heterologous enzyme pathway capable of producing a 1,3-fatty diol
(iv) a heterologous enzyme pathway capable of producing a hydroxy fatty acid;
(v) a heterologous enzyme pathway capable of producing a hydroxy fatty ester; and
(vi) a heterologous enzyme pathway capable of producing a fatty diol; and
wherein
the at least one heterologous hydroxylating enzyme is selected from a heterologous hydroxylase enzyme and a heterologous hydratase enzyme or a combination thereof.

38. The method of claim 37,
wherein
the heterologous hydroxylase enzyme is a member selected from the group consisting of omega-hydroxylases (o-hydroxylases), mid-chain hydroxylases, and subterminal hydroxylases.

39. The method of claim 37,
wherein
the heterologous enzyme pathway capable of producing bifunctional fatty acid derivative molecule is the heterologous enzyme pathway capable of producing a 1,3-fatty diol.

40. The method of claim 39,
wherein
the heterologous enzyme pathway capable of producing a 1,3-fatty diol comprises;
(i) a heterologous thioesterase and
(ii) a heterologous carboxylic acid reductase.

41. The method of claim 40,
wherein
the heterologous enzyme pathway capable of producing a 1,3-fatty diol further comprises;
(iii) a heterologous alcohol dehydrogenase.

42. The method of claim 41,
wherein
the heterologous enzyme pathway capable of producing a 1,3-fatty diol further comprises:
(i) a heterologous PhaG thioesterase from Pseudomonas putida,
(ii) a heterologous CarB carboxylic acid reductase from Mycobacterium smegmatis, and
(iii) a heterologous AlrA alcohol dehydrogenase from Acinetobacter baylyi.

43. The method of claim 40,
wherein
the heterologous hydroxylase enzyme is a cyp102A subterminal-hydroxylase from Bacillus licheniformis, and
wherein
the method produces multifunctional molecules selected from the group consisting of 1,3,9 decanetriol, 1,3,8 decanetriol, 1,3,7 decanetriol, 1,3,11 dodecanetriol, 1,3,10 dodecanetriol, 1,3,9 dodecanetriol, (z5)1,3,11 dodecenetriol, (z5)1,3,10 dodecenetriol and (z5)1,3,9 dodecenetriol.

44.-60. (canceled)


SEQUENCE LISTING (64 sequences)

SEQ ID NO: 1 - DNA, 1413 bp, Marinobacter aquaeolei
atgccaacactgcccagaacatttgacgacattcagtcccgactgattaacgccacctccagggtggtgccgatgcagaggcaaattcagggactgaaattcttaatgagcgccaagaggaagaccttcggcccacgccgaccgatgcccgaattcgttgaaacacccatcccggacgttaacacgctggcccttgaggacatcgatgtcagcaatccgtttttataccggcagggtcagtggcgcgcctatttcaaacggttgcgtgatgaggcgccggtccattaccagaagaacagccctttcggccccttctggtcggtaactcggtttgaagacatcctgttcgtggataagagtcacgacctgttttccgccgagccgcaaatcattctcggtgaccctccggaggggctgtcggtggaaatgttcatagcgatggatccgccgaaacacgatgtgcagcgcagctcggtgcagggagtagtggcaccgaaaaacctgaaggagatggaggggctgatccgatcacgcaccggcgatgtgcttgacagcctgcctacagacaaaccctttaactgggtacctgctgtttccaaggaactcacaggccgcatgctggcgacgcttctggattttccttacgaggaacgccacaagctggttgagtggtcggacagaatggcaggtgcagcatcggccaccggcggggagtttgccgatgaaaatgccatgtttgacgacgcggcagacatggcccggtctttctccaggctttggcgggacaaggaggcgcgccgcgcagcaggcgaggagcccggtttcgatttgatcagcctgttgcagagcaacaaagaaacgaaagacctgatcaatcggccgatggagtttatcggtaatttgacgctgctcatagtcgccggcaacgatacgacgcgcaactcgatgagtggtggcctggtggccatgaacgaattccccagggaatttgaaaaattgaaggcaaaaccggagttgattccgaacatggtgtcggaaatcatccgctggcaaacgccgctggcctatatgcgccgaatcgccaagcaggatgtcgaactgggcggccagaccatcaagaagggtgatcgagttgtcatgtggtacgcgtcgggtaaccgggacgagcgcaaatttgacaaccccgatcagttcatcattgatcgcaaggacgcacgaaaccacatgtcgttcggctatggggttcaccgttgcatgggcaaccgtctggctgaactgcaactgcgcatcctctgggaagaaatactcaagcgttttgacaacatcgaagtcgtcgaagagcccgagcgggtgcagtccaacttcgtgcggggctattccaggttgatggtcaaactgacaccgaacagttaa

SEQ ID NO: 2 - protein, 470 aa, Marinobacter aquaeolei
MPTLPRTFDDIQSRLINATSRVVPMQRQIQGLKFLMSAKRKTFGPRRPMPEFVETPIPDVNTLALEDIDVSNPFLYRQGQWRAYFKRLRDEAPVHYQKNSPFGPFWSVTRFEDILFVDKSHDLFSAEPQIILGDPPEGLSVEMFIAMDPPKHDVQRSSVQGVVAPKNLKEMEGLIRSRTGDVLDSLPTDKPFNWVPAVSKELTGRMLATLLDFPYEERHKLVEWSDRMAGAASATGGEFADENAMFDDAADMARSFSRLWRDKEARRAAGEEPGFDLISLLQSNKETKDLINRPMEFIGNLTLLIVAGNDTTRNSMSGGLVAMNEFPREFEKLKAKPELIPNMVSEIIRWQTPLAYMRRIAKQDVELGGQTIKKGDRVVMWYASGNRDERKFDNPDQFIIDRKDARNHMSFGYGVHRCMGNRLAELQLRILWEEILKRFDNIEVVEEPERVQSNFVRGYSRLMVKLTPNS

SEQ ID NO: 3 - DNA, 2400 bp, Artificial Sequence
  Description of Artificial Sequence Synthetic polynucleotide
atgccaacactgcccagaacatttgacgacattcagtcccgactgattaacgccacctccagggtggtgccgatgcagaggcaaattcagggactgaaattcttaatgagcgccaagaggaagaccttcggcccacgccgaccgatgcccgaattcgttgaaacacccatcccggacgttaacacgctggcccttgaggacatcgatgtcagcaatccgtttttataccggcagggtcagtggcgcgcctatttcaaacggttgcgtgatgaggcgccggtccattaccagaagaacagccctttcggccccttctggtcggtaactcggtttgaagacatcctgttcgtggataagagtcacgacctgttttccgccgagccgcaaatcattctcggtgaccctccggaggggctgtcggtggaaatgttcatagcgatggatccgccgaaacacgatgtgcagcgcagctcggtgcagggagtagtggcaccgaaaaacctgaaggagatggaggggctgatccgatcacgcaccggcgatgtgcttgacagcctgcctacagacaaaccctttaactgggtacctgctgtttccaaggaactcacaggccgcatgctggcgacgcttctggattttccttacgaggaacgccacaagctggttgagtggtcggacagaatggcaggtgcagcatcggccaccggcggggagtttgccgatgaaaatgccatgtttgacgacgcggcagacatggcccggtctttctccaggctttggcgggacaaggaggcgcgccgcgcagcaggcgaggagcccggtttcgatttgatcagcctgttgcagagcaacaaagaaacgaaagacctgatcaatcggccgatggagtttatcggtaatttgacgctgctcatagtcgccggcaacgatacgacgcgcaactcgatgagtggtggcctggtggccatgaacgaattccccagggaatttgaaaaattgaaggcaaaaccggagttgattccgaacatggtgtcggaaatcatccgctggcaaacgccgctggcctatatgcgccgaatcgccaagcaggatgtcgaactgggcggccagaccatcaagaagggtgatcgagttgtcatgtggtacgcgtcgggtaaccgggacgagcgcaaatttgacaaccccgatcagttcatcattgatcgcaaggacgcacgaaaccacatgtcgttcggctatggggttcaccgttgcatgggcaaccgtctggctgaactgcaactgcgcatcctctgggaagaaatactcaagcgttttgacaacatcgaagtcgtcgaagagcccgagcgggtgcagtccaacttcgtgcggggctattccaggttgatggtcaaactgacaccgaacagtgtactccatcgtcatcaacctgtcaccatcggcgagccggccgctcgtgctgtgagccgcacggtgaccgttgagcgtcttgatcgcattgccgacgatgtccttcgcctggtccttcgcgatgctggaggtaaaaccctcccgacgtggacgcctggcgctcacatcgacctggatctgggtgctctgagccgtcagtattcgctctgcggcgctccggatgctccgtcgtacgaaatcgccgtgcacttagatccggaaagccgtggtggaagccgctatattcatgaacagctggaagttggaagtccgctgcgtatgcgtggcccacgcaaccatttcgccctggatccgggtgcggaacattacgtgtttgttgccgggggtatcggcatcacgccggtgctggcaatggcggatcatgcccgtgcgcgtggttggtcgtacgaactgcattattgtggtcgtaatcgtagcggtatggcttacctggaacgcgtcgcgggacatggtgaccgcgctgccttgcacgtatctgaagaaggcacccgcattgatctggcggcattacttgctgaaccggcgccgggcgtgcaaatctacgcctgcggtccgggccgtttattagcgggtcttgaagacgcgtctcgtaattggccggatggcgcgcttcatgtggagcatttcacttcgagtttagccgctttggatccggatgtcgaacatgcctttgatttggagctgcgtgactctggccttaccgttcgcgtcgagccaactcagaccgttttagacgctttgcgtgcgaacaatatcgacgtcccgtcggattgcgaagaggggctgtgtggttcttgcgaagtagccgttctggatggcgaggttgatcaccgtgataccgttctgactaaggccgagcgcgccgcgaatcgtcagatgatgacttgctgcagtcgtgcatgcggtgatcgtctggcgctgcgcctctaa

SEQ ID NO: 4 - protein, 799 aa, Artificial Sequence
  Description of Artificial Sequence Synthetic polypeptide
MPTLPRTFDDIQSRLINATSRVVPMQRQIQGLKFLMSAKRKTFGPRRPMPEFVETPIPDVNTLALEDIDVSNPFLYRQGQWRAYFKRLRDEAPVHYQKNSPFGPFWSVTRFEDILFVDKSHDLFSAEPQIILGDPPEGLSVEMFIAMDPPKHDVQRSSVQGVVAPKNLKEMEGLIRSRTGDVLDSLPTDKPFNWVPAVSKELTGRMLATLLDFPYEERHKLVEWSDRMAGAASATGGEFADENAMFDDAADMARSFSRLWRDKEARRAAGEEPGFDLISLLQSNKETKDLINRPMEFIGNLTLLIVAGNDTTRNSMSGGLVAMNEFPREFEKLKAKPELIPNMVSEIIRWQTPLAYMRRIAKQDVELGGQTIKKGDRVVMWYASGNRDERKFDNPDQFIIDRKDARNHMSFGYGVHRCMGNRLAELQLRILWEEILKRFDNIEVVEEPERVQSNFVRGYSRLMVKLTPNSVLHRHQPVTIGEPAARAVSRTVTVERLDRIADDVLRLVLRDAGGKTLPTWTPGAHIDLDLGALSRQYSLCGAPDAPSYEIAVHLDPESRGGSRYIHEQLEVGSPLRMRGPRNHFALDPGAEHYVFVAGGIGITPVLAMADHARARGWSYELHYCGRNRSGMAYLERVAGHGDRAALHVSEEGTRIDLAALLAEPAPGVQIYACGPGRLLAGLEDASRNWPDGALHVEHFTSSLAALDPDVEHAFDLELRDSGLTVRVEPTQTVLDALRANNIDVPSDCEEGLCGSCEVAVLDGEVDHRDTVLTKAERAANRQMMTCCSRACGDRLALRL

SEQ ID NO: 5 - DNA, 2400 bp, Artificial Sequence
  Description of Artificial Sequence Synthetic polynucleotide
atgccaacactgcccagaacatttgacgacattcagtcccgactgattaacgccacctccagggtggtgccgatgcagaggcaaattcagggactgaaattcttaatgagcgccaagaggaagaccttcggcccacgccgaccgatgcccgaattcgttgaaacacccatcccggacgttaacacgctggcccttgaggacatcgatgtcagcaatccgtttttataccggcagggtcagtggcgcgcctatttcaaacggttgcgtgatgaggcgccggtccattaccagaagaacagccctttcggccccttctggtcggtaactcggtttgaagacatcctgttcgtggataagagtcacgacctgttttccgccgagccgcaaatcattctcggtgaccctccggaggggctgtcggtggaaatgttcatagcgatggatccgccgaaacacgatgtgcagcgcagctcggtgcagggagtagtggcaccgaaaaacctgaaggagatggaggggctgatccgatcacgcaccggcgatgtgcttgacagcctgcctacagacaaaccctttaactgggtacctgctgtttccaaggaactcacaggccgcatgctggcgacgcttctggattttccttacgaggaacgccacaagctggttgagtggtcggacagaatggcaggtgcagcatcggccaccggcggggagtttgccgatgaaaatgccatgtttgacgacgcggcagacatggcccggtctttctccaggctttggcgggacaaggaggcgcgccgcgcagcaggcgaggagcccggtttcgatttgatcagcctgttgcagagcaacaaagaaacgaaagacctgatcaatcggccgatggagtttatcggtaatttgacgctgctcatagtcgccggcaacgatacgacgcgcaactcgatgagtggtggcctggtggccatgaacgaattccccagggaatttgaaaaattgaaggcaaaaccggagttgattccgaacatggtgtcggaaatcatccgctggcaaacgccgctggcctatatgcgccgaatcgccaagcaggatgtcgaactgggcggccagaccatcaagaagggtgatcgagttgtcatgtggtacgcgtcgggtaaccgggacgagcgcaaatttgacaaccccgatcagttcatcattgatcgcaaggacgcacgaaaccacatgtcgttcggctatggggttcaccgttgcatgggcaaccgtctggctgaactgcaactgcgcatcctctgggaagaaatactcaagcgttttgacaacatcgaagtcgtcgaagagcccgagcgggtgcagtccaacttcgtgcggggctattccaggttgatggtcaaactgacaccgaacagtgtactccatcgtcatcaacctgtcaccatcggcgagccggccgctcgtgctgtgagccgcacggtgaccgttgagcgtcttgatcgcattgccgacgatgtccttcgcctggtccttcgcgatgctggaggtaaaaccctcccgacgtggacgcctggcgctcacatcgacctggatctgggtgctctgagccgtcagtattcgctctgcggcgctccggatgctccgtcgtacgaaatcgccgtgcacttagatccggaaagccgtggtggaagccgctatattcatgaacagctggaagttggaagtccgctgcgtatgcgtggcccacgcaaccatttcgccctggatccgggtgcggaacattacgtgtttgttgccgggggtatcggcatcacgccggtgctggcaatggcggatcatgcccgtgcgcgtggttggtcgtacgaactgcattattgtggtcgtaatcgtagcggtatggcttacctggaacgcgtcgcgggacatggtgaccgcgctgccttgcacgtatctgaagaaggcacccgcattgatctggcggcattacttgctgaaccggcgccgggcgtgcaaatctacgcctgcggtccgggccgtttattagcgggtcttgaagacgcgtctcgtaattggccggatggcgcgcttcatgtggagcatttcacttcgagtttagccgctttggatccggatgtcgaacatgcctttgatttggagctgcgtgactctggccttaccgttcgcgtcgagccaactcagaccgttttagacgctttgcgtgcgaacaatatcgacgtcccgtcggattgcgaagaggggctgtgtggttcttgcgaagtagccgttctggatggcgaggttgatcaccgtgataccgttctgactaaggccgagcgcgccgcgaatcgtcagatgatgacttgctgcagtcgtgcatgcggtgatcgtctggtcctgcgcctctaa

SEQ ID NO: 6 - protein, 799 aa, Artificial Sequence [sequence omitted]

SEQ ID NO: 7 - DNA, 3180 bp, Artificial Sequence
  Description of Artificial Sequence Synthetic polynucleotide
atgccaacactgcccagaacatttgacgacattcagtcccgactgattaacgccacctccagggtggtgccgatgcagaggcaaattcagggactgaaattcttaatgagcgccaagaggaagaccttcggcccacgccgaccgatgcccgaattcgttgaaacacccatcccggacgttaacacgctggcccttgaggacatcgatgtcagcaatccgtttttataccggcagggtcagtggcgcgcctatttcaaacggttgcgtgatgaggcgccggtccattaccagaagaacagccctttcggccccttctggtcggtaactcggtttgaagacatcctgttcgtggataagagtcacgacctgttttccgccgagccgcaaatcattctcggtgaccctccggaggggctgtcggtggaaatgttcatagcgatggatccgccgaaacacgatgtgcagcgcagctcggtgcagggagtagtggcaccgaaaaacctgaaggagatggaggggctgatccgatcacgcaccggcgatgtgcttgacagcctgcctacagacaaaccctttaactgggtacctgctgtttccaaggaactcacaggccgcatgctggcgacgcttctggattttccttacgaggaacgccacaagctggttgagtggtcggacagaatggcaggtgcagcatcggccaccggcggggagtttgccgatgaaaatgccatgtttgacgacgcggcagacatggcccggtctttctccaggctttggcgggacaaggaggcgcgccgcgcagcaggcgaggagcccggtttcgatttgatcagcctgttgcagagcaacaaagaaacgaaagacctgatcaatcggccgatggagtttatcggtaatttgacgctgctcatagtcgccggcaacgatacgacgcgcaactcgatgagtggtggcctggtggccatgaacgaattccccagggaatttgaaaaattgaaggcaaaaccggagttgattccgaacatggtgtcggaaatcatccgctggcaaacgccgctggcctatatgcgccgaatcgccaagcaggatgtcgaactgggcggccagaccatcaagaagggtgatcgagttgtcatgtggtacgcgtcgggtaaccgggacgagcgcaaatttgacaaccccgatcagttcatcattgatcgcaaggacgcacgaaaccacatgtcgttcggctatggggttcaccgttgcatgggcaaccgtctggctgaactgcaactgcgcatcctctgggaagaaatactcaagcgttttgacaacatcgaagtcgtcgaagagcccgagcgggtgcagtccaacttcgtgcggggctattccaggttgatggtcaaactgacaccgaacagttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaa

SEQ ID NO: 8 - protein, 1059 aa, Artificial Sequence
  Description of Artificial Sequence Synthetic polypeptide
MPTLPRTFDDIQSRLINATSRVVPMQRQIQGLKFLMSAKRKTFGPRRPMPEFVETPIPDVNTLALEDIDVSNPFLYRQGQWRAYFKRLRDEAPVHYQKNSPFGPFWSVTRFEDILFVDKSHDLFSAEPQIILGDPPEGLSVEMFIAMDPPKHDVQRSSVQGVVAPKNLKEMEGLIRSRTGDVLDSLPTDKPFNWVPAVSKELTGRMLATLLDFPYEERHKLVEWSDRMAGAASATGGEFADENAMFDDAADMARSFSRLWRDKEARRAAGEEPGFDLISLLQSNKETKDLINRPMEFIGNLTLLIVAGNDTTRNSMSGGLVAMNEFPREFEKLKAKPELIPNMVSEIIRWQTPLAYMRRIAKQDVELGGQTIKKGDRVVMWYASGNRDERKFDNPDQFIIDRKDARNHMSFGYGVHRCMGNRLAELQLRILWEEILKRFDNIEVVEEPERVQSNFVRGYSRLMVKLTPNSSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 9 - DNA, 1254 bp, Marine gamma proteobacterium HTCC2143
atgggaagcttggagcgtatctccatgttggattacgatcctgcaacgatgccattagaggatattgatgtatctgacgtagacttatggatcaacgacgccaagtgggattatttgacgcgcttgcgtaatgacgctcccgtccattactgtaagagttccgaattcggcccttactggtccatcacgcgcttcgatgacatcatgaaagtagagaagaattgggaggttttctcctctttccctagtatcacaattagtgatcctgctgaagattcggacttcactgcgccaaccttcattgccatggacccaccaaagcacgacgaccagcgtcgcgctgtgcaaaacgtagtggcacctccaaaccttaaagagttagaatcgactattcgttcacgcgcagtcaacatcctggattcacttcctatcggagagacttttaattgggttgatcgcgtttcaattgaactgactacacagatgttagctaccttgtttgattttccttttgaggaccgttataagctgacgcattggagcgatgtcacgtttgctcgcctgggagcgggtctgatcgaaacagaggaggagcgtcagcaagaactgattgagtgtttgaactatttcttacaactttggaaggaacgtgaagagaacccggtgggaaacgaccttatcagtatgatgattcgtggagatgcgactaaaaacttaagtcctcaagagtaccttgggaacatcttacttcttattgtaggagggaatgacacaacccgtaatagcattacaggaggactgttggctctgaacgaaaatcccgctgaatatgagaaactgcgttcccatcctgaacacatcccgaacatggtatcggaaatcatccgctgggtaacgcctttggctcacatgcgtcgtacagctactcgtgattatgagttcgaagggcaacagattaagaagggggataaaattatcatgtggtatgcaagtggcaaccgtgatgagcgcacaatccccaatcccaacaagtttttgatcgatcgcgagcgcgcccgtaaccacatcagtttcggcttcggtttgcaccgctgcatggggaatcgtttagctgaaatgcaactgcgtgtcttgtgggaggaaatccaaaaacgctttaagacaattgaggttgtcggagcaccagaacgcattccttcgtgttttgtccacgggatttcttccctgccagtgcgtttacactcttattactaa

SEQ ID NO: 10 - protein, 417 aa, Marine gamma proteobacterium HTCC2143
MGSLERISMLDYDPATMPLEDIDVSDVDLWINDAKWDYLTRLRNDAPVHYCKSSEFGPYWSITRFDDIMKVEKNWEVFSSFPSITISDPAEDSDFTAPTFIAMDPPKHDDQRRAVQNVVAPPNLKELESTIRSRAVNILDSLPIGETFNWVDRVSIELTTQMLATLFDFPFEDRYKLTHWSDVTFARLGAGLIETEEERQQELIECLNYFLQLWKEREENPVGNDLISMMIRGDATKNLSPQEYLGNILLLIVGGNDTTRNSITGGLLALNENPAEYEKLRSHPEHIPNMVSEIIRWVTPLAHMRRTATRDYEFEGQQIKKGDKIIMWYASGNRDERTIPNPNKFLIDRERARNHISFGFGLHRCMGNRLAEMQLRVLWEEIQKRFKTIEVVGAPERIPSCFVHGISSLPVRLHSYY

SEQ ID NO: 11 - DNA, 1281 bp, Marine gamma proteobacterium HTCC2080
atgaaccaggcagtaacgcgtaaagatggtttgccagaccccctgtccactcctctggaccaattggacattgctgacccacgtcgtttcgaattcgacacctggcagccattgtttgagcgtctgcgctccgaggctccggtccactaccaagcacagggacctgcaggtcccgctccccacggcgacttctggtctgtaactcgtttcgaagatattgtcgaggttgaaaaaaattgggaggcgtttagttccgagccaagcatcgccatccttgacccggaaccagacatgtccgtacagatgttcatcgcaacagatccaccccttcatgatgaccaacgccgtgcggtacaaggtgcggtcgcaccaaaaaatttacaggaatttgaggccctgattcgtcagcgcacgcaggaaactctggatgggctgccacttggggagactttcaattgggtcgaccgtgtaagcatcgatttgactaccaaaatgcttgcgacattatttgatttcccccaggagcagcgccaattattgactcactggtccgacgccgcgacttcagatgcgcgcatcaccgcagggcgcgggctgtcgattgatgaacgtcgtaccatcatgggggagatgctgcaaagctttacccagctgtggcacgaccacaaagcgaatgaccgtgagtccttcgatttaatccgtatgttacagcgtgatccaaagacccaaaatatggtagacgagccattttcttacctgggtaaccttatgttacttatcgttggggggaacgacacaactcgtaattccatgacgggaggtgtattgcatttgcatcaaaaccccagcgaaatggcgaaactgaaggccaatcacgggttgattccctcgatggtttcggaaatcatccgttatcagacgccattgccacacatgcgccgtacagccacacgcgatgtcgaattaaacggaaccgttatcccgaaaggcggtcgtgtggtattgtggtatgttagcggaaatcgtgactctaatgcaattcccgaggcagacaagttcatcatcgatcgcccgaatgtacgtaaccatttgtcattcgggatgggtattcatcgctgcatggggaatcgtttggctgaaatgcaattacgcattgtttgggaagaaatccttgagcgttttgagcgcattgaagttgtaggaccagcagttcgcacttgtaattcctttgttcgtggctatcacgaacttccggttcgtcttcaccctaaggtttactaa

SEQ ID NO: 12 - protein, 426 aa, Marine gamma proteobacterium HTCC2080
MNQAVTRKDGLPDPLSTPLDQLDIADPRRFEFDTWQPLFERLRSEAPVHYQAQGPAGPAPHGDFWSVTRFEDIVEVEKNWEAFSSEPSIAILDPEPDMSVQMFIATDPPLHDDQRRAVQGAVAPKNLQEFEALIRQRTQETLDGLPLGETFNWVDRVSIDLTTKMLATLFDFPQEQRQLLTHWSDAATSDARITAGRGLSIDERRTIMGEMLQSFTQLWHDHKANDRESFDLIRMLQRDPKTQNMVDEPFSYLGNLMLLIVGGNDTTRNSMTGGVLHLHQNPSEMAKLKANHGLIPSMVSEIIRYQTPLPHMRRTATRDVELNGTVIPKGGRVVLWYVSGNRDSNAIPEADKFIIDRPNVRNHLSFGMGIHRCMGNRLAEMQLRIVWEEILERFERIEVVGPAVRTCNSFVRGYHELPVRLHPKVY

SEQ ID NO: 13 - DNA, 1248 bp, Congregibacter litoralis
atgaacgtcgctcaagatctgcctcatccatcggatttagctttggaagacatcgacgtaagcgattcgcgcatctaccaacaggatgcatggcgcccctactttgaacgtctgcgcaaggaggaccctgttcactacgtagcagactcacagttcgggcctttctggtccatcacccgctgggaggatattgtagcggtggattccaacttcgaggatttttctagtgaaccggccatcgtcattggagacaacagcgaagagctgcccattgccaatttcattagcatggaccctcccaagcatgacgttcaacgccgcgccgtccagggcgtagtggccccaaaaaacttggcggagatggaggcgctgatccgctcccgcgtggtagaaattttggatggcttacctgtgggagagacattcaactgggtcgaccgcgtctcgatcaatcttacaacgcaaatgctggctacacttttcgactttccattcgaagaacgttttaaactgacctactggagcgatttgggcgccggcagccctgaaattgcgggtggcgatgtcgatcctgaggaacgtttagctggtttacatgactgcttagaaacattcactcgcatctggcacgaacgcaaagacgacggtgtcgagactttggaccttattcgcatgttgcaacgtgacccgaatacgaaggacatggtcgatgatccaatggaatatcttggaaatctgctgcttctgatcattggtggaaatgacacgacccgtaatagcgctagcgctggcgttctggcgttgaatcaaaaccctgccgaatatgaaaaattgcgtgctaaccccgcacttatcccgagtatggtatcagaggtgattcgttggcagacgcccttaatgcacatgcgtcgcactgctaacaaggatgttgaatttcagggcaagcagattcgcaaaggtgacaaggtcgttatgtggtatcttagtggcaaccatgatgaaacagctatcgaaaaccccaattccttcatcattgatcgtgaaaatcctcgtaaacatatgtcttttgggttcggggttcaccgctgcatgggtaaccgccttgccgagatgcaacttcgtgttctttgggaggaaattatgcaacgtttttcaaaggtagaagtcgtaggggaaccagagcgtgttaagtccaactttgtgcgtgggattagcaatttgccagtccgtgtgcaccctctttactaa

SEQ ID NO: 14 - protein, 415 aa, Congregibacter litoralis
MNVAQDLPHPSDLALEDIDVSDSRIYQQDAWRPYFERLRKEDPVHYVADSQFGPFWSITRWEDIVAVDSNFEDFSSEPAIVIGDNSEELPIANFISMDPPKHDVQRRAVQGVVAPKNLAEMEALIRSRVVEILDGLPVGETFNWVDRVSINLTTQMLATLFDFPFEERFKLTYWSDLGAGSPEIAGGDVDPEERLAGLHDCLETFTRIWHERKDDGVETLDLIRMLQRDPNTKDMVDDPMEYLGNLLLLIIGGNDTTRNSASAGVLALNQNPAEYEKLRANPALIPSMVSEVIRWQTPLMHMRRTANKDVEFQGKQIRKGDKVVMWYLSGNHDETAIENPNSFIIDRENPRKHMSFGFGVHRCMGNRLAEMQLRVLWEEIMQRFSKVEVVGEPERVKSNFVRGISNLPVRVHPLY

SEQ ID NO: 15 - DNA, 1416 bp, Limnobacter sp.
atgtcaacgcaatcaaagacattcgaccagattcaaacacgcgtaatcaatgcgacagcaaaggttattccaatgcacttgcagattcaaggtttaaaattccttatgcgcgcgaagaaaaaaaccattggggcccgccgcccaactccctcctttgtggagtacccactgccagatgtcgggactttacgcattgacgatattgatgtctcaaaccctttcttataccgccagggtcaatggcgcgcatatttcaagcgccttcgtgatgaagcacccgttcatttccaaaagaattcacccttcggtccgttctggagcgttacccgttatgaggatatccttttcgtagacaaacatcacgatttattttcggcagagcccgttatcatcttaggagacccaccggagggtctttcggtagagatgtttatcgcaatggatcctcctaagcacgatgctcaacgtagttcagtacaaggggtggtggccccccaaaacttaaaggagatggagagcctgattcgttcacgcacgggagatgtgttagattcccttcccgtcggccagccttttgactgggttcccacagtttcgaaggagctgactggacgcatgcttgcgactctgttagattttccgtacgacgagcgtgacaagctgatttattggtcggatcttctggctggtgcggcgtccgccaccgggggagagtttactgatgaggaagcgatgtttgatgcagcggcagatatggcacgcgactttagccgtttatggcgcgacaaacaggcgcgccgcgcagctggggaagctcccggcttcgatttaattagcctgcttcagagcagcgaagatacgcgtgacctgatcaatcgcccgatggagtttatcggcaacttggcattgttaattgttgggggaaacgatacaacacgtaactccatgagtggtggccttctggcaatgaatcaatttcctaaggagtttaaaaagctgaaggcaaacccagcattgatccctaatatggtgtcggaaattattcgctggcagacacctctggcctacatgcgtcgtattgctaaacaagatgttgaacttggcggacgcactatcaagaaaggagatcgcgtagtcatgtggtatgcctccggtaaccgcgacgaacgtaagttcaccgaccccgatcaatttttgattgatcgcagtggggcccgcaaccacttgtccttcggatatggagtacaccgttgtatgggtaatcgtcttgcggagcttcaactgcgtattttgtgggaagaattgttaaagcgtttcgaaaatattgaagttgtgggcgagcctgagcgtgttcaatcaaactttgttcgcggatattcagagatgatggtgaaattaactccactgcgttactaa

SEQ ID NO: 16 - protein, 471 aa, Limnobacter sp.
MSTQSKTFDQIQTRVINATAKVIPMHLQIQGLKFLMRAKKKTIGARRPTPSFVEYPLPDVGTLRIDDIDVSNPFLYRQGQWRAYFKRLRDEAPVHFQKNSPFGPFWSVTRYEDILFVDKHHDLFSAEPVIILGDPPEGLSVEMFIAMDPPKHDAQRSSVQGVVAPQNLKEMESLIRSRTGDVLDSLPVGQPFDWVPTVSKELTGRMLATLLDFPYDERDKLIYWSDLLAGAASATGGEFTDEEAMFDAAADMARDFSRLWRDKQARRAAGEAPGFDLISLLQSSEDTRDLINRPMEFIGNLALLIVGGNDTTRNSMSGGLLAMNQFPKEFKKLKANPALIPNMVSEIIRWQTPLAYMRRIAKQDVELGGRTIKKGDRVVMWYASGNRDERKFTDPDQFLIDRSGARNHLSFGYGVHRCMGNRLAELQLRILWEELLKRFENIEVVGEPERVQSNFVRGYSEMMVKLTPLRY

SEQ ID NO: 17 - DNA, 1272 bp, Caulobacter sp.
atgagcgatggtgcaattgatttacgcgcggaggcgcgcgccaaggcgtatgcgatgcccttagaggatatcaaccttgcagataccgaattgtggcgcacagatacggtgtggccgtatctggagcgcttgcgtaaggaagaccccgttcatttacaccctgctcatcaccatcccgacggagcgttttggagtattactaaatatgcagatatcatggccgtcgacattaatcatgaggtattttcaagtgaaccgtcgatcaccatctttgaccccaaagaggattttacccttcctatgttcattgcgatggacccgccaaagcatgatgtgcaacgcaagactgtcagtccgattgtgagtccggcaaacttacacttgatggaacctcttatccgcagtcgcatcaccaaaacgcttgacgaacttcctattggggagccatttgattgggtggatcgcgtctctatcgagcttacaactcagatgttggcgacccttttcgacttcccatgggacgagcgccgcaaactgacacgctggtccgatattgcaacagcagtacccgaaagcggcttatttgttacggacgattatgaaaccgagcgccgcatggaattattcgggtgcgtcgattatttcactcgtctttggaacgaacgtgtgaatgccccacctaagggtgatctgatctctatgttagcccatggggaggccacccgtaatatggaccgtatggagtaccttgggaacttattacttcttattattggagggaacgacacaactcgtaacactatgacaggctcgatcctggcgatgaatcaaaaccccgaccaattacgcaaattacgtgaaaacccgagtctgattccgtccatggtcagtgaaaccattcgctggcagactccactgtcaaacatgcgccgcacagccacccaagactttgagttaggtgggaaattgattaagaaaggtgataaagtgctgatctggtatgcctccggtaatcgtgatgaagaagctatcgaaaacccagaagcgtacattattgaccgtgagcgtcctcgtaatcatctttcgtttggtttcggtatccaccgttgcgtcgggaatcgcctggcagaattgcaattgcgtattctgtgggaagaaattttaccccgttttccagagattcaggtactggaggatccgcagcgtgttccgtcagttctgattcgtgggtactcctccatgcccgtcatgatccctgcacgctcatactaa

SEQ ID NO: 18 - protein, 423 aa, Caulobacter sp.
MSDGAIDLRAEARAKAYAMPLEDINLADTELWRTDTVWPYLERLRKEDPVHLHPAHHHPDGAFWSITKYADIMAVDINHEVFSSEPSITIFDPKEDFTLPMFIAMDPPKHDVQRKTVSPIVSPANLHLMEPLIRSRITKTLDELPIGEPFDWVDRVSIELTTQMLATLFDFPWDERRKLTRWSDIATAVPESGLFVTDDYETERRMELFGCVDYFTRLWNERVNAPPKGDLISMLAHGEATRNMDRMEYLGNLLLLIIGGNDTTRNTMTGSILAMNQNPDQLRKLRENPSLIPSMVSETIRWQTPLSNMRRTATQDFELGGKLIKKGDKVLIWYASGNRDEEAIENPEAYIIDRERPRNHLSFGFGIHRCVGNRLAELQLRILWEEILPRFPEIQVLEDPQRVPSVLIRGYSSMPVMIPARSY

SEQ ID NO: 19 - DNA, 1395 bp, Mycobacterium marinum
atgtcgaacattcgtgaagcagtaacggcaaaggcgcaagccactattccaatggatcgtattatccagggagcgcacttgtatgatcgtacacgccgctgggtaaccggcacgaatggtgagaaaattttcattgaacgcccgatcccgcctgccgacgaagtcgagcttacagacatcgacttaagtaatcccttcctttaccgccagggccgctggaagtcttattacgagcgccttcgtaacgaagcgcctgtccactaccaagcgcattcggccttcgggcctttctggtctgttactcgtcacgcagatattgtggcagtcgataagaaccatgaagtttttagcagtgaaccgtttatcgtgattgggtccccaccacgctttttggatatcgcgatgttcatcgctatggatccacccaaacatgatcgccaacgccaggcagttcagggcgtcgtggcaccaaagaatttacgtgaaatggaaggattaattcgcgagcgcgtcgtggacgtgttggatgccctgccccttggcgaacctttcaattgggtgcagcacgtctcaattgaattgactgcgcgcatgttagctacattattggactttccttttgaacagcgtcgcaagttggtccagtggtccgacttggcgacgagcatggaacaagcaaatggcggtcctagcgataatgacgaaatcttccgcggtatggtggacatggcgcgtgggttatctgcccattggcgcgacaaagcagcacgtactgcggcaggagaacttccaggattcgaccttatcactatgttacagagcgacgaatccacgaaggacctgattgaccgcccgatggagtttttaggtaatcttgttcttcttatcgtgggaggtaatgatactacccgtaattctatgagtggtggcgtattggctctgaacgagttcccagatcagtttgagaaactgaaggctaacccagaactgattcctaatatggtatctgagattattcgctggcaaacaccgttggctcacatgcgtcgcattgccaaagccgatacggtgctgaatgggcaatttattcgcaagggagacaaagttttgatgtggtatgcttcaggtaatcgcgatgagcgtgtatttgaccgccccgatgacttgattatcgaccgcgcaaatgcccgtaaccatatttcgttcgggttcggagttcatcgctgtatgggaaatcgtcttgctgagatgcagttacgcatcttatgggaggaacttttaccacgctttgagaacattgaagtagtgggggaacctgagtacgtccaatctaatttcgtgcgtgggatcagcaaactgatggtccgtttgacccccaagggcggtgcctactaa

SEQ ID NO: 20 - protein, 464 aa, Mycobacterium marinum
MSNIREAVTAKAQATIPMDRIIQGAHLYDRTRRWVTGTNGEKIFIERPIPPADEVELTDIDLSNPFLYRQGRWKSYYERLRNEAPVHYQAHSAFGPFWSVTRHADIVAVDKNHEVFSSEPFIVIGSPPRFLDIAMFIAMDPPKHDRQRQAVQGVVAPKNLREMEGLIRERVVDVLDALPLGEPFNWVQHVSIELTARMLATLLDFPFEQRRKLVQWSDLATSMEQANGGPSDNDEIFRGMVDMARGLSAHWRDKAARTAAGELPGFDLITMLQSDESTKDLIDRPMEFLGNLVLLIVGGNDTTRNSMSGGVLALNEFPDQFEKLKANPELIPNMVSEIIRWQTPLAHMRRIAKADTVLNGQFIRKGDKVLMWYASGNRDERVFDRPDDLIIDRANARNHISFGFGVHRCMGNRLAEMQLRILWEELLPRFENIEVVGEPEYVQSNFVRGISKLMVRLTPKGGAY

SEQ ID NO: 21 - DNA, 1299 bp, Marine gamma proteobacterium HTCC2148
atgccggaagttgcattggacaactcccaggataaacttcagcaggatcgtatcgagaactacgacgcgacggtcgtagacaaggacccttatgacgttgacatcgcggacttcaacatgattaacccccaccttttctcttctaacaccctttggccctggttcaaacgcctgcgtgaggaggagccagtccattattgtcgcgactccgtatttggtccgtattggagcgttacgagttatgatgctatcatgaaagtagacacgtcacatgacatctttagctcggagccgaacattaccgtaggcgatttacaagaggactttcctttagcaacttttatcgccatggatcgtccgaagcacgacgaacaacgcgccgtagtaaatcctgctgtgcgcggacagcagttgcgcgactttgagccccttattcgcgagcgcactcaacgcgtactggattctttaccggtgggcgaagaattcaattgggtggaaaaggtctctattgaattaactacctgtatgctggctaccttgtttgactttccgttcgaggaccgtgccaaattaacccgttggtctgatgtgacgttcgcaatccctggactgggtgtagtggatagtgaagaacagcgtcgtgaggaacttttggagtgccttgcggccttcaccgaactttggaaccaacgtgtcaacgaagaaccgcgcggggacttcattagtctgctggctcatggcagcgcaacgcaggaaatggatccctttgaatatctgggcaacatccttcttttgatcattggtggtaatgacacaacacgtaattcgatgagcggaagcgtgttgtgtcaaaacctgtacccagagcaatttgataagttaaaggcacaaccggagctgattccatctatggttagtgagacgattcgttggcaaactcccctggcctatatgcgtcgcaccgcaaatcaagacacaatcttaggggataagcaaatcaagaaaggtgataaggttttgatgtggtatgtgagcggcaatcgcgatgaatctcactttgaaaatcctgacgactatatcatcgatcgccctaacgtccgcagccacctttcatttggttttgggatccatcgttgtatgggcaatgcggtcggtgagatgcagttgcgtgtcctgtgggaggagatcctgcaacgtttcgaccgtattgaggtagttggtgatgttgaacgcgtgccttcctcattcgtaaaggggtatagcagcttacctgtgattctgcaccctaaaaagtactaa

SEQ ID NO: 22 - protein, 432 aa, Marine gamma proteobacterium HTCC2148
MPEVALDNSQDKLQQDRIENYDATVVDKDPYDVDIADFNMINPHLFSSNTLWPWFKRLREEEPVHYCRDSVFGPYWSVTSYDAIMKVDTSHDIFSSEPNITVGDLQEDFPLATFIAMDRPKHDEQRAVVNPAVRGQQLRDFEPLIRERTQRVLDSLPVGEEFNWVEKVSIELTTCMLATLFDFPFEDRAKLTRWSDVTFAIPGLGVVDSEEQRREELLECLAAFTELWNQRVNEEPRGDFISLLAHGSATQEMDPFEYLGNILLLIIGGNDTTRNSMSGSVLCQNLYPEQFDKLKAQPELIPSMVSETIRWQTPLAYMRRTANQDTILGDKQIKKGDKVLMWYVSGNRDESHFENPDDYIIDRPNVRSHLSFGFGIHRCMGNAVGEMQLRVLWEEILQRFDRIEVVGDVERVPSSFVKGYSSLPVILHPKKY

SEQ ID NO: 23 - DNA, 1479 bp, Patulibacter medicamentivorans
atgtttgaacaaacaaccacgaagcgcgagaccatgacaacgaacagcacgttattccagcgtacaaaggtccgcgtgactgacacggttcaagctactgtcccggtagaccgtgtaatccagtctgtggccctgacgttgaaagcaaaacgcttggctgggatgatgaaagcattgcgtttcgaagaacgtcccattcctgatccggcagatgttccactggaggaaatcgacgtctctaatccttttatgaaccgccaaggacaatggtatccgtattttgcgcgtttacgcgaggaggcgcctgtgcattatcaacccaagtcgccttttgggccgttctggtcggttacgcgttacgcggacattcaagcggttgattcaaacgcagaggtctttagtgcagaaccgtatatcgtcttaggccctccaccgttcaacgccgaaatgttcatcgccatggaccctcccaaacacgacgtccagcgtcgtgcagtccaaggtgtggtcgcgccgaagaacttaaaagaaatggagagtttaatccgcgaacgcgtacaggatgtattagataatttaccgacagaccgcccctttgattgggtcgaacgtgtcagtaaagaaatcacagggcgcatgttagccaccttattggatttcccattcgaggagcgccataagttgacgtattggagtgacacgatcagtggctcagcagacgcaactggtggcacgacccatcaagacgagttgttccctgccgcagttgatttggcacgcaatttcaccgccctgtggcacgataaggcagctcgtcgcgctgcgggcgaaccagatgggtttgaccttattacacttatgcagacatcggaggacacaaaagaccttatcaaaaagccgtttgagtttctggggaatttgactttattgattgttggtggaaatgacacaactcgcaactcgatgtcaggaggcgttctggcacttaaccgctttccagatgaatttgagaagttgaaagcggacccaaaactgattccgaacatggtatccgagatcatccgttggcagactcccttggcatatatgcgccgcgtagctaaaaaagacgttcattttggcgggcaatttattcgcaaaggtgacgttgtattaatgtggtacgcctctgggaatcgtgacgagcgcaagtttgaaaacgccgacgacttcatcatcgaccgtaagaatgcacgcaaccacatgtctttcggattcggagtccaccgttgcatgggtaaccgcttagctgagatgcagcttcgtatcctttgggaagagcttttggaacgttttgaccgcatcgacgtaattggcgaaccagggtatgtacagtcaaactttgtcaaagggtactccgacatgcaggtccaattaacgcctaagcccgctcgcgaacaaacacctgtccgcgttgaggccacttcgggtgcatactaa

SEQ ID NO: 24 - protein, 492 aa, Patulibacter medicamentivorans
MFEQTTTKRETMTTNSTLFQRTKVRVTDTVQATVPVDRVIQSVALTLKAKRLAGMMKALRFEERPIPDPADVPLEEIDVSNPFMNRQGQWYPYFARLREEAPVHYQPKSPFGPFWSVTRYADIQAVDSNAEVFSAEPYIVLGPPPFNAEMFIAMDPPKHDVQRRAVQGVVAPKNLKEMESLIRERVQDVLDNLPTDRPFDWVERVSKEITGRMLATLLDFPFEERHKLTYWSDTISGSADATGGTTHQDELFPAAVDLARNFTALWHDKAARRAAGEPDGFDLITLMQTSEDTKDLIKKPFEFLGNLTLLIVGGNDTTRNSMSGGVLALNRFPDEFEKLKADPKLIPNMVSEIIRWQTPLAYMRRVAKKDVHFGGQFIRKGDVVLMWYASGNRDERKFENADDFIIDRKNARNHMSFGFGVHRCMGNRLAEMQLRILWEELLERFDRIDVIGEPGYVQSNFVKGYSDMQVQLTPKPAREQTPVRVEATSGAY

SEQ ID NO: 25 - DNA, 1332 bp, Paraglaciecola psychrophila
atgaactcgctgacgaacaccacggctgagcctgtcgcgacaagttctatcacaccgacccctgctgtgaaattcatcgaacaacctattgccgatgtttctacggtggctctggaggacatcgatgtgtcgaatccattcatgttccgtcagaacaagtggcaatcgtactttaaacgtttgcgcgatgagtgcccggttcattatcaaaaaaattcacctttcggggcattctggtcggtaacccgtttcgaggatattatgtttgtggataagaatcacacgctgtttagttccgaacctgccatcgtgatcggagaccgcccagctgactatatgcttgacatgtttattgcaatggacccgcctaaacatgatgctcagcgtcaggctgtgcagagcgcggttgcccccaaaaacctggcggagatggaggaactgattcgcgagcgtacagtagatgtcctgaacgatcttccggtaggtgaatcgttcgactgggttgagaaagtaagtgtggaactgactactcgcatgctggcgacactgtttgacttcccgtacgaaaagcgccataagctgccttactggtcggatcttgcgtccggctcccccgaaatgactggtggcttagtgcaagacgatgaacgcgtggcaggcatcacggaccttatgacagagttctcccagctttggcacatcaaggcggcacagaaagcagcgggtgaacagggaggctttgatcttatttcattgatgcaggccaatgacaacacaaaaaacatggtcgacaccccgttagagttcctgggtaaccttgtgctgctgattgttggtggcaacgacaccactcgcaacagtatgactggcggcgtcgatgcgctgaacgagtttcctcaagagttcattaagctgaaaaatgacccgagcttaattcccaatatggtatctgaaatcatccgctggcagactccccttgctcacatgcgtcgcatcgcgacagaagacgtggaattaaatgggaagacgattaagaagggagacaaggttgttatgtggtacgtgtccggaaatcgtgatgaacgtgtaattaataatcctgatcaattcgtcattgaccgcgacaaagcccgtaatcatctgtcgtttggatttgggattcatcgttgcatgggtaaccgcttagcggaaatgcaactgcgcatcctttgggaggaaatcttacaacgttttgagaatatcgaagtgattaacaagcccaaatacgttcagtctaacttcgtcaagggatatacggaacttcgtgttaaattgacagccaaagtgtactaa

SEQ ID NO: 26 - protein, 443 aa, Paraglaciecola psychrophila
MNSLTNTTAEPVATSSITPTPAVKFIEQPIADVSTVALEDIDVSNPFMFRQNKWQSYFKRLRDECPVHYQKNSPFGAFWSVTRFEDIMFVDKNHTLFSSEPAIVIGDRPADYMLDMFIAMDPPKHDAQRQAVQSAVAPKNLAEMEELIRERTVDVLNDLPVGESFDWVEKVSVELTTRMLATLFDFPYEKRHKLPYWSDLASGSPEMTGGLVQDDERVAGITDLMTEFSQLWHIKAAQKAAGEQGGFDLISLMQANDNTKNMVDTPLEFLGNLVLLIVGGNDTTRNSMTGGVDALNEFPQEFIKLKNDPSLIPNMVSEIIRWQTPLAHMRRIATEDVELNGKTIKKGDKVVMWYVSGNRDERVINNPDQFVIDRDKARNHLSFGFGIHRCMGNRLAEMQLRILWEEILQRFENIEVINKPKYVQSNFVKGYTELRVKLTAKVY

SEQ ID NO: 27 - DNA, 1275 bp, Afipia broomeae
atgcacggtacattagaccccgcccgccattcagaacttaatgccgcacgcgagaaagcctacagtacgccgatcgagcagtttaatcccggcgacccagaccttttccgcaacgatacattctggccgtacttcgaacgtttgcgtaaggaggacccagtgcactattgtaaagattctcaattcggggcctattggtccgtgatgaaatataacgatattatgcatgttgagacgaatcatcaaatttacagcagtgaagccagccttggtggcattactattcgcgacgctcctccggaattccgtcgtccaatgtttattgcaatggaccaaccaaaacacagcgcccagcgcaagacggtcgctccgatgtttacacccacacatttggatgaactggccggaacaatccgccagcgtgctgcacaatgcttagacgcacttcctcgtaatgcaactttcgactgggtagatcgcgtaagcatcgaacttacgactcagatgctggcgactctttttgattttccctgggaggaccgtcgcaaattgacgcgctggtccgatgtagctaccgccttgcctggtaacactgacatcatcgcgtctgaggatgcccgtcaagcggagcttcaggagtgtgctgcatatttctctcgtttgtggaacgaacgcattaacgcgccccccaaatcagatcttctttcaatgatggcccatgccgaagcaactcgcgacatggaccccaagacgtttcttggcaacctgatcttgttgatcgtgggagggaacgacacgacacgtaattcactgtccggaggattatacgctttgaacgaaaacccagccgaataccaaaagttacgtagtaatcacgccttagtggactcgatggtgccagaggtgatccgctggcagacaccgttggcacatatgcgtcgcacagccctgcaagacacagagttgcgcggcaagcacattaaaaagggagataaggttgtcatgtggtacgtatctggcaatcgcgacgatgaagtgattgaaaatccagacagtttcattatcgatcgcgcacgtccccgccaacacctgtcgttcggctttggaatccaccgctgtgtcggcatccgtcttgctgagttacagttgaagatcgtctgggaagagatccttaaacgcttcgataacatcgaggtagtagaacccccacaccgcgtttactcctcctttgtgaaagggtacgaaacactgcccgtgcgtttggcatcatactaa

SEQ ID NO: 28 - protein, 424 aa, Afipia broomeae
MHGTLDPARHSELNAAREKAYSTPIEQFNPGDPDLFRNDTFWPYFERLRKEDPVHYCKDSQFGAYWSVMKYNDIMHVETNHQIYSSEASLGGITIRDAPPEFRRPMFIAMDQPKHSAQRKTVAPMFTPTHLDELAGTIRQRAAQCLDALPRNATFDWVDRVSIELTTQMLATLFDFPWEDRRKLTRWSDVATALPGNTDIIASEDARQAELQECAAYFSRLWNERINAPPKSDLLSMMAHAEATRDMDPKTFLGNLILLIVGGNDTTRNSLSGGLYALNENPAEYQKLRSNHALVDSMVPEVIRWQTPLAHMRRTALQDTELRGKHIKKGDKVVMWYVSGNRDDEVIENPDSFIIDRARPRQHLSFGFGIHRCVGIRLAELQLKIVWEEILKRFDNIEVVEPPHRVYSSFVKGYETLPVRLASY

SEQ ID NO: 29 - DNA, 1392 bp, Rhodococcus ruber
atgaagattcccgaggctattacagccaaagtgcaatctacgattcccatggatctgcagattcagggtgcacatttgtacgataaaacacgtcgctgggtgacggggacaaatggtgaaaaattgtttgtggagagccctatccctcccgttgaggacgttgagcttgcagatattgatttatcgaatccttttctttatcgtcaaggacgctggcagtcctacttcgaacgcttgcgcaacgaagctcctgtccactatcagcctaactcggccttcggtccgttttggtccgtaacccgtcacgcagacatcgtcgcagtggataaaaatcacgagctgttctcggccgaaccctttatcgtgattggagccccaccgcgtttccttgacattgccatgtttatcgcaatggacccgccgcgccacgatgcacaacgtgccgcagtacaaggggtggttgcccccaagaacttacgcgaaatggaaggtttgatccgctcccgtgtgcaagaagttttggatgacctgccggtagaccaaccctttgattgggtccaaaatgttagtatcgagttaacggctcgcatgttagccacattattggactttccgtatgaacaacgccgtaagttggtgtactggtcagacctggcgacctctctggagcaggcgaacgggggccccagcgataatgacgaagtgtttcccggaatgcgcgatatggctcgtgggttatcagcattatggcatgacaaagccgctcgcatggctgcgggggaagaaccaggctttgatctgatcactatgttgcagagcaatgagaacacgaaggatcttatcgatcgtcctatggaatttctgggcaatctggtgttactgattgtaggaggaaacgacacgacgcgcaactctatgtccggaggagtccttgcgttgaaccgcttcccagatcagttcgagaagcttaaggctaacccagatttgattccaaatatgggttcagaaattattcgttggcaaacacctttggcttacatgcgtcgtgttgcgaaagccgataccattttgaacggtcagtttatccgtaagggggataaggttgtgatgtggtatgcaagtggtaatcgtgatgagcgcgtgttcgaacgccccgatgaatttattattgaccgtgctaacgcccgtaaccacatctcttttggcttcggggtccaccgttgtatgggtaatcgtttggccgaattgcaattacgtatcctttgggaagaattattgcctcgctttgacaatatcgaggtagtaggggagcctgagtacgttcagtcgaattttgtacgcggtattagtaaaatgatggtacgtctgacccccaaatccagtgcctactaa

SEQ ID NO: 30 - protein, 463 aa, Rhodococcus ruber
MKIPEAITAKVQSTIPMDLQIQGAHLYDKTRRWVTGTNGEKLFVESPIPPVEDVELADIDLSNPFLYRQGRWQSYFERLRNEAPVHYQPNSAFGPFWSVTRHADIVAVDKNHELFSAEPFIVIGAPPRFLDIAMFIAMDPPRHDAQRAAVQGVVAPKNLREMEGLIRSRVQEVLDDLPVDQPFDWVQNVSIELTARMLATLLDFPYEQRRKLVYWSDLATSLEQANGGPSDNDEVFPGMRDMARGLSALWHDKAARMAAGEEPGFDLITMLQSNENTKDLIDRPMEFLGNLVLLIVGGNDTTRNSMSGGVLALNRFPDQFEKLKANPDLIPNMGSEIIRWQTPLAYMRRVAKADTILNGQFIRKGDKVVMWYASGNRDERVFERPDEFIIDRANARNHISFGFGVHRCMGNRLAELQLRILWEELLPRFDNIEVVGEPEYVQSNFVRGISKMMVRLTPKSSAY

SEQ ID NO: 31 - DNA, 1392 bp, Gordonia paraffinivorans
atgcagatccttgaccgtgtcgtcgagacggtgcaggccaatattccggtcgaccgccaggtgcaggcgttacaactgtttcacaaagcacgtggtcgcctggtaggggaatcacgtccagagccgtttgtagagaagcccatccccccagttgatgaagtatcgctggatgccattgacatgtctaatcccttcatgtatcgccaaggccagtgggcgccatacttcgcgcgcttgcgtgaagaagcgccagttcattaccagcccaattcccgttttgggccgttttggtcggtgacccgttacgaagacatcctgaccgtcgataaagaccacgagaccttcagtgcagaaccatttatcgtaattgggacgccgccccctggattggatgtagagatgtttattgctatggacccgccgcgccacgatgtgcaacgccgcgcggtccagggagtcgtagccccaaaaaacttaaaagaaatggaaggactgatccgcgagcgtgtttgtgaagtgttagataatcttcccgtaggtcagccatttaactgggtcgatcgcgttagtgtagagatcactgctcgcaccttagcgacgatcttagatttcccctatgagcagcgccgtaatttagttcgctggagtgacctggccgcaggatccgaagaagccacaggtggtgccagcgatccagacgtcgtctaccaggcggcgcttgagatggtacgtgctttctcagagttatggcacgataaggccgcacgtcgtgccgcaggcgaggaaccaggctttgatttgatctcaatgttacaatccaatgaggacaccaaggatttggttaaacgtccgatggaattcattgggaatcttacattactgattgtcggtggaaacgacaccactcgcaatagcatgtcgggtggagtgtacgcgctgaataagtttcctggagagttcgagaaacttaaggcagacccgtctttgattccgaatatggtttctgagatcattcgttggcagacgcccttggcgtacatgcgtcgcgtcgcgactaaagacaccatccttaatggccaattcattcgtaaaggcgacaagttggtgatgtggtatgcatccggaaaccgcgatgagtctaagttcgagcgtgcggatgagttgattatcgaccgccccaacgctcgtaatcacatggcgtttgggtatggggtgcaccgctgcatgggcaatcgtcttgccgagctgcaacttcgcatcttatgggaagaattactggaacgcttcgacgacatcaaggtcttggaggaaccagagtatgttcagtctaactttgttcgcggttactctaagttgatggtggagctgacggcgaagaagacgtcttactaa

SEQ ID NO: 32 - protein, 463 aa, Gordonia paraffinivorans
MQILDRVVETVQANIPVDRQVQALQLFHKARGRLVGESRPEPFVEKPIPPVDEVSLDAIDMSNPFMYRQGQWAPYFARLREEAPVHYQPNSRFGPFWSVTRYEDILTVDKDHETFSAEPFIVIGTPPPGLDVEMFIAMDPPRHDVQRRAVQGVVAPKNLKEMEGLIRERVCEVLDNLPVGQPFNWVDRVSVEITARTLATILDFPYEQRRNLVRWSDLAAGSEEATGGASDPDVVYQAALEMVRAFSELWHDKAARRAAGEEPGFDLISMLQSNEDTKDLVKRPMEFIGNLTLLIVGGNDTTRNSMSGGVYALNKFPGEFEKLKADPSLIPNMVSEIIRWQTPLAYMRRVATKDTILNGQFIRKGDKLVMWYASGNRDESKFERADELIIDRPNARNHMAFGYGVHRCMGNRLAELQLRILWEELLERFDDIKVLEEPEYVQSNFVRGYSKLMVELTAKKTSY

SEQ ID NO: 33 - DNA, 1497 bp, Acinetobacter sp.
atgaactcggtggcagagatttttgagaagattactcaaacggtcactagtacagcagcagacgttgccacaacagtgactgacaaggtgaaatcgaacgaacagtttcagacgggaaagcaattcttacacggacaagttacccgcttcgtacctttgcacacacaggtccgcggtattcagtggatgcaaaaggctaagttccgcgtgtttaacgtgcaagagtttcccgcatttattgagcaacctattcctgaggtcgccacgcttgctcttgcggaaatcgacgtttccaacccctttttgtacaaacagaagaaatggcagtcttatttcaagcgcttgcgtgatgaagcgcccgtgcattatcaggcgaactctcccttcggcgcattctggtcagtcacccgttatgacgacattgtctatgtcgacaagaatcacgagatcttttcggctgaaccagtaattgccattgggaatacgccgcccggcttaggtgcagagatgtttatcgcgatggacccaccgaagcacgatgtccaacgccaggctgtccaggatgtagtcgcaccgaaaaacttgaaagaattagagggcttgattcgccttcgtgttcaggaagtactggaccaattgcccacggatcaaccctttgattgggtgcaaaatgtatccatcgaattgacggcgcgtatgttggcaaccttgtttgattttccatacgaaaagcgtcacaagttagtagagtggtcagatctgatggccgggactgcggaggcaacagggggtacagtgactaacctggatgagattttcgacgctgctgtcgacgcggctaaacactttgctgaattgtggcatcgcaaagccgctcagaagtctgcaggagcggagatgggttatgatttgatttctcttatgcagagtaatgaggcaaccaaagatttgatctatcgccccatggaatttatgggaaatctggtgttattaatcgtaggtggtaatgacactacacgtaatagtatgacgggtggtgtttatgcgttgaatctttttccaaatgagtttgtaaagttgaagaataatccctcactgattccgaatatggtcagcgaaattatccgctggcaaacacctctggcatatatgcgccgcatcgccaagcaggacgtcgagttaaatgggcaaacaattaaaaaaggtgacaaagtggtaatgtggtatgtttcggggaaccgtgatgaacgtgtgattgagcgcccagatgaacttatcattgaccgtaagggtgctcgtaatcacctttctttcggtttcggagtgcatcgctgcatgggaaaccgtttagcggagatgcagcttcgtatcttgtgggaagagctgcttcaacgctttgaaaatatcgaagttctgggggagccggaaatcgtgcaatcgaactttgtccgcggttacgccaaaatgatggtgaaactgaccgcgaaggcctactaa

SEQ ID NO: 34 - protein, 498 aa, Acinetobacter sp.
MNSVAEIFEKITQTVTSTAADVATTVTDKVKSNEQFQTGKQFLHGQVTRFVPLHTQVRGIQWMQKAKFRVFNVQEFPAFIEQPIPEVATLALAEIDVSNPFLYKQKKWQSYFKRLRDEAPVHYQANSPFGAFWSVTRYDDIVYVDKNHEIFSAEPVIAIGNTPPGLGAEMFIAMDPPKHDVQRQAVQDVVAPKNLKELEGLIRLRVQEVLDQLPTDQPFDWVQNVSIELTARMLATLFDFPYEKRHKLVEWSDLMAGTAEATGGTVTNLDEIFDAAVDAAKHFAELWHRKAAQKSAGAEMGYDLISLMQSNEATKDLIYRPMEFMGNLVLLIVGGNDTTRNSMTGGVYALNLFPNEFVKLKNNPSLIPNMVSEIIRWQTPLAYMRRIAKQDVELNGQTIKKGDKVVMWYVSGNRDERVIERPDELIIDRKGARNHLSFGFGVHRCMGNRLAEMQLRILWEELLQRFENIEVLGEPEIVQSNFVRGYAKMMVKLTAKAY

SEQ ID NO: 35 - DNA, 1254 bp, Sphingopyxis macrogoltabida
atggagcacaccggtcaatctgcggcagccacaatgccactggatagcatcgatgtgtccatcccagaacttttttataacgattcggtaggagagtatttcaagcgtttacgcaaggatgaccccgtgcactattgtgcagattccgcgttcggcccatattggtccatcactaagtataacgatatcatgcacgtggacacaaaccatgacattttttctagcgacgccggatacgggggtattattattgacgacggcattcaaaagggtggggacggtgggttagatttaccgaatttcatcgctatggatcgtccccgtcatgacgagcaacgtaaggctgtgagcccaatcgtggctcctgcgaacttggctgcccttgagggaacgatccgcgagcgtgtcagcaagactctggacggtctgccggttggtgaggagttcgactgggttgaccgtgtatcaatcgagattactacgcagatgttagcgactttgttcgactttccgttcgaagagcgccgtaaactgacccgctggagtgatgtaacgaccgccgcacccggaggcggagtcgtggagagttgggaccaacgtaagaccgaattacttgagtgcgcggcctattttcaggttctttggaatgagcgtgtgaacaaagaccccggcaacgatcttattagtatgcttgcacatagtcctgctactcgtaacatgacacctgaagaatacttgggtaatgttctgcttttgatcgtaggcgggaacgacaccacgcgtaactcaatgactggtggcgtattggctctgcacaagaacccggatcaatttgcaaagctgaaagctaaccccgcccttgttgaaacgatggtccccgagatcatccgttggcaaacgcctttagcgcatatgcgtcgtacggcaattgcggactcggagctgggtggtaagactattcgcaagggtgataaagtcgtgatgtggtactattcaggtaaccgcgacgatgaagttattgatcgtcccgaagaattcattattgaccgccctcgcccccgtcaacacctttccttcggtttcggcatccatcgctgtgttgggaatcgcttagctgaaatgcaacttcgtattctgtgggaagaaattcttacgcgcttcagtcgcattgaggtgatggcggaacctgagcgtgtgcgctcgaattttgttcgcggttacgcgaaaatgatggtccgtgttcatgcttactaa

SEQ ID NO: 36 - protein, 417 aa, Sphingopyxis macrogoltabida
MEHTGQSAAATMPLDSIDVSIPELFYNDSVGEYFKRLRKDDPVHYCADSAFGPYWSITKYNDIMHVDTNHDIFSSDAGYGGIIIDDGIQKGGDGGLDLPNFIAMDRPRHDEQRKAVSPIVAPANLAALEGTIRERVSKTLDGLPVGEEFDWVDRVSIEITTQMLATLFDFPFEERRKLTRWSDVTTAAPGGGVVESWDQRKTELLECAAYFQVLWNERVNKDPGNDLISMLAHSPATRNMTPEEYLGNVLLLIVGGNDTTRNSMTGGVLALHKNPDQFAKLKANPALVETMVPEIIRWQTPLAHMRRTAIADSELGGKTIRKGDKVVMWYYSGNRDDEVIDRPEEFIIDRPRPRQHLSFGFGIHRCVGNRLAEMQLRILWEEILTRFSRIEVMAEPERVRSNFVRGYAKMMVRVHAY

SEQ ID NO: 37 - DNA, 1266 bp, Mycobacterium sp.
atgacggaaatgacagtcgcagcctccgacgcgactaatgcggcttatggtatggccctggaagatatcgatgtcagtaaccctgtgctgttccgtgacaacacatggcacccttactttaaacgcttacgcgaggaggacccagttcattattgcaaatccagtatgttcgggccctattggtcagtgacaaaatatcgtgatattatggccgtagaaaccaatccgaaggtctttagctctgaggcgaaatctggcggaatcacaattatggacgacaacgcggctgctagtctgccaatgtttattgcgatggatcctccgaaacatgacgtacaacgtaaaacggtttcgccgatcgtcgcgccagagaacttggccacaatggagtcagtgattcgtcagcgcacggctgatttacttgacgggttacccattaacgaggagttcgactgggtccatcgtgtctctatcgagctgaccacgaagatgctggccacgcttttcgatttcccttgggacgatcgcgccaaactgacacgctggtccgacgttacaactgcgcttcccggagggggcattattgatagtgaggaacagcgtatggcggagttgatggagtgtgcgacatatttcactgaattgtggaatcagcgtgtgaatgccgagccaaagaacgatttaatttccatgatggctcacagcgagtcaacccgtcatatggctccggaggagtacttagggaatatcgtactgctgattgttggtggaaacgatactacacgcaattcgatgacagggggggtgcttgcgttaaacgaatttccggatgaatatcgtaagttatccgccaaccctgccttgattagttcgatggtatcagaaatcattcgttggcaaaccccacttagtcacatgcgtcgtacagccctggaagatatcgaatttggtggtaaacatatccgccagggtgacaaagttgtgatgtggtacgtctctggtaaccgtgacccggaagctatcgataatcccgacacattcattattgaccgcgctaagccgcgccagcatttatccttcgggttcgggattcatcgttgtgtaggcaaccgtttagctgaattgcagttaaatatcctttgggaggagattttaaaacgttggccggatccactgcagatccaagtgttacaggaacccacccgtgttctgagccccttcgttaagggctacgagagtctgccggtacgtatcaatgcgtactaa

SEQ ID NO: 38 - protein, 421 aa, Mycobacterium sp.
MTEMTVAASDATNAAYGMALEDIDVSNPVLFRDNTWHPYFKRLREEDPVHYCKSSMFGPYWSVTKYRDIMAVETNPKVFSSEAKSGGITIMDDNAAASLPMFIAMDPPKHDVQRKTVSPIVAPENLATMESVIRQRTADLLDGLPINEEFDWVHRVSIELTTKMLATLFDFPWDDRAKLTRWSDVTTALPGGGIIDSEEQRMAELMECATYFTELWNQRVNAEPKNDLISMMAHSESTRHMAPEEYLGNIVLLIVGGNDTTRNSMTGGVLALNEFPDEYRKLSANPALISSMVSEIIRWQTPLSHMRRTALEDIEFGGKHIRQGDKVVMWYVSGNRDPEAIDNPDTFIIDRAKPRQHLSFGFGIHRCVGNRLAELQLNILWEEILKRWPDPLQIQVLQEPTRVLSPFVKGYESLPVRINAY

SEQ ID NO: 39 - DNA, 1320 bp, Candidatus Microthrix parvicella
atgaccgatgacacgaagccgcgcattgatttcgacccatcgattcgtacgccagagatggaaatggccgaagttggggcgggggttccagatgctgctgacttaaaacttacagacttaaatcccgctaatccacatttgtttaaggaggaccgctggcacgatcatttcgctcgcttacgtgcggaggacccggtccatcttaatgaaatcgagactgcgggtcgctactggtctatcacgaagtatgacgatgtgcgcgccgtcgacggcgattggcaaactttctcgtcggcacaaggtatgacgttggggctgcgccctgatcccgaccgcccgaatccgctggtacaaatcacccctttcattgcgatggacccgccggagcacacagcacaacgtaaaactgttcgcagcgtgtctgctccgtccaaccttcgtaacttagaacccttgatccgcgaacgtactgttgcagtgcttgattctttacccgaaggagaaaccttcgactgggtcgatacggtcagtattgagctgacaacccttttacttgccacattatttgactttccgcttgaggatcgtcgcaagcttacccgctggtccgacattgttttcgccgtgcccgggtcgggtggagttgtagaaacgcagcaacagaaaatcgaagagcttatggagtgcgtcacttactttgaggggttatgggaagagcgtcgtggtggtgacgggacagacttagtttcgatgttggccaacggggaagcaactaaagacatgcccaccatttcgcatttgggtaatctgttgctgttaatcgtgggagggaacgatacaacccgcaataccatgacaggttcggtgtacggattgaacaaatatccagatcaatatgacaaattaactgcggaccctggcttgattagcacctttgttccggagattattcgctggcagacaccactgtcttatatgcgtcgcacagccacgaaagattgtacaatccgtgataaacagatccttgaaggcgaccaagtgcttatgtggtatatttctgctaatcgtgatgaggacgtttttgtagacgctgagcgcatcgacttagaccgctctaatgcggaccgccaactggcgttcggctacggaatccacttctgcatggggtcgcgtcttgctgaattgcaactgcgcattttatgggaagaggtacttgcacgtttcgaacgtattgagcttcaagccgagcctgagcgcacacttagttcctttgttcatgggtacacgaaactgcctgtaactgttactcgccgttactaa

SEQ ID NO: 40 - protein, 439 aa, Candidatus Microthrix parvicella
MTDDTKPRIDFDPSIRTPEMEMAEVGAGVPDAADLKLTDLNPANPHLFKEDRWHDHFARLRAEDPVHLNEIETAGRYWSITKYDDVRAVDGDWQTFSSAQGMTLGLRPDPDRPNPLVQITPFIAMDPPEHTAQRKTVRSVSAPSNLRNLEPLIRERTVAVLDSLPEGETFDWVDTVSIELTTLLLATLFDFPLEDRRKLTRWSDIVFAVPGSGGVVETQQQKIEELMECVTYFEGLWEERRGGDGTDLVSMLANGEATKDMPTISHLGNLLLLIVGGNDTTRNTMTGSVYGLNKYPDQYDKLTADPGLISTFVPEIIRWQTPLSYMRRTATKDCTIRDKQILEGDQVLMWYISANRDEDVFVDAERIDLDRSNADRQLAFGYGIHFCMGSRLAELQLRILWEEVLARFERIELQAEPERTLSSFVHGYTKLPVTVTRRY

SEQ ID NO: 41 - DNA, 1257 bp, Afipia sp.
atgcaatcgacgcaacgtggggcccgtgattttgcgacacgtctgccattggacgcgattgacgtatccgatccacaactgtatcaggacgatacctggcgtcctcttttcgcgcgccttcgcgcggaagacccggtccactattgtcgcgactcggcgtttggtccctactggagtgtaacgacgtacgacgatatcttgaaagtggaacttgatcattcaacgtactcatccagctcggaactgggaggcattcaggtgaccgatcaaccgaagggaaaggaaactatttctttcattcgtatggacccacctggccacactgcgcaacgccgtatcgttgctcctatcgtcgcccccacacacttggcaaactttgagccagtgatccgtgagcgcacagcccgtgttcttgacggcctgcctcgtaatgaaaccttcgattgggctgaccgcgtctccgtcgaacttaccgcaatgatgttagcaactcttttcgactttcccatggaagaacgtcgcaagttaacttactggtccgacgtagcgattgctaacattaactcgccggagagccctatcacatctgaggatgagcgctcggagaaattaggggagatggccgcgtgcttcaaggctttgtgggatcgccgtgcagcagtcgagcccaagttcgaccttgtatcaatgttggcgcatggggcagcgactcgtgatatgggggtccgtgaattaacaggtactatcggtttgctgatcgtggggggtaatgatacaacccgtaattccatgacgggtggagtactggcgcttcacgactatcccgaggaggcagaaaagttgcgcggcaatccagccttaatcccgtcgttagtgtccgaaattattcgttatcaatccccggttttgcacatgcgtcgtacggcccgtgttgatgctgagattggaggaaagaccatccgcgctggggacaaggtcgtcatgtggtacatttccgggaaccgtgatgagaaaaagatcgagcacgcagatcgttttgtgatcgaccgtgccaagccccgtcaacatttggctttcggggccggcgttcaccgttgtgttggagatcgccttgccgaattgcagctgcgcattctttgggaagcaatcctggaacgcggctttgttattgacgtagttggggaaccaaagcgtctttattccaatttcatccgtggctttcgctctcttcccgtgcgcattcgcactgcctactaa

SEQ ID NO: 42 - protein, 418 aa, Afipia sp.
MQSTQRGARDFATRLPLDAIDVSDPQLYQDDTWRPLFARLRAEDPVHYCRDSAFGPYWSVTTYDDILKVELDHSTYSSSSELGGIQVTDQPKGKETISFIRMDPPGHTAQRRIVAPIVAPTHLANFEPVIRERTARVLDGLPRNETFDWADRVSVELTAMMLATLFDFPMEERRKLTYWSDVAIANINSPESPITSEDERSEKLGEMAACFKALWDRRAAVEPKFDLVSMLAHGAATRDMGVRELTGTIGLLIVGGNDTTRNSMTGGVLALHDYPEEAEKLRGNPALIPSLVSEIIRYQSPVLHMRRTARVDAEIGGKTIRAGDKVVMWYISGNRDEKKIEHADRFVIDRAKPRQHLAFGAGVHRCVGDRLAELQLRILWEAILERGFVIDVVGEPKRLYSNFIRGFRSLPVRIRTAY

SEQ ID NO: 43 - DNA, 1242 bp, Blastomonas sp.
atggcaacagtgctgaaggagcccggcgcggcgttgaactacgatatgagtgatgcgtcctggtatgtggaggatcgctggcaagaaccattccgtcaaatgcgtgagcaagacccgattcactggactgaaaatggcatgttcggtagtttctggaatgtaactaatcacaaagcgatccaacacgtcgaagcattgccagaaattttcagttcctcctacgaacatggagggattacactggcagaccgtatcgacgacgggacggaactggttatgccaatgttcattgccatggaccgtccgaaacacacaggacaacgtcgtactgtagctccggccttcacccctacagaaatgaagcgtatgtctgacgatatccgccgtcgcactgccgaaattttagacggccttccatgggaccagccgttcgattgggttgatcgtgtcagtatcgagcttacgacacagatgctggctattttgttcgacttcccatgggaggatcgtcgtaaactgaccgaatggagtgattgggccggagatattgaattgattcattcggaggaaatgcgtcaagagcgcttaaaacatctgtacgatatgggcgcgtactttaaaaagctttgggatgccaaaatcaacgcagaaccaaccccagatttaatctctatgatgatccattccgacgccatgagcgagatggatgaatttgagtttatgggtaacttaatcctgttaatcgtcggtggcaacgatacgacgcgtaattcaatgtcgggtcttgtgtatggcctgcagcagttcccggatcaacgcgagaagcttgaacaaaatccggcgcttatccctaatgcggtgcaagagatcattcgctggcagacacccttagcccatatgcgccgcacagcactggaagattatgacttattcggcaagactattcgcaaaggagataaactggcactttggtacatttcagggaaccgtgacgagagtgtgtttgaggatgcggataaaatcatcgtagatcgtgaaaatgctcgccgccaccttgcgttcggatatggtatccaccgttgtgtcggcgctcgtttagccgaattgcagattgcgattctgttagaagagatggcgaaacgtcgtatgcgtgttaatgtccttgaagagccagttcgcgttcgcgcgtgttttgtgcatgggtatcgctccatgcaagtttccttatcaaagtactactaa

SEQ ID NO: 44 - protein, 413 aa, Blastomonas sp.
MATVLKEPGAALNYDMSDASWYVEDRWQEPFRQMREQDPIHWTENGMFGSFWNVTNHKAIQHVEALPEIFSSSYEHGGITLADRIDDGTELVMPMFIAMDRPKHTGQRRTVAPAFTPTEMKRMSDDIRRRTAEILDGLPWDQPFDWVDRVSIELTTQMLAILFDFPWEDRRKLTEWSDWAGDIELIHSEEMRQERLKHLYDMGAYFKKLWDAKINAEPTPDLISMMIHSDAMSEMDEFEFMGNLILLIVGGNDTTRNSMSGLVYGLQQFPDQREKLEQNPALIPNAVQEIIRWQTPLAHMRRTALEDYDLFGKTIRKGDKLALWYISGNRDESVFEDADKIIVDRENARRHLAFGYGIHRCVGARLAELQIAILLEEMAKRRMRVNVLEEPVRVRACFVHGYRSMQVSLSKYY

SEQ ID NO: 45 - DNA, 1278 bp, Candidatus Phaeomarinobacter ectocarpi
atgacgaccgccaatcaaactagcccaaatggagccattgacgtgaacgatatccctttggcagagttagatgtgagccaacctcatctgtttaagaacgacacctggcgcccatggttcgcacgcctgcgtgctgaggcgcccgtccattatcttgccgatagcgaaaacggacctttctggtcggtcacgtcacacgatatgactaaagcggtcgacgcaaaccataaggtcttctcatccgaggagggcggcattgccatcgtcgacccacagcctttggacggtgagcaattaatgcgtgacccttcgtttatctcaatggatgagccaaagcatgctacacaacgcaaggccgtgtcgccggctgtagctcccaaaaaccttgcagagctggaacctttgattcgcgagcgtgccgctgacatccttgataacctgccagtcggggaaacctttaactgggtcgaccgtgtttcagttgaacttacagcgcgcatgcttgcgacgttgttcgacttcccttatgaacgccgtcgtgacttgattcgctggagtgatgtggccaccgctgtacctaaagtgactggggaggcaaatgacatgggtgcccgccgcgatgcgcttattgaatgcgctactacattctaccaattatggcaggaacgcgcggcacaacctccgaagttcgacttcgtcagcatgttagcccatggggaagcgacaaaacacctttcagaagacccattattgatgttggggaacatcatcttattgatcgtgggtggcaatgacactacacgtaattcgatttccgggggtgttgttgctttgaaccagtatccagaagagtatcagaagttgcgtgatactcccgccctgatccccaatatggtggcggagacggtacgctggcaaacccctgttatccacatgcgccgtaccgcccttgaggacgttgagctgggcggtaagacgatccgtaaaggagacaaagtcgtgatgtggtaccttagtggtaaccgtgatgaggcagtcttcccagacgcagaccgcttgatcattgaccgcccaaatgcacgccagcatgtatcgtttggattcggcgtgcaccgttgcatgggcaaccgtttagctgaaatgcaacttcgtgtcttgtgggaggagatcatgaaacgctttcatacagtcgaagttgtgggggaagttgagcgccttagcaacaactttatccgtgggatcgcgtcagtccctgtgcgtttgcacccgttatactaa

SEQ ID NO: 46 - protein, 425 aa, Candidatus Phaeomarinobacter ectocarpi
MTTANQTSPNGAIDVNDIPLAELDVSQPHLFKNDTWRPWFARLRAEAPVHYLADSENGPFWSVTSHDMTKAVDANHKVFSSEEGGIAIVDPQPLDGEQLMRDPSFISMDEPKHATQRKAVSPAVAPKNLAELEPLIRERAADILDNLPVGETFNWVDRVSVELTARMLATLFDFPYERRRDLIRWSDVATAVPKVTGEANDMGARRDALIECATTFYQLWQERAAQPPKFDFVSMLAHGEATKHLSEDPLLMLGNIILLIVGGNDTTRNSISGGVVALNQYPEEYQKLRDTPALIPNMVAETVRWQTPVIHMRRTALEDVELGGKTIRKGDKVVMWYLSGNRDEAVFPDADRLIIDRPNARQHVSFGFGVHRCMGNRLAEMQLRVLWEEIMKRFHTVEVVGEVERLSNNFIRGIASVPVRLHPLY

SEQ ID NO: 47 - DNA, 1260 bp, Candidatus Phaeomarinobacter ectocarpi
atgtcgcaagctgcggcagagacccctagcacagtcgatcatcaggagcgtgcatggtctatgcctctggaagatatcaacgtggctgacggtgcacttttccaagacgatgctatttggccctactttgaacgtcttcgcaaggaagcaccggttcataagggacatagcgacgagttcggtgactattggagtgtgactcgttatgaagacattatggcggtggacaccaatcatcatgttttctcctcggagggggccattacccttgcagatccgttggaagatttccgtgctccaatgttcatcgcaatggatcccccgaagcacgacaaacagcgtattactgtccaaccgatcgtcgccccaaaaaatctgcaaaactgggagggcttgatccgtgagcgtaccggcttaattctggatcaactgccccgcaacgagacgtttgattgggtcgataaagtaagcattgaacttaccactatgatgcttgccacgttattcgatttccctttcgaggagcgccgtcgcttgacccgttggtctgatgtcgccacagggcgtgataatcccgaaatttataaaagcgaagaacagtggcgtggggagcttatggagtgtttagaggcgttcactgggttatggaacgaccgcgttaactcagacacgcccggaaacgatttaatttcgatgctggcgagcggggaatctactaagaatatggatcccatggagtatttaggcaacattattctgcttattgtcgggggaaatgatactactcgcaactcaatgacgggatctgtttacgccttaaacaagtttgcgggcgaatatgataagttaattgctgatccttcactgattcccaatctttcgagcgagatcattcgctggcaaacaccattggcacatatgcgtcgtactgcacttgaagacattgagctgaacggccagatgatcaaaaaaggagacaaagtggccatgtggtacgtatccggaaaccgcgacacggctgtgttcgagaacgctgatgacgtaattattgaccgccccaatgcacgtcgccagatgtccttcgggtatgggattcaccgctgcgttggaaatcgccttggtgaattacagattaagatcctgtgggaggaactgctgaagcgttttcccaaaatcgaggtcatggaagaaccaacgcgcacacgttcaccattcgtcaagggatacacgtatatgcccgttcgtattccggcctactaa

SEQ ID NO: 48 - protein, 419 aa, Candidatus Phaeomarinobacter ectocarpi
MSQAAAETPSTVDHQERAWSMPLEDINVADGALFQDDAIWPYFERLRKEAPVHKGHSDEFGDYWSVTRYEDIMAVDTNHHVFSSEGAITLADPLEDFRAPMFIAMDPPKHDKQRITVQPIVAPKNLQNWEGLIRERTGLILDQLPRNETFDWVDKVSIELTTMMLATLFDFPFEERRRLTRWSDVATGRDNPEIYKSEEQWRGELMECLEAFTGLWNDRVNSDTPGNDLISMLASGESTKNMDPMEYLGNIILLIVGGNDTTRNSMTGSVYALNKFAGEYDKLIADPSLIPNLSSEIIRWQTPLAHMRRTALEDIELNGQMIKKGDKVAMWYVSGNRDTAVFENADDVIIDRPNARRQMSFGYGIHRCVGNRLGELQIKILWEELLKRFPKIEVMEEPTRTRSPFVKGYTYMPVRIPAY

SEQ ID NO: 49 - DNA, 1269 bp, Pseudomonas putida
atgaacgcaaatgataacgtcgtaatcgtcgggacgggacttgcgggggtagaagttgcatttggcttacgcgccagtggatgggagggtaatattcgcctggttggagatgcgactgttattccacaccacttgcccccgttgagcaaagcgtacttagccggtaaggcgactgcagagtcattatatttacgtactccggacgcctatgcagcacaaaacatccaattgcttggaggtacgcaggttacagcgattaatcgtgaccgtcaacaagtcatcctgtctgatggccgtgcgttggactatgaccgcttagtgctggcaactggtggccgcccgcgcccgcttcctgtggcaagtggtgctgtcgggaaagcgaataatttccgctatctgcgtactcttgaagacgccgagtgcatccgccgccagttaatcgccgataatcgtctggtggttattggtggaggttacattggtctggaagtagccgcgaccgcaatcaaggcgaacatgcatgttacattattggatacggccgcacgtgtgcttgaacgcgtaaccgccccgccagtatccgcattctatgaacacttacatcgcgaggcaggggttgatatccgtactgggacccaggtctgtggatttgaaatgtcgacagatcagcagaaagttactgcggtcttgtgcgaagacgggacccgccttccggcggatcttgtgatcgcaggaattggtctgatccccaattgcgaattagcatccgcagctggcttacaagtggataacggcatcgtgattaacgaacacatgcagacgagtgaccctcttatcatggcggttggcgactgtgcgcgctttcatagccaactttacgaccgttgggtgcgtattgagtccgtacccaacgcgctggaacaagctcgtaagatcgcggctattttgtgcggtaaagtgcctcgtgatgaagcagctccttggttctggagtgaccaatacgagattggattgaagatggtaggattatcagaaggctacgatcgcattattgtccgcggctcacttgctcagcccgatttctccgtcttctatcttcagggagatcgcgtgttggctgtcgatacagttaatcgtcctgtggaattcaatcaaagcaaacagattatcacggatcgtttacccgtagagcctaacttactgggcgacgaaagcgtcccacttaaggagatcattgcagccgcgaaggctgagttatcgagtgcataa

SEQ ID NO: 50 - protein, 422 aa, Pseudomonas putida
MNANDNVVIVGTGLAGVEVAFGLRASGWEGNIRLVGDATVIPHHLPPLSKAYLAGKATAESLYLRTPDAYAAQNIQLLGGTQVTAINRDRQQVILSDGRALDYDRLVLATGGRPRPLPVASGAVGKANNFRYLRTLEDAECIRRQLIADNRLVVIGGGYIGLEVAATAIKANMHVTLLDTAARVLERVTAPPVSAFYEHLHREAGVDIRTGTQVCGFEMSTDQQKVTAVLCEDGTRLPADLVIAGIGLIPNCELASAAGLQVDNGIVINEHMQTSDPLIMAVGDCARFHSQLYDRWVRIESVPNALEQARKIAAILCGKVPRDEAAPWFWSDQYEIGLKMVGLSEGYDRIIVRGSLAQPDFSVFYLQGDRVLAVDTVNRPVEFNQSKQIITDRLPVEPNLLGDESVPLKEIIAAAKAELSSA

SEQ ID NO: 51 - DNA, 324 bp, Pseudomonas putida
atgtctaaagttgtctatgtctcccatgacggaacccgtcgtgagttagacgttgctgatggtgtgtcactgatgcaagcagcggtcagcaatggaatttatgatattgtgggcgactgtggaggttctgcctcttgcgccacttgtcatgtttacgtaaacgaagcgtttacggataaggtccctgccgccaacgaacgtgagattggtatgttagagtgcgttaccgccgagttaaagccgaattcgcgtttgtgttgtcagatcattatgacaccggagttagatggcattgttgtcgatgtccctgaccgtcaatggtaa

SEQ ID NO: 52 - protein, 107 aa, Pseudomonas putida
MSKVVYVSHDGTRRELDVADGVSLMQAAVSNGIYDIVGDCGGSASCATCHVYVNEAFTDKVPAANEREIGMLECVTAELKPNSRLCCQIIMTPELDGIVVDVPDRQW

SEQ ID NO: 53 - DNA, 1206 bp, Pseudomonas putida
atgcttgagaaacacagagttctggattccgctccagagtacgtagataaaaagaaatatctctggatactatcaactttgtggccggctactccgatgatcggaatctggcttgcaaatgaaactggttgggggattttttatgggctggtattgctcgtatggtacggcgcacttccattgcttgatgcgatgtttggtgaggactttaataatccgcctgaagaagtggtgccgaaactagagaaggagcggtactatcgagttttgacatatctaacagttcctatgcattacgctgcattaattgtgtcagcatggtgggtcggaactcagccaatgtcttggcttgaaattggtgcgcttgccttgtcactgggtatcgtgaacggactagcgctcaatacaggacacgaactcggtcacaagaaggagacttttgatcgttggatggccaaaattgtgttggctgtcgtagggtacggtcacttctttattgagcataataagggtcatcaccgtgatgtcgctacaccgatggatcctgcaacatcccggatgggagaaagcatttataagttttcaatccgtgagatcccaggagcatttattcgtgcttgggggcttgaggaacaacgcctttcgcgccgtggccaaagcgtttggagtttcgataatgaaatcctccaaccaatgatcatcacagttattctttacgccgttctccttgccttgtttggacctaagatgctggtgttcctgccgattcaaatggctttcggttggtggcagctgaccagtgcgaactatattgaacattacggcttgctccgtcaaaaaatggaggacggtcgatatgagcatcaaaagccgcaccattcttggaatagtaatcacatcgtctctaatctagtgctgttccaccttcagcggcactcggatcaccacgcgcatccaacacgttcttatcagtcacttcgggattttcccggcctgccggctcttccgacgggttaccctggtgcatttttgatggcgatgattcctcagtggtttagatcagttatggatcccaaggtagtagattgggctggtggtgaccttaataagatccaaattgatgattcgatgcgagaaacctatttgaaaaaatttggcactagtagtgctggtcatagttcgagtacctctgcggtagcatcgtag

SEQ ID NO: 54 - protein, 401 aa, Pseudomonas putida
MLEKHRVLDSAPEYVDKKKYLWILSTLWPATPMIGIWLANETGWGIFYGLVLLVWYGALPLLDAMFGEDFNNPPEEVVPKLEKERYYRVLTYLTVPMHYAALIVSAWWVGTQPMSWLEIGALALSLGIVNGLALNTGHELGHKKETFDRWMAKIVLAVVGYGHFFIEHNKGHHRDVATPMDPATSRMGESIYKFSIREIPGAFIRAWGLEEQRLSRRGQSVWSFDNEILQPMIITVILYAVLLALFGPKMLVFLPIQMAFGWWQLTSANYIEHYGLLRQKMEDGRYEHQKPHHSWNSNHIVSNLVLFHLQRHSDHHAHPTRSYQSLRDFPGLPALPTGYPGAFLMAMIPQWFRSVMDPKVVDWAGGDLNKIQIDDSMRETYLKKFGTSSAGHSSSTSAVAS

SEQ ID NO: 55 - DNA, 522 bp, Pseudomonas putida
atggctagctataaatgcccggattgtaattatgtttatgatgagagtgcgggtaatgtgcatgaggggttttctccaggtacgccttggcaccttattcctgaggattggtgctgccccgattgcgccgttcgagacaagcttgacttcatgttaattgagagcggcgtaggtgaaaagggcgtcacctcaacccatacttcgccaaatttatccgaggttagtggcacaagtttaactgctgaagcagtggttgcgccgacaagcttagagaaattgcctagtgccgacgttaaaggccaagatctatataaaactcaacctccaaggtctgatgcccaaggcgggaaagcatacttgaagtggatatgtattacttgtggccatatatatgatgaggcgttgggcgatgaggccgagggttttactccaggtactcgctttgaggatattcctgatgactggtgctgtccggattgcggggctacgaaagaagactatgtgctctacgaggaaaagtga

SEQ ID NO: 56 - protein, 173 aa, Pseudomonas putida
MASYKCPDCNYVYDESAGNVHEGFSPGTPWHLIPEDWCCPDCAVRDKLDFMLIESGVGEKGVTSTHTSPNLSEVSGTSLTAEAVVAPTSLEKLPSADVKGQDLYKTQPPRSDAQGGKAYLKWICITCGHIYDEALGDEAEGFTPGTRFEDIPDDWCCPDCGATKEDYVLYEEK

SEQ ID NO: 57 - DNA, 1158 bp, Pseudomonas putida
atggcaatcgttgttgttggcgctggtacagctggagtaaatgctgcgttctggcttcgtcaatatggttataaaggggaaattaggatttttagcagggagtctgtggcgccttatcagcggcctcctctatccaaggcttttctgacaagtgagattgcagaatccgcagtgccattaaagccagaaggtttttatacgaataacaatattaccatttcgttaaatacaccgattgtatcaatcgacgtggggcgtaagatagtttcttctaaagatggaaaagaatacgcgtatgaaaaattgattcttgcaacacctgctagcgcacgtaggttaacctgcgaggggtctgaactgtctggggtctgctatttacgcagtatggaagacgccaaaaatttacgtaggaaacttgtggagagtgcgtctgttgttgtgttgggcggcggagtaatcgggcttgaagtcgcctcagctgcggtgggcttagggaagagggtcacagtgatagaagccaccccgcgtgtaatggcgcgcgtggttacgccggcagcagcaaacttagtcagagcccgcctggaggctgaaggaattgagttcaagctgaatgcgaaattaacgtctataaagggcaggaatggccatgttgaacaatgcgtacttgaaagtggagaagaaattcaggcggatctgattgtagttggaatcggtgctatcccagagctagagctggcaactgaggcggcccttgaagtgagtaatggtgttgtggtcgatgatcagatgtgtacatcggatacaagtatatatgcaatcggcgactgcgcaatggctagaaatcctttttggggaacgatggtacgtttagagacaattcataatgcggttacacacgctcaaattgtcgcaagtagcatctgtggcacatcaacaccagcaccaaccccaccacggttctggtctgatcttaaagggatggcgctgcaaggacttggtgctctaaaggactacgataaactcgttgttgcaattaataacgaaactcttgaactagaagtccttgcgtacaagcaggagcgactgattgcaactgagacaataaatttgcctaaacgtcaaggtgcgcttgcagggagtataaaattacctgattag

SEQ ID NO: 58 - protein, 385 aa, Pseudomonas putida
MAIVVVGAGTAGVNAAFWLRQYGYKGEIRIFSRESVAPYQRPPLSKAFLTSEIAESAVPLKPEGFYTNNNITISLNTPIVSIDVGRKIVSSKDGKEYAYEKLILATPASARRLTCEGSELSGVCYLRSMEDAKNLRRKLVESASVVVLGGGVIGLEVASAAVGLGKRVTVIEATPRVMARVVTPAAANLVRARLEAEGIEFKLNAKLTSIKGRNGHVEQCVLESGEEIQADLIVVGIGAIPELELATEAALEVSNGVVVDDQMCTSDTSIYAIGDCAMARNPFWGTMVRLETIHNAVTHAQIVASSICGTSTPAPTPPRFWSDLKGMALQGLGALKDYDKLVVAINNETLELEVLAYKQERLIATETINLPKRQGALAGSIKLPD

SEQ ID NO: 59 - DNA, 3225 bp, Bacillus licheniformis
atgaacaagttagatggaattccaatccctaaaacttacgggccgctcggcaacctgcctttgcttgacaaaaacagggtctcccagtcactttggaaaatcgcggatgagatggggcctatctttcaatttaagtttgcggatgcgattggggtttttgtgtccagccatgaactggttaaagaagtctctgaagaatcccgttttgacaaaaacatggggaaggggctattgaaagttcgcgagttcagcggagacgggctctttacaagctggacggaagaacccaattggcggaaagcccacaacatccttctgccgagcttcagccagaaagcgatgaagggataccatcccatgatgcaggatatcgccgtccagctcattcaaaagtggtcccgtctcaatcaggatgaaagcattgatgtgccggacgatatgacgcggctgacgctggacacgatcggcttatgcgggtttaactaccgctttaacagcttctaccgtgaagggcagcatccgtttattgagagcatggtccggggtttgagcgaagcgatgagacagacgaagcgcttcccgctgcaggataagctgatgattcaaacgaagcgccggtttaacagcgatgtcgagtcgatgttttctcttgttgaccggatcatcgctgaccggaagcaggccgagagtgaaagcggaaatgacctcttgtcgcttatgcttcatgcgaaagaccctgagaccggcgaaaaactggatgatgagaatatccgctatcaaattattacatttttgattgccggacacgagacgacgagcggtttattatcgtttgcaatctatctgctcctgaagcatccggataagcttaagaaagcgtatgaagaagcagaccgcgtgctgaccgatcccgtcccatcctacaaacaggttcagcagctgaaatacatccgaatgattttgaatgaatcgataaggctttggccgacggcaccggctttctctctttatgcaaaagaagaaacggttatcgggggaaaatatttgattccaaaaggacagagcgttacagtgctcatcccaaaactgcacagagatcaaagcgtctggggagaagatgccgaggcattccggcctgaacggttcgagcagatggacagcattccggcgcacgcatacaaaccgtttggcaacggccaaagggcatgcatcggcatgcagttcgcccttcatgaagcgacgcttgtgctcggcatgattcttcagtactttgatcttgaagatcatgcaaactaccaattgaagatcaaagaatcgctgacattaaaaccggatggtttcacaatccgggtgaggccgaggaaaaaagaagcaatgacggcgatgccgggcgctcagcctgaagagaacggacggcaggaagaacggccttccgcaccggcggcggaaaatacgcacggaacccctcttcttgtgctctacggttcaaatctcggcacagccgaagagattgcgaaggagcttgctgaagaagcgcgtgagcaagggtttcacagccggacggcggagcttgatcaatacgcaggcgccatcccggcagaaggggctgttatcattgtgacggcttcctataacggaaacccgcccgattgcgcaaaggaatttgtcaattggcttgagcatgatcagacagacgatttgcgtggtgtcaaatatgcggtattcggctgcggtaaccgcagctgggccagcacctaccagcggattccgcgcctgattgacagcgtattggaaaaaaaaggcgcccaaaggctgcacaagcttggagaaggggatgcaggcgatgattttgaaggacagtttgagtcatggaaatatgatctgtggccgcttttaagaaccgaattttcattggccgaacccgagccgaatcaaacagaaacagacaggcaagccttatctgtcgagttcgtaaacgcacctgcggcttcgccgctggctaaagcttatcaggtgttcacagcgaagatatcggcaaaccgagaactgcagtgtgaaaagagcgggagaagcacaaggcatattgaaatatcgcttcctgaaggcgccgcatatcaggagggagaccatctcggtgtgctaccgcaaaatagcgaagtgctgattggccgcgtttttcagcggtttgggctgaacggaaatgaacaaattctgattagcggccggaatcaagcatcacatttgcctttggagaggcccgttcatgtcaaagacctttttcaacattgcgtcgagctccaggaaccggccacaagggcccagatacgcgagctggcggctcatactgtttgtccgcctcatcagcgcgagcttgaagacctgctgaaagatgacgtctataaggatcaagtgttgaataagcggctgacaatgcttgacctgcttgagcaatacccggcctgtgaactgccgttcgcccgttttctggcgcttcttcctccgctaaaaccgaggtactattcgatttccagttcgccgcagcttaacccgcggcaaacaagcatcaccgtctctgtcgtaagtggcccggcgttgagcggccgcgggcattataagggagttgcatcgaactatctcgccggccttgagccgggagacgcgatttcgtgtttcatcagagagcctcagtcaggcttccggcttcccgaagatcctgaaacaccggtgatcatggtcgggccgggcaccggaatcgccccttaccgcggatttcttcaggcgcgccgcatccagcgcgatgccggtgtgaagctcggtgaagcgcatttgtacttcggctgccgccgtccgaacgaagattttctgtatcgagacgagttggagcaagcggaaaaggacggaatcgtccatctgcatacagcgttttcccggcttgagggccggccgaaaacatatgtgcaagatttgctcagagaggatgcagccttgctgattcacttgttgaacgaaggcggccgcctgtatgtgtgcggagacggaagccgcatggctccagctgttgaacaagctttgtgcgaggcgtatcgcatagtacagggtgcgagtcgggaagagtcgcaaagctggctgtccgcacttttagaagaagggcgctatgcaaaggatgtatgggacggcggcgtttcccaacataatgtgaaggcggactgcattgcaagaacgtaa

SEQ ID NO: 60 - protein, 1074 aa, Bacillus licheniformis
MNKLDGIPIPKTYGPLGNLPLLDKNRVSQSLWKIADEMGPIFQFKFADAIGVFVSSHELVKEVSEESRFDKNMGKGLLKVREFSGDGLFTSWTEEPNWRKAHNILLPSFSQKAMKGYHPMMQDIAVQLIQKWSRLNQDESIDVPDDMTRLTLDTIGLCGFNYRFNSFYREGQHPFIESMVRGLSEAMRQTKRFPLQDKLMIQTKRRFNSDVESMFSLVDRIIADRKQAESESGNDLLSLMLHAKDPETGEKLDDENIRYQIITFLIAGHETTSGLLSFAIYLLLKHPDKLKKAYEEADRVLTDPVPSYKQVQQLKYIRMILNESIRLWPTAPAFSLYAKEETVIGGKYLIPKGQSVTVLIPKLHRDQSVWGEDAEAFRPERFEQMDSIPAHAYKPFGNGQRACIGMQFALHEATLVLGMILQYFDLEDHANYQLKIKESLTLKPDGFTIRVRPRKKEAMTAMPGAQPEENGRQEERPSAPAAENTHGTPLLVLYGSNLGTAEEIAKELAEEAREQGFHSRTAELDQYAGAIPAEGAVIIVTASYNGNPPDCAKEFVNWLEHDQTDDLRGVKYAVFGCGNRSWASTYQRIPRLIDSVLEKKGAQRLHKLGEGDAGDDFEGQFESWKYDLWPLLRTEFSLAEPEPNQTETDRQALSVEFVNAPAASPLAKAYQVFTAKISANRELQCEKSGRSTRHIEISLPEGAAYQEGDHLGVLPQNSEVLIGRVFQRFGLNGNEQILISGRNQASHLPLERPVHVKDLFQHCVELQEPATRAQIRELAAHTVCPPHQRELEDLLKDDVYKDQVLNKRLTMLDLLEQYPACELPFARFLALLPPLKPRYYSISSSPQLNPRQTSITVSVVSGPALSGRGHYKGVASNYLAGLEPGDAISCFIREPQSGFRLPEDPETPVIMVGPGTGIAPYRGFLQARRIQRDAGVKLGEAHLYFGCRRPNEDFLYRDELEQAEKDGIVHLHTAFSRLEGRPKTYVQDLLREDAALLIHLLNEGGRLYVCGDGSRMAPAVEQALCEAYRIVQGASREESQSWLSALLEEGRYAKDVWDGGVSQHNVKADCIART

SEQ ID NO: 61 - DNA, 1770 bp, Stenotrophomonas maltophilia
atgtactacagcagtggcaactacgaagcctttgcgcgcccgcgcaagcccgccggtgtcgatgacaagcgcgcatggttcgtcggttcgggcctggcctcgctggccggcgccgcgttcctggtgcgcgacggccgcatggccggtgagcgcatcaccattctcgagcagcagcggattgccggaggcgcgctggatggcctgaaggtgcctgaaaagggcttcgtgatccgcggtggacgcgagatggaagaccacttcgagtgtctgtgggatctgttccgctcgattccgtcgctggagattgaagatgccagcgtgctggacgagttctactggctgaacaaggacgaccccaactattcgctgcagcgtgccacgatcaatcgcggtgaggatgcgcacaccgacggcctgttcaccctgaccgagcaggcgcagaaggacatcatcgcgctgttcctggccacccggcaggagatggagaacaagcgcatcgacgaggtgctgggccgcgacttcctggacagcaacttctggctgtactggcgcaccatgttcgccttcgaggaatggcattcggcgctggagatgaagctgtacctgcatcgcttcatccaccatatcggcggcctgccggatttctcggcgctgaagttcaccaagtacaaccagtacgaatcgctggtgctgccgctggtgaagtggctgcaggaccacggcgtggtgttccagtacggcaccgaggtgaccgacgtcgacttcgatctggcggccggccgcaagcaggccacgcgcatccactggacgcgtgacggtgtagccggtggcgtggatctgagcgcggatgaccttgtgttcatgacgatcggttcgttgaccgagaactcggacaacggcgaccaccgcacggctgcgcgtttgaatgaaggcgcggcgccggcctgggacctgtggcgacgtattgccgccagggatcctgcgttcgggcgcccggatgtgtttggcgcgcatatcccgcagaccaagtgggaatcggcgacggtgaccacgctggatgcacgcattccggcctacatccagaagatcgccaagcgcgacccgttcagtggcaaggtggtgaccggcggcatcgtcagtgtgcgtgactcgcgctggttgatgagctggacggtgaaccgccagccgcatttcaagaaccagcccaaggatcagatcgtggtctgggtgtattcgctgttcgtggatacgcctggagactacgtgaagaagccgatgcaggattgcaccggcgaggagatcacccgtgagtggctgtaccacctgggcgtgccggtggaagaaatcgacgaattggccgcgaccggcgcgaaaacggtgccggtgatgatgccgtacatcaccgcgttcttcatgccacgccaggccggtgatcgcccggacgtggtgccggagggtgcggtgaacttcgccttcatcggccagtttgccgaatcgaagcagcgcgactgcatcttcaccaccgagtattcggtgcgcacaccgatggaagcggtgtataccctgctgggcatcgagcgcggcgtgccggaggtgttcaattccacctatgacgtgcgctcgttgctggcggcgaccggtcgcctgcgcgatggcagggaactggatattcccggcccggcgttcctgcgcaacctgctgatgaacaagctggacaagacccagatcggtggcctcctgcgcgagttcaagctggtgcaggaggactga

SEQ ID NO: 62 - protein, 589 aa, Stenotrophomonas maltophilia
MYYSSGNYEAFARPRKPAGVDDKRAWFVGSGLASLAGAAFLVRDGRMAGERITILEQQRIAGGALDGLKVPEKGFVIRGGREMEDHFECLWDLFRSIPSLEIEDASVLDEFYWLNKDDPNYSLQRATINRGEDAHTDGLFTLTEQAQKDIIALFLATRQEMENKRIDEVLGRDFLDSNFWLYWRTMFAFEEWHSALEMKLYLHRFIHHIGGLPDFSALKFTKYNQYESLVLPLVKWLQDHGVVFQYGTEVTDVDFDLAAGRKQATRIHWTRDGVAGGVDLSADDLVFMTIGSLTENSDNGDHRTAARLNEGAAPAWDLWRRIAARDPAFGRPDVFGAHIPQTKWESATVTTLDARIPAYIQKIAKRDPFSGKVVTGGIVSVRDSRWLMSWTVNRQPHFKNQPKDQIVVWVYSLFVDTPGDYVKKPMQDCTGEEITREWLYHLGVPVEEIDELAATGAKTVPVMMPYITAFFMPRQAGDRPDVVPEGAVNFAFIGQFAESKQRDCIFTTEYSVRTPMEAVYTLLGIERGVPEVFNSTYDVRSLLAATGRLRDGRELDIPGPAFLRNLLMNKLDKTQIGGLLREFKLVQED

SEQ ID NO: 63 - DNA, 1986 bp, Stenotrophomonas maltophilia
atgagccagcccaccgcaccgggacgcaacgcaggggccacgccggccttcgagcacgagccggacagcaccggcggctactggtccaaccggccggagaacacactgccaccgccggacatgatgggggcctacatgcgcaaccggccgctgccgccggaggacgtggcgcagcgcaaggcctacatcatcggcaccggcatcgccgggttggcggcggcgttctacctgatccgcgacggcggcatgccgccggccaacatcacgctgctggacagcctggagatcgaaggcggttcgctggatggcgcgggggatgccgagcagggctacctgatccgcggcggccgcgagatgaactggaactacgacaatttctgggacctgttccaggatgtgccggcactcgaactgccggccggcttcagcgtgctcgacgagtaccgcgccgtcaacgacaacgatccgaactggtccaaggcgcggctgctgcaccagcagggcaaggtcaaggatttcgccacgttcgggttgagccgcggccagcaatgggagctggtcaagctgctgctcaagcgcaaggaagacctggacgacgtcaccatcgaggactacttcagcgaaggctttctgcagagcaacttctggttcttctggcgctcgatgttcgccttcgagaactggcagagcctgctcgagatgaagctgtacatgcatcggttcctggatgccatcgacggtctgaacgacatgtccgcgctggtgtttcccaagtacaaccaatacgagagttttgtggtgccgctgtcgcggatgctgcgcgcgcagggcgtcaacgtgcagttcgatacccgcgtccacgacctggagatggcggtggacgggcagtcacgcaccgtcaccgcgctgcgctgccgggtggccggcaacgagaccacgctgccggttgcggcgggcgacctggtgttcgcgctcaccggttcgatgaccgaaggcacggcgtacggcgacatggacaccgtgccgccgctggcgcgcgaccgccgggacccgggcgaggacagtgactgggcgctgtggcgcaatctcgcgcggcagtcgccgatcttcggcaagccggagaagttctacggcgacgtggaccgctcgatgtgggagtcggccacgttgacctgccgcccctcgccgctggtggacaagatccgcacgttgtcggtcaacgatccgtactccgggcgcaccgtgaccggtggggtcatcaccatcaccgattccaactgggtgctcagcttcaccgtcaaccgccagccgcatttcgtggaccagcccaaggacgtgctggtggtatgggtctatgccctgttgatggatcaggacggcaaccacatcaaaaagccgatgccggcgtgtaccggacgcgaggtgctggccgaactgtgccaccacctgggcatcggcgaccagatcgatgcggtggccgccgcgaccagggtgcggctggcgttgatgccgtacatcaccgcgcagttcatgccgcgtgccgctggcgaccgtccgcacgtggtaccggccggctgcaccaacctgggcctgctcggccagttcgtggaaacgcgcaacgatgtgatcttcacgatggaaagctcgatccgcacggcgcgtgtggcggtgtacaccctgctggggctgcgcaagcaggtaccggacctgagcccgacccagtacgacatccgcaatctgatcaaagcggcacgggcgttgaacaacaacgcgccgttccccggcgaacggctgctgcaccgtctgctcggcaacagttattacgcccacatcctgccgccgctgccacagcctgaaaagggccgggaggccttccttgaagaggagctgtcgtggttgtcaggcaagggcagcgtggtgctgaaggacctgtctgcacggctggatcggcttggcgaaacgctgggcaggcggcgcgcgccctga

SEQ ID NO: 64 - protein, 661 aa, Stenotrophomonas maltophilia
MSQPTAPGRNAGATPAFEHEPDSTGGYWSNRPENTLPPPDMMGAYMRNRPLPPEDVAQRKAYIIGTGIAGLAAAFYLIRDGGMPPANITLLDSLEIEGGSLDGAGDAEQGYLIRGGREMNWNYDNFWDLFQDVPALELPAGFSVLDEYRAVNDNDPNWSKARLLHQQGKVKDFATFGLSRGQQWELVKLLLKRKEDLDDVTIEDYFSEGFLQSNFWFFWRSMFAFENWQSLLEMKLYMHRFLDAIDGLNDMSALVFPKYNQYESFVVPLSRMLRAQGVNVQFDTRVHDLEMAVDGQSRTVTALRCRVAGNETTLPVAAGDLVFALTGSMTEGTAYGDMDTVPPLARDRRDPGEDSDWALWRNLARQSPIFGKPEKFYGDVDRSMWESATLTCRPSPLVDKIRTLSVNDPYSGRTVTGGVITITDSNWVLSFTVNRQPHFVDQPKDVLVVWVYALLMDQDGNHIKKPMPACTGREVLAELCHHLGIGDQIDAVAAATRVRLALMPYITAQFMPRAAGDRPHVVPAGCTNLGLLGQFVETRNDVIFTMESSIRTARVAVYTLLGLRKQVPDLSPTQYDIRNLIKAARALNNNAPFPGERLLHRLLGNSYYAHILPPLPQPEKGREAFLEEELSWLSGKGSVVLKDLSARLDRLGETLGRRRAP